(19) United States
(12) Patent Application Publication
(10) Pub. No.: US 2026/0041693 A1
(43) Pub. Date: Feb. 12, 2026

(54) COMBINATION THERAPY OF CDK7 INHIBITORS WITH OTHER ANTI-CANCER THERAPIES

(71) Applicant: QURIENT CO., LTD., GYEONGGI-DO (KR)

(72) Inventors: Kiyean NAM, GYEONGGI-DO (KR); Jaeseung KIM, SEOUL (KR); Yeejin JEON, GYEONGGI-DO (KR); Donghoon YU, GYEONGGI-DO (KR); Won-gyun AHN, GYEONGGI-DO (KR); Seung-Joo LEE, GYEONGGI-DO (KR)

(21) Appl. No.: 19/100,289

(22) PCT Filed: Aug. 2, 2023
(86) PCT No.: PCT/EP2023/071458

§ 371 Date: Jan. 31, 2025

Related U.S. Application Data
(60) Provisional application No. 63/395,549, filed on Aug. 5, 2022.

(51) Int. Cl. A61K 31/55; A61K 31/519; A61K 31/53; A61K 31/5365; A61K 31/5377; A61K 31/5386; A61K 45/06; A61P 35/00

(52) U.S. Cl. CPC A61K 31/55; A61K 31/519; A61K 31/53; A61K 31/5365; A61K 31/5377; A61K 31/5386; A61K 45/06; A61P 35/00

(57) ABSTRACT

The present disclosure relates to combinations of cyclin-dependent kinase 7 (CDK7) inhibitors and other therapeutic treatments, in particular other anti-cancer agents, and uses of such combination(s) in the treatment of cancers.

The present disclosure relates to combinations of cyclin-dependent kinase 7 (CDK7) inhibitors and other therapeutic treatments, in particular other anti-cancer agents, and the uses of such combination(s) in the treatment of cancers.

### BACKGROUND OF THE INVENTION

CDK7 is a master regulator of cell cycle progression and is also a component of the general transcription factor TFIIH, which regulates RNA polymerase-II-mediated transcription. CDK7 inhibition can lead to DNA damage and genomic instability by arresting cell cycle and inducing replicative stress. Because of its role as a master regulator of cell cycle and transcription, CDK7 is an attractive therapeutic target for cancer therapy.

Various CDK7-inhibitors have been described in the art. For example, the inhibitor samuraciclib has been described to target proliferation pathways to inhibit advanced prostate cancer (Constantin et al., Oncogene 2022; http://doi.org/101101/2022.06.29.497030.

A semi-synthetic flavone derivative, alvocidib (flavopiridol) which inhibits CDK1, 2, 4, 6, 7, and 9 was the first CDK inhibitor to enter clinical trials. Limited clinical activity was seen in the majority of trials, however modest responses against chronic lymphocytic leukemia (CLL) and mantel cell lymphoma were shown (Byrd et al., 2006, Blood, 109(2), pp. 399-404).

WO 2019/197546 describes pyrazolo[1,5-a][1,3,5]triazine and pyrazolo[1,5-a]pyrimidine derivatives which are selective CDK7-inhibitors.

BS-181 is another example of a selective CDK7-inhibitor which is structurally related to the PAN-CDK-inhibitor roscovitine [Ali et al., 2009, Cancer Research, 69(15), pp. 6208-6215.

Another selective CDK7-inhibitor, SY-1365, developed by Syros Pharmaceuticals was used in a phase I clinical trial for the treatment of advanced solid tumors (Q et al. 2019, Cancer Research, https://doi.org/10.1158/0008-5472). However, such clinical trial was discontinued.

There remains a need in the art for new treatment modalities of proliferative diseases, in particular cancer that are effective in such treatment.

There also remains a need to improve existing therapies and to provide new therapies in the field of cancer-therapy.

In a first aspect, the present invention relates to a combination of an inhibitor of cyclin-dependent kinase 7 and an anti-cancer agent which is different from said inhibitor of cyclin-dependent kinase 7,
wherein said inhibitor of cyclin-dependent kinase 7 is a compound having the general formula I

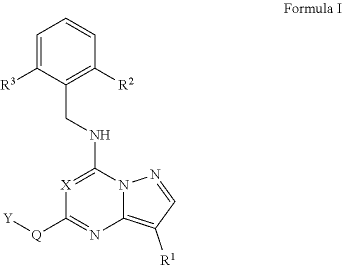

wherein
X is, independently at each occurrence, selected from CH and N;
Q is either absent or independently, at each occurrence, selected from the group consisting of —NH—, —NH(CH₂)—, —NH(CH₂)₂—, —NH(C═O)—, —NHSO₂—, —O—, —O(CH₂)—, —(C═O)—, —(C═O)NH— and —(C═O)(CH₂)—;
Y is, independently at each occurrence, selected from the group consisting of halogen, C1-C3 haloalkyl, C3-C8 cycloalkyl, aryl, heteroaryl, heterocyclyl, —S(═O)₂R⁴, C1-C6 alkyl and C1-C6 alkyl substituted with one or two of —OR⁶, —N(R⁶)R⁶, aryl, heteroaryl and heterocyclyl;
Wherein C3-C8 cycloalkyl is optionally substituted with one or two of R⁴, R⁵ and —(C═O)R⁶, wherein heterocyclyl is optionally substituted with one or two of R⁴, R⁵ and —(C═O)R⁶, and wherein aryl or heteroaryl is optionally substituted with one or two of R⁴, C1-C6 alkyl, —OR⁶, —N(R⁶)R⁶, —(C═O)R⁶, halogen, heteroaryl and heterocyclyl;
R¹ is, at each occurrence, independently selected from the group consisting of halogen, C1-C6 alkyl, C3-C10 cycloalkyl, —CN, —(C═O)CH₃ and C1-C3 haloalkyl, any of which is optionally substituted;
R² is, at each occurrence, independently selected from any structure of the following group A

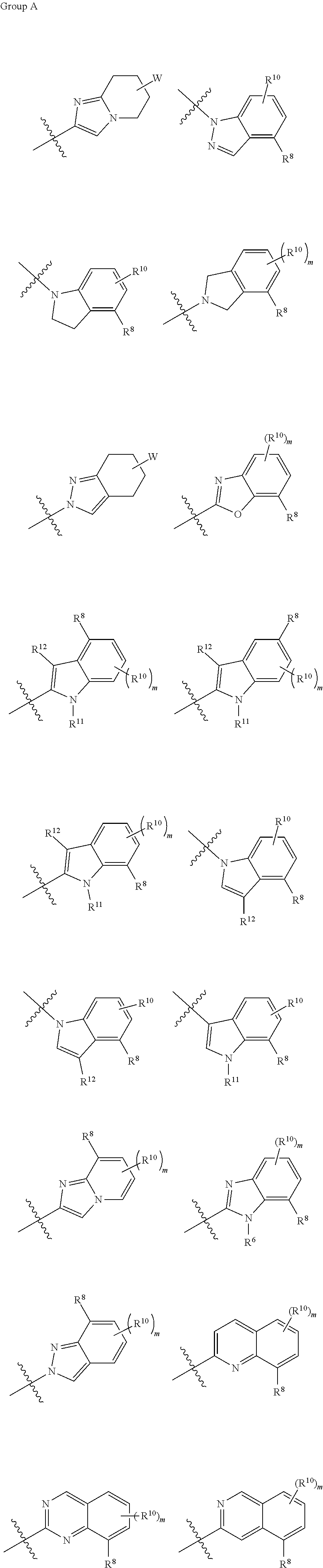

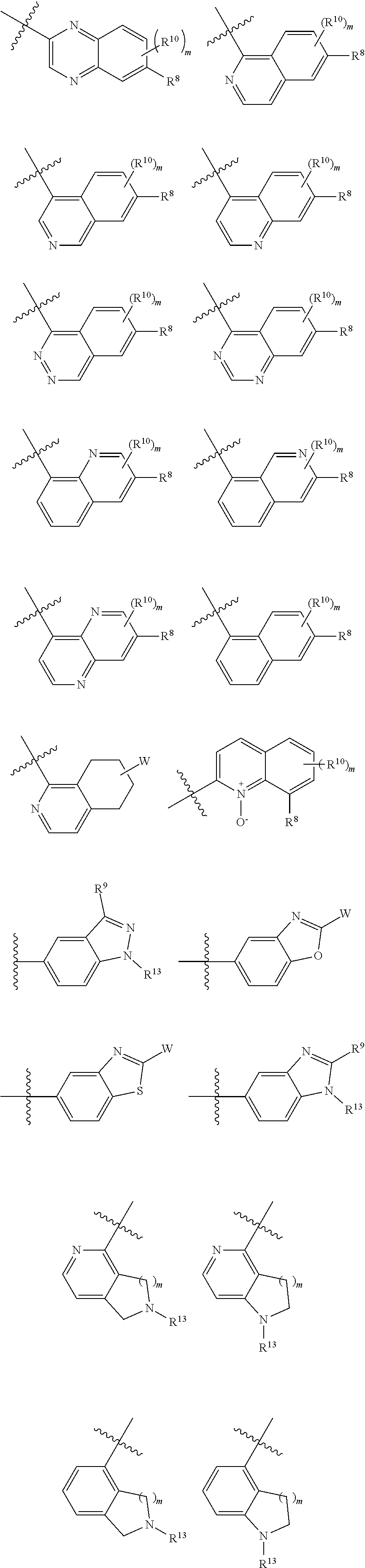

wherein m is, independently at each occurrence, selected from 1, 2 and 3;
W is any structure of the following group B;

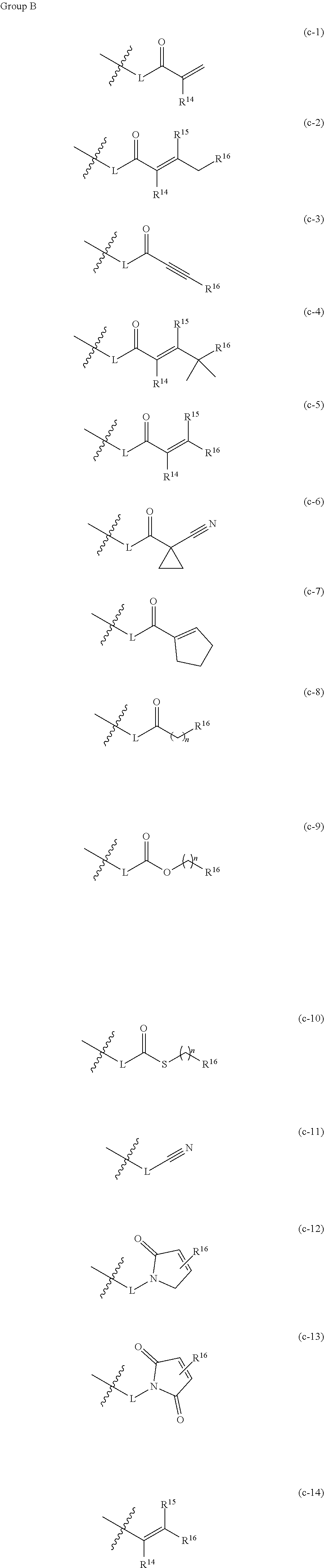

L is absent or, at each occurrence, independently selected from the group consisting of —O— and —NH—;
wherein n is, independently at each occurrence, selected from 1, 2 and 3;
R³ is, at each occurrence, independently selected from the group consisting of hydrogen, halogen, C1-C6 alkyl, C1-C3 haloalkyl, —OR⁶, —CN and C1-C6 alkyl substituted with —OH, —OR⁶ or —NHR⁶;
R⁴ is either absent or independently, at each occurrence, selected from the group consisting of hydrogen, —OR⁶, halogen, C1-C3 haloalkyl, —CN, —N(R⁶)R⁶, (═O), —NH(C═O)R⁶, —(C═O)NH₂, —S(═O)₂N(R⁶)R⁶, aryl, heteroaryl, heterocyclyl, C1-C6 alkyl and C1-C6 alkyl substituted with —OR⁶, —NH₂ or —S(═O)₂N(R⁶)R⁶;
R⁵ is, independently, at each occurrence, selected from the group consisting of hydrogen, halogen, C1-C3 haloalkyl, —CN, —OR⁶, —N(R⁶)R⁶, (═O), S(═O)₂N(R⁶)R⁶, aryl, heteroaryl, heterocyclyl, C1-C6 alkyl and C1-C6 alkyl substituted with —OH, —NH₂ or —S(═O)₂N(R⁶)R⁶;
wherein both R⁴ and R are (═O) if attached to a single sulfur atom that forms part of Y being a heterocycle;
or wherein R⁴ and R⁵, together with the structure to which they are attached, form an aromatic ring, a heteroaromatic ring, a saturated or unsaturated heterocyclic ring, or a fused or bridged ring structure of any of an aromatic ring, a heteroaromatic ring, and a saturated or unsaturated heterocyclic ring;
R⁶ is, at each occurrence, independently selected from the group consisting of hydrogen, C1-C6 alkyl, C3-C6 cycloalkyl, C1-C3 haloalkyl, heteroaryl, heterocyclyl, heteroaryl substituted with one or two of halogen, —OR⁷, —N(R⁷)R⁷, C1-C6 alkyl and C1-C6 alkyl substituted with —OH, —NH₂;
heterocyclyl substituted with one or two of halogen, —OR⁷, —N(R⁷)R⁷, C1-C6 alkyl and C1-C6 alkyl substituted with —OH or —NH₂;
R⁷ is, at each occurrence, independently selected from the group consisting of hydrogen, C1-C6 alkyl, C3-C10 cycloalkyl and W, as defined above;
R⁸ is, at each occurrence, independently selected from hydrogen and W, as defined above;
Wherein if R⁷ is W, R⁸ is hydrogen;
R⁹ is, at each occurrence, independently selected from hydrogen and W, as defined above;
R¹⁰ is, at each occurrence, independently selected from the group consisting of hydrogen, halogen, C1-C6 alkyl, C1-C3 haloalkyl, —NH₂, —OR⁶, —CN and W, as defined above;
Wherein if R¹⁰ is W, R⁸ is hydrogen;
R¹¹ is, at each occurrence, independently selected from the group consisting of hydrogen, C1-C6 alkyl and C1-C3 haloalkyl;
R¹² is, at each occurrence, independently selected from the group consisting of hydrogen, halogen, C1-C6 alkyl, C1-C3 haloalkyl, —NH₂, —OR⁶ and —CN;
R¹³ is, at each occurrence, independently selected from the group consisting of hydrogen, C1-C6 alkyl, C3-C10 cycloalkyl and W, as defined above;
Wherein if R¹³ is W, R⁹ is hydrogen;
R¹⁴ and R¹⁵ are, at each occurrence, independently selected from the group consisting of hydrogen, halogen, C1-C6 alkyl, C1-C3 haloalkyl, —OR⁶, heterocyclyl and —CN;
R¹⁶ is, at each occurrence, independently selected from the group consisting of hydrogen, halogen, C1-C6 alkyl, C3-C10 cycloalkyl, —N(R⁶)₂, —NR¹³R¹⁴, —NR¹³CH₂(CO)NH₂, heterocyclyl, —OR⁶ and —CN.
or an enantiomer, stereoisomeric form, mixture of enantiomers, diastereomer, mixture of diastereomer, racemate of the above mentioned compounds or a pharmaceutically acceptable salt thereof.

In one embodiment, said anti-cancer agent is selected from
a) target-specific compounds selected from the group consisting of immune checkpoint inhibitors, in particular monoclonal antibodies and antibody fragments directed at immune checkpoints; poly-ADP-ribose-polymerase (PARP) inhibitors; monoclonal antibodies and antibody fragments not directed at immune checkpoints; tyrosine kinase inhibitors; immunotoxins; MEK inhibitors; KRAS inhibitors; c-MET inhibitors; FGFR inhibitors; proteasome inhibitors; cyclin-dependent kinase inhibitors; mTOR inhibitors; retinoids; immunomodulatory agents; histone deacetylase inhibitors; proteolysis targeting chimera compounds (PROTACs); siRNA; antibody-drug-conjugates (ADCs); antibody-siRNA-conjugates (ARCs); DNA damage response inhibitors, and target-specific fusion proteins; and
b) cytotoxic non-specific compounds selected from taxanes, alkylating agents, nucleoside analogues, antifolates, topoisomerase inhibitors, anthracyclines, podophyllotoxins, vinca alkaloids, and platinum compounds;
c) hormonal anti-cancer agents selected from hormones; hormone antagonists; hormone receptor antagonists; hormone receptor degraders and aromatase inhibitors;
wherein, preferably said hormones are selected from medroxprogesteron; anastrozole, letrozole, exemestane; megestrol; raloxifene; estramustine; gonadotropin-releasing hormones, such as leuprolide, goserelin, triptorelin, histrelin, abarelix; androgens, such as testolactone, fluoxymesterone; antiandrogens, such as enzalutamide, bicalutamide, apalutamide, darolutamide, nilutamide, flutamide;
and wherein, preferably said hormone antagonists are selected from gonadotropin-releasing hormone antagonists, such as degarelix;
and wherein, preferably, said hormone receptor antagonists are selected from fulvestrant, tamoxifen, toremifene; and
wherein, preferably, said hormone receptor degraders are selected from selective estrogen receptor degraders, and selective androgen receptor degraders; more preferably from giredestrant, amcenestrant, fulvestrant, AZD9833, rintodestrant, LSZ102, LY3484356, elacestrant, ZN-c5, D-0502, SHR9549, and bavdegalutamide;
and wherein, preferably, said aromatase inhibitors are selected from anastrozole, letrozole, exemestane, vorozole, formestane, fadrozole, testolactone, and aminoglutethimide; and
d) radiopharmaceuticals.

In one embodiment, said combination is a composition in which said inhibitor of cyclin-dependent kinase 7 and said anti-cancer agent are present together, being either physically mixed with each other or being kept separate from each other by at least one physical separation barrier between said inhibitor of cyclin-dependent kinase 7 and said anti-cancer agent wherein said at least one physical separation barrier forms part of said combination, e.g. wherein said inhibitor of cyclin-dependent kinase 7 and said anti-cancer agent are kept in separate containers or compartments or chambers or dosage units, which separate containers, compartments, chambers and dosage units form part of said combination.

In one embodiment, said anti-cancer agent is a target-specific compound selected from immune checkpoint inhibitors, in particular monoclonal antibodies directed at immune checkpoints; poly-ADP-ribose-polymerase (PARP) inhibitors; other monoclonal antibodies not directed at immune checkpoints; tyrosine kinase inhibitors; DNA damage response inhibitors; and antibody-cytokine fusion proteins.

In one embodiment, said target-specific compound is selected from anti-PD1 antibodies, anti-PD-L1 antibodies, anti-CTLA-4 antibodies, anti-LAG-3 antibodies, anti-VEGF antibodies, anti-VEGFR antibodies, anti-EGFR antibodies, anti-HER2 antibodies, anti-CD52 antibodies, anti-CD33 antibodies, anti-CD30 antibodies, anti-CD20 antibodies, anti-TIM3 antibodies, anti-TIGIT antibodies, anti-41BB antibodies, anti-OX40 antibodies, anti-CD40 antibodies, anti-CD27 antibodies, anti-GITR antibodies, anti-ICOS antibodies, anti-Siglec antibodies, and anti-PVRIG antibodies.

In one embodiment, said target-specific compound is selected from anti-human-PD1 antibodies, in particular pembrolizumab, nivolumab, cemiplimab, spartalizumab, atezolizumab, avelumab, durvalumab, ipilimumab, tremelimumab, relatlimab, bevacizumab, ramucirumab, cetuximab, panitumumab, pertuzumab, trastuzumab, trastuzumab-emtansine, alemtuzumab, gemtuzumab, gemtuzumab-ozoamicin, brentuximab, brentuximab-vedotin, ibritumomab, ibritumomab-tiuxetan, rituximab, obinutuzmab, tositumomab, ofatumumab, pidilizumab, toripalimab, sintilimab, camrelizumab, tislelizumab, zimberelimab, prolgolimab, dostarlimab; wherein, preferably, said target-specific compound is pembrolizumab.

In one embodiment, said target-specific compound is selected from poly-ADP-ribose-polymerase (PARP) inhibitors, in particular olaparib, pamiparib, and niraparib; tyrosine kinase inhibitors, in particular afatinib, aflibercept, axitinib, bosutinib, cabozantinib, ceritinib, crizotinib, dasatinib, erlotinib, gefitinib, ibrutinib, imatinib, lapatinib, lenvatinib, nilotinib, pazopanib, ponatinib, regorafenib, ruxolitinib, sorafenib, sunitinib, vandetanib, anlotinib, apatinib, osimertinib, and alectinib; MEK inhibitors, in particular cobimetinib, and trametinib; KRAS inhibitors, in particular sotorasib, and adagrasib; c-MET inhibitors, in particular savolitinib; FGFR inhibitors, in particular erdaftinib, pemigatinib, and vofatamab; DNA damage response inhibitors selected from WEE1 inhibitors and ATR inhibitors, in particular adavosertib, berzosertib and volasertib.

In one embodiment, said anti-cancer agent is a cytotoxic non-specific compound selected from
a) taxanes, preferably selected from docetaxel, carbazitaxel, and paclitaxel;
b) alkylating agents, preferably selected from bendamustine, busulfan, carmustine, chlorambucil, chlormethine, cyclophosphamide, dacarbazine, fotemustine, ifosfamide, lomustine, melphalan, streptozotocin, and temozolomide;
c) nucleoside analogues, preferably selected from azacitidine, capecitabine, carmofur, cladribine, clofarabine, cytarabine, decitabine, floxuridine, fludarabine, fluoruracil, gemcitabine, mercaptopurine, nelarabine, pentostatin, tegafur, and tioguanine;
d) antifolates, preferably selected from methotrexate, pemetrexed, and raltitrexed;
e) topoisomerase inhibitors, preferably selected from irinotecan, and topotecan;
f) anthracyclines, preferably selected from daunorubicin, doxorubicin, epirubicin, idarubicin, mitoxantrone, and valrubicin;
g) podophyllotoxins, preferably selected from etoposide and teniposide;
h) vinca alkaloids, preferably selected from vinblastine, vincristine, vindesine, vinflunine, and vinorelbine;
i) platinum compounds, preferably selected from cisplatin, carboplatin, oxaliplatin, nedaplatin, heptaplatin, and lobaplatin.

In one embodiment, the compound is a compound having the general formula Ia

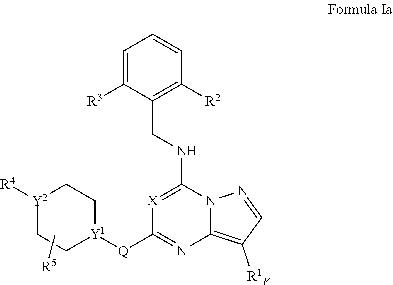

wherein
X is, independently at each occurrence, selected from CH and N;
Y¹ is, independently at each occurrence, selected from CH, C(OH) and N;
Y² is, independently at each occurrence, selected from CH, C(OH) and N;
Q is absent or, at each occurrence, independently selected from the group consisting of —NH—, —NH(CH₂)—, —NH(C═O)—, —NHSO₂—, —O—, —O(CH₂)—, —(C═O)— and —(C═O)(CH₂)—;
R¹ is, at each occurrence, independently selected from the group consisting of halogen, C1-C6 alkyl, C3-C10 cycloalkyl, —CN, —(C═O)CH₃ and C1-C3 haloalkyl, any of which is optionally substituted;
R² is, at each occurrence, independently selected from any structure of the following group A,

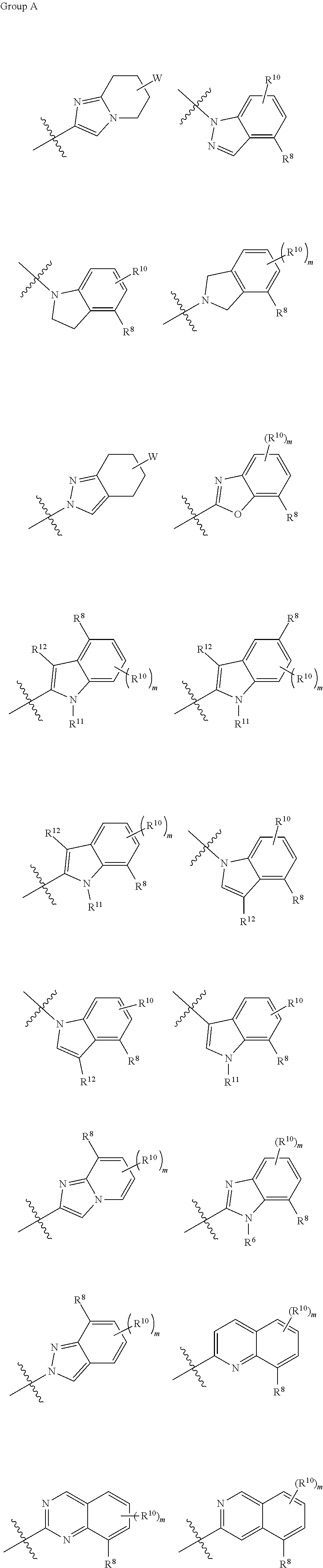

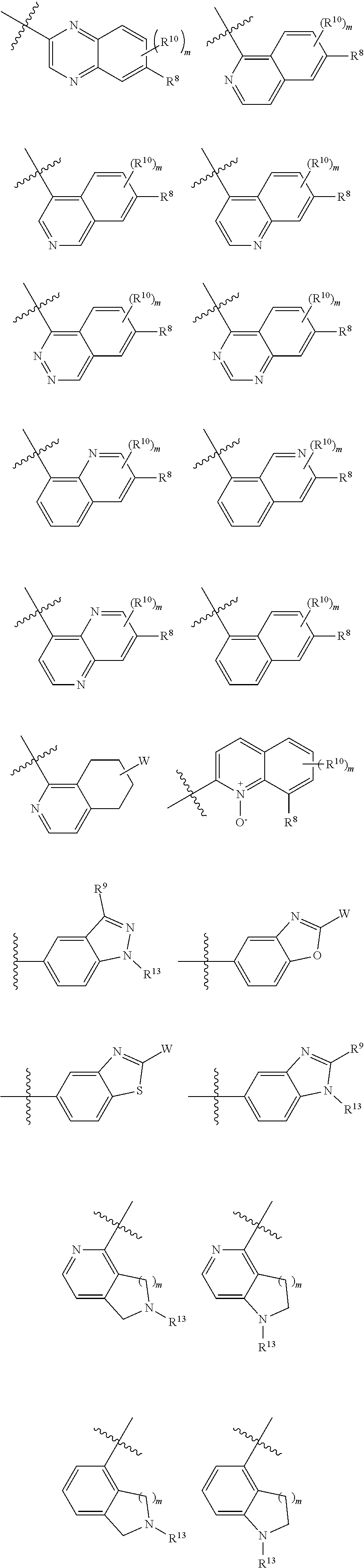

wherein m=1, 2 or 3;
W is any structure of the following group B′;

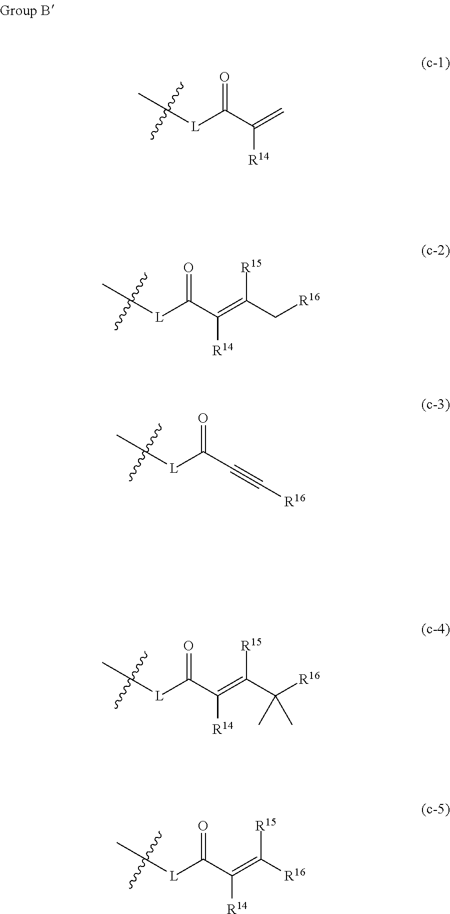

L is absent or, at each occurrence, independently selected from the group consisting of —O— and —NH—;
R³, is, at each occurrence, independently selected from the group consisting of hydrogen, halogen, C1-C6 alkyl, C1-C3 haloalkyl, —OR⁶, —CN and C1-C6 alkyl substituted with —OH, —OR⁶ or —NHR⁶;
R⁴ is either absent or independently, at each occurrence, selected from the group consisting of hydrogen, —OR⁶, halogen, C1-C3 haloalkyl, —CN, —N(R⁶)R⁶, (═O), —NH(C═O)R⁶, —(C═O)NH₂, —S(═O)₂N(R⁶)R⁶, aryl, heteroaryl, heterocyclyl, C1-C6 alkyl and C1-C6 alkyl substituted with —OR⁶, —NH₂ or —S(═O)₂N(R⁶)R⁶;
R⁵ is, independently, at each occurrence, selected from the group consisting of hydrogen, halogen, C1-C3 haloalkyl, —CN, —OR⁶, —N(R⁶)R⁶, (═O), S(═O)₂N(R⁶)R⁶, aryl, heteroaryl, heterocyclyl, C1-C6 alkyl and C1-C6 alkyl substituted with —OH, —NH₂ or —S(═O)₂N(R⁶)R⁶;
wherein both R⁴ and R⁵ are (═O) if attached to a single sulfur atom that forms part of Y being a heterocycle;
or wherein R⁴ and R⁵, together with the structure to which they are attached, form an aromatic ring, a heteroaromatic ring, a saturated or unsaturated heterocyclic ring, or a fused or bridged ring structure of any of an aromatic ring, a heteroaromatic ring, and a saturated or unsaturated heterocyclic ring;
R⁶ is, at each occurrence, independently selected from the group consisting of hydrogen, C1-C6 alkyl, C3-C6 cycloalkyl, C1-C3 haloalkyl, heteroaryl, heterocyclyl, heteroaryl substituted with one or two of halogen, —OR⁷, —N(R⁷)R⁷, C1-C6 alkyl and C1-C6 alkyl substituted with —OH, —NH₂;
heterocyclyl substituted with one or two of halogen, —OR⁷, —N(R⁷)R⁷, C1-C6 alkyl and C1-C6 alkyl substituted with —OH or —NH₂;
R⁷ is, at each occurrence, independently selected from the group consisting of hydrogen, C1-C6 alkyl, C3-C10 cycloalkyl and W, as defined above;
R⁸ is, at each occurrence, independently selected from hydrogen and W, as defined above;
Wherein if R⁷ is W, R⁸ is hydrogen;
R⁹ is, at each occurrence, independently selected from hydrogen and W, as defined above;
R¹⁰ is, at each occurrence, independently selected from the group consisting of hydrogen, halogen, C1-C6 alkyl, C1-C3 haloalkyl, —NH₂, —OR⁶, —CN and W, as defined above;
wherein if R¹⁰ is W, R⁸ is hydrogen;
R¹¹ is, at each occurrence, independently selected from the group consisting of hydrogen, C1-C6 alkyl and C1-C3 haloalkyl;
R¹² is, at each occurrence, independently selected from the group consisting of hydrogen, halogen, C1-C6 alkyl, C1-C3 haloalkyl, —NH₂, —OR⁶ and —CN;
R¹³ is, at each occurrence, independently selected from the group consisting of hydrogen, C1-C6 alkyl, C3-C10 cycloalkyl and W, as defined above;
wherein if R¹³ is W, R⁹ is hydrogen;
R¹⁴ and R¹⁵ are, at each occurrence, independently selected from the group consisting of hydrogen, halogen, C1-C6 alkyl, C1-C3 haloalkyl, —OR⁶, heterocyclyl and —CN;
R¹⁶ is, at each occurrence, independently selected from the group consisting of hydrogen, halogen, C1-C6 alkyl, C3-C10 cycloalkyl, —N(R⁶)₂, —NR¹³R¹⁴, heterocyclyl, —OR⁶ and —CN;
or an enantiomer, stereoisomeric form, mixture of enantiomers, diastereomer, mixture of diastereomer, racemate of the above mentioned compounds or a pharmaceutically acceptable salt thereof.

In one embodiment, at least one of or exactly one of R², R⁷, R⁸, R⁹, R¹⁰ and R¹³ is W, as defined in claim 1, or is a structure containing W, as defined in claim 1.

In one embodiment, R¹ is C1-C6 alkyl or C1-C3 haloalkyl.

In one embodiment, R² is

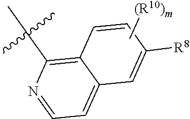

In one embodiment, R¹⁰ is hydrogen; m is 1; R⁸ is W; W is (c-1) or (c.-2) or (c-3), preferably (c-1); L is —NH—; R¹⁴ and R¹⁵ are, independently, at each occurrence, hydrogen, halogen, or C1-C6 alkyl, wherein, preferably, R¹⁴ is halogen; wherein R¹⁶ is hydrogen, halogen, C1-C6 alkyl, —N(R⁶), —NR¹³R¹⁴, wherein, preferably, R¹⁶ is —N(R⁶)₂ or —NR¹³R¹⁴.

In one embodiment, said compound is a compound having a structure selected from structures 1-198, as defined in the column entitled “Structure” of table 1 herein.

In one preferred embodiment, said compound is a compound having a structure selected from compounds 3, 14, 47, and 156 as defined herein.

In a further aspect, the present invention also relates to a combination of the present invention, as defined herein, for use in a method of prevention and/or treatment of cancer in a patient having, or suspected of having, cancer.

In one embodiment of this aspect of the present invention, said method of prevention and/or treatment comprises administering an effective amount of said inhibitor of cyclin-dependent kinase 7 together with an effective amount of said anti-cancer agent to a patient having, or suspected of having, cancer.

In one embodiment of this aspect of the present invention, in said method of prevention and/or treatment, said inhibitor of cyclin-dependent kinase 7 is administered before or after administration of said anti-cancer agent to said patient, or wherein both said inhibitor of cyclin-dependent kinase 7 and said anti-cancer agent are administered concomitantly or synchronously or in a temporally overlapping manner to said patient, or wherein said inhibitor of cyclin-dependent kinase 7 is administered adjunctively to said anti-cancer agent to said patient, or wherein said anti-cancer agent is administered adjunctively to said inhibitor of cyclin-dependent kinase 7, to said patient.

In one embodiment of this aspect of the present invention, said method of prevention and/or treatment comprises administering said combination in conjunction with radiation therapy.

In one embodiment, said cancer is a cancer selected from the group comprising or consisting of: renal cell carcinoma (RCC), kidney cancer, hereditary papillary renal cancer, sporadic papillary renal cancer, non-squamous non-small-cell lung carcinoma (non-squamous NSCLC), squamous non-small-cell lung carcinoma (squamous NSCLC), small-cell lung carcinoma (SCLC), triple-negative breast cancer, colorectal cancer, melanoma, pancreatic ductal adenocarcinoma, esophageal cancer, head and neck squamous cell carcinoma (HNSCC), urothelial cancer, adenocarcinoma, choroidal melanoma, acute leukemia, acoustic neurinoma, ampullary carcinoma, anal carcinoma, astrocytoma, basal cell carcinoma, pancreatic cancer, Desmoid tumor, bladder cancer, bronchial carcinoma, estrogen dependent and independent breast cancer, Burkitt's lymphoma, corpus cancer, Carcinoma unknown primary tumor (CUP-syndrome), small intestine cancer, small intestinal tumors, ovarian cancer, endometrial carcinoma, ependymoma, epithelial cancer types, Ewing's tumors, gastrointestinal tumors, gastric cancer, gallbladder cancer, gall bladder carcinomas, uterine cancer, cervical cancer, cervix, glioblastomas, gynecologic tumors, ear, nose and throat tumors, hematologic tumor, hairy cell leukemia, urethral cancer, skin cancer, skin testis cancer, brain tumors (gliomas), brain metastases, testicle cancer, hypophysis tumor, carcinoids, Kaposi's sarcoma, laryngeal cancer, germ cell tumor, bone cancer, head and neck tumors (tumors of the ear, nose and throat area), colon carcinoma, craniopharyngiomas, oral cancer (cancer in the mouth area and on lips), cancer of the central nervous system, liver cancer, liver metastases, leukemia, eyelid tumor, lung cancer, lymphomas, stomach cancer, malignant melanoma, malignant neoplasia, malignant tumors gastrointestinal tract, breast carcinoma, rectal cancer, medulloblastomas, meningiomas, Hodgkin's/Non-Hodgkin's lymphoma, mycosis fungoides, nasal cancer, neurinoma, neuroblastoma, oligodendroglioma, osteolytic carcinomas and osteoplastic carcinomas, osteosarcomas, ovarian carcinoma, pancreatic carcinoma, penile cancer, plasmacytoma, prostate cancer, pharyngeal cancer, rectal carcinoma, retinoblastoma, vaginal cancer, thyroid carcinoma, T-cell lymphoma, thymoma, tube carcinoma, eye tumors, urethral cancer, urologic tumors, urothelial carcinoma, vulva cancer, wart appearance, soft tissue tumors, soft tissue sarcoma, Nephroblastoma, cervical carcinoma, tongue cancer, invasive ductal carcinoma, invasive lobular carcinoma, ductal carcinoma in situ, lobular carcinoma in situ, small-cell lung carcinoma, non-small-cell lung carcinoma, bronchial adenoma, pleuropulmonary blastoma, mesothelioma, brain stem glioma, hypothalamic glioma, cerebellar astrocytoma, cerebral astrocytoma, neuroectodermal tumor, pineal tumors, sarcoma of the uterus, salivary gland cancers, anal gland adenocarcinomas, mast cell tumors, pelvis tumor, ureter tumor, intraocular melanoma, hepatocellular carcinoma, cholangiocarcinoma, mixed hepatocellular cholangiocarcinoma, squamous cell carcinoma, Merkel cell skin cancer, non-melanoma skin cancer, hypopharyngeal cancer, nasopharyngeal cancer, oropharyngeal cancer, oral cavity cancer, squamous cell cancer, oral melanoma, AIDS-related lymphoma, cutaneous T-cell lymphoma, lymphoma of the central nervous system, malignant fibrous histiocytoma, lymph sarcoma, rhabdomyosarcoma, malignant histiocytosis, fibroblastic sarcoma, hemangiosarcoma, hemangiopericytoma, leiomyosarcoma (LMS), canine mammary carcinoma, and feline mammary carcinoma.

In a further aspect, the present invention also relates to an inhibitor of cyclin-dependent kinase 7 having the general formula I, as defined herein, for use in a method of prevention and/or treatment of cancer, wherein, in said method, said inhibitor of cyclin-dependent kinase 7 is administered to a patient having, or suspected of having, cancer, and wherein said administering of said inhibitor of cyclin-dependent kinase 7 to said patient is in conjunction with administration of radiation therapy.

In a further aspect, the present invention also relates to a method of prevention and/or treatment of cancer in a patient, said method comprising administering a combination of an inhibitor of cyclin-dependent kinase 7 with an anti-cancer agent, said combination being as defined herein, to a patient having, or suspected of having, cancer.

In a further aspect, the present invention also relates to use of a combination as defined herein, for the manufacture of a medicament for the prevention and/or treatment of cancer in a patient.

In a further aspect, the present invention also relates to a pharmaceutical composition comprising a combination, as defined herein, for preventing and/or treating cancer in a patient having, or suspected of having, cancer.

The present inventors have surprisingly found that a combination of the highly specific CDK7-inhibitors of the pyrazolo[1,5-a][1,3,5]triazine- and pyrazolo[1,5-a]pyrimidine-class with other anti-cancer agents is highly efficient with respect to an improvement of anti-tumor efficacy that is considerably improved in comparison to the respective monotherapy/monotherapies. In particular, it has turned out that the combination of such CDK7-inhibitors with other anti-cancer agents greatly improves treatment efficacy of such other anti-cancer agent which is not to be expected, in view of the seemingly totally unrelated mechanisms involved. As an example, the CDK7-inhibitors according to the present invention greatly improve efficacy of immune checkpoint inhibitors, such as PD1-inhibitors or PD-L1-inhibitors, and of poly-ADP-ribose-polymerase (PARP) inhibitors. Furthermore, the combination of the CDK7-inhibitors according to the present invention with cytotoxic non-specific compounds, such as taxanes, or with hormonal anti-cancer agents, such as hormone receptor antagonists, greatly improves the efficacy of the respective monotherapy of such other anti-cancer agents on their own. Surprisingly, such improvement effect seems to be irrespective of the specific type and nature of the other anti-cancer agent, as a result of which the data of the present invention can be extrapolated to other anti-cancer agents and other anti-cancer treatment modalities. Furthermore, the present inventors also expect that such combinations in accordance with embodiments of the present invention will restore sensitivity of cancer cells that have otherwise been resistant to the respective monotherapy.

In preferred embodiments according to the present invention, the cyclin-dependent kinase 7-inhibitor is combined with an immune checkpoint inhibitor, in particular an antibody or antibody fragment directed at an immune checkpoint. Preferably, such immune checkpoint is PD1 or PD-L1. In a preferred embodiment of this aspect of the present invention, such immune checkpoint inhibitor is an anti-PD1-antibody, or an anti-PD-L1-antibody. As an example, such antibody may be an anti-human-PD1-antibody. In a particularly preferred embodiment, such anti-PD1-antibody is pembrolizumab, nivolumab, or cemiplimab. In another embodiment according to the present invention, the cyclin-dependent kinase 7-inhibitor is combined with an inhibitor of poly-ADP-ribose-polymerase (PARP), such as olaparib, pamiparib, or niraparib.

In another preferred embodiment, the CDK7-inhibitor(s) according to the present invention is combined with a cytotoxic non-specific compound which is preferably a taxane, in particular docetaxel, carbacitaxel, or paclitaxel, wherein docetaxel is particularly preferred.

In yet another preferred embodiment according to the present invention, the CDK7-inhibitor(s) according to the present invention is combined with a hormonal anti-cancer agent, preferably a hormone, hormone antagonist, a hormone receptor antagonist, a hormone receptor degrader or an aromatase inhibitor. More preferably, such hormonal anti-cancer agent is a hormone receptor antagonist, which, even more preferably is fulvestrant, tamoxifen, toremifene, letrozole or anastrozole.

The terms “of the [present] invention”, “in accordance with the invention”, “according to the invention” and the like, as used herein, are intended to refer to all aspects and embodiments of the invention described and/or claimed herein. As used herein, the term “comprising” is to be construed as encompassing both “including” and “consisting of”, both meanings being specifically and explicitly intended, and hence, individually disclosed embodiments in accordance with the present invention. Where used herein, “and/or” is to be taken as a specific disclosure of each of the two specified features or components with or without the order. For example, “A” and/or “B” is to be taken as a specific disclosure of each of (i) A, (ii) B and (iii) A and B, just as if each is set out individually herein. Where an indefinite or definite article is used, wherein referring to a singular noun, e. g. “a”, “an” or “the”, this includes a plural of that noun unless something else is specifically stated. Likewise, such disclosure equally is meant to be taken as a specific disclosure of a single individual entity initiated by “a”, “an” or “the”.

The term “CDK7-inhibitor”, as used herein, is meant to refer to an inhibitor of cyclin-dependent kinase 7, which is specific for such cyclin-dependent kinase 7 and does not inhibit or only inhibits to a much smaller extent any of the other cyclin-dependent kinases. In other words, a CDK7-inhibitor, in accordance with the present invention, is not an inhibitor that would have inhibiting activities against multiple cyclin-dependent kinases. More specifically, and preferably, a CDK7-inhibitor in accordance with the present invention is not a PAN-CDK-inhibitor.

The term “target-specific”, as used herein in the context of a compound, relates to the capacity of such compound and/or a molecular structure forming part of such compound, to bind to a certain structure, such as a ligand, an antigen, specifically an epitope, by specific interaction. For example, such term target-specific may be used in conjunction with an antibody, an antigen-binding peptide, an antigen-binding protein, or a surface molecule of an immune cell, such as a cytotoxic T-cell.

The term “combination” as used herein, preferably refers to a composition in which said inhibitor of cyclin-dependent kinase 7 and said anti-cancer agent are present together, being either physically mixed with each other or being kept separate from each other by at least one physical separation barrier between said inhibitor and said anti-cancer agent, wherein said at least one physical separation barrier forms part of said combination. As an example, said inhibitor of cyclin-dependent kinase 7 and said anti-cancer agent may be kept in separate containers or compartments or chambers or dosage units which separate containers, compartments, chambers and dosage units nevertheless form part of said combination. In another embodiment, such “combination” refers to a scenario, wherein said inhibitor of cyclin-dependent kinase 7 and said anti-cancer agent are kept separately in different, separate containers, compartments, chambers or dosage units that are confectioned or arranged in such a manner that they are separated by more than one physical separation barrier, but are nevertheless intended to be administered in conjunction with each other.

The term “in conjunction with each other”, as used herein, is preferably meant to refer to an administration, wherein two agents or treatment modalities, or one agent and one treatment modality, are administered to a patient together, preferably at the same time, one after the other in any desired useful order, or in an overlapping manner. Such co-administration is intended to achieve elevated levels of the respective agent or effective treatment by the respective treatment modality at the same time or, at least, in an overlapping manner, such that both agents or treatment modalities, or the one agent and the one treatment modality, can exert their anti-cancer-effect together.

The term “antibody” as used herein, is meant to refer to a substantially intact antibody, an antibody fragment, e. g. a Fab fragment, a F(ab′)2 fragment, a single chain Fv fragment, a diabody, a triabody, a tetrabody, a bispecific antibody, a nanobody, and other peptide/proteinaceous molecules that retain binding affinity to the ligand for which such “antibody” is specific. In one embodiment, said antibody is a monoclonal antibody. In one embodiment, such antibody is a human antibody or a humanized antibody or a rodent antibody, such as a mouse-antibody which may or may not additionally be humanized.

The term “fusion protein”, as used herein, preferably and specifically is meant to refer to antibody-cytokine fusion proteins. In one embodiment, in such fusion protein, an intact antibody, or an antibody fragment (e.g. an intact IgG, an Fc fragment, a Fab fragment or a scFv fragment) is linked to a cytokine monomer (e.g. IL-2 or IFN-alpha or GM-CSF) or cytokine homomultimer (IFN-gamma or TNF) or cytokine heteromultimer (e.g. IL-12 or IL-27). Examples of suitable antibody-cytokine fusion proteins that may be used in embodiments of the present invention are disclosed in Jin et al., 2022; Signal Transduction and Targeted Therapy; 7: 39; https://doi.org/10.1108/s41392-021-00868-x.

The term “radiopharmaceutical”, as used herein, refers to a drug containing a radioactive isotope, more specifically to a target-specific drug containing a radioactive isotope. Typically, the target-specific drug is specific for a particular cell-type or tissue type, more specifically for cancer cell(s) or cancer tissue(s) and specifically binds thereto or specifically interacts therewith, and the radioactive isotope which forms part of the radiopharmaceutical, is thus brought into vicinity to such cell(s) or tissue(s) and damages such cell(s) or tissue(s) by emitting radiation thereto. Examples of target-specific drugs within radiopharmaceuticals are antibodies and antibody fragments, in particular monoclonal antibodies and antibody fragments, poly-ADP-ribose-polymerase (PARP) inhibitors; tyrosine kinase inhibitors; and immunotoxins. Specific examples of radiopharmaceuticals are metastron, zevalin, xofigo, lutathera, azedra, and pluvicto.

The term “radiation therapy”, as used herein, is meant to include external beam radiation therapy, brachytherapy as well as treatment with a radiopharmaceutical, and combinations thereof. Hence, when in accordance with one aspect of the present invention, as defined in claim 21, an inhibitor of cyclin-dependent kinase 7, as defined herein, is administered to a patient “in conjunction with administration of radiation therapy”, such co-administration is meant to include scenarios wherein such inhibitor is administered in conjunction with either a) external beam radiation therapy, or b) with brachytherapy or c) with treatment with a radiopharmaceutical, or with d) any combination of a)-c). In one embodiment, such co-administration is meant to refer to an administration of said inhibitor together with external beam radiation therapy (and no brachytherapy and no treatment with a radiopharmaceutical). In another embodiment, such co-administration is meant to refer to an administration of said inhibitor together with brachytherapy (and no external beam radiation therapy and no treatment with a radiopharmaceutical). In yet another embodiment, such co-administration is meant to refer to an administration of said inhibitor together with treatment with a radiopharmaceutical (and no brachytherapy and no external beam radiation therapy).

As used herein, “external beam radiation therapy” is different from and does not include therapy which is based on or involves the administration of a radiopharmaceutical or of other radioactive material to or into the patient's body. Instead “external beam radiation therapy”, as used herein, refers to uses of a beam, preferably a collimated or focused beam, of ionizing radiation from outside of a patient's body to or into a patient's body to treat a disorder or disease, preferably a cancerous disease. In a preferred embodiment, “external beam radiation therapy” involves irradiation by X-rays, gamma-rays, protons, neutrons, electrons or heavy ions, preferably X-rays. In a further preferred embodiment, “external beam radiation therapy” is selected from treatment modalities including but not limited to three-dimensional conformal radiation therapy (3D-CRT), intensity modulated radiation therapy (IMRT), image-guided radiation therapy (IGRT), and stereotactic radiation therapy (SRT).

As used herein, the term “brachytherapy”, is meant to refer to a type of internal radiation therapy in which a suitable implant, such as a seed, capsule, bolus, ribbon, strip, stick, needle, bar, plaster, or wire, is placed into a patient's body, preferably into a tumor or cancerous tissue or in the vicinity thereof, and such implant contains a source of radiation. As a result of the implant being placed into a patient's body, radiation is emitted from the implant and into the part of the body where such implant has been placed. Placement of the implant may be temporal or permanent, depending on the desired type, intensity and duration of treatment. Examples of brachytherapy include, but are not limited to low-dose rate (LDR) implants, high-dose rate (HDR) implants and permanent implants.

The term “optionally substituted” as used herein is meant to indicate that a hydrogen atom where present and attached to a member atom within a group, or several such hydrogen atoms, may be replaced by a suitable group, such as halogen including fluorine, C₁-C₃ alkyl, C₁-C₃ haloalkyl, methylhydroxyl, COOMe, C(O)H, COOH, OMe, or OCF₃.

The term “alkyl” refers to a monovalent straight, branched or cyclic chain, saturated aliphatic hydrocarbon radical having a number of carbon atoms in the specified range. Thus, for example, “C₁-C₆ alkyl” refers to any of the hexyl alkyl and pentyl alkyl isomers as well as n-, iso-, sec-, and t-butyl, n- and isopropyl, cyclic propyl, ethyl and methyl.

The term “alkenyl” refers to a monovalent straight or branched chain aliphatic hydrocarbon radical containing one carbon-carbon double bond and having a number of carbon atoms in the specified range. Thus, for example, “C₂-C₆ alkenyl” refers to all of the hexenyl and pentenyl isomers as well as 1-butenyl, 2-butenyl, 3-butenyl, isobutenyl, 1-propenyl, 2-propenyl, and ethenyl (or vinyl).

The term “cycloalkyl”, alone or in combination with any other term, refers to a group, such as optionally substituted or non-substituted cyclic hydrocarbon, having from three to eight carbon atoms, unless otherwise defined. Thus, for example, “C₃-C₈ cycloalkyl” refers to cyclopropyl, cyclobutyl, cyclopentyl, cyclohexyl, cycloheptyl, and cyclooctyl.

The term “haloalkyl” refers to an alkyl group, as defined herein that is substituted with at least one halogen. Examples of straight or branched chained “haloalkyl” groups useful in the present invention include, but are not limited to, methyl, ethyl, propyl, isopropyl, n-butyl, and t-butyl substituted independently with one or more halogens. The term “haloalkyl” should be interpreted to include such substituents such as —CHF₂, —CF₃, —CH₂—CH₂—F, —CH₂—CF₃, and the like.

The term “heteroalkyl” refers to an alkyl group where one or more carbon atoms have been replaced with a heteroatom, such as, O, N, or S. For example, if the carbon atom of alkyl group which is attached to the parent molecule is replaced with a heteroatom (e.g., O, N, or S) the resulting heteroalkyl groups are, respectively, an alkoxy group (e.g., —OCH₃, etc.), an amine (e.g., —NHCH₃, —N(CH₃)₂, etc.), or thioalkyl group (e.g., —SCH₃, etc.). If a non-terminal carbon atom of the alkyl group which is not attached to the parent molecule is replaced with a heteroatom (e.g., O, N, or S) and the resulting heteroalkyl groups are, respectively, an alkyl ether (e.g., —CH₂CH₂—O—CH₃, etc.), alkyl amine (e.g., —CH₂NHCH₃, —CH₂N(CH₃)₂, etc.), or thioalkyl ether (e.g., —CH₂—S—CH₃).

The term “halogen” refers to fluorine, chlorine, bromine, or iodine.

The term “phenyl” as used herein is meant to indicate that optionally substituted or non-substituted phenyl group.

The term “benzyl” as used herein is meant to indicate that optionally substituted or non-substituted benzyl group.

The term “heteroaryl” refers to (i) optionally substituted 5- and 6-membered heteroaromatic rings and (ii) optionally substituted 9- and 10-membered bicyclic, fused ring systems in which at least one ring is aromatic, wherein the heteroaromatic ring or the bicyclic, fused ring system contains from 1 to 4 heteroatoms independently selected from N, O, and S, where each N is optionally in the form of an oxide and each S in a ring which is not aromatic is optionally S(O) or S(O)₂. Suitable 5- and 6-membered heteroaromatic rings include, for example, pyridyl, pyrrolyl, pyrazinyl, pyrimidinyl, pyridazinyl, triazinyl, thienyl, furanyl, imidazolyl, pyrazolyl, triazolyl, tetrazolyl, oxazolyl, isooxazolyl, oxadiazolyl, thiazolyl, isothiazolyl, and thiadiazolyl. Suitable 9- and 10-membered heterobicyclic, fused ring systems include, for example, benzofuranyl, indolyl, indazolyl, naphthyridinyl, isobenzofuranyl, benzopiperidinyl, benzisoxazolyl, benzoxazolyl, chromenyl, quinolinyl, isoquinolinyl, cinnolinyl, quinazolinyl, tetrahydroquinolinyl, tetrahydroisoquinolinyl, isoindolyl, benzodioxolyl, benzofuranyl, imidazo[1,2-a]pyridinyl, benzotriazolyl, dihydroindolyl, dihydroisoindolyl, indazolyl, indolinyl, isoindolinyl, quinoxalinyl, quinazolinyl, 2,3-dihydrobenzofuranyl, and 2,3-dihydrobenzo-1,4-dioxinyl.

The term “heterocyclyl” refers to (i) optionally substituted 4- to 8-membered, saturated and unsaturated but non-aromatic monocyclic rings containing at least one carbon atom and from 1 to 4 heteroatoms, (ii) optionally substituted bicyclic ring systems containing from 1 to 6 heteroatoms, and (iii) optionally substituted tricyclic ring systems, wherein each ring in (ii) or (iii) is independent of fused to, or bridged with the other ring or rings and each ring is saturated or unsaturated but nonaromatic, and wherein each heteroatom in (i), (ii), and (iii) is independently selected from N, O, and S, wherein each N is optionally in the form of an oxide and each S is optionally oxidized to S(O) or S(O)₂. Suitable 4- to 8-membered saturated heterocyclyls include, for example, azetidinyl, piperidinyl, morpholinyl, thiomorpholinyl, thiazolidinyl, isothiazolidinyl, oxazolidinyl, isoxazolidinyl, pyrrolidinyl, imidazolidinyl, piperazinyl, tetrahydrofuranyl, tetrahydrothienyl, pyrazolidinyl, hexahydropyrimidinyl, thiazinanyl, thiazepanyl, azepanyl, diazepanyl, tetrahydropyranyl, tetrahydrothiopyranyl, dioxanyl, and azacyclooctyl. Suitable unsaturated heterocyclic rings include those corresponding to the saturated heterocyclic rings listed in the above sentence in which a single bond is replaced with a double bond. It is understood that the specific rings and ring systems suitable for use in the present invention are not limited to those listed in this and the preceding paragraphs. These rings and ring systems are merely representative.

### Pharmaceutically Acceptable Salts

Examples of pharmaceutically acceptable addition salts include, without limitation, the non-toxic inorganic and organic acid addition salts such as the acetate derived from acetic acid, the aconate derived from aconitic acid, the ascorbate derived from ascorbic acid, the benzenesulfonate derived from benzensulfonic acid, the benzoate derived from benzoic acid, the cinnamate derived from cinnamic acid, the citrate derived from citric acid, the embonate derived from embonic acid, the enantate derived from enanthic acid, the formate derived from formic acid, the fumarate derived from fumaric acid, the glutamate derived from glutamic acid, the glycolate derived from glycolic acid, the hydrochloride derived from hydrochloric acid, the hydrobromide derived from hydrobromic acid, the lactate derived from lactic acid, the maleate derived from maleic acid, the malonate derived from malonic acid, the mandelate derived from mandelic acid, the methanesulfonate derived from methane sulphonic acid, the naphthalene-2-sulphonate derived from naphtalene-2-sulphonic acid, the nitrate derived from nitric acid, the perchlorate derived from perchloric acid, the phosphate derived from phosphoric acid, the phthalate derived from phthalic acid, the salicylate derived from salicylic acid, the sorbate derived from sorbic acid, the stearate derived from stearic acid, the succinate derived from succinic acid, the sulphate derived from sulphuric acid, the tartrate derived from tartaric acid, the toluene-p-sulphonate derived from p-toluene sulphonic acid, and the like. Such salts may be formed by procedures well known and described in the art.

Other acids such as oxalic acid, which may not be considered pharmaceutically acceptable, may be useful in the preparation of salts useful as intermediates in obtaining a chemical compound of the invention and its pharmaceutically acceptable acid addition salt.

In another embodiment, the compounds of the invention are used in their respective free base form according to the present invention.

Metal salts of a chemical compound of the invention include alkali metal salts, such as the sodium salt of a chemical compound of the invention containing a carboxy group.

The chemical compounds within combinations of the invention may be provided in unsolvated or solvated forms together with a pharmaceutically acceptable solvent(s) such as water, ethanol, and the like. Solvated forms may also include hydrated forms such as the monohydrate, the dihydrate, the hemihydrate, the trihydrate, the tetrahydrate, and the like. In general, solvated forms are considered equivalent to unsolvated forms for the purposes of this invention.

Preferred CDK7-inhibitors that form part of combinations according to the present invention are the ones as listed and shown in table 1 in the column entitled “Structure” herein:

TABLE 1
Summary of compounds 1-198 in terms of their structures and exemplary corresponding characteristics

| #cpds | Structure | Characterization Data |
|---|---|---|
| 1 | 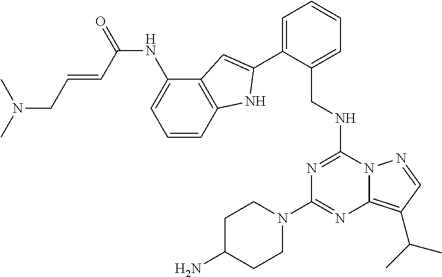 [SMILES: CC(C)c1cnn2c(NCc3ccccc3-c3cc4c(NC(=O)/C=C/CN(C)C)cccc4[nH]3)nc(N3CCC(N)CC3)nc12] | White powder; ¹H-NMR (DMSO-d₆, 400 MHz): δ 8.65 (1H, brs), 7.89 (1H, brs), 7.62 (1H, s), 7.48-7.59 (3H, m), 7.31-7.43 (2H, m), 7.16-7.22 (1H, m), 7.09-7.15 (1H, m), 6.95-7.06 (1H, m), 6.61 (1H, t, J = 5.6 Hz), 6.56 (1H, s), 6.21 (1H, d, J = 15.2 Hz), 4.83 (2H, d, J = 5.2 Hz), 4.55- 4.65 (2H, m), 3.10-3.20 (2H, m), 2.98-3.08 (1H, m), 2.80- 2.89 (3H, m), 2.30 (6H, s), 1.77-1.79 (2H, m), 1.28-1.32 (8H, m); LCMS: 100%, MS (ESI): m/z 629.4 [M + Na]⁺ |
| 2 | 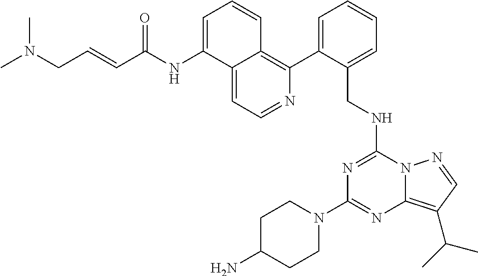 [SMILES: CC(C)c1cnn2c(NCc3ccccc3-c3nccc4c(NC(=O)/C=C/CN(C)C)cccc34)nc(N3CCC(N)CC3)nc12] | white powder; ¹H-NMR (CDCl3, 400 MHz): δ 8.47-8.49 (2H, s), 7.97-7.99 (1H, m), 7.61-7.63 (1H, m), 7.55-7.58 (1H, m), 7.52 (1H, s), 7.38-7.49 (4H, m), 7.22-7.25 (1H, m), 7.04-7.11 (1H, m), 6.63-6.65 (1H, m), 6.35-6.40 (1H, m), 4.72-4.77 (1H, m), 4.78-4.55 (2H, m), 4.26-4.30 (1H, m), 3.19-3.21 (2H, m), 2.92-3.02 (2H, m), 2.76-2.83 (2H, m), 2.35 (6H, s), 1.79-7.82 (2H, m), 1.14-1.29 (8H, m); LCMS: 95.3%, MS (ESI): m/z 619.4 [M + H]+. |
| 3 | 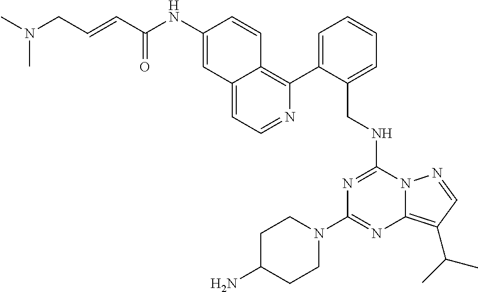 [SMILES: CC(C)c1cnn2c(NCc3ccccc3-c3nccc4cc(NC(=O)/C=C/CN(C)C)ccc34)nc(N3CCC(N)CC3)nc12] | white powder; ¹H-NMR (DMSO-d6, 400 MHz): δ 10.51 (1H, brs), 8.44-8.50 (3H, m), 7.75 (1H, d, J = 5.6 Hz), 7.64 (1H, s), 7.55-7.60 (1H, m), 7.46-7.53 (3H, m), 7.42 (1H, t, J = 8.0 Hz), 7.33 (1H, d, J = 7.2 Hz), 6.78-6.85 (1H, m), 6.36 (1H, d, J = 15.2 Hz), 4.25-4.43 (4H, m), 3.08 (2H, d, J = 5.2 Hz), 2.72-2.86 (4H, m), 2.19 (6H, s), 1.60-1.70 (2H, m), 1.19 (6H, d, J = 7.2 Hz), 1.05-1.10 (2H, m); LCMS: 98.9%, MS (ESI): m/z 619.4 [M + H] +. |
| 4 | 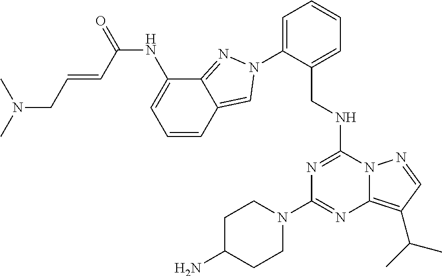 [SMILES: CC(C)c1cnn2c(NCc3ccccc3-n3cc4cccc(NC(=O)/C=C/CN(C)C)c4n3)nc(N3CCC(N)CC3)nc12] | yellow powder; ¹H-NMR (CDCl3, 400 MHz): δ 9.22 (1H, brs), 8.59 (1H, t, J = 5.8 Hz), 8.42 (1H, d, J = 7.6 Hz), 8.19 (1H, s), 7.70 (1H, s), 7.66 (1H, d, J = 6.8 Hz), 7.40-7.47 (4H, m), 7.16-7.20 (1H, m), 7.04-7.08 (1H, m), 6.61 (1H, d, J = 15.2 Hz), 4.83-4.88 (2H, m), 4.58 (2H, d, J = 6.0 Hz), 3.38-3.40 (1H, m), 3.27 (2H, d, J = 6.4 Hz), 2.98-3.05 (3H, m), 2.56 (6H, s), 2.21-2.38 (2H, m), 1.65-1.68 (2H, m), 1.25-1.29 (8H, m); LCMS: 95.6%, MS (ESI): m/z 608.3 [M + H]⁺. |
| 5 | 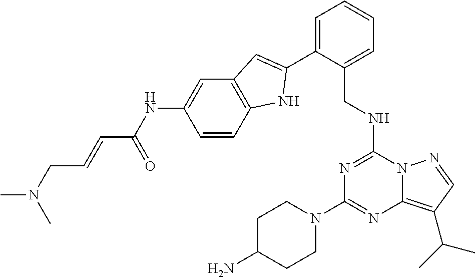 [SMILES: CC(C)c1cnn2c(NCc3ccccc3-c3cc4cc(NC(=O)/C=C/CN(C)C)ccc4[nH]3)nc(N3CCC(N)CC3)nc12] | white powder; ¹H-NMR (DMSO-d₆, 400 MHz): δ 8.72 (1H, s), 7.87 (1H, s), 7.58 (2H, s), 7.46-7.54 (2H, m), 7.33-7.40 (2H, m), 7.18-7.26 (2H, m), 6.90-7.01 (1H, m), 6.64 (1H, brs), 6.54 (1H, s), 6.13 (1H, d, J = 15.6 Hz), 4.82 (2H, s), 4.55-4.65 (2H, m), 3.11 (2H, d, J = 5.2 Hz), 2.98- 3.05 (1H, m), 2.79-2.88 (3H, m), 2.28 (6H, s), 1.70-1.80 (2H, m), 1.29 (8H, d, J = 6.8 Hz); LCMS: 100%, MS (ESI): m/z 629.2 [M + Na]⁺ |
| 6 | 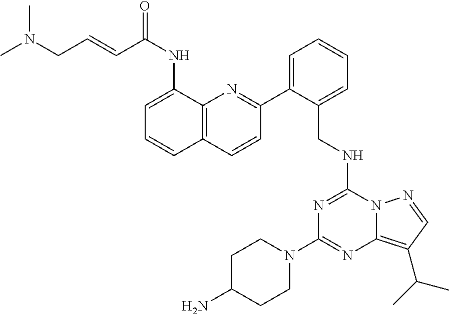 [SMILES: CC(C)c1cnn2c(NCc3ccccc3-c3ccc4cccc(NC(=O)/C=C/CN(C)C)c4n3)nc(N3CCC(N)CC3)nc12] | yellow powder; ¹H NMR (DMSO-d₆, 400 MHz): δ 10.08 (1H, brs), 8.89 (1H, t, J = 5.6 Hz), 8.69 (1H, d, J = 7.6 Hz), , 8.50 (1H, d, J = 8.4 Hz), 7.88 (1H, d, J = 8.4 Hz), 7.60-7.85 (7H, m), 7.43-7.49 (2H, m), 6.73-6.80 (1H, m), 6.50 (1H, d, J = 16.0 Hz), 5.04 (2H, d, J = 6.4 Hz ), 4.30-4.33 (2H, m), 3.11-3.17 (1H, m), 3.00 (2H, d, J = 6.4 Hz), 2.83-2.92 (1H, m), 2.62-2.67 (2H, m), 2.11 (6H, s), 1.71-1.75 (2H, m), 1.07-1.27 (8H, m); LCMS: 100%, MS (ESI): m/z 619.4 [M + H] ⁺. |
| 7 | 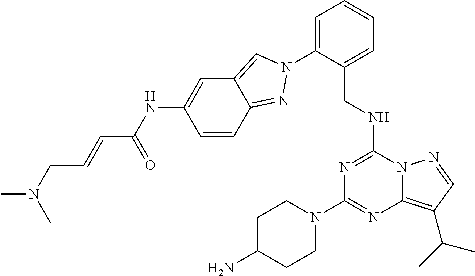 [SMILES: CC(C)c1cnn2c(NCc3ccccc3-n3cc4cc(NC(=O)/C=C/CN(C)C)ccc4n3)nc(N3CCC(N)CC3)nc12] | brown powder; ¹H-NMR (DMSO-d₆, 400 MHz): δ 10.2 (1H, brs), 8.93-8.97 (1H, m), 8.71 (1H, s), 8.38 (1H, d, J = 2.8 Hz), 7.77 (1H, s), 7.67 (1H, d, J = 9.6 Hz), 7.56- 7.72 (2H, m), 7.45-7.52 (2H, m), 7.39-7.44 (1H, m), 6.71-6.80 (1H, m), 6.38 (1H, d, J = 15.2 Hz), 4.61 (2H, d, J = 5.6 Hz), 4.39-4.50 (2H, m), 3.15-3.26 (2H, m), 2.85-2.93 (2H, m), 2.72-2.83(2H, m), 2.17-2.35 (6H, m), 1.81-1.90 (2H, m), 1.14-1.34 (8H, m); LCMS: 96.5 %, MS (ESI): m/z = 608.3 [M + H]⁺. |
| 8 | 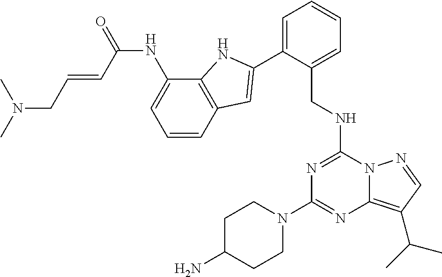 [SMILES: CC(C)c1cnn2c(NCc3ccccc3-c3cc4cccc(NC(=O)/C=C/CN(C)C)c4[nH]3)nc(N3CCC(N)CC3)nc12] | white powder; ¹H-NMR (CDCl3, 400 MHz): δ 10.44 (1H, brs), 7.61 (1H, brs), 7.54-7.63 (3H, m), 7.48 (1H, d, J = 7.6 Hz), 7.32-7.41 (2H, m), 6.93-7.07 (2H, m), 6.79 (1H, d, J = 7.2 Hz), 6.57-6.68 (2H, m), 6.21 (1H, d, J = 14.8 Hz), 4.90 (2H, d, J = 5.6 Hz), 4.58-4.68 (2H, m), 3.12 (2H, d, J = 5.6 Hz), 3.98-3.06 (1H, m), 2.80-2.91 (3H, m), 2.29 (6H, s), 1.74-1.84 (2H, m), 1.29 (6H, d, J = 6.8 Hz), 1.20- 1.26 (2H, m); LCMS: 100%, MS (ESI): m/z 629.3[M + Na]⁺. |
| 9 | 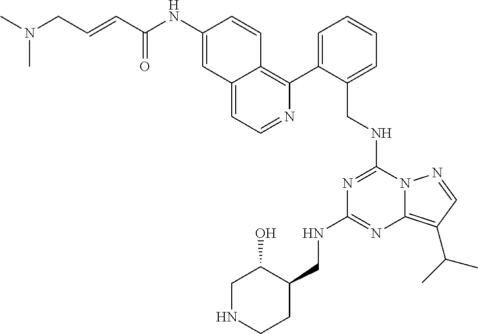 [SMILES: CC(C)c1cnn2c(NCc3ccccc3-c3nccc4cc(NC(=O)/C=C/CN(C)C)ccc34)nc(NC[C@H]3CCNC[C@@H]3O)nc12] | (3R, 4R); pale-yellow powder; ¹H NMR (400 MHz, DMSO-d₆): δ 10.91-11.17 (2H, m), 9.03 (1H, br s), 8.82 (1H, br s), 8.38-8.58 (3H, m), 7.79-7.89 (1H, m), 7.70- 7.77 (1H, m), 7.62-7.67 (1H, m), 7.50-7.57 (2H, m), 7.42- 7.49 (1H, m), 7.37 (1H, br d, J = 7.2 Hz), 6.85-6.98 (1H, m), 6.63 (1H, d, J = 15.6 Hz), 5.67-5.96 (1H, m), 4.33- 4.50 (2H, m), 3.96 (2H, d, J = 6.8 Hz), 3.42-3.57 (2H, m), 2.97-3.25 (3H, m), 2.81-2.90 (1H, m), 2.77 (6H, s), 2.58- 2.69 (1H, m), 1.39-1.90 (3H, m), 1.19 (6H, br d, J = 6.4 Hz); LCMS: 100%, MS (ESI): m/z 649.3 [M + H]⁺ |
| 10 | 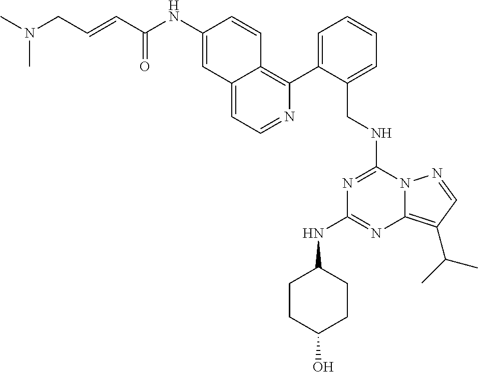 | (1R, 4R); yellow powder; ¹H-NMR (400 MHz, CD₃OD): δ 8.67 (1H, s), 8.42 (1H, br d, J = 6.4 Hz), 8.24 (1H, br d, J = 6.4 Hz), 7.84-7.93 (2H, m), 7.78 (1H, d, J = 9.2 Hz), 7.68 (1H, br s), 7.49-7.62 (3H, m), 6.95-7.10 (1H, m), 6.72 (1H, br d, J = 15.2 Hz), 4.70-5.10 (2H, m), 4.07 (2H, br d, J = 6.8 Hz) 3.47-3.65 (2H, m), 2.91-3.01 (6H, m), 2.77- 2.87 (1H, m), 1.87-2.10 (3H, m), 1.65-1.72 (1H, m), 1.30- 1.51 (4H, m), 1.12-1.23 (6H, m); LCMS: 100%, MS (ESI): m/z 634.3 [M + H]⁺. |
| 11 | 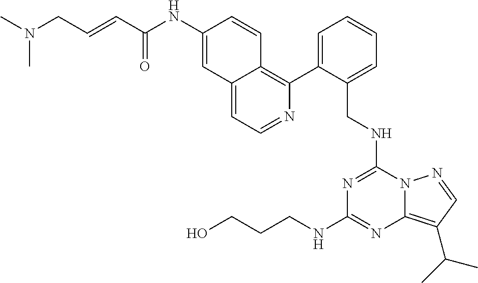 [SMILES: CC(C)c1cnn2c(NCc3ccccc3-c3nccc4cc(NC(=O)/C=C/CN(C)C)ccc34)nc(NCCCO)nc12] | yellow powder; ¹H NMR (400 MHz, CD₃OD): δ 8.42 (1H, s), 8.37 (1H, d, J = 6.0 Hz), 7.68-7.77 (2H, m), 7.61-7.67 (1H, m), 7.46-7.60 (4H, m), 7.40 (1H, d, J = 7.6 Hz), 6.92- 7.02 (1H, m), 6.64 (1H, d, J = 15.2 Hz), 5.01-5.04 (1H, m), 4.39-4.43 (1H, m), 4.06 (2H, d, J = 6.4 Hz), 3.65 (2H, t, J = 6.0 Hz), 3.33-3.41 (2H, m), 2.97 (6H, s), 2.82-2.91 (1H, m), 1.74-1.80 (2H, m), 1.15-1.25 (6H, m); LCMS: 100%, MS (ESI): m/z 594.3 [M + H]⁺. |
| 12 | 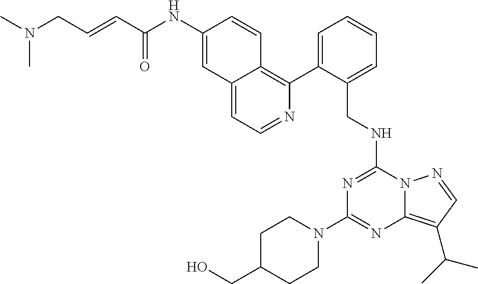 [SMILES: CC(C)c1cnn2c(NCc3ccccc3-c3nccc4cc(NC(=O)/C=C/CN(C)C)ccc34)nc(N3CCC(CO)CC3)nc12] | yellow solid; ¹H-NMR (400 MHz, CD₃OD): δ 8.66 (1H, s), 6.42-6.44 (1H, m), 8.21-8.23 (1H, m), 7.80-7.87 (3H, m), 7.59-7.71 (4H, m), 7.00-7.07 (1H, m), 6.74-6.78 (1H, d, J = 15.6 Hz), 4.95-5.02 (1H, m), 4.71-4.81 (1H, m), 4.31- 3.41 (2H, m), 4.08 (2H, d, J = 7.2 Hz), 3.46-3.47 (2H, m), 3.08-3.13 (2H, m), 2.96-2.97 (6H, m), 1.85-1.97 (2H, m), 1.29-1.38 (2H, m), 1.16-1.19 (6H, m); LCMS: 100.0%, MS (ESI): 634.3 m/z [(M + H)]⁺. |
| 13 | 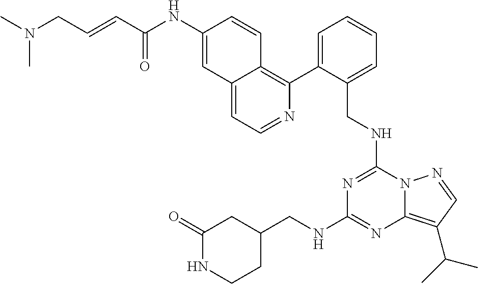 [SMILES: CC(C)c1cnn2c(NCc3ccccc3-c3nccc4cc(NC(=O)/C=C/CN(C)C)ccc34)nc(NCC3CCNC(=O)C3)nc12] | Racemic mixture; off-white powder; ¹H NMR (400 MHz, CD₃OD): δ 8.27-8.42 (2H, m) 7.25-7.75 (8H, m), 6.91- 7.07 (1H, m), 6.33 (1H, d, J = 15.2 Hz), 4.80-4.87 (1H, m), 4.60-4.67 (2H, m), 4.28-4.37 (1H, m), 3.47-3.56 (1H, m), 3.26 (2H, d, J = 6.4 Hz), 3.05-3.13 (1H, m), 2.85-2.92 (1H, m), 2.45-2.54 (2H, m), 2.36 (6H, s), 2.01-2.11 (1H, m), 1.65-1.75 (2H, m), 1.15-1.26 (6H, m); LCMS: 95.9%, MS (ESI): m/z 324.2 [M/2 + H]⁺. |
| 14 | 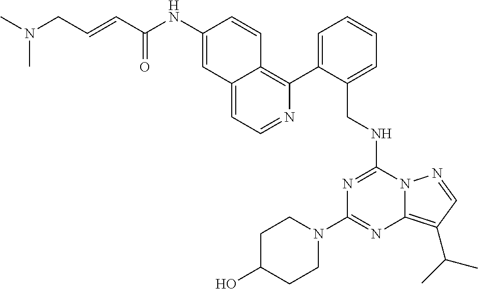 [SMILES: CC(C)c1cnn2c(NCc3ccccc3-c3nccc4cc(NC(=O)/C=C/CN(C)C)ccc34)nc(N3CCC(O)CC3)nc12] | light yellow powder; ¹H NMR (400 MHz, CD₃OD): δ 8.32 (1H, d, J = 6.0 Hz), 8.29 (1H, s), 7.63-7.68 (1H, m), 7.53- 7.61 (2H, m), 7.49 (1H, t, J = 6.8 Hz), 7.36-7.40 (2H, m), 7.31-7.35 (2H, m), 6.91-7.00 (1H, m), 6.38-6.42 (1H, m), 4.34-4.38 (1H, m), 4.13-4.25 (2H, m), 3.74-3.82 (1H, m), 3.49 (2H, d, J = 6.8 Hz), 2.98-3.13 (2H, m), 2.85 (1H, m), 2.53 (6H, s), 1.81 (2H, m), 1.23-1.45 (3H, m), 1.19 (6H, d, J = 6.8 Hz); LCMS: 100%, MS (ESI): m/z 620.2 [M + H]⁺. |
| 15 | 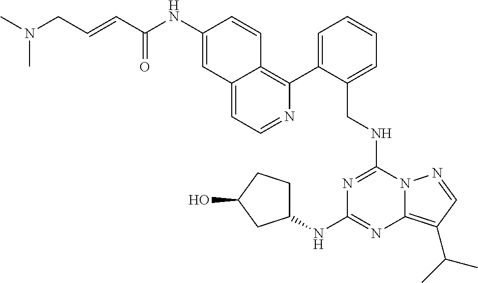 | (1S, 3S); light yellow solid; ¹H-NMR (MeOD, 400 MHz): δ 8.69-8.71 (1H, m), 8.42-8.45 (1H, m), 8.25-8.27 (1H, m), 7.80-7.88 (3H, m), 7.58-7.72 (4H, m), 6.99-7.07 (1H, m), 6.73 (1H, d, J = 15.2 Hz), 5.01-5.06 (1H, m), 4.91- 4.94 (1H, m), 4.71-4.78 (1H, m), 4.39-4.44 (1H, m), 4.26- 4.29 (1H, m), 4.07(2H, d, J = 7.2 Hz), 2.96 (6H, s), 2.84- 2.88 (1H, m), 2.06-2.32 (2H, m), 1.95-1.99 (1H, m), 1.61- 1.80 (3H, m), 1.18-1.21 (6H, m); LCMS: 99.7%, MS (ESI):620.2 m/z [M + H]⁺. |
| 16 | 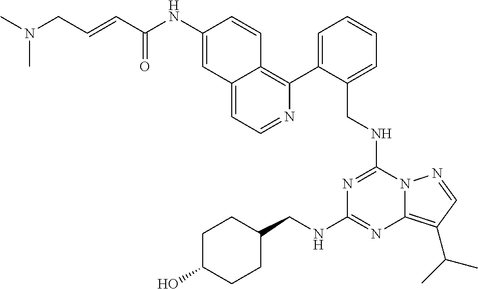 | (1R, 4R); white powder; ¹H NMR (CD₃OD, 400 MHz): δ 8.66 (1H, d, J = 2.0 Hz), 8.43 (1H, d, J = 6.8 Hz), 8.23 (1H, d, J = 6.8 Hz), 7.76-7.88 (3H, m), 7.66-7.74 (1H, m), 7.55- 7.63 (3H, m), 6.97-7.08 (1H, m), 6.71 (1H, d, J = 15.6 Hz), 4.89-4.90 (2H, m), 4.07 (2H, d, J = 6.4 Hz), 3.45-3.54 (1H, m), 3.17-3.26 (1H, m), 3.04 (1H, m), 2.96 (6H, s), 2.84 (1H, m), 1.88-2.05 (2H, m), 1.67-1.83 (2H, m), 1.36-1.49 (1H, m), 1.28-1.35 (1H, m), 1.14-1.21 (7H, m), 0.84-1.11 (2H, m); LCMS: 99.7%, MS (ESI): m/z 648.2 [M + H]⁺; HPLC (254 nm): 100%. |
| 17 | 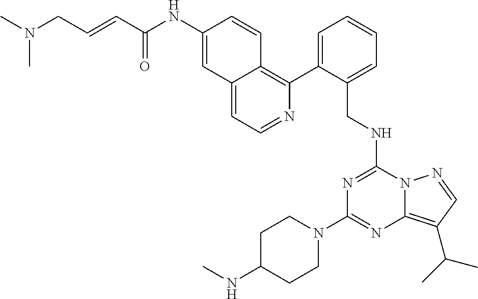 [SMILES: CNC1CCN(c2nc(NCc3ccccc3-c3nccc4cc(NC(=O)/C=C/CN(C)C)ccc34)n3ncc(C(C)C)c3n2)CC1] | yellow powder; ¹H NMR (400 MHz, CD₃OD): δ 8.67 (1H, s), 8.38-8.45 (1H, br s), 8.18-8.26 (1H, m), 7.78-7.88 (3H, m), 7.55-7.73 (4H, m), 6.97-7.08 (1H, m), 6.74 (1H, d, J = 15.2 Hz), 4.95-5.03 (1H, m), 4.66-4.76 (1H, m), 4.48-4.59 (2H, m), 4.07 (2H, d, J = 6.8 Hz), 3.39-3.54 (1H, m), 3.03-3.24 (3H, m), 2.96 (6H, s), 2.76 (3H, s), 2.14- 2.31 (2H, m), 1.46-1.80 (2H, m), 1.19 (6H, d, J = 6.8 Hz); LCMS: 99.7%, MS (ESI): m/z 633.3 [M + H]⁺ |
| 18 | 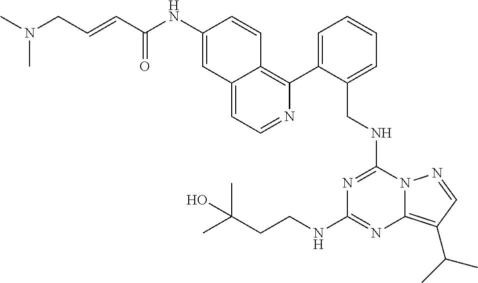 [SMILES: CC(C)c1cnn2c(NCc3ccccc3-c3nccc4cc(NC(=O)/C=C/CN(C)C)ccc34)nc(NCCC(C)(C)O)nc12] | light yellow solid; ¹H-NMR (CD₃OD, 400 MHz): δ 8.69 (1H, s), 8.44 (1H, d, J = 6.4 Hz), 8.23 (1H, d, J = 6.8 Hz), 7.81-7.87 (3H, m), 7.68-7.72 (2H, m), 7.59-7.60 (2H, m), 6.99-7.07 (1H, m), 6.73 (1H, d, J = 15.2 Hz), 5.04-5.08 (1H, m), 4.70-4.74 (1H, m), 4.07 (2H, d, J = 6.4 Hz), 3.41- 3.44 (2H, m), 2.96 (6H, s), 2.82-2.86 (1H, m), 1.69-1.73 (2H, m), 1.28 (6H, s), 1.21 (6H, d, J = 6.8 Hz); LCMS: 98.2%, MS (ESI): 622.3 m/z [(M + H)]⁺. |
| 19 | 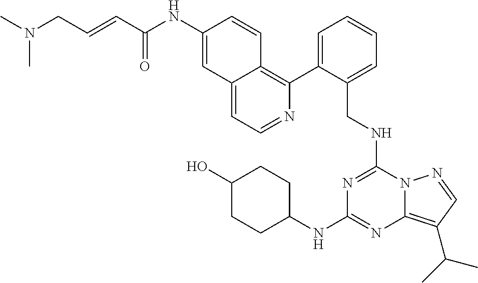 [SMILES: CC(C)c1cnn2c(NCc3ccccc3-c3nccc4cc(NC(=O)/C=C/CN(C)C)ccc34)nc(NC3CCC(O)CC3)nc12] | White solid; ¹H NMR (400 MHz, DMSO-d₆): δ 11.24- 11.45 (2H, m), 9.42-9.66 (1H, m), 8.61-8.66 (1H, m), 8.41-8.47 (1H, m), 8.18-8.23 (1H, m), 7.72-7.75 (2H, m), 7.67-7.69 (1H, m), 7.54-7.60 (1H, m), 7.41-7.52 (2H, m), 6.91-7.01 (1H, m), 6.65 (1H, m), 4.74-4.83 (1H, m), 4.50-4.61 (1H, m), 3.95-4.02 (2H, m), 2.72-2.81 (6H, m), 1.76-1.91 (1H, m), 1.44-1.68 (5H, m), 1.21-1.38 (2H, m), 1.10-1.16 (6H, m); LCMS: 100%, MS (ESI): m/z 634.3 [M + H]⁺. |
| 20 | 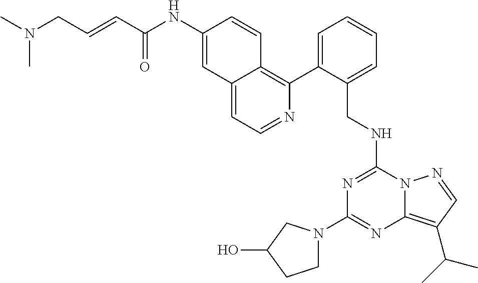 [SMILES: CC(C)c1cnn2c(NCc3ccccc3-c3nccc4cc(NC(=O)/C=C/CN(C)C)ccc34)nc(N3CCC(O)C3)nc12] | Racemic mixture; off-white powder; ¹H NMR (400 MHz, CD₃OD): δ 8.64-8.70 (1H, m), 8.40-8.51 (1H, m), 8.17- 8.28 (1H, m), 7.81-7.92 (3H, m), 7.68-7.75 (2H, m), 7.58- 7.66 (2H, m), 7.01-7.09 (1H, m), 6.77 (1H, d, J = 15.2 Hz), 5.11-5.20 (1H, m), 4.53-4.72 (2H, m), 4.09 (2H, d, J = 7.2 Hz), 3.38-3.86 (4H, m), 3.08-3.21 (1H, m), 2.93 (6H, s), 2.03-2.32 (2H, m), 1.14-1.26 (6H, m); HPLC: 100%, MS (ESI): m/z 606.2 [M + H]⁺. |
| 21 | 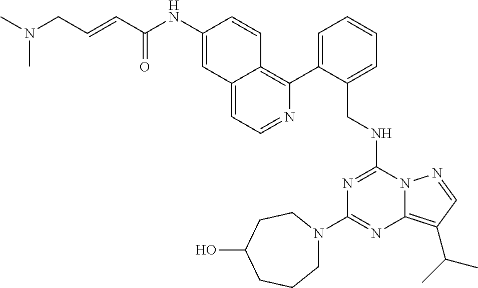 [SMILES: CC(C)c1cnn2c(NCc3ccccc3-c3nccc4cc(NC(=O)/C=C/CN(C)C)ccc34)nc(N3CCCC(O)CC3)nc12] | Racemic mixture; light yellow solid; ¹H-NMR (MeOD, 400 MHz): δ 8.67 (1H, s), 8.41-8.43 (1H, m), 8.18-8.23 (1H, m), 7.80-7.87 (3H, m), 7.59-7.68 (4H, m), 6.99-7.07 (1H, m), 6.74 (1H, d, J = 15.2 Hz), 4.95-4.99 (1H, m), 4.72-4.81 (1H, m), 4.07 (2H, d, J = 7.2 Hz), 3.42-3.93 (4H, m), 3.09-3.14 (1H, m), 2.96 (6H, s), 2.07-2.22 (1H, m), 1.43-1.98 (5H, m), 1.16-1.19 (6H, m); LCMS: 99.8%, MS (ESI):634.2 m/z [(M + H)]⁺ |
| 22 | 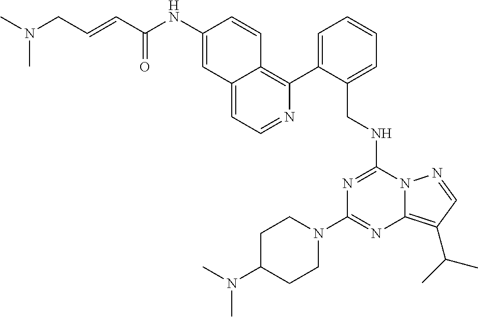 [SMILES: CC(C)c1cnn2c(NCc3ccccc3-c3nccc4cc(NC(=O)/C=C/CN(C)C)ccc34)nc(N3CCC(N(C)C)CC3)nc12] | Light yellow solid; ¹H NMR (400 MHz, CD₃OD): δ 8.68 (1H, s), 8.42 (1H, d, J = 6.0 Hz), 8.21 (1H, d, J = 6.4 Hz), 7.82-7.89 (3H, m), 7.65-7.75 (2H, m), 7.59-7.64 (2H, m), 7.00-7.11 (1H, m), 6.74 (1H, d, J = 15.6 Hz), 4.98-5.07 (1H, m), 4.68-4.75 (1H, m), 4.54-4.62 (2H, m), 4.07 (2H, d, J = 6.8 Hz), 3.59-3.69 (1H, m), 3.08-3.28 (3H, m), 2.98 (6H, s), 2.94 (6H, s), 2.21-2.29 (2H, m), 1.72-1.87 (2H, m), 1.29-1.33 (2H, m), 1.19-1.24 (6H, m); LCMS: 97.9%, MS (ESI): m/z 647.3 [M + H]⁺ |
| 23 | 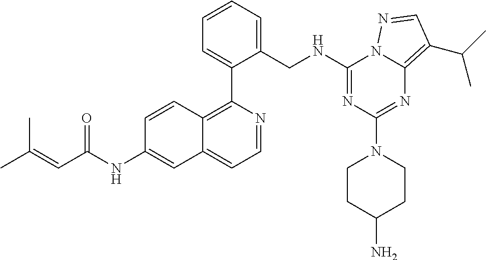 [SMILES: CC(C)=CC(=O)Nc1ccc2c(-c3ccccc3CNc3nc(N4CCC(N)CC4)nc4c(C(C)C)cnn34)nccc2c1] | yellow powder; ¹H NMR (400 MHz, CD₃OD): δ 8.58 (1H, s), 8.38 (1H, s), 8.10-8.19 (1H, m), 7.80-7.90 (2H, m), 7.66-7.74 (2H, m), 7.52-7.63 (3H, m), 6.02 (1H, s), 5.00- 5.09 (1H, m), 4.65-4.72 (1H, m), 4.48-4.56 (2H, m), 3.46- 3.59 (1H, m), 2.97-3.23 (3H, m), 2.33 (3H, s), 2.10-2.22 (2H, m), 2.01 (3H, s), 1.47-1.76 (2H, m), 1.21 (6H, d, J = 6.8 Hz); HPLC: 96.1%, MS (ESI): m/z 590.2 [M + H]⁺ |
| 24 | 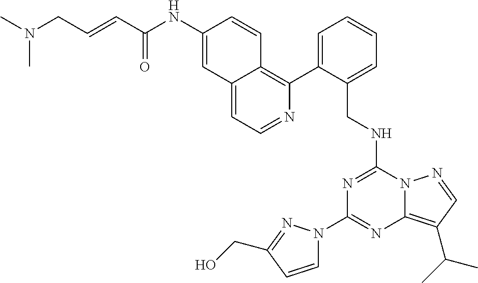 [SMILES: CC(C)c1cnn2c(NCc3ccccc3-c3nccc4cc(NC(=O)/C=C/CN(C)C)ccc34)nc(-n3ccc(CO)n3)nc12] | Yellow powder; ¹H NMR (CD₃OD, 400 MHz): δ 8.51 (1H, s), 8.37 (1H, d, J = 2.8 Hz), 8.11 (1H, d, J = 6.8 Hz), 7.77- 7.89 (4H, m), 7.62-7.71 (3H, m), 7.57 (1H, d, J = 6.8 Hz), 6.97-7.05 (1H, m), 6.58-6.63 (2H, m), 5.03-5.10 (1H, m), 4.76-4.81 (1H, m), 4.74 (2H, s), 4.09 (2H, d, J = 7.2 Hz), 3.08-3.17 (1H, m), 2.99 (6H, s), 1.29-1.34 (6H, m); LCMS: 99.2%, MS (ESI): 617.2 m/z [M + H]⁺ |
| 25 | 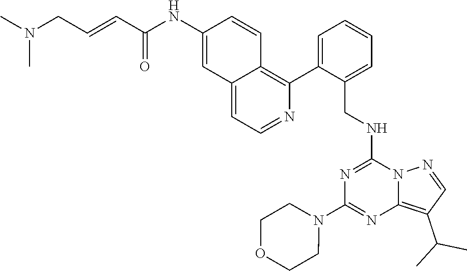 [SMILES: CC(C)c1cnn2c(NCc3ccccc3-c3nccc4cc(NC(=O)/C=C/CN(C)C)ccc34)nc(N3CCOCC3)nc12] | off-white powder; ¹H NMR (400 MHz, CD₃OD): δ 8.65 (1H, d, J = 1.6 Hz), 8.41 (1H, d, J = 6.8 Hz), 8.21 (1H, d, J = 6.4 Hz), 7.77-7.89 (3H, m), 7.55-7.73 (4H, m), 6.97- 7.12 (1H, m), 6.75 (1H, d, J = 15.2 Hz), 4.97-5.03 (1H, m), 4.69-4.76 (1H, m), 4.07 (2H, d, J = 6.8 Hz), 3.66-3.85 (8H, m), 3.04-3.16 (1H, m), 2.96 (6 H, s), 1.16-1.21 (6H, m); HPLC: 99.4%, MS (ESI): m/z 606.2 [M + H]⁺ |
| 26 | 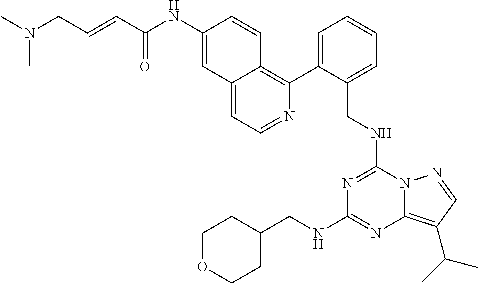 [SMILES: CC(C)c1cnn2c(NCc3ccccc3-c3nccc4cc(NC(=O)/C=C/CN(C)C)ccc34)nc(NCC3CCOCC3)nc12] | White powder; ¹H NMR (CD₃OD, 400 MHz): δ 8.70 (1H, s), 8.46 (1H, d, J = 7.2 Hz), 8.22-8.28 (1H, m), 7.82-7.89 (3H, m), 7.57-7.76 (4H, m), 7.00-7.09 (1H, m), 6.71 (1H, d, J = 15.2 Hz), 4.96-5.02 (1H, m), 4.75-4.83 (1H, m), 4.08 (2H, d, J = 6.4 Hz), 3.84-4.01 (2H, m), 3.32-3.39 (2H, m), 3.23-3.28 (1H, m), 3.06-3.21 (1H, m), 2.98 (6H, s), 2.83-2.88 (1H, m), 1.73-1.80 (1H, m), 1.55-1.64 (2H, m), 1.25-1.32 (1H, m), 1.18-1.24 (6H, m); LCMS: 100%, MS (ESI): m/z 634.2 [M + H]⁺ |
| 27 | 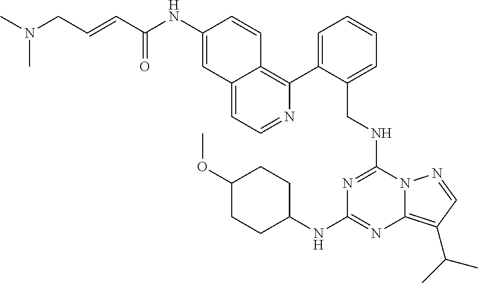 [SMILES: COC1CCC(Nc2nc(NCc3ccccc3-c3nccc4cc(NC(=O)/C=C/CN(C)C)ccc34)n3ncc(C(C)C)c3n2)CC1] | Light yellow solid; ¹H-NMR (CD₃OD, 400 MHz): δ 8.88- 6.89 (1H, m), 8.41-8.43 (1H, m), 8.26-8.28 (1H, m), 7.78- 7.88 (3H, m), 7.67-7.71 (1H, m), 7.55-7.60 (3H, m), 6.99- 7.07 (1H, m), 6.71 (1H, d, J = 15.2 Hz ), 4.95-4.99 (1H, m), 4.78-4.82 (1H, m), 4.07 (2H, d, J = 7.2 Hz), 3.52-3.57 (1H, m), 3.40 (3H, s), 3.22-3.28 (1H, m), 2.96 (6H, s), 2.80-2.87 (1H, m), 2.03-2.17 (3H, m), 1.71-1.73 (1H, m), 1.27-1.48 (4H, m), 1.16-1.19 (6H, m); LCMS: 100.0%, MS (ESI): 648.3 m/z [(M + H)]⁺ |
| 28 | 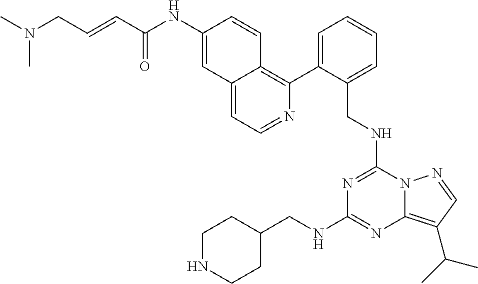 [SMILES: CC(C)c1cnn2c(NCc3ccccc3-c3nccc4cc(NC(=O)/C=C/CN(C)C)ccc34)nc(NCC3CCNCC3)nc12] | Light yellow solid; ¹H-NMR (CD₃OD, 400 MHz): δ 8.68 (1H, s), 8.45 (1H, d, J = 6.4 Hz), 8.23 (1H, d, J = 6.8 Hz), 7.82-7.83 (3H, m), 7.68-7.72 (1H, m), 7.59-7.65 (3H, m), 6.99-7.05 (1H, m), 6.72 (1H, d, J = 15.2 Hz), 5.04-5.08 (1H, m), 4.77-4.81 (1H, m), 4.07 (2H, d, J = 6.8 Hz), 3.37- 3.48 (3H, m), 3.18-3.27 (1H, m), 2.96-3.02 (8H, m), 2.83- 2.88 (1H, m), 1.93-1.96 (m, 3H), 1.49-1.55 (2H, m), 1.18- 1.21 (6H, m); LCMS: 100.0%, MS (ESI): 633.3 m/z [(M + H)]⁺ |
| 29 | 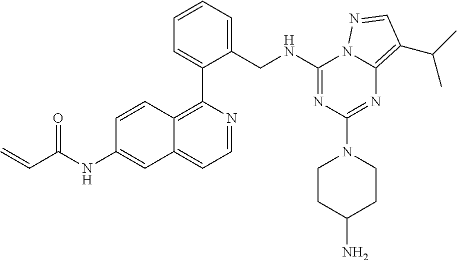 [SMILES: C=CC(=O)Nc1ccc2c(-c3ccccc3CNc3nc(N4CCC(N)CC4)nc4c(C(C)C)cnn34)nccc2c1] | off-white powder; ¹H NMR (400 MHz, CD₃OD): δ 8.59 (1H, d, J = 2.0 Hz), 8.38 (1H, d, J = 6.4 Hz), 8.16 (1H, d, J = 6.4 Hz), 7.79-7.88 (2H, m), 7.66-7.78 (2H, m), 7.57-7.62 (2H, m), 7.53 (1H, s), 6.51-6.58 (2H, m), 5.94 (1H, dd, J = 8.0, 3.6 Hz), 4.98-5.09 (1H, m), 4.65-4.74 (1H, m), 4.49-4.57 (2H, m), 3.43-3.54 (1H, m), 2.95-3.21 (3H, m), 2.04-2.21 (2H, m), 1.44-1.76 (2H, m), 1.20 (6H, d, J = 6.8 Hz); HPLC: 100%, MS (ESI): m/z 562.2 [M + H]⁺. |
| 30 | 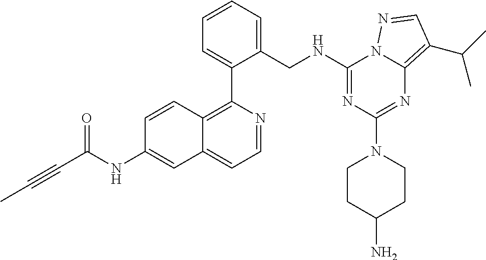 [SMILES: CC#CC(=O)Nc1ccc2c(-c3ccccc3CNc3nc(N4CCC(N)CC4)nc4c(C(C)C)cnn34)nccc2c1] | Light yellow powder; ¹H NMR (CD₃OD, 400 MHz): δ 8.43 (1H, s), 8.39 (1H, d, J = 6.4 Hz), 8.17 (1H, d, J = 6.4 Hz), 7.78-7.84 (2H, m), 7.62-7.71 (2H, m), 7.56-7.62 (2H, m), 7.55 (1H, s), 4.91-5.05 (1H, m), 4.63-4.71 (1H, m), 4.47-4.551 (2H, m), 3.43-3.56 (1H, m), 3.10-3.29 (2H, m), 3.00-3.08 (1H, m), 2.02-2.21 (5H, m), 1.63- 1.76 (1H, m), 1.51-1.59 (1H, m), 1.21-1.27 (6H, m); LCMS: 96.8%, MS (ESI): m/z 574.2 [M + H]⁺ |
| 31 | 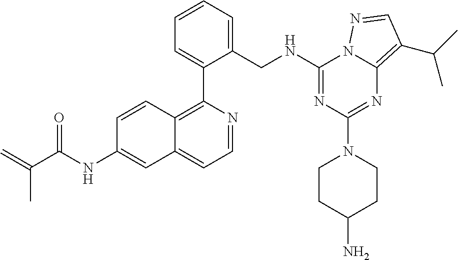 [SMILES: C=C(C)C(=O)Nc1ccc2c(-c3ccccc3CNc3nc(N4CCC(N)CC4)nc4c(C(C)C)cnn34)nccc2c1] | Pale yellow powder; ¹H NMR (CD₃OD, 400 MHz): δ 8.55 (1H, d, J = 2.0 Hz), 8.37-8.42 (1H, m), 8.20 (1H, d, J = 6.4 Hz), 7.77-7.92 (3H, m), 7.64-7.72 (1H, m), 7.54- 7.62 (3H, m), 5.98 (1H, s), 5.71 (1H, d, J = 1.2 Hz), 4.99- 5.07 (1H, m), 4.67-4.73 (1H, m), 4.47-4.54 (1H, m), 3.44-3.56 (1H, m), 3.17-3.24 (1H, m), 3.03-3.09 (1H, m), 2.08-2.21 (2H, m), 2.06 (3H, s), 1.64-1.78 (1H, m), 1.55-1.60 (1H, m), 1.16-1.21 (6H, m); LCMS: 100%, MS (ESI): m/z 576.2 [M + H]⁺ |
| 32 | 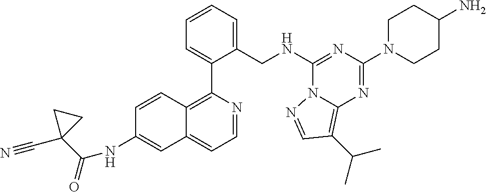 [SMILES: CC(C)c1cnn2c(NCc3ccccc3-c3nccc4cc(NC(=O)C5(C#N)CC5)ccc34)nc(N3CCC(N)CC3)nc12] | White powder; ¹H NMR (CD₃OD, 400 MHz): δ 8.48 (1H, d, J = 1.6 Hz), 8.40 (1H, d, J = 6.8 Hz), 8.19 (1H, d, J = 6.8 Hz), 7.80-7.89 (3H, m), 7.63-7.72 (2H, m), 7.55-7.62 (2H, m), 4.98-5.07 (1H, m), 4.68-4.75 (1H, m), 4.51-4.57 (2H, m), 3.46-3.52 (1H, m), 3.13-3.23 (2H, m), 3.04-3.09 (1H, m), 2.08-2.17 (2H, m), 1.74-1.87 (4H, m), 1.55-1.72 (2H, m), 1.23-1.28 (6H, m); LCMS: 99.8%, MS (ESI): 601.2 m/z [M + H]⁺ |
| 33 | 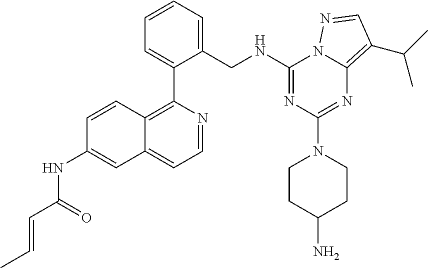 [SMILES: C/C=C/C(=O)Nc1ccc2c(-c3ccccc3CNc3nc(N4CCC(N)CC4)nc4c(C(C)C)cnn34)nccc2c1] | Yellow powder; ¹H NMR (CD₃OD, 400 MHz): δ 8.57 (1H, d, J = 2.0 Hz), 8.41 (1H, d, J = 6.4 Hz), 8.19 (1H, d, J = 6.8 Hz), 7.73-7.88 (3H, m), 7.66-7.71 (1H, m), 7.56- 7.62 (3H, m), 7.06-7.17 (1H, m), 6.25 (1H, dd, J = 14.8, 2.0 Hz), 4.99-5.06 (1H, m), 4.66-4.73 (1H, m), 4.45- 4.52 (2H, m), 3.47-3.58 (1H, m), 3.18-3.38 (2H, m, overlap with CD3OD signal), 3.0-3.09 (1H, m), 2.10- 2.19 (2H, m), 1.98 (3H, dd, J = 6.8, 1.6 Hz), 1.65-1.80 (1H, m), 1.49-1.64 (1H, m), 1.18 (6H, d, J = 6.8 Hz); LCMS: 100%, MS (ESI): m/z 576.3 [M + H]⁺ |
| 34 | 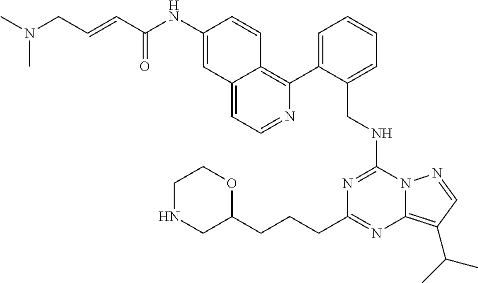 [SMILES: CC(C)c1cnn2c(NCc3ccccc3-c3nccc4cc(NC(=O)/C=C/CN(C)C)ccc34)nc(CCCC3CNCCO3)nc12] | Racemic mixture; off-white powder; ¹H NMR (400 MHz, CD₃OD): δ 8.71 (1H, s), 8.48 (1H, d, J = 6.4 Hz), 8.27 (1H, d, J = 6.0 Hz), 7.81-7.92 (3H, m), 7.56-7.75 (4H, m), 7.00- 7.13 (1H, m), 6.77 (1H, d, J = 15.2 Hz), 5.06-5.14 (1H, m), 4.72-4.79 (1H, m), 4.06-4.21 (3H, m), 3.86-3.97 (2H, m), 3.43-3.59 (2H, m), 3.25-3.33 (3H, m), 3.14-3.26 (1H, m), 2.98 (6H, s), 2.83-2.94 (1H, m), 1.57-1.82 (2H, m), 1.21 (6H, d, J = 6.4 Hz); HPLC: 100%, MS (ESI): m/z 649.3[M + H]⁺ |
| 35 | 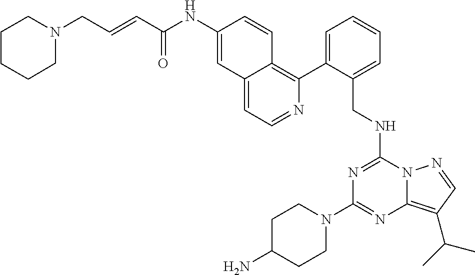 [SMILES: CC(C)c1cnn2c(NCc3ccccc3-c3nccc4cc(NC(=O)/C=C/CN5CCCCC5)ccc34)nc(N3CCC(N)CC3)nc12] | yellow powder; ¹H NMR (400 MHz, CD₃OD): δ 8.69 (1H, s), 8.47 (1H, s), 8.26 (1H, d, J = 5.2 Hz), 7.80-7.93 (3H, m), 7.56-7.76 (4H, m), 7.02-7.14 (1H, m), 6.76 (1H, d, J = 14.8 Hz), 5.08-5.18 (1H, m), 4.69-4.78 (1H, m), 4.47- 4.56 (2H, m), 4.03-4.08 (2H, m), 3.48-3.68 (5H, m), 2.94- 3.18 (3H, m), 2.11-2.23 (2H, m), 2.00-2.07 (2H, m), 1.80- 1.92 (4H, m), 1.51-1.67 (2H, m), 1.20 (6H, d, J = 6.0 Hz); HPLC: 97.1%, MS (ESI): m/z 659.3 [M + H]⁺ |
| 36 | 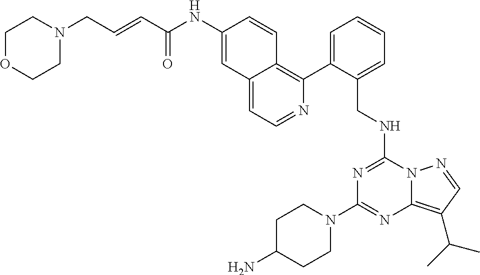 [SMILES: CC(C)c1cnn2c(NCc3ccccc3-c3nccc4cc(NC(=O)/C=C/CN5CCOCC5)ccc34)nc(N3CCC(N)CC3)nc12] | yellow powder; ¹H NMR (400 MHz, CD₃OD): δ 8.69 (1H, s), 8.45 (1H, d, J = 5.2 Hz), 8.25 (1H, d, J = 6.4 Hz), 7.81- 7.96 (3H, m), 7.59-7.76 (4H, m), 7.02-7.17 (1H, m), 6.79 (1H, d, J = 15.2 Hz), 5.01-5.08 (1H, m), 4.73-4.78 (1H, m), 4.48-4.55 (2H, m), 4.05-4.21 (4H, m), 3.79-3.92 (2H, m), 3.48-3.63 (4H, m), 3.12-3.28 (4H, m), 2.11-2.27 (2H, m), 1.56-1.85 (2H, m), 1.20 (6H, d, J = 6.0 Hz); HPLC: 100%, MS (ESI): m/z 661.3 [M + H]⁺ |
| 37 | 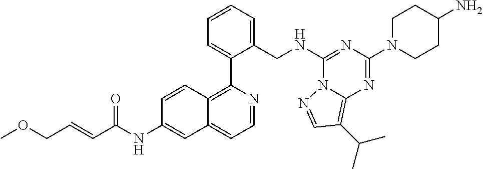 [SMILES: COC/C=C/C(=O)Nc1ccc2c(-c3ccccc3CNc3nc(N4CCC(N)CC4)nc4c(C(C)C)cnn34)nccc2c1] | off-white powder; ¹H NMR (CD₃OD, 400 MHz): δ 8.56 (1H, s), 8.31-8.36 (1H, m), 8.08-8.13 (1H, m), 7.77-7.90 (2H, m), 7.42-7.75 (5H, m), 7.06-7.13 (1H, m), 6.46 (1H, d, J = 15.2 Hz), 4.64-4.71 (1H, m), 4.52-4.58 (2H, m), 4.14-4.23 (2H, m), 3.44-3.53 (4H, m), 2.87-3.19 (3H, m), 2.04-2.13 (2H, m), 1.29-1.72 (3H, m), 1.20 (6H, d, J = 6.8 Hz); LCMS: 99.8%, MS (ESI):606.2 m/z [M + H]+ |
| 38 | 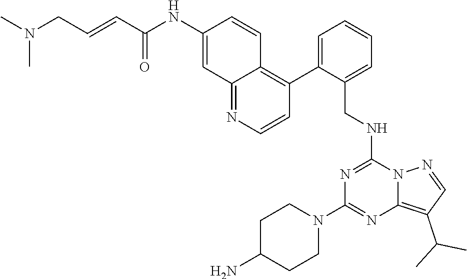 [SMILES: CC(C)c1cnn2c(NCc3ccccc3-c3ccnc4cc(NC(=O)/C=C/CN(C)C)ccc34)nc(N3CCC(N)CC3)nc12] | yellow powder; ¹H NMR (400 MHz, DMSO-d₆): δ ppm 10.95 (1H, brs) 8.87 (1H, d, J = 4.8 Hz), 8.65-8.72 (1H, m), 8.64 (1H, s), 8.13 (2H, brs), 7.65-7.75 (2H, m), 7.39- 7.57 (3H, m), 7.24-7.38 (3H, m), 6.82-6.95 (1H, m), 6.62 (1H, d, J = 14.8 Hz), 4.18-4.48 (4H, m), 3.76-3.88 (2H, m), 3.19-3.26 (1H, m), 2.83-2.94 (1H, m), 2.55-2.76 (9H, m), 1.86 (2H, m), 1.28-1.39 (2H, m), 1.20 (6H, d, J = 6.8 Hz); LCMS: 100%, MS (ESI): m/z 619.3 [M + H]⁺ |
| 39 | 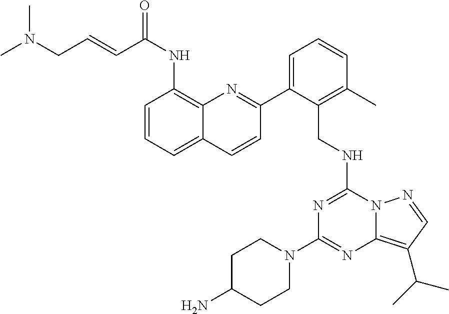 [SMILES: Cc1cccc(-c2ccc3cccc(NC(=O)/C=C/CN(C)C)c3n2)c1CNc1nc(N2CCC(N)CC2)nc2c(C(C)C)cnn12] | yellow solid; ¹H-NMR (CD₃OD, 400 MHz): δ 8.70-8.72 (1H, m), 8.29-8.33 (1H, m), 7.78 (1H, s), 7.61-7.72 (3H, m), 7.42-7.48 (2H, m), 7.32-7.34 (1H, m), 6.86-6.92 (1H, m), 6.72-6.75 (1H, m), 4.32-4.36 (2H, m), 4.03 (2H, d, J = 7.2 Hz), 3.45-3.49 (1H, m), 3.02-3.15 (3H, m), 2.94 (6H, s), 2.61 (3H, s), 2.09-2.12 (2H, m), 1.55-1.61 (2H, m), 1.26 (6H, d, J = 6.8 Hz); LCMS: 100.0%, MS (ESI): 633.3 m/z [(M + H)]⁺ |
| 40 | 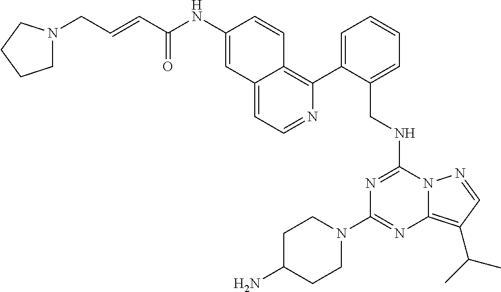 [SMILES: CC(C)c1cnn2c(NCc3ccccc3-c3nccc4cc(NC(=O)/C=C/CN5CCCC5)ccc34)nc(N3CCC(N)CC3)nc12] | off-white powder; ¹H NMR (400 MHz, CD₃OD): δ 8.63 (1H, s), 8.32-8.37 (1H, m), 8.14 (1H, d, J = 6.4 Hz), 7.80- 7.87 (2H, m), 7.72-7.76 (1H, m), 7.68 (1H, t, J = 8.0 Hz ), 7.54-7.63 (2H, m), 7.48 (1H, s), 6.96-7.15 (1H, m), 6.72 (1H, d, J = 15.20 Hz), 4.66-4.73 (2H, m), 4.53-4.59 (2H, m), 4.16 (2H, d, J = 6.8 Hz), 3.69-3.74 (2H, m), 3.42-3.53 (1H, m), 2.93-3.25 (5H, m), 2.03-2.32 (6H, m), 1.43-1.69 (2H, m), 1.20 (6H, d, J = 7.2 Hz); HPLC: 100%, MS (ESI): m/z 645.3 [M + H]⁺ |
| 41 | 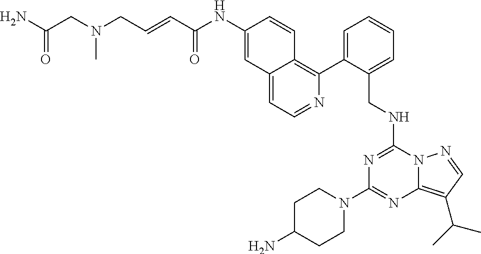 [SMILES: CC(C)c1cnn2c(NCc3ccccc3-c3nccc4cc(NC(=O)/C=C/CN(C)CC(N)=O)ccc34)nc(N3CCC(N)CC3)nc12] | yellow powder; ¹H NMR (400 MHz, CD₃OD): δ 8.63 (1H, s), 8.37 (1H, d, J = 6.4 Hz), 8.16 (1H, d, J = 6.2 Hz), 7.73- 7.88 (3H, m), 7.50-7.71 (4H, m), 6.95-7.08 (1H, m), 6.72 (1H, d, J = 15.2 Hz), 4.95-5.03 (1H, m), 4.65-4.71 (1H, m), 4.46-4.54 (2H, m), 3.99-4.19 (4H, m), 3.42-3.50 (1H, m), 3.10-3.17 (2H, m), 2.91-3.07 (4H, m), 2.05-2.13 (2H, m), 1.43-1.74 (2H, m), 1.16 (6H, d, J = 6.8 Hz); HPLC: 100%, MS (ESI): m/z 662.3 [M + H]⁺ |
| 42 | 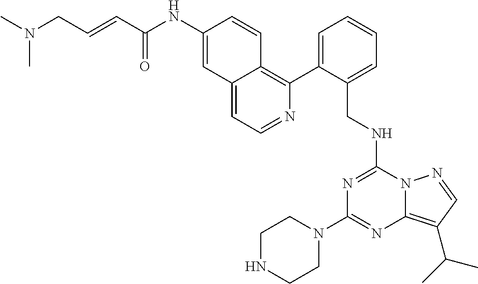 [SMILES: CC(C)c1cnn2c(NCc3ccccc3-c3nccc4cc(NC(=O)/C=C/CN(C)C)ccc34)nc(N3CCNCC3)nc12] | pale yellow powder; ¹H NMR (CD₃OD, 400 MHz): δ 8.61 (1H, d, J = 1.6 Hz), 8.33 (1H, d, J = 6.8 Hz), 8.11 (1H, d, J = 6.4 Hz), 7.77-7.85 (2H, m), 7.72-7.76 (1H, m), 7.64-7.70 (1H, m), 7.54-7.63 (2H, m), 7.48 (1H, s), 6.97-7.07 (1H, m), 6.72 (1H, d, J = 15.2 Hz), 4.96-5.03 (2H, m), 4.67-4.74 (1H, m), 4.05-4.09 (2H, m), 3.91- 4.00 (4H, m), 3.26-3.32 (4H, m), 2.94-2.99 (7H, m), 1.20 (6H, d, J = 7.2 Hz); LCMS: 100%, MS (ESI): m/z 605.6 [M + H]⁺ |
| 43 | 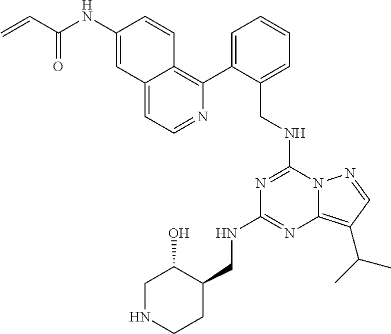 [SMILES: C=CC(=O)Nc1ccc2c(-c3ccccc3CNc3nc(NC[C@H]4CCNC[C@@H]4O)nc4c(C(C)C)cnn34)nccc2c1] | (3R, 4R); off-white powder; ¹H NMR (400 MHz, CD₃OD): δ 8.54 (1H, s), 8.33-8.37 (1H, m), 8.06-8.12 (1H, m), 7.67-7.78 (2H, m), 7.51-7.64 (3H, m), 7.44-7.50 (2H, m), 6.38-6.44 (2H, m), 5.80-5.86 (1H, m), 4.94-5.05 (2H, m), 4.52-4.72 (2H, m), 3.49-3.75 (2H, m), 3.32-3.38 (1H, m), 2.64-2.95 (3H, m), 1.85-2.01 (1H, m), 1.71-1.79 (1H, m), 1.42-1.50 (1H, m), 1.09 (6H, d, J = 6.8 Hz); HPLC: 97.5%, MS (ESI): m/z 592.3 [M + H]⁺ |
| 44 | 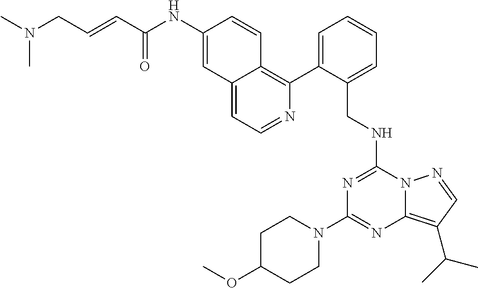 [SMILES: COC1CCN(c2nc(NCc3ccccc3-c3nccc4cc(NC(=O)/C=C/CN(C)C)ccc34)n3ncc(C(C)C)c3n2)CC1] | pale yellow powder; ¹H NMR (400 MHz, CD₃OD): δ 8.64 (1H, s), 8.37 (1H, d, J = 6.8 Hz), 8.16 (1H, d, J = 6.8 Hz), 7.76-7.86 (3H, m), 7.65-7.70 (1H, m), 7.54-7.64 (3H, m), 6.98-7.07 (1H, m), 6.72 (1H, d, J = 15.2 Hz), 4.95-5.01 (1H, m), 4.81-4.87 (1H, m), 4.68-4.74 (1H, m), 4.07 (2H, d, J = 7.2 Hz), 3.85-3.99 (2H, m), 3.52- 3.64 (3H, m), 3.40 (3H, s), 3.00-3.09 (1H, m), 2.96 (6H, s), 2.91 (1H, s), 1.83-2.05 (3H, m), 1.57-1.69 (2H, m), 1.15-1.20 (6H, m); LCMS: 99.4%, MS (ESI): m/z 634.3 [M + H]⁺ |
| 45 | 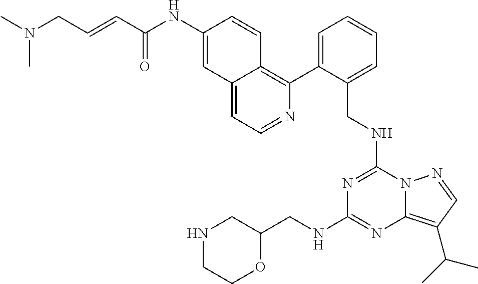 [SMILES: CC(C)c1cnn2c(NCc3ccccc3-c3nccc4cc(NC(=O)/C=C/CN(C)C)ccc34)nc(NCC3CNCCO3)nc12] | Racemic mixture; yellow solid; ¹H-NMR (CD₃OD, 400 MHz): δ 8.65-8.69 (1H, m), 8.43-8.47 (1H, m), 8.18-8.24 (1H, m), 7.78-7.88 (3H, m), 7.65-7.72 (2H, m), 7.57-7.61 (2H, m), 6.98-7.06 (1H, m), 6.73 (1H, d, J = 15.2 Hz), 4.99-5.11 (2H, m), 4.70-4.81 (1H, m), 3.84-4.12 (5H, m), 3.38-3.65 (3H, m), 2.81-3.24 (9H, m), 1.16-1.22 (6H, m); LCMS: 100%, MS (ESI):657.2 m/z [M + Na]⁺ |
| 46 | 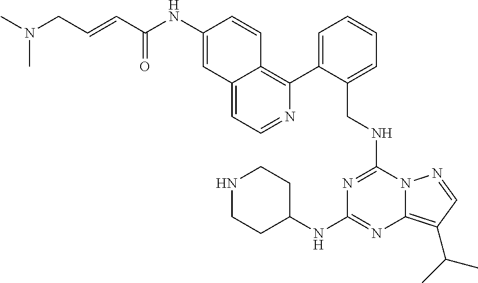 [SMILES: CC(C)c1cnn2c(NCc3ccccc3-c3nccc4cc(NC(=O)/C=C/CN(C)C)ccc34)nc(NC3CCNCC3)nc12] | yellow powder; ¹H NMR (CD₃OD, 400 MHz): δ 8.63 (1H, s), 8.39-8.45 (1H, m), 8.11-8.17 (1H, m), 7.50-7.91 (7H, m), 6.96-7.13 (1H, m), 6.72 (1H, d, J = 15.4 Hz), 5.09- 5.16 (1H, m), 3.97-4.16 (3H, m), 3.37-3.62 (3H, m), 3.12- 3.27 (1H, m), 2.77-3.05 (8H, m), 2.24-2.31 (1H, m), 1.68- 2.05 (3H, m), 1.12-1.29 (6H, m); LCMS: 100%, MS (ESI): 619.3 m/z [M + H]⁺ |
| 47 | 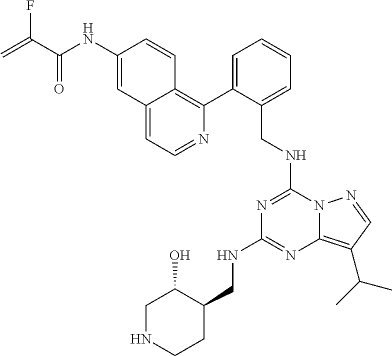 [SMILES: C=C(F)C(=O)Nc1ccc2c(-c3ccccc3CNc3nc(NC[C@H]4CCNC[C@@H]4O)nc4c(C(C)C)cnn34)nccc2c1] | (3R, 4R); yellow solid; ¹H-NMR (CD₃OD, 400 MHz): δ 8.64-8.68 (1H, m), 8.51 (1H, dd, J = 6.4, 3.2 Hz), 8.27- 8.31 (1H, m), 7.81-7.92 (3H, m), 7.65-7.72 (2H, m), 7.57- 7.62 (2H, m), 5.87 (1H, dd, J = 46.0, 3.6 Hz), 5.46 (1H, dd, J = 14.8, 3.6 Hz), 5.06-5.13 (1H, m), 4.65-4.75 (1H, m), 3.69-3.76 (1H, m), 3.54-3.62 (1H, m), 3.34-3.47 (3H, m), 2.78-3.01 (3H, m), 1.98-2.05 (1H, m), 1.81-1.92 (1H, m), 1.53-1.65 (1H, m), 1.20 (6H, d, J = 6.8 Hz); LCMS: 99.4%, MS (ESI): 610.2 m/z [M + H]⁺ |
| 48 | 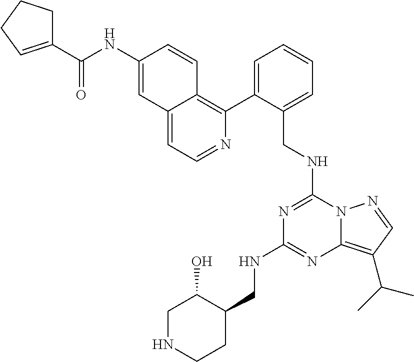 [SMILES: CC(C)c1cnn2c(NCc3ccccc3-c3nccc4cc(NC(=O)C5=CCCC5)ccc34)nc(NC[C@H]3CCNC[C@@H]3O)nc12] | (3R, 4R); yellow solid; ¹H-NMR (CD₃OD, 400 MHz): δ 8.58-8.61 (1H, m), 8.44 (1H, dd, J = 6.4, 2.4 Hz), 8.17- 8.22 (1H, m), 7.79-7.91 (3H, m), 7.56-7.72 (4H, m), 6.92 (1H, d, J = 2.0 Hz), 5.05-5.13 (1H, m), 4.68-4.75 (1H, m), 3.69-3.76 (1H, m), 3.37-3.59 (4H, m), 2.60-3.01 (7H, m), 1.96-2.07 (3H, m), 1.79-1.88 (1H, m), 1.52-1.63 (1H, m), 1.18 (6H, d, J = 6.8 Hz); LCMS: 98.3%, MS (ESI): 632.2 m/z [M + H]⁺ |
| 49 | 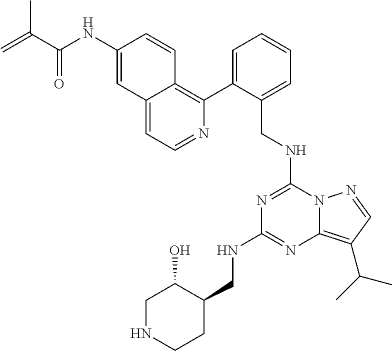 [SMILES: C=C(C)C(=O)Nc1ccc2c(-c3ccccc3CNc3nc(NC[C@H]4CCNC[C@@H]4O)nc4c(C(C)C)cnn34)nccc2c1] | (3R, 4R); yellow solid; ¹H-NMR (CD₃OD, 400 MHz): δ 8.52-8.59 (1H, m), 8.39-8.45 (1H, m), 8.04-8.13 (1H, m), 7.52-7.88 (7H, m), 5.94 (1H, s), 5.69 (1H, s), 5.08-5.17 (1H, m), 4.62-4.73 (1H, m), 3.34-3.76 (5H, m), 2.76-3.05 (3H, m), 1.52-2.10 (6H, m), 1.20 (6H, d, J = 6.8 Hz); LCMS: 98.9%, MS (ESI): 606.2 m/z [M + H]⁺ |
| 50 | 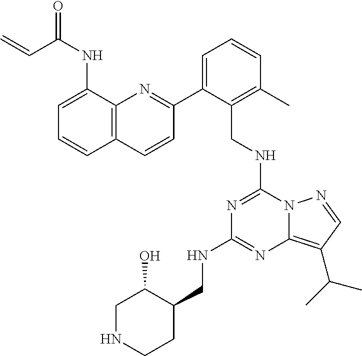 [SMILES: C=CC(=O)Nc1cccc2ccc(-c3cccc(C)c3CNc3nc(NC[C@H]4CCNC[C@@H]4O)nc4c(C(C)C)cnn34)nc12] | (3R, 4R); light yellow powder; ¹H-NMR (CD₃OD, 400 MHz): δ 8.73 (1H, d, J = 8.0 Hz), 8.32 (1H, d, J = 8.8 Hz), 7.74 (1H, s), 7.59-7.69 (3H, m), 7.46-7.48 (2H, m), 7.35- 7.37 (1H, m), 6.49-6.56 (1H, m), 6.32-6.36 (1H, m), 5.69- 5.76 (1H, m), 4.91-4.95 (3H, m), 4.84-4.86 (1H, m), 3.60- 3.66 (1H, m), 3.34-3.39 (1H, m), 3.25-3.28 (1H, m), 2.84- 2.94 (2H, m), 2.69-2.75 (1H, m), 2.57 (3H, s), 1.88-1.94 (1H, m), 1.73-1.77 (1H, m), 1.43-1.47 (1H, m), 1.26 (6H, d, J = 6.8 Hz); LCMS: 100.0%, MS (ESI):606.3 m/z [(M + H)]⁺ |
| 51 | 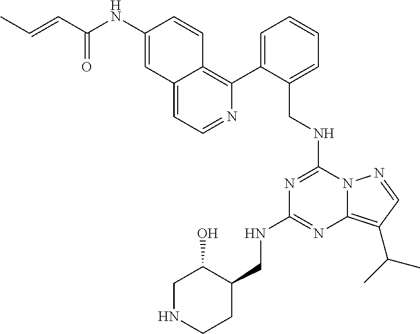 [SMILES: C/C=C/C(=O)Nc1ccc2c(-c3ccccc3CNc3nc(NC[C@H]4CCNC[C@@H]4O)nc4c(C(C)C)cnn34)nccc2c1] | (3R, 4R); yellow solid; ¹H-NMR (CD₃OD, 400 MHz): δ 8.59-8.63 (1H, m), 8.42-8.47 (1H, m), 8.17-8.22 (1H, m), 7.55-7.73 (7H, m), 7.06-7.12 (1H, m), 6.22 (1H, d, J = 15.2 Hz), 5.05-5.12 (1H, m), 4.64-4.75 (1H, m), 3.69- 3.76 (1H, m), 3.30-3.58 (4H, m), 2.89-3.01 (1H, m), 2.75- 2.84 (2H, m), 1.95-2.04 (4H, m), 1.80-1.91 (1H, m), 1.52- 1.63 (1H, m), 1.18 (6H, d, J = 6.8 Hz); LCMS: 100%, MS (ESI): 606.3 m/z [M + H]⁺ |
| 52 | 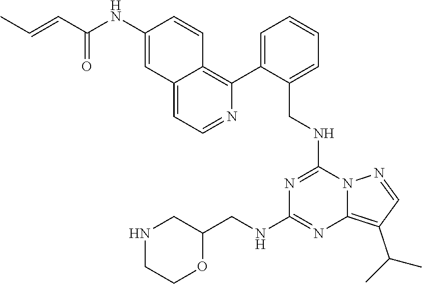 [SMILES: C/C=C/C(=O)Nc1ccc2c(-c3ccccc3CNc3nc(NCC4CNCCO4)nc4c(C(C)C)cnn34)nccc2c1] | Racemic mixture; yellow solid; ¹H-NMR (CD₃OD, 400 MHz): δ 8.54-8.61 (1H, m), 8.42 (1H, d, J = 6.8 Hz), 8.14- 8.18 (1H, m), 7.55-7.87 (7H, m), 7.06-7.14 (1H, m), 6.22 (1H, d, J = 15.2 Hz), 5.03-5.13 (1H, m), 4.67-4.72 (1H, m), 3.81-4.15 (3H, m), 3.34-3.61 (3H, m), 2.79-3.25 (3H, m), 1.97 (3H, d, J = 6.8 Hz), 1.16-1.21 (6H, m); LCMS: 100%, MS (ESI): 592.3 m/z [M + H]⁺ |
| 53 | 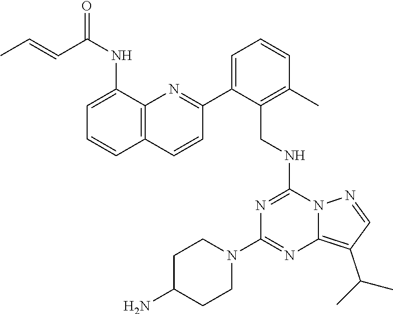 | Yellow powder; ¹H-NMR (CD₃OD, 400 MHz): δ 8.54 (1H, d, J = 7.6 Hz), 8.45 (1H, d, J = 8.8 Hz), 7.65-7.77 (4H, m), 7.45-7.52 (2H, m), 7.35-7.37 (1H, m), 6.91-6.97 (1H, m), 6.22 (1H, d, J = 14.8 Hz), 4.93-4.94 (2H, m), 3.64-3.65 (1H, m), 3.32-3.36 (2H, m), 3.26-3.28 (2H, m), 2.85-2.92 (2H, m), 2.74-2.77 (1H, m), 2.62 (3H, s), 1.87-1.95 (3H, m), 1.70-1.77 (1H, m), 1.44-1.52 (1H, m), 1.25 (6H, d, J = 6.8 Hz), 0.85-0.91 (1H, m); LCMS: 98.5%, MS (ESI): 620.2 m/z [(M + H)]⁺ |
| 54 | 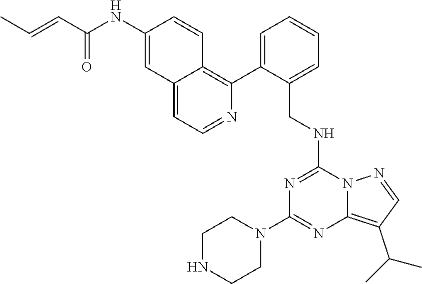 [SMILES: C/C=C/C(=O)Nc1ccc2c(-c3ccccc3CNc3nc(N4CCNCC4)nc4c(C(C)C)cnn34)nccc2c1] | yellow solid; ¹H-NMR (CD₃OD, 400 MHz): δ 8.57 (1H, d, J = 2.0 Hz), 8.38 (1H, d, J = 6.8 Hz), 8.16 (1H, d, J = 6.8 Hz), 7.79-7.86 (2H, m), 7.66-7.74 (2H, m), 7.57-7.62 (3H, m), 7.07-7.14 (1H, m), 6.25 (1H, dd, J = 15.2, 1.6 Hz), 5.01-5.05 (1H, m), 4.68-4.72 (1H, m), 4.01-4.05 (4H, m), 3.33-3.36 (4H, m), 3.00-3.07 (1H, m), 1.98 (3H, dd, J = 6.8, 1.6 Hz), 1.20 (6H, dd, J = 6.8, 1.6 Hz); LCMS: 100.0%, MS (ESI): 562.2 m/z [(M + H)]⁺ |
| 55 | 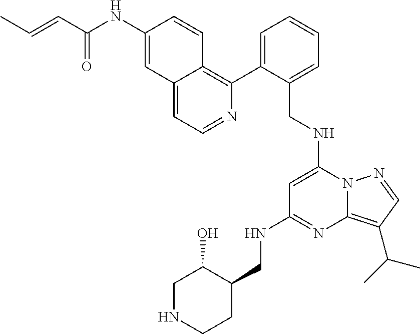 [SMILES: C/C=C/C(=O)Nc1ccc2c(-c3ccccc3CNc3cc(NC[C@H]4CCNC[C@@H]4O)nc4c(C(C)C)cnn34)nccc2c1] | (3R, 4R); yellow powder; ¹H-NMR (CD₃OD, 400 MHz): δ 8.46-8.52 (1H, m), 8.36-8.38 (1H, m), 8.05-8.08 (1H, m), 7.78-7.87 (2H, m), 7.65-7.69 (2H, m), 7.49-7.55 (3H, m), 7.05-7.12 (1H, m), 6.24 (1H, dd, J = 15.2, 1.2 Hz), 4.99- 5.03 (1H, m), 4.65-4.72 (1H, m), 3.67-3.73 (1H, m), 3.38- 3.48 (4H, m), 2.98-3.05 (1H, m), 2.81-2.91 (2H, m), 2.4- 2.09 (1H, m), 1.97 (3H, dd, J = 6.8, 1.2 Hz), 1.81-1.86 (1H, m), 1.62-1.68 (1H, m), 1.17-1.22 (6H, m); LCMS: 99.4%, MS (ESI): 605.3 m/z [(M + H)]⁺ |
| 56 | 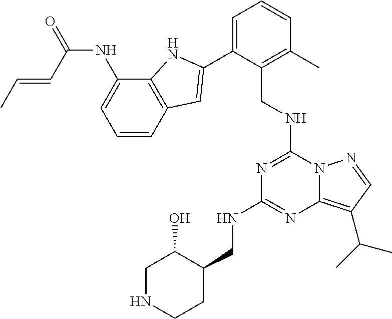 [SMILES: C/C=C/C(=O)Nc1cccc2cc(-c3cccc(C)c3CNc3nc(NC[C@H]4CCNC[C@@H]4O)nc4c(C(C)C)cnn34)[nH]c12] | (3R, 4R); off-white powder; ¹H NMR (CD₃OD, 400 MHz): δ 7.85 (1H, s), 7.36-7.45 (3H, m), 7.34 (1H, d, J = 8.0 Hz), 7.19 (1H, d, J = 7.6 Hz), 6.97-7.05 (1H, m), 6.60-6.74 (1H, m), 6.17 (1H, d, J = 15.2 Hz), 4.93-4.97 (2H, m), 3.55- 3.61 (1H, m), 3.45-3.54 (1H, m), 3.35-3.43 (1H, m), 3.23- 3.27 (1H, m), 3.03-3.09 (1H, m), 2.94-3.01 (1H, m), 2.57- 2.68 (2H, m), 2.54 (3H, s), 1.88 (3H, d, J = 6.4 Hz), 1.64- 1.84 (2H, m), 1.34-1.45 (1H, m), 1.30 (6H, d, J = 6.8 Hz); LCMS: 100%, MS (ESI): m/z 608.3 [M + H]⁺ |
| 57 | 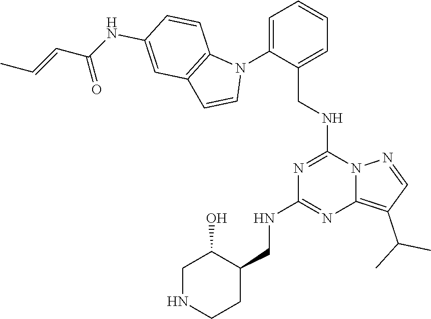 [SMILES: C/C=C/C(=O)Nc1ccc2c(ccn2-c2ccccc2CNc2nc(NC[C@H]3CCNC[C@@H]3O)nc3c(C(C)C)cnn23)c1] | (3R, 4R); white powder; ¹H NMR (CD₃OD, 400 MHz): δ 7.93 (1H, s), 7.68-7.84 (2H, m), 7.59 (2H, m), 7.36 (1H, d, J = 7.2 Hz), 7.23-7.30 (1H, m), 6.89-7.04 (2H, m), 6.77- 6.83 (1H, m), 6.48-6.56 (1H, m), 6.18 (1H, d, J = 14.8 Hz), 4.51-4.69 (2H, m), 3.60-3.69 (2H, m), 3.41-3.52 (1H, m), 3.13-3.19 (1H, m), 2.70-3.02 (4H, m), 1.89-1.99 (4H, m), 1.74-1.82 (1H, m), 1.41-1.57 (1H, m), 1.20-1.34 (6H, m); LCMS: 99.9%, MS (ESI): 594.2 m/z [M + H]⁺ |
| 58 | 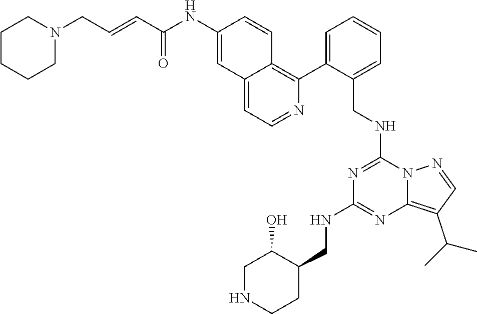 [SMILES: CC(C)c1cnn2c(NCc3ccccc3-c3nccc4cc(NC(=O)/C=C/CN5CCCCC5)ccc34)nc(NC[C@H]3CCNC[C@@H]3O)nc12] | (3R, 4R); Yellow solid; ¹H-NMR (CD₃OD, 400 MHz): δ 8.66-8.70 (1H, m), 8.45 (1H, dd, J = 6.8, 2.4 Hz), 8.19- 8.24 (1H, m), 7.80-7.88 (3H, m), 7.64-7.71 (2H, m), 7.56- 7.61 (2H, m), 7.01-7.08 (1H, m), 6.70 (1H, d, J = 15.2 Hz), 5.02-5.07 (1H, m), 4.69-4.79 (1H, m), 4.02 (2H, d, J = 7.2 Hz), 3.70-3.76 (1H, m), 3.53-3.60 (3H, m), 3.31-3.46 (3H, m), 2.76-3.07 (5H, m), 1.96-2.03 (3H, m), 1.78-1.91 (4H, m), 1.51-1.62 (2H, m), 1.19 (6H, d, J = 7.2 Hz); LCMS: 100%, MS (ESI): 689.3 m/z [M + H]⁺. |
| 59 | 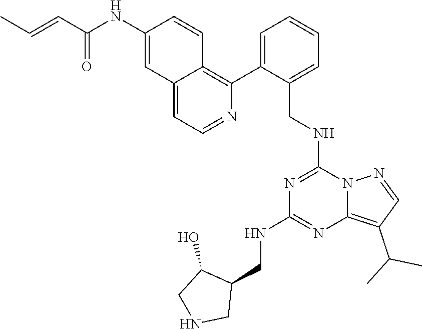 [SMILES: C/C=C/C(=O)Nc1ccc2c(-c3ccccc3CNc3nc(NC[C@H]4CNC[C@@H]4O)nc4c(C(C)C)cnn34)nccc2c1] | Mixture of 2 trans isomer; yellow solid; ¹H-NMR (CD₃OD, 400 MHz): δ 8.55-8.62 (1H, m), 8.41-8.46 (1H, m), 8.15-8.19 (1H, m), 7.56-7.87 (7H, m), 7.06-7.18 (1H, m), 6.21 (1H, d, J = 16.0 Hz), 5.09-5.21 (1H, m), 4.67- 4.82 (2H, m), 4.26-4.35 (1H, m), 3.41-3.62 (3H, m), 3.12- 3.21 (2H, m), 2.80-2.86 (1H, m), 2.38-2.59 (1H, m), 1.97 (3H, dd, J = 7.2, 1.6 Hz), 1.18 (6H, d, J = 6.8 Hz). LCMS: 100%, MS (ESI): 592.2 m/z [M + H]⁺. |
| 60 | 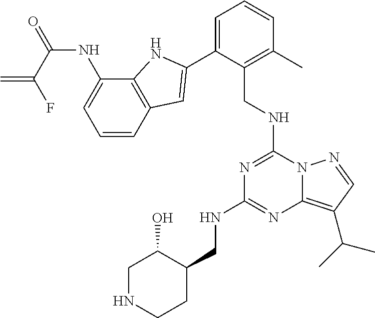 [SMILES: C=C(F)C(=O)Nc1cccc2cc(-c3cccc(C)c3CNc3nc(NC[C@H]4CCNC[C@@H]4O)nc4c(C(C)C)cnn34)[nH]c12] | (3R, 4R); gray powder; ¹H NMR (MeOD, 400 MHz): δ ppm 7.85 (1H, s), 7.34-7.48 (4H, m), 7.18 (1H, d, J = 7.6 Hz), 7.03-7.10 (1H, m), 6.49 (1H, s), 5.34-5.52 (1H, m), 5.21 (1H, dd, J = 15.2, 3.2 Hz), 4.92-4.95 (2H, m), 3.50- 3.62 (2H, m), 3.37-3.45 (1H, m), 3.20-3.27 (1H, m), 2.93- 3.05 (2H, m), 2.50-2.61 (5H, m), 1.67-1.83 (2 H, m), 1.33-1.42 (1H, m), 1.30 (6H, d, J = 6.8 Hz); LCMS: 100%, MS (ESI): m/z 612.3 [M + H]+ |
| 61 | 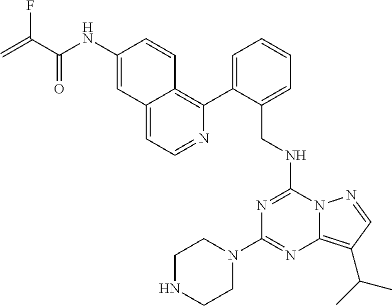 [SMILES: C=C(F)C(=O)Nc1ccc2c(-c3ccccc3CNc3nc(N4CCNCC4)nc4c(C(C)C)cnn34)nccc2c1] | Yellow solid;¹H-NMR (CD₃OD, 400 MHz): δ 8.56 (1H, d, J = 2.0 Hz), 8.36 (1H, d, J = 6.4 Hz), 8.15 (1H, d, J = 6.8 Hz), 7.76-7.83 (3H, m), 7.61-7.67 (1H, m), 7.53-7.60 (2H, m), 7.44 (1H, s), 5.85 (1H, dd, J = 46.0, 3.6 Hz), 5.44 (1H, dd, J = 15.2, 3.6 Hz), 5.00 (1H, d, J = 15.6 Hz), 4.63 (1H, d, J = 15.6 Hz), 3.89-4.00 (4H, m), 3.21-3.29 (4H, m, overlap with water signal), 2.87-2.95 (1H, m), 1.15-1.21 (6H, m); LCMS: 100%, MS (ESI): 566.2 m/z [M + H]⁺ |
| 62 | 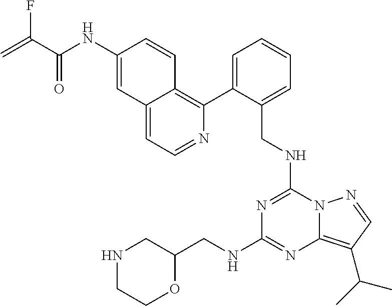 [SMILES: C=C(F)C(=O)Nc1ccc2c(-c3ccccc3CNc3nc(NCC4CNCCO4)nc4c(C(C)C)cnn34)nccc2c1] | Racemic mixture; Yellow solid; ¹H-NMR (CD₃OD, 400 MHz): δ 8.62-8.65 (1H, m), 8.44-8.49 (1H, m), 8.20-8.24 (1H, m), 7.79-7.90 (3H, m), 7.65-7.71 (2H, m), 7.54-7.61 (2H, m), 5.85 (1H, dd, J = 46.4, 3.6 Hz), 5.44 (1H, dd, J = 14.8, 3.6 Hz), 5.03-5.13 (1H, m), 4.65-4.76 (1H, m), 3.81-4.12 (3H, m), 3.32-3.61 (3H, m), 2.78-3.21 (4H, m), 1.16-1.21 (6H, m); LCMS: 99.3%, MS (ESI): 596.2 m/z [M + H]⁺ |
| 63 | 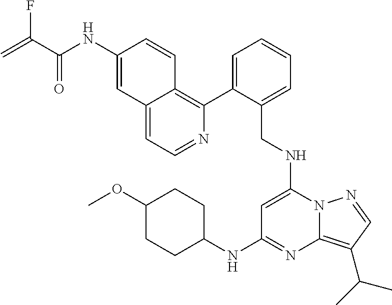 [SMILES: C=C(F)C(=O)Nc1ccc2c(-c3ccccc3CNc3cc(NC4CCC(OC)CC4)nc4c(C(C)C)cnn34)nccc2c1] | off-white powder; ¹H NMR (400 MHz, CD₃OD): δ 8.56 (1H, s), 8.44 (1H, d, J = 6.8 Hz), 8.24 (1H, d, J = 6.4 Hz), 7.92-8.00 (1H, m), 7.81-7.91 (2H, m), 7.73 (1H, t, J = 7.2 Hz), 7.62 (1H, d, J = 7.6 Hz), 7.52-7.56 (1H, m), 7.46 (1H, s), 5.85 (1H, dd, J = 46.4, 4.0 Hz), 5.48 (1H, dd, J = 15.2, 4.0 Hz), 4.90-5.02 (2H, m), 4.73-4.83 (1H, m), 3.41 (3H, s), 3.23-3.32 (2H, m), 2.85-2.94 (1H, m), 1.99-2.24 (3H, m), 1.65-1.76 (1H, m), 1.30-1.55 (4H, m), 1.15-1.24 (6H, m); HPLC: 98.5% MS (ESI): m/z 608.2 [M + H]⁺ |
| 64 | 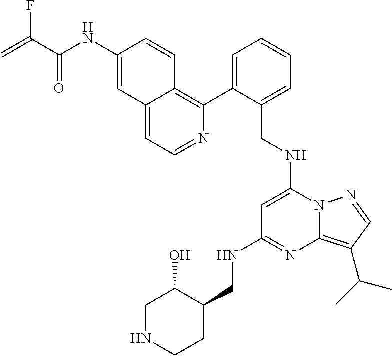 [SMILES: C=C(F)C(=O)Nc1ccc2c(-c3ccccc3CNc3cc(NC[C@H]4CCNC[C@@H]4O)nc4c(C(C)C)cnn34)nccc2c1] | (3R, 4R); light yellow solid; ¹H-NMR (CD₃OD, 400 MHz): δ 8.52-8.60 (1H, m), 8.44 (1H, J = 6.8 Hz), 8.22-8.26 (1H, m), 7.81-7.92 (3H, m), 7.67-7.72 (1H, m), 7.56-7.63 (2H, m), 7.48 (1H, s), 5.87 (1H, dd, J = 46.0, 2.8 Hz), 5.46 (1H, dd, J = 15.2, 4.0 Hz), 5.07-5.12 (1H, m), 4.73-4.79 (1H, m), 3.69-3.75 (1H, m), 3.37-3.56 (4H, m), 2.99-3.12 (1H, m), 2.81-2.97 (1H, m), 2.06-2.12 (1H, m), 1.82-1.89 (1H, m), 1.64-1.73 (1H, m), 1.17-1.24 (6H, m); LCMS: 99.7%; MS (ESI): 609.2 m/z [(M + H)]⁺ |
| 65 | 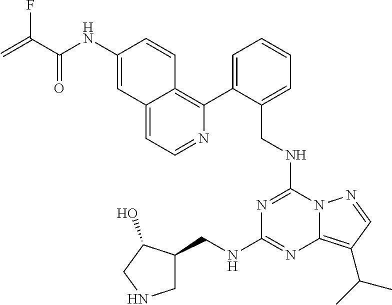 [SMILES: C=C(F)C(=O)Nc1ccc2c(-c3ccccc3CNc3nc(NC[C@H]4CNC[C@@H]4O)nc4c(C(C)C)cnn34)nccc2c1] | Mixture of 2 trans isomer; Yellow solid; ¹H-NMR (CD₃OD, 400 MHz): δ 8.60-8.67 (1H, m), 8.48-8.52 (1H, m), 8.24-8.28 (1H, m), 7.81-7.93 (3H, m), 7.56-7.73 (4H, m), 5.85 (1H, dd, J = 46.0, 3.6 Hz), 5.44 (1H, dd, J = 14.8, 3.6 Hz), 5.09-5.22 (1H, m), 4.67-4.77 (1H, m), 4.36-4.35 (1H, m), 3.46-3.62 (3H, m), 3.10-3.21 (2H, m), 2.81-2.88 (1H, m), 2.41-2.62 (1H, m), 1.16-1.22 (6H, m); LCMS: 100%, MS (ESI): 596.2 m/z [M + H^{]+} |
| 66 | 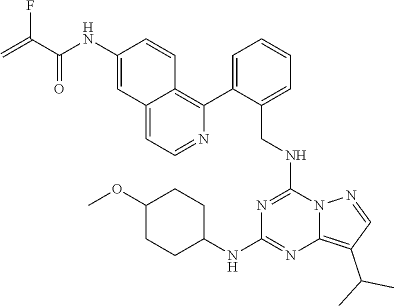 [SMILES: C=C(F)C(=O)Nc1ccc2c(-c3ccccc3CNc3nc(NC4CCC(OC)CC4)nc4c(C(C)C)cnn34)nccc2c1] | Yellow solid; ¹H-NMR (CD₃OD, 400 MHz): δ 8.63 (1H, s), 8.43 (1H, d, J = 6.8 Hz), 8.29 (1H, d, J = 6.8 Hz), 7.82-7.92 (3H, m), 7.65-7.71 (1H, m), 7.57-7.62 (2H, m), 7.54 (1H, s), 5.85 (1H, dd, J = 46.4, 3.6 Hz), 5.45 (1H, dd, J = 14.8, 3.6 Hz), 4.92-4.99 (1H, m), 4.72-4.80 (1H, m), 3.51-3.58 (1H, m), 3.39 (3H, s), 3.22-3.31 (1H, m, overlap with CD3OD signal), 2.76-2.83 (1H, m), 2.01-2.18 (3H, m), 1.69-1.73 (1H, m), 1.27-1.46 (4H, m), 1.15-1.21 (6H, m); LCMS: 100%, MS (ESI): 609.2 m/z [M + H]⁺ |
| 67 | 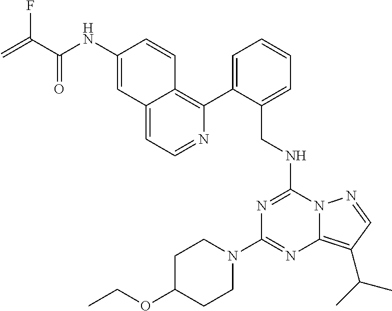 [SMILES: C=C(F)C(=O)Nc1ccc2c(-c3ccccc3CNc3nc(N4CCC(OCC)CC4)nc4c(C(C)C)cnn34)nccc2c1] | White powder; H NMR (CDCl₃, 400 MHz): δ 8.54 (1H, d, J = 5.6 Hz), 8.30 (1H, s), 8.04 (1H, brs), 7.59 (1H, d, J = 8.8 Hz), 7.55 (2H, d, J = 6.0 Hz), 7.44 (1H, s), 7.30-7.42 (3H, m), 7.25 (1H, d, J = 8.8 Hz), 6.92-6.96 (1H, m), 5.85 (1H, dd, J = 47.6, 3.6 Hz), 5.26 (1H, dd, J = 14.8, 3.6 Hz), 4.76-4.85 (1H, m), 4.12-4.31 (3H, m), 3.42-3.53 (3H, m), 3.09-3.18 (2H, m), 2.83-2.96 (1H, m), 1.80-1.87 (2H, m), 1.43-1.51 (2H, m, overlap with water signal), 1.15-1.21 (9H, m); LCMS: 98.8%, MS (ESI): m/z 609.3 [M + H]⁺ |
| 68 | 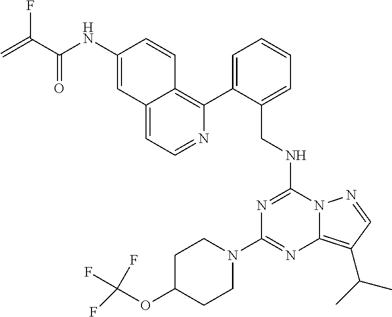 [SMILES: C=C(F)C(=O)Nc1ccc2c(-c3ccccc3CNc3nc(N4CCC(OC(F)(F)F)CC4)nc4c(C(C)C)cnn34)nccc2c1] | Off-white powder; ¹H NMR (CDCl₃, 400 MHz): δ 8.62 (1H, d, J = 6.0 Hz), 8.41 (1H, s), 8.06-8.15 (1H, m), 7.61- 7.75 (3H, m), 7.31-7.57 (5H, m), 7.02-7.23 (1H, m), 5.95 (1H, dd, J = 47.6, 3.2 Hz), 5.37 (1H, dd, J = 14.8, 3.2 Hz), 4.85-4.96 (1H, m), 4.46-4.54 (1H, m), 4.07-4.36 (2H, m), 3.47-3.60 (2H, m), 29.5-3.04 (1H, m), 1.92-2.05 (2H, m), 1.68-1.80 (2H, m), 1.27 (6H, d, J = 6.8 Hz); LCMS: 97.3%, MS (ESI): 649.1 m/z [M + H] + |
| 69 | 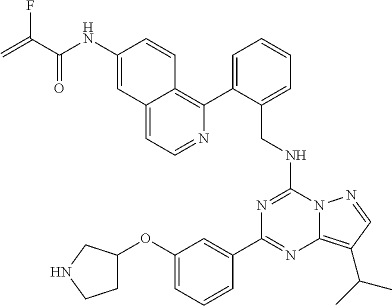 [SMILES: C=C(F)C(=O)Nc1ccc2c(-c3ccccc3CNc3nc(-c4cccc(OC5CCNC5)c4)nc4c(C(C)C)cnn34)nccc2c1] | Racemic mixture; off-white powder; ¹H NMR (400 MHz, CD₃OD) δ 8.39 (1H, s), 7.97 (1H, d, J = 6.4 Hz), 7.83-7.92 (2H, m), 7.73-7.80 (4H, m), 7.61-7.72 (3H, m), 7.56 (1H, d, J = 7.6 Hz), 7.44 (1H, t, J = 8.0 Hz), 7.12-7.16 (1H, m), 5.85 (1H, dd, J = 40.0, 4.0 Hz), 5.79 (1H, dd, J = 14.8, 3.6 Hz), 5.35-5.38 (1H, m), 5.03-5.07 (1H, m), 3.64-3.69 (1H, m), 3.18-3.27 (2H, m), 3.11-3.17 (1H, m), 2.41-2.48 (2H, m), 2.02-2.08 (1H, m), 1.58-1.64 (1H, m), 1.28-1.34 (6H, m); HPLC: 96.9% MS (ESI): m/z 643.2 [M + H]⁺ |
| 70 | 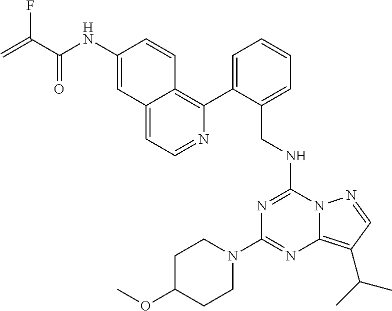 [SMILES: C=C(F)C(=O)Nc1ccc2c(-c3ccccc3CNc3nc(N4CCC(OC)CC4)nc4c(C(C)C)cnn34)nccc2c1] | Yellow powder; ¹H NMR (CDCl₃, 400 MHz): δ 8.47 (1H, d, J = 6.0 Hz), 8.28 (1H, d, J = 1.2 Hz), 8.04-8.08 (1H, m), 7.59 (1H, d, J = 9.2 Hz), 7.52-7.57 (2H, m), 7.35- 7.46 (3H, m), 7.26-7.34 (2H, m), 5.85 (1H, dd, J = 47.6, 3.6 Hz), 5.27 (1H, dd, J = 15.2, 3.6 Hz), 4.78-4.85 (1H, m), 4.08-4.24 (3H, m), 3.32-3.39 (4H, m), 3.19-3.30 (2H, m), 2.85-2.96 (1H, m), 1.79-1.84 (2H, m), 1.45- 1.58 (2H, m, overlap with water signal), 1.17 (6H, d, J = 6.8 Hz); LCMS: 98%, MS (ESI): m/z 595.2 [M + H]⁺ |
| 71 | 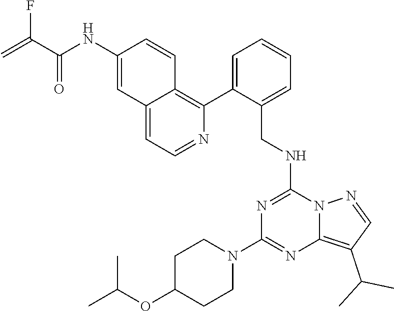 [SMILES: C=C(F)C(=O)Nc1ccc2c(-c3ccccc3CNc3nc(N4CCC(OC(C)C)CC4)nc4c(C(C)C)cnn34)nccc2c1] | Yellow powder; ¹H NMR (CD₃OD, 400 MHz): δ 8.62 (1H, d, J = 2.0 Hz), 8.42 (1H, d, J = 7.6 Hz), 8.21 (1H, d, J = 6.4 Hz), 7.79-7.91 (3H, m), 7.56-7.74 (4H, m), 5.94 (1H, dd, J = 46.4, 3.6 Hz), 5.48 (1H, dd, J = 14.8, 3.6 Hz), 4.94-5.02 (1H, m), 4.67-4.75 (1H, m), 3.91-4.02 (2H, m), 3.74-3.88 (2H, m), 3.46-3.59 (2H, m), 2.98- 3.07 (1H, m), 1.82-1.93 (2H, m), 1.47-1.65 (2H, m), 1.18- 1.23 (12H, m); LCMS: 99.9%, MS (ESI):623.2 m/z [M + H]+ |
| 72 | 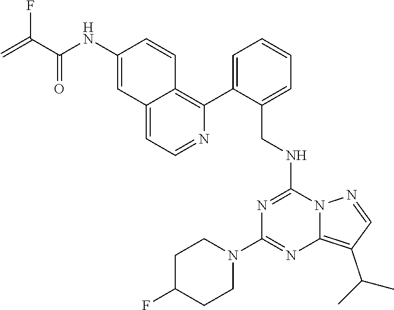 [SMILES: C=C(F)C(=O)Nc1ccc2c(-c3ccccc3CNc3nc(N4CCC(F)CC4)nc4c(C(C)C)cnn34)nccc2c1] | White solid; ¹H NMR (CDCl₃, 400 MHz,) δ 8.62 (1H, d, J = 5.6 Hz), 8.38 (1H, d, J = 2.0 Hz), 8.16-8.21 (1H, m), 7.69 (1H, d, J = 8.8 Hz), 7.60-7.65 (2H, m), 7.54 (1H, s), 7.43-7.49 (3H, m), 7.39-7.42 (1H, m), 7.09-7.16 (1H, m), 5.91 (1H, dd, J = 47.6, 3.6 Hz), 5.35 (1H, dd, J = 15.2, 3.6 Hz), 4.76-4.95 (2H, m), 4.18-4.29 (1H, m), 3.71-3.92 (4H, m), 2.93-3.05 (1H, m), 1.78-1.94 (4H, m), 1.26 (6H, d, J = 6.8 Hz); LCMS: 100.0%, MS (ESI): m/z 583.1 [M + H]+ . |
| 73 | 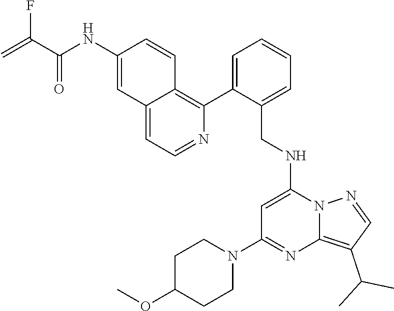 [SMILES: C=C(F)C(=O)Nc1ccc2c(-c3ccccc3CNc3cc(N4CCC(OC)CC4)nc4c(C(C)C)cnn34)nccc2c1] | Yellow powder; ¹H-NMR (CD₃OD, 400 MHz): δ 8.41 (1H, d, J = 5.6 Hz), 8.36 (1H, d, J = 1.6 Hz), 7.66-7.72 (2H, m), 7.54-7.59 (1H, m), 7.46-7.52 (2H, m), 7.35- 7.42 (3H, m), 5.78 (1H, dd, J = 46.4, 3.6 Hz), 5.36 (1H, dd, J = 14.8, 3.2 Hz), 5.02 (1H, s), 4.48-4.55 (1H, m), 4.35-4.41 (1H, m), 3.80-3.95 (2H, m), 3.42-3.48 (1H, m), 3.38 (3H, s), 3.04-3.15 (2H, m), 2.91-2.98 (1H, m), 1.86-1.92 (2H, m), 1.38-1.51 (2H, m), 1.22 (6H, d, J = 6.8 Hz); LCMS: 98.5%, MS (ESI):594.2m/z [(M + H)]+. |
| 74 | 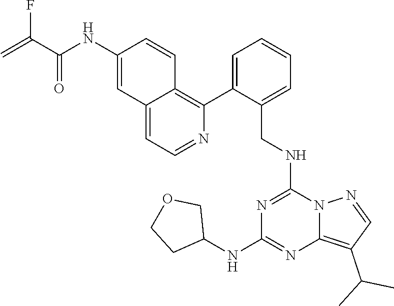 [SMILES: C=C(F)C(=O)Nc1ccc2c(-c3ccccc3CNc3nc(NC4CCOC4)nc4c(C(C)C)cnn34)nccc2c1] | White powder; ¹H NMR (CDCl₃, 400 MHz): δ 8.60 (1H, brs), 8.38-8.41 (1H, m), 8.23 (1H, brs), 7.72 (1H, d, J = 9.2 Hz), 7.58-7.63 (2H, m), 7.56 (1H, s), 7.38-7.51 (4H, m), 5.90 (1H, dd, J = 47.6, 3.6 Hz), 5.84 (1H, d, J = 15.2, 3.2 Hz), 4.73-4.89 (2H, m), 4.48-4.55 (1H, m), 4.19- 4.28 (1H, m), 3.91-4.03 (2H, m), 3.83-3.89 (1H, m), 3.66-3.71 (1H, m), 2.92-3.03 (1H, m), 2.23-2.31 (1H, m), 1.79-1.87 (1H, m), 1.22-1.29 (7H, m); LCMS: 97.4%, MS (ESI): m/z 567.2 [M + H]+ |
| 75 | 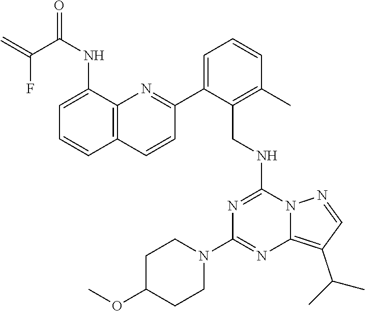 [SMILES: C=C(F)C(=O)Nc1cccc2ccc(-c3cccc(C)c3CNc3nc(N4CCC(OC)CC4)nc4c(C(C)C)cnn34)nc12] | White solid; ¹H-NMR (CDCl₃, 400 MHz): δ 10.59 (1H, brs), 8.82-8.84 (1H, m), 8.18 (1H, d, J = 8.8 Hz), 7.56- 7.61 (3H, m), 7.50 (1H, s), 7.35-7.40 (3H, m), 6.24 (1H, t, J = 4.8 Hz), 5.82 (1H, dd, J = 46.8, 3.2 Hz), 5.21 (1H, dd, J = 14.8, 3.2 Hz), 4.83-4.87 (2H, m), 4.14-4.19 (2H, m), 3.35-3.40 (4H, m), 3.14-3.21 (2H, m), 2.96-2.99 (1H, m), 2.55 (3H, s), 1.82-1.85 (2H, m), 1.41-1.49 (2H, m), 1.26 (6H, d, J = 6.8 Hz); LCMS: 100.0%, MS (ESI): 609.2 m/z [(M + H)]+. |
| 76 | 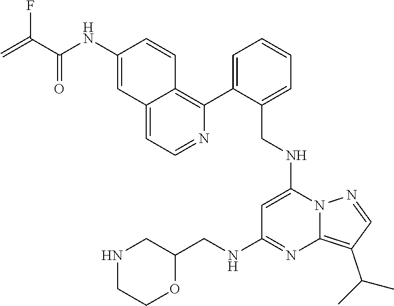 [SMILES: C=C(F)C(=O)Nc1ccc2c(-c3ccccc3CNc3cc(NCC4CNCCO4)nc4c(C(C)C)cnn34)nccc2c1] | off-white powder; ¹H NMR (400 MHz, CD₃OD) δ 8.53- 8.62 (1H, m), 8.41-8.45 (1H, m), 8.22 (1H, d, J = 6.4 Hz), 7.79-7.97 (3H, m), 7.68-7.74 (1H, m), 7.56-7.66 (2H, m), 7.50 (1H, d, J = 7.6 Hz), 5.79-5.97 (1H, m), 5.46 (1H, dd, J = 14.8, 3.6 Hz), 4.93-5.00 (2H, m, overlap with water signal), 3.96-4.17 (2H, m), 3.81-3.88 (1H, m), 3.52-3.64 (1H, m), 3.41-3.51 (2H, m), 2.84-3.24 (4H, m), 1.10-1.29 (6H, m); HPLC: 96.4% MS (ESI): m/z 595.2 [M + H]+. |
| 77 | 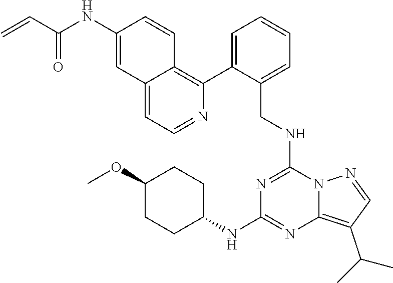 [SMILES: C=CC(=O)Nc1ccc2c(-c3ccccc3CNc3nc(N[C@H]4CC[C@H](OC)CC4)nc4c(C(C)C)cnn34)nccc2c1] | White solid; ¹H-NMR (CDCl₃, 400 MHz): δ 8.58 (1H, d, J = 5.6 Hz), 8.41-8.48 (1H, m), 7.77 (1H, brs), 7.53-7.68 (4H, m), 7.39-7.48 (3H, m), 7.30-7.34 (1H, m), 7.23 (1H, brs), 6.52 (1H, dd, J = 12.6, 1.2 Hz), 6.31 (1H, dd, J = 16.8, 10.0 Hz), 5.85 (1H, dd, J = 10.0, 1.2 Hz), 4.79- 4.91 (1H, m), 4.14-4.22 (1H, m), 3.68-3.75 (1H, m), 3.38 (3H, s), 3.14-3.22 (1H, m), 2.92-3.02 (1H, m), 1.81-2.13 (5H, m, overlap with water signal), 1.15-1.38 (9H, m) .; LCMS: 100%, MS (ESI): 591.2 m/z [M + H]+. |
| 78 | 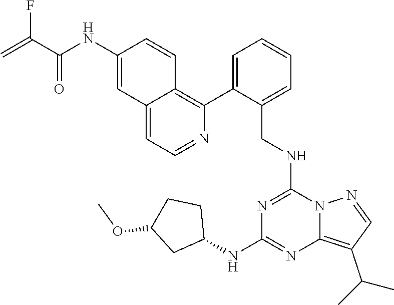 | Cis or trans form; yellow powder; ¹H NMR (CD₃OD, 400 MHz): δ 8.64-8.67 (1H, m), 8.43-8.51 (1H, m), 8.23-8.29 (1H, m), 7.78-7.93 (3H, m), 7.56-7.74 (4H, m), 5.88 (1H, dd, J = 46.0, 2.0 Hz), 5.48 (1H, dd, J = 15.2, 4.0 Hz), 4.97-5.09 (1H, m), 4.66-4.76 (1H, m), 4.16-4.25 (1H, m), 3.93-4.02 (1H, m), 3.39 (3H, s), 2.77- 2.84 (1H, m), 1.63-2.16 (5H, m), 1.53-1.62 (1H, m), 1.18- 1.26 (6H, m); LCMS: 97.9%, MS (ESI): 595.2 m/z [M + H]+ |
| 79 | 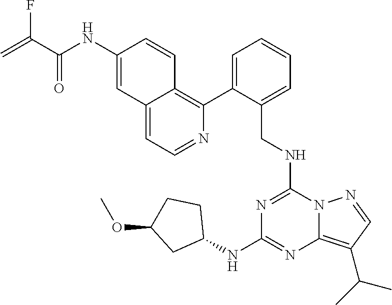 [SMILES: C=C(F)C(=O)Nc1ccc2c(-c3ccccc3CNc3nc(N[C@H]4CC[C@H](OC)C4)nc4c(C(C)C)cnn34)nccc2c1] | Cis or trans form; yellow powder; ¹H NMR (CD₃OD, 400 MHz): δ 8.64-8.67 (1H, m), 8.43-8.51 (1H, m), 8.23-8.29 (1H, m), 7.78-7.93 (3H, m), 7.56-7.74 (4H, m), 5.88 (1H, dd, J = 46.0, 2.0 Hz), 5.48 (1H, dd, J = 15.2, 4.0 Hz), 4.97-5.09 (1H, m), 4.66-4.76 (1H, m), 4.16-4.25 (1H, m), 3.93-4.02 (1H, m), 3.39 (3H, s), 2.77- 2.84 (1H, m), 1.63-2.16 (5H, m), 1.53-1.62 (1H, m), 1.18- 1.26 (6H, m); LCMS: 99.8%, MS (ESI): 595.2 m/z [M + H]+ |
| 80 | 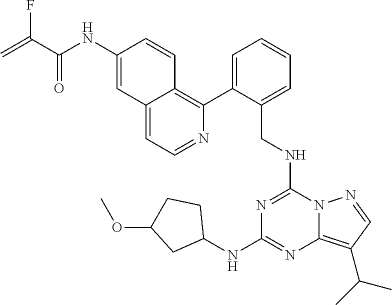 [SMILES: C=C(F)C(=O)Nc1ccc2c(-c3ccccc3CNc3nc(NC4CCC(OC)C4)nc4c(C(C)C)cnn34)nccc2c1] | Racemic mixture; yellow powder; yellow powder; ¹H NMR (CD₃OD, 400 MHz): δ 8.64-8.67 (1H, m), 8.43- 8.51 (1H, m), 8.23-8.29 (1H, m), 7.78-7.93 (3H, m), 7.56-7.74 (4H, m), 5.88 (1H, dd, J = 46.0, 2.0 Hz), 5.48 (1H, dd, J = 15.2, 4.0 Hz), 4.97-5.09 (1H, m), 4.66-4.76 (1H, m), 4.16-4.25 (1H, m), 3.93-4.02 (1H, m), 3.39 (3H, s), 2.77-2.84 (1H, m), 1.63-2.16 (5H, m), 1.53-1.62 (1H, m), 1.18-1.26 (6H, m); LCMS: 99.8%, MS (ESI): 595.2 m/z [M + H]+ |
| 81 | 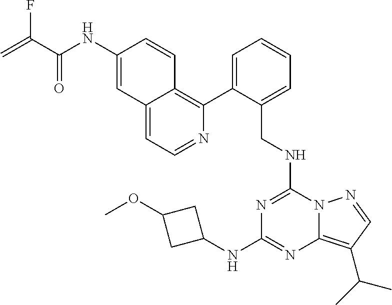 [SMILES: C=C(F)C(=O)Nc1ccc2c(-c3ccccc3CNc3nc(NC4CC(OC)C4)nc4c(C(C)C)cnn34)nccc2c1] | White solid; ¹H NMR (CD₃OD, 400 MHz,) δ 8.58-8.64 (1H, m), 8.39-8.42 (1H, m), 8.10-8.18 (1H, m), 7.78- 7.85 (3H, m), 7.61-7.69 (2H, m), 7.56-7.60 (2H, m), 5.85 (1H, dd, J = 46.0, 3.2 Hz), 5.45 (1H, dd, J = 14.8, 3.2 Hz), 4.98-5.05 (1H, m), 4.63-4.75 (1H, m), 3.67- 3.78 (2H, m), 3.28 (3H, s), 2.76-2.84 (2H, m), 2.53-2.61 (1H, m), 1.83-2.02 (2H, m), 1.17-1.22 (6H, m); LCMS: 100.0%, MS (ESI): m/z 581.2 [M + H]+ . |
| 82 | 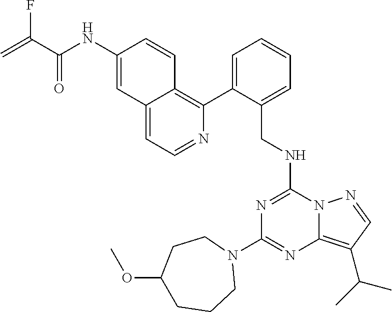 [SMILES: C=C(F)C(=O)Nc1ccc2c(-c3ccccc3CNc3nc(N4CCCC(OC)CC4)nc4c(C(C)C)cnn34)nccc2c1] | Yellow solid; ¹H NMR (CDCl₃, 400 MHz) δ 8.56-8.61 (1H, m), 8.44-8.53 (1H, m), 8.22-8.27 (1H, m), 7.92- 7.97 (1H, m), 7.81-7.85 (2H, m), 7.54-7.71 (4H, m), 5.87 (1H, dd, J = 46.0, 3.6 Hz), 5.46 (1H, dd, J = 15.2, 3.6 Hz), 3.42-3.96 (7H, m), 3.34 (3H, s), 3.03-3.15 (1H, m), 1.47-2.13 (5H, m), 1.20-1.24 (1H, m), 1.14-1.22 (6H, m); LCMS: 99.2%, MS (ESI): m/z 609.2 [M + H]+ |
| 83 | 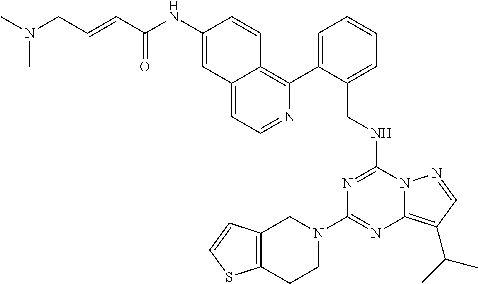 [SMILES: CC(C)c1cnn2c(NCc3ccccc3-c3nccc4cc(NC(=O)/C=C/CN(C)C)ccc34)nc(N3CCc4sccc4C3)nc12] | Yellow powder; ¹H NMR (CD₃OD, 400 MHz): δ 8.55 (1H, d, J = 2.0 Hz), 8.03-8.12 (1H, m), 7.97 (1H, d, J = 6.4 Hz), 7.82-7.85 (2H, m), 7.63-7.67 (2H, m), 7.50- 7.54 (3H, m), 7.29 (1H, d, J = 5.2 Hz), 6.93-7.02 (1H, m), 6.88 (1H, d, J = 4.8 Hz), 6.62 (1H, d, J = 15.2 Hz), 4.68-4.75 (1H, m), 4.01-4.09 (3H, m), 3.89-3.97 (1H, m), 3.56-3.61 (1H, m), 3.43-3.48 (1H, m), 2.91-3.02 (9H, m), 1.17-1.22 (6H, m) .; LCMS: 100%, MS (ESI): m/z 658.2 [M + H]+ |
| 84 | 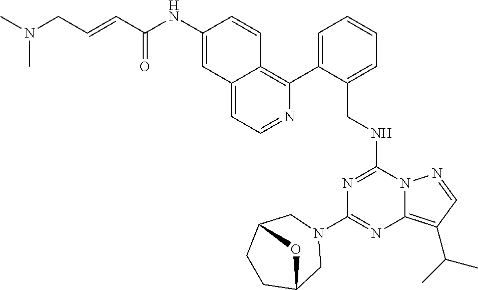 [SMILES: CC(C)c1cnn2c(NCc3ccccc3-c3nccc4cc(NC(=O)/C=C/CN(C)C)ccc34)nc(N3C[C@H]4CC[C@@H](C3)O4)nc12] | Yellow powder; ¹H NMR (400 MHz, CD₃OD) δ 8.67 (1H, d, J = 1.6 Hz), 8.33-8.42 (1H, m), 8.18-8.25 (1H, m), 7.80- 7.91 (3H, m), 7.67-7.72 (1H, m), 7.57-7.65 (3H, m), 6.99- 7.10 (1H, m), 6.76 (1H, d, J = 15.6 Hz), 4.73-4.84 (2H, m), 4.40-4.55 (2H, m), 4.09 (2H, d, J = 6.8 Hz), 3.97-4.06 (2H, m), 3.35-3.38 (2H, m, overlap with CD3OD signal), 3.05-3.19 (1H, m), 2.98 (6H, s), 1.47-2.14 (4 H, m), 1.14- 1.26 (6H, m); HPLC (254 nm): 100% MS (ESI): m/z 632.2 [M + H]+ |
| 85 | 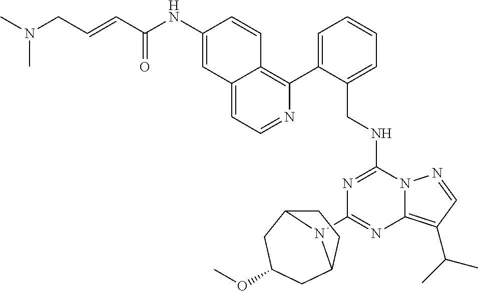 | off-white powder; ¹H NMR (CD₃OD, 400 MHz): δ 8.62 (1H, s), 8.27-8.46 (1H, m), 8.11-8.18 (1H, m), 7.70-7.88 (3H, m), 7.63-7.69 (1H, m), 7.50-7.58 (3H, m), 6.96- 7.07 (1H, m), 6.72 (1H, d, J = 15.2 Hz), 4.87-4.92 (2H, m, overlap with water signal), 4.63-4.72 (1H, m), 4.07 (2H, d, J = 6.8 Hz), 3.46-3.49 (1H, m), 3.30-3.33 (4H, m), 3.01-3.12 (1H, m), 2.96 (6H, s), 1.51-2.43 (8H, m), 1.17 (6H, d, J = 6.4 Hz); LCMS: 100%, MS (ESI): m/z 660.3 [M + H]+ |
| 86 | 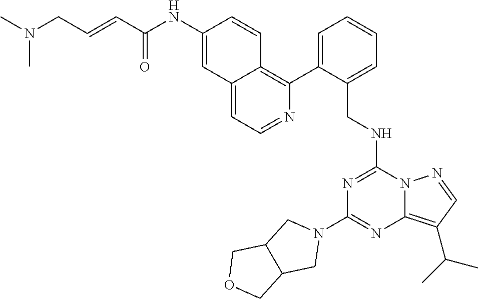 [SMILES: CC(C)c1cnn2c(NCc3ccccc3-c3nccc4cc(NC(=O)/C=C/CN(C)C)ccc34)nc(N3CC4COCC4C3)nc12] | Yellow solid;¹H NMR (CD₃OD, 400 MHz): δ 8.62-8.66 (1H, m), 8.40 (1H, d, J = 6.4 Hz), 8.21 (1H, d, J = 6.4 Hz), 7.81-7.88 (3H, m), 7.68-7.72 (2H, m), 7.58-7.61 (2H, m), 6.99-7.07 (1H, m), 6.75 (1H, d, J = 14.8 Hz), 4.80-4.84 (1H, m, overlap with water signal), 4.72-4.75 (1H, m), 4.07 (2H, d, J = 6.8 Hz), 3.66-3.88 (9H, m), 3.08-3.15 (2H, m), 2.96 (6H, s), 1.16-1.21 (6H, m); LCMS: 100.0%, MS (ESI): 632.2m/z [M + H]+ |
| 87 | 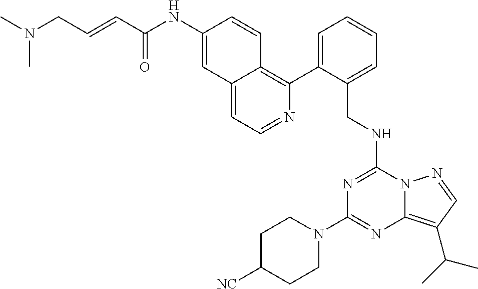 [SMILES: CC(C)c1cnn2c(NCc3ccccc3-c3nccc4cc(NC(=O)/C=C/CN(C)C)ccc34)nc(N3CCC(C#N)CC3)nc12] | White powder; ¹H NMR (CD₃OD, 400 MHz): δ 8.29-8.33 (2H, m), 7.64-7.68 (1H, m), 7.57-7.62 (2H, m), 7.51-7.55 (1H, m), 7.32-7.43 (4H, m), 6.96-7.02 (1H, m), 6.32 (1H, d, J = 15.6 Hz), 4.92-4.95 (1H, m, overlap with water signal), 4.36-4.43 (1H, m), 3.86-3.94 (2H, m), 3.38-3.47 (2H, m), 3.23 (2H, d, J = 6.8 Hz), 2.96-3.03 (1H, m), 2.83- 2.90 (1H, m), 2.34 (6H, s), 1.80-1.92 (2H, m), 1.64-1.78 (2H, m), 1.17-1.23 (6H, m); LCMS: 99.2%, MS (ESI): 629.2 m/z [M + H]+ |
| 88 | 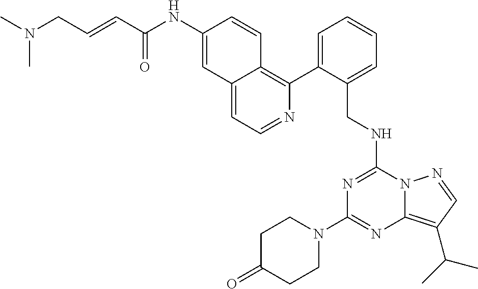 [SMILES: CC(C)c1cnn2c(NCc3ccccc3-c3nccc4cc(NC(=O)/C=C/CN(C)C)ccc34)nc(N3CCC(=O)CC3)nc12] | off-white solid; ¹H NMR (CD₃OD, 400 MHz) δ 8.58 (1H, s), 8.21-8.28 (1H, m), 7.99-8.04 (1H, m), 7.77-7.81 (2H, m), 7.62-7.73 (2H, m), 7.52-7.59 (2H, m), 7.41-7.45 (1H, m), 6.97-7.06 (1H, m), 6.63-6.72 (1H, m), 4.67-4.73 (2H, m), 4.07 (2H, d, J = 6.8 Hz), 3.54-3.70 (4H, m), 2.96 (6H, s), 1.93-2.16 (2H, m), 1.63-1.78 (2H, m), 1.21- 1.28 (6H, m); LCMS: 100.0%, MS (ESI): m/z 618.2 [M + H]+ |
| 89 | 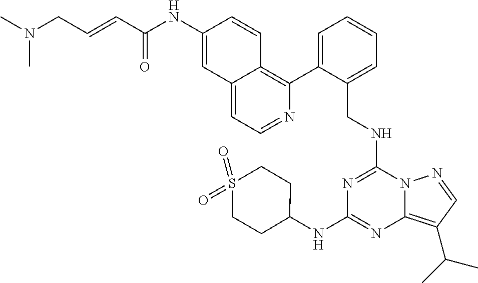 [SMILES: CC(C)c1cnn2c(NCc3ccccc3-c3nccc4cc(NC(=O)/C=C/CN(C)C)ccc34)nc(NC3CCS(=O)(=O)CC3)nc12] | White powder; ¹H NMR (CD₃OD, 400 MHz): δ 8.30- 8.35 (2H, m), 7.63-7.66 (1H, m), 7.55-7.62 (2H, m), 7.48-7.53 (1H, m), 7.40-7.45 (2H, m), 7.32-7.38 (2H, m), 6.91-7.01 (1H, m), 6.39 (1H, d, J = 15.6 Hz), 4.95- 4.98 (1H, m, overlap with water signal), 4.30-4.37 (1H, m), 3.88-3.95 (1H, m), 3.44 (2H, d, J = 6.4 Hz), 3.18- 3.24 (2H, m), 3.08-3.16 (2H, m), 2.1-2.90 (1H, m), 2.50 (6H, s), 2.26-2.35 (2H, m), 2.01-2.20 (2H, m), 1.16-1.22 (6H, m); LCMS: 100%, MS (ESI): m/z 668.3 [M + H]+ |
| 90 | 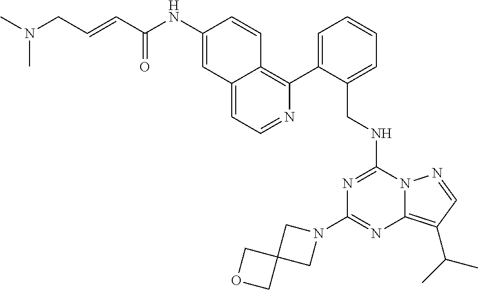 [SMILES: CC(C)c1cnn2c(NCc3ccccc3-c3nccc4cc(NC(=O)/C=C/CN(C)C)ccc34)nc(N3CC4(COC4)C3)nc12] | white solid; ¹H NMR (CD₃OD, 400 MHz) δ 8.30 (1H, d, J = 1.6 Hz), 8.26 (1H, d, J = 6.0 Hz), 7.63-7.66 (1H, m), 7.55-7.60 (2H, m), 7.48-7.53 (1H, m), 7.31-7.47 (4H, m), 6.91-7.02 (1H, m), 6.33 (1H, d, J = 15.6 Hz), 4.86-4.90 (1H, m, overlap with water signal), 4.79- 4.84 (4H, m), 4.35-4.41 (1H, m), 3.95-4.07 (4H, m), 3.28 (2H, d, J = 6.4 Hz), 2.88-2.97 (1H, m), 2.37 (6H, s), 1.16-1.21 (6H, m); LCMS: 100.0%, MS (ESI): m/z 618.1 [M + H]+ |
| 91 | 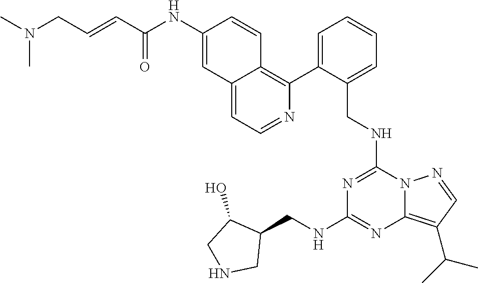 [SMILES: CC(C)c1cnn2c(NCc3ccccc3-c3nccc4cc(NC(=O)/C=C/CN(C)C)ccc34)nc(NC[C@H]3CNC[C@@H]3O)nc12] | Yellow solid; ¹H-NMR (CD₃OD, 400 MHz): δ 8.65-8.68 (1H, m), 8.42-8.47 (1H, m), 8.18-8.21 (1H, m), 7.80- 7.87 (3H, m), 7.67-7.73 (2H, m), 7.56-7.62 (2H, m), 6.98-7.05 (1H, m), 6.68-6.75 (1H, m), 5.03-5.18 (1H, m), 4.72-4.78 (1H, m), 4.28-4.36 (1H, m), 4.06 (2H, d, J = 7.2 Hz), 3.31-3.62 (4H, m), 3.12-3.23 (2H, m), 2.95 (6H, s), 2.83-2.92 (1H, m), 2.38-2.61 (1H, m), 1.18-1.22 (6H, m); LCMS: 100%, MS (ESI): 635.2 m/z [M + H]+ |
| 92 | 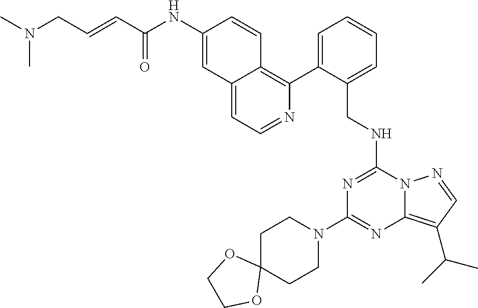 [SMILES: CC(C)c1cnn2c(NCc3ccccc3-c3nccc4cc(NC(=O)/C=C/CN(C)C)ccc34)nc(N3CCC4(CC3)OCCO4)nc12] | White solid; ¹H NMR (CD₃OD, 400 MHz) δ 8.28-8.32 (2H, m), 7.64-7.67 (1H, m), 7.54-7.61 (2H, m) 7.46-7.52 (1H, m), 7.31-7.39 (4H, m), 6.91-6.99 (1H, m), 6.37 (1H, d, J = 15.2 Hz), 4.79-4.84 (1H, m, overlap with water signal), 4.36-4.41 (1H, m), 3.98 (4H, s), 3.60-3.74 (4H, m), 3.37-3.43 (2H, m), 2.81-2.91 (1H, m), 2.44-2.52 (6H, m), 1.52-1.64 (4H, m), 1.19 (6H, d, J = 5.6 Hz); LCMS: 100.0%, MS (ESI): m/z 662.3 [M + H]+ |
| 93 | 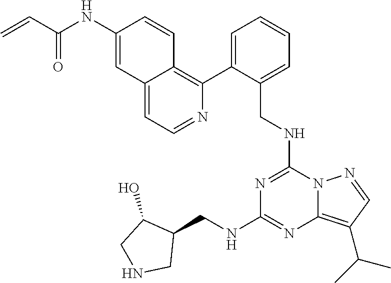 [SMILES: C=CC(=O)Nc1ccc2c(-c3ccccc3CNc3nc(NC[C@H]4CNC[C@@H]4O)nc4c(C(C)C)cnn34)nccc2c1] | Yellow solid; ¹H NMR (CD₃OD, 400 MHz): δ 8.58-8.64 (1H, m), 8.40-8.46 (1H, m), 8.19 (1H, d, J = 6.8 Hz), 7.63-7.89 (5H, m), 7.55-7.62 (2H, m), 6.48-6.52 (2H, m), 5.89-5.94 (1H, m), 5.06-5.23 (1H, m), 4.66-4.76 (1H, m), 4.26-4.36 (1H, m), 3.34-3.61 (4H, m), 3.12- 3.21 (2H, m), 2.78-2.85 (1H, m), 2.38-2.61 (1H, m), 1.15-1.21 (6H, m); LCMS: 100%, MS (ESI): 578.2 m/z [M + H]+ |
| 94 | 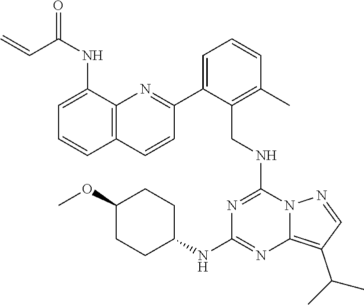 [SMILES: C=CC(=O)Nc1cccc2ccc(-c3cccc(C)c3CNc3nc(N[C@H]4CC[C@H](OC)CC4)nc4c(C(C)C)cnn34)nc12] | White solid; ¹H NMR (CDCl₃, 400 MHz): δ 9.77 (1H, s), 8.88 (1H, dd, J = 6.0, 2.8 Hz), 8.23 (1H, d, J = 8.4 Hz), 7.55-7.61 (4H, m), 7.34-7.43 (3H, m), 6.62 (1H, brs), 6.30-6.44 (2H, m), 5.58 (1H, dd, J = 9.2, 2.0 Hz), 4.68 (2H, d, J = 5.2 Hz), 4.58-4.63 (1H, m), 3.73-3.76 (1H, m), 3.35 (3H, s), 3.12-3.18 (1H, m), 2.96-3.03 (1H, m), 2.53 (3H, s), 2.03-2.13 (4H, m), 1.13-1.35 (10H, m); LCMS: 98.7%, MS (ESI): 606.3 m/z [(M + H)]+ |
| 95 | 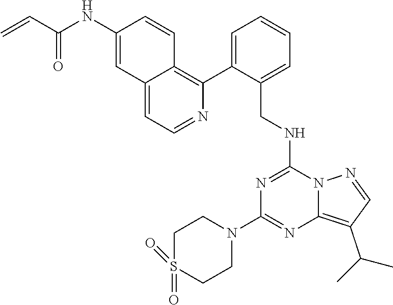 [SMILES: C=CC(=O)Nc1ccc2c(-c3ccccc3CNc3nc(N4CCS(=O)(=O)CC4)nc4c(C(C)C)cnn34)nccc2c1] | White solid; ¹H NMR (CDCl₃, 400 MHz) δ 8.56 (1H, d, J = 5.6 Hz), 8.39 (1H, d, J = 1.6 Hz), 7.68 (1H, d, J = 8.8 Hz), 7.60-7.63 (2H, m), 7.55-7.59 (1H, m), 7.45-7.49 (3H, m), 7.41-7.45 (1H, m), 7.37 (1H, dd, J = 8.8, 2.0 Hz), 6.54 (1H, dd, J = 16.8, 1.2 Hz), 6.30 (1H, dd, J = 16.8, 10.4 Hz), 5.88 (1H, dd, J = 10.4, 1.2 Hz), 4.74-4.85 (1H, m), 4.24-4.36 (5H, m), 2.93-3.05 (5H, m), 1.27 (6 H, d, J = 6.8 Hz); LCMS: 100.0%, MS (ESI): m/z 597.1 [M + H]+ |
| 96 | 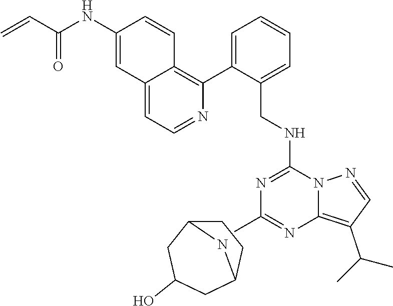 | Light yellow powder; ¹H NMR (CDCl₃, 400 MHz): δ 8.61 (1H, d, J = 5.6 Hz), 8.40 (1H, brs), 7.62-7.70 (3H, m), 7.53-7.57 (2H, m), 7.38-7.49 (3H, m), 7.28-7.35 (1H, m), 6.53 (1H, d, J = 16.8 Hz), 6.27-6.35 (1H, m), 5.87 (1H, d, J = 10.4 Hz), 4.80-4.86 (1H, m), 4.55-4.64 (2H, m), 4.21-4.27 (1H, m), 4.02-4.11 (1H, m), 2.95- 3.04 (1H, m), 2.15-2.26 (3H, m), 1.98-2.06 (2H, m), 1.71-1.75 (2H, m), 1.50-1.55 (1H, m), 1.26 (6H, d, J = 7.2 Hz); LCMS: 100.0%, MS (ESI): 589.1 m/z [(M + H)]+ |
| 97 | 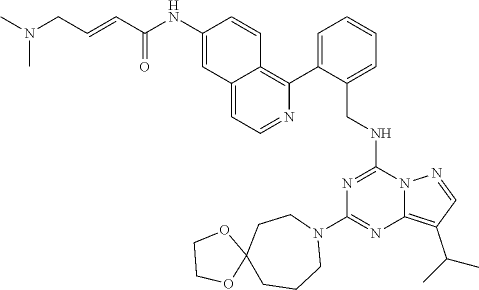 [SMILES: CC(C)c1cnn2c(NCc3ccccc3-c3nccc4cc(NC(=O)/C=C/CN(C)C)ccc34)nc(N3CCCC4(CC3)OCCO4)nc12] | Light yellow solid; ¹H NMR (CD₃OD, 400 MHz): δ 8.28- 8.31 (2H, m), 7.43-7.65 (4H, m), 7.28-7.39 (4H, m), 6.92-7.00 (1H, m), 6.31 (1H, d, J = 15.2 Hz), 4.73-4.80 (1H, m, overlap with water signal), 4.36-4.42 (1H, m), 3.89 (4H, s), 3.49-3.82 (4H, m), 3.23 (2H, d, J = 6.8 Hz), 2.78-2.87 (1H, m), 2.33 (6H, s), 1.56-1.91 (6H, m), 1.15-1.20 (6H, m); LCMS: 99.0%, MS (ESI): 676.2 m/z [M + H]+ |
| 98 | 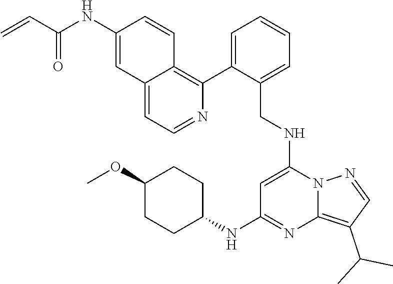 [SMILES: C=CC(=O)Nc1ccc2c(-c3ccccc3CNc3cc(N[C@H]4CC[C@H](OC)CC4)nc4c(C(C)C)cnn34)nccc2c1] | White powder; ¹H NMR (CDCl₃, 400 MHz): δ 8.49 (1H, d, J = 5.6 Hz), 8.38 (1H, brs), 7.55-7.65 (2H, m), 7.43- 7.52 (3H, m), 7.29-7.42 (3H, m), 7.14-7.18 (1H, m, overlap with CDCl3 signal), 6.45 (1H, d, J = 16.8 Hz), 6.17-6.28 (2H, m), 5.78 (1H, d, J = 7.2 Hz), 4.71 (1H, s), 4.12-4.35 (2H, m), 3.21-3.50 (4H, m), 3.03-3.16 (1H, m), 2.89-2.97 (1H, m), 1.79-2.12 (4H, m), 1.02-1.33 (10H, m); LCMS: 99.9%, MS (ESI): 590.2 m/z [M + H]+ |
| 99 | 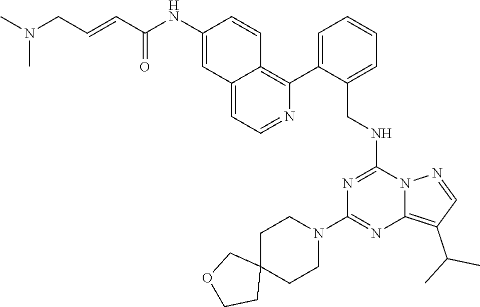 [SMILES: CC(C)c1cnn2c(NCc3ccccc3-c3nccc4cc(NC(=O)/C=C/CN(C)C)ccc34)nc(N3CCC4(CCOC4)CC3)nc12] | Yellow powder; ¹H NMR (400 MHz, CD₃OD) δ 8.65 (1H, d, J = 1.60 Hz), 8.35 (1H, d, J = 6.80 Hz), 8.15 (1H, d, J = 6.40 Hz), 7.79-7.88 (3H, m), 7.56-7.73 (4H, m), 6.97-7.12 (1H, m), 6.75 (1H, d, J = 15.20 Hz), 4.75 (2H, br d, J = 15.60 Hz), 4.09 (2H, d, J = 7.00 Hz), 3.93 (2H, t, J = 7.20 Hz) 3.60-3.78 (6H, m), 3.03-3.10 (1H, m), 2.98 (6H, s), 1.90 (2H, t, J = 7.20 Hz), 1.65 (4H, br s), 1.21 (6H, dd, J = 6.80, 5.60 Hz); HPLC: 100% MS (ESI): m/z 660.2 [M + H]+ |
| 100 | 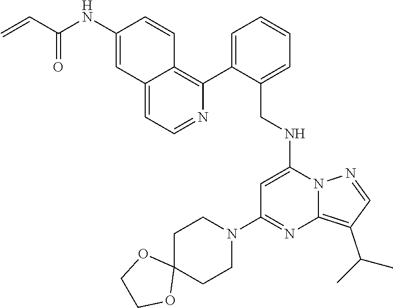 [SMILES: C=CC(=O)Nc1ccc2c(-c3ccccc3CNc3cc(N4CCC5(CC4)OCCO5)nc4c(C(C)C)cnn34)nccc2c1] | White powder; ¹H NMR (400 MHz, CD₃OD) δ 8.56 (1H, d, J = 5.60 Hz), 8.42 (1H, s), 7.36-7.68 (7H, m), 6.53 (1H, d, J = 17.20 Hz), 6.24-6.38 (2H, m), 5.87 (1H, d, J = 10.40 Hz), 5.16 (1H, s), 4.35 (2H, d, J = 5.60 Hz), 4.01 (4H, s), 3.62-3.74 (4H, m), 2.99-3.12 (1H, m), 1.68-1.79 (4H, m), 1.30 (6H, d, J = 7.00 Hz); HPLC: 97.2% MS (ESI): m/z 604.1 [M + H]+ |
| 101 | 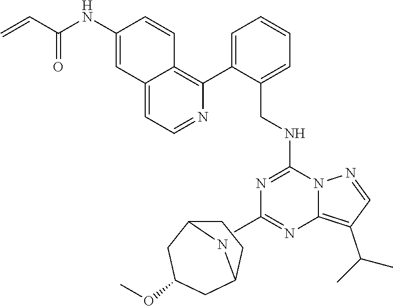 | White powder; ¹H NMR (CDCl₃, 400 MHz): δ 8.56 (1H, d, J = 5.6 Hz), 8.43 (1H, s), 7.36-7.65 (8H, m), 7.20 (1H, dd, J = 2.0 and 9.1Hz), 6.52 (1H, d, J=16.8 Hz), 6.24- 6.34 (2H, m), 5.86 (1H, d, J = 10.5 Hz), 5.00 (1H, s), 4.22-4.42 (4H, m), 3.72 (1H, br d, J = 3.6 Hz), 3.34 (1H, br s), 3.28 (3H, s), 3.02 (1H, quin, J = 6.8 Hz), 2.10 (2H, br d, J = 7.1 Hz), 1.91-2.05 (3H, m), 1.66-1.91 (3H, m), 1.27 (6H, d, J = 6.9 Hz); LCMS: 100%, MS (ESI): m/z 602.5 [M + H]+ |
| 102 | 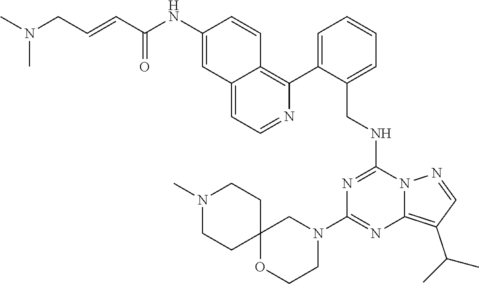 [SMILES: CC(C)c1cnn2c(NCc3ccccc3-c3nccc4cc(NC(=O)/C=C/CN(C)C)ccc34)nc(N3CCOC4(CCN(C)CC4)C3)nc12] | Yellow solid; ¹H NMR (CD₃OD, 400 MHz): δ 8.61 (1H, d, J = 2.0 Hz), 8.34 (1H, d, J = 6.8 Hz), 8.12 (1H, d, J = 6.8 Hz), 7.64-7.86 (4H, m), 7.53-7.60 (2H, m), 7.47 (1H, s), 6.97-7.06 (1H, m), 6.71 (1H, d, J = 15.2 Hz), 4.95- 5.01 (1H, m), 4.68-4.74 (1H, m), 4.06 (2H, d, J = 7.2 Hz), 3.46-3.81 (6H, m), 3.34-3.40 (2H, m), 3.14-3.25 (2H, m), 2.93-3.02 (7H, m), 2.87 (3H, s), 2.09-2.21 (2H, m), 1.79-1.92 (2H, m), 1.14-1.19 (6H, m); LCMS: 98.6%, MS (ESI): 689.4 m/z [M + H]+ |
| 103 | 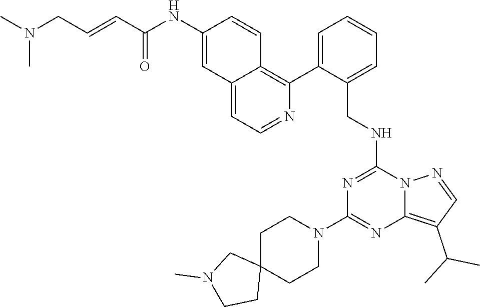 [SMILES: CC(C)c1cnn2c(NCc3ccccc3-c3nccc4cc(NC(=O)/C=C/CN(C)C)ccc34)nc(N3CCC4(CCN(C)C4)CC3)nc12] | Pale yellow solid; ¹H NMR (400 MHz, CD₃OD) δ 8.61 (1H, s), 8.39 (1H, d, J = 6.4Hz), 8.17 (1H, d, J = 6.4Hz), 7.79-7.81 (3H, m), 7.64-7.67 (1H, m), 7.54-7.63 (3H, m), 6.99-7.05 (1H, m), 6.76 (1H, d, J = 14.8Hz), 4.95-4.99 (1H, m), 4.68-4.71 (1H, m), 4.08 (2H, d, J = 7.2Hz), 3.76- 3.81 (3H, m), 3.67-3.73 (3H, m), 3.02-3.06 (2H, m), 2.99 (3H, s), 2.96 (6H, s), 2.17-2.20 (1H, m), 2.06-2.11 (1H, m), 1.72-1.77 (4H, m), 1.19 (6H, d, J = 6.8 Hz); LCMS: 99.8%, MS (ESI): 673.4 m/z [M + H]+ |
| 104 | 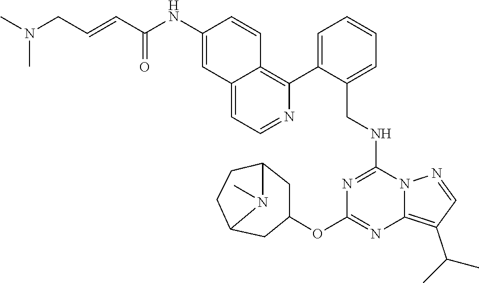 [SMILES: CC(C)c1cnn2c(NCc3ccccc3-c3nccc4cc(NC(=O)/C=C/CN(C)C)ccc34)nc(OC3CC4CCC(C3)N4C)nc12] | Yellow powder; ¹H NMR (CD₃OD, 400 MHz) δ 8.64 (1H, d, J = 2 Hz), 8.36 (1H, d, J = 6.8 Hz), 8.14 (1H, d, J = 6.4 Hz), 7.77-7.86 (2H, m), 7.69-7.75 (1H, m), 7.61-7.69 (2H, m), 7.58 (1H, s), 7.55 (1H, d, J = 7.6 Hz), 6.97-7.08 (1H, m), 6.70 (1H, d, J = 14.8 Hz), 5.09 (1H, m), 5.01 (1H, d, J = 15.6 Hz), 4.64 (1H, d, J = 15.6 Hz), 4.07 (2H, d, J = 7.6 Hz), 3.93 (2H, m), 2.96 (6H, s), 2.89 (1H, m), 2.83 (3H, s), 2.03-2.58 (8H, m), 1.21 (6H, d, J = 6.8 Hz); LCMS: 100%, MS (ESI): m/z 660.5 [M + H]+ |
| 105 | 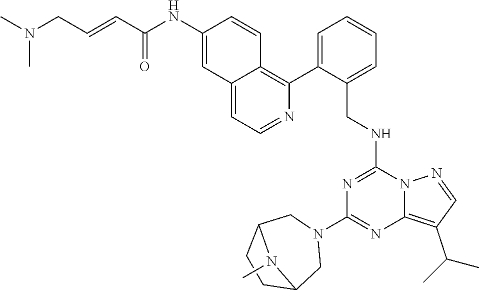 [SMILES: CC(C)c1cnn2c(NCc3ccccc3-c3nccc4cc(NC(=O)/C=C/CN(C)C)ccc34)nc(N3CC4CCC(C3)N4C)nc12] | White solid; ¹H NMR (CD₃OD, 400MHz) δ 8.54 (1H, s), 8.32 (1H, d, J = 6.8Hz), 8.09 (1H, d, J = 6.8Hz), 7.77-7.82 (2H, m), 7.61-7.64 (2H, m), 7.49-7.53 (2H, m), 7.33 (1H, s), 6.97-7.03 (1H, m), 6.71 (1H, d, J = 15.2Hz), 4.95-5.00 (2H, m), 4.39-4.68 (2H, m), 4.00-4.08 (4H, m), 3.31- 3.44 (1H, m), 2.96 (6H, s), 2.86 (3H, s), 2.81-2.86 (1H, m), 2.21-2.26 (2H, m), 1.88-1.93 (1H, m), 1.67-1.72 (2H, m), 1.27-1.32 (2H, m), 1.18 (6H, d, J = 6.8 Hz); LCMS: 99.5%, MS (ESI): m/z 645.4 [M + H]+ |
| 106 | 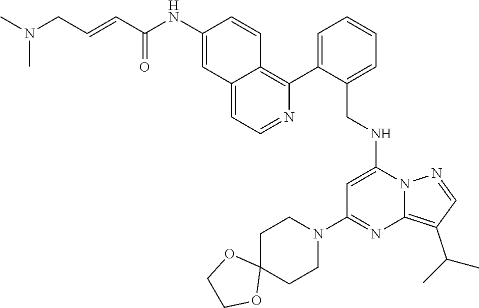 [SMILES: CC(C)c1cnn2c(NCc3ccccc3-c3nccc4cc(NC(=O)/C=C/CN(C)C)ccc34)cc(N3CCC4(CC3)OCCO4)nc12] | White powder; ¹H NMR (400 MHz, CD₃OD) δ 8.32- 8.47 (2H, m), 7.68-7.74 (2H, m), 7.56-7.62 (1H, m), 7.52 (1H, t, J = 7.60 Hz), 7.34-7.41 (4H, m), 6.99 (1H, dt, J = 15.37, 6.49 Hz), 6.34 (1H, d, J = 15.60 Hz), 5.03 (1H, s), 4.55 (1H, br d, J = 15.56 Hz), 4.37-4.45 (1H, m), 4.00 (4H, s), 3.50-3.71 (4H, m), 3.24 (2H, d, J = 5.60 Hz), 2.91-2.99 (1H, m), 2.34 (6H, s), 1.56-1.71 (4H, m), 1.23 (6H, d, J = 8.00 Hz); HPLC: 99.4% MS (ESI): m/z 661.3 [M + H]+ |
| 107 | 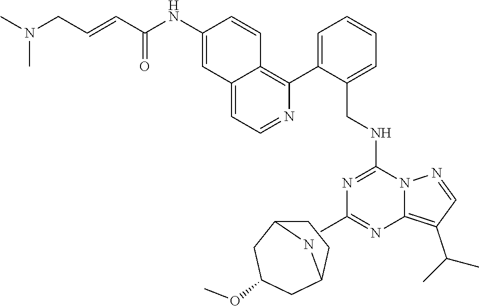 | Yellow powder; ¹H NMR (CD₃OD, 400 MHz): δ 8.57 (1H, d, J = 1.8 Hz), 8.37 (1H, d, J = 6.5 Hz), 8.07 (1H, d, J = 6.3 Hz), 7.84-7.90 (1H, m), 7.80 (1H, t, J = 7.2 Hz), 7.64-7.74 (2H, m), 7.49-7.58 (3H, m), 6.94-7.07 (1H, m), 6.73 (1H, d, J = 15.3 Hz), 4.99 (1H, s), 4.94 (1H, br s), 4.65-4.75 (1H, m), 4.49 (2H, br s), 4.06 (2H, d, J = 7.3 Hz), 3.46-3.52 (1H, m), 3.33 (3H, s), 3.11-3.21 (1H, m), 2.96 (6H, s), 2.30 (2H, br d, J = 7.5 Hz), 1.92-2.11 (5H, m), 1.68 (1H, br s), 1.18 (6H, dd, J = 6.9 and 12.2 Hz) .. LCMS: 98.2%, MS (ESI): m/z 659.5 [M + H]+ |
| 108 | 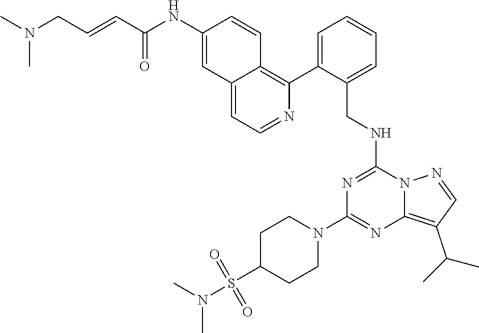 [SMILES: CC(C)c1cnn2c(NCc3ccccc3-c3nccc4cc(NC(=O)/C=C/CN(C)C)ccc34)nc(N3CCC(S(=O)(=O)N(C)C)CC3)nc12] | White powder; ¹H NMR (CD₃OD, 400 MHz): δ 8.65 (1H, s), 8.35-8.47 (1H, m), 8.22 (1H, d, J = 6.40 Hz), 7.78-7.92 (3H, m), 7.53-7.73 (4H, m), 6.94-7.11 (1H, m), 6.74 (1H, d, J = 14.40 Hz), 4.95-5.02 (1H, m), 4.75-4.82 (1H, m), 4.34-4.55 (2H, m), 4.07 (2H, d, J = 7.20 Hz), 3.51-3.63 (1H, m), 3.02-3.25 (3H, m), 2.96 (12H, s). 1.52-2.27 (4H, m), 1.18 (6H, t, J = 6.78 Hz); HPLC: 100%, MS (ESI): 711.5 m/z [M + H]+ |
| 109 | 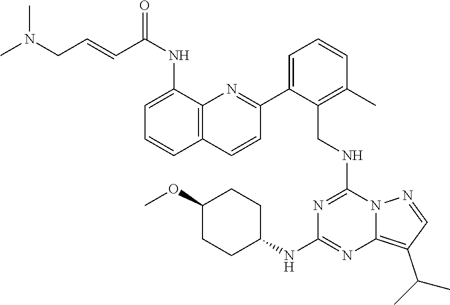 [SMILES: CO[C@H]1CC[C@H](Nc2nc(NCc3c(C)cccc3-c3ccc4cccc(NC(=O)/C=C/CN(C)C)c4n3)n3ncc(C(C)C)c3n2)CC1] | White powder; ¹H NMR (CD₃OD, 400 MHz) δ8.67 (1H, d, J = 8.0 Hz) 8.29 (1H, d, J = 8.4 Hz) 7.65 (2H, d, J = 9.2 Hz) 7.53-7.59 (2H, m) 7.40-7.44 (2H, m) 7.34-7.39 (1H, m) 6.78-6.91 (1H, m) 6.27 (1H, s) 4.76 (2H, s) 3.33 (3H, s) 3.14 (3H, s) 2.92 (1H, t, J = 8.4 Hz) 2.56 (3H, s) 2.33 (6H, s) 2.02 (4H, d, J = 12.0 Hz) 1.29 (2H, s) 1.24 (6H, d, J = 6.8 Hz) 1.17 (2H, s); LCMS: 92.5%, MS (ESI): m/z 662.5 [M + H]+ |
| 110 | 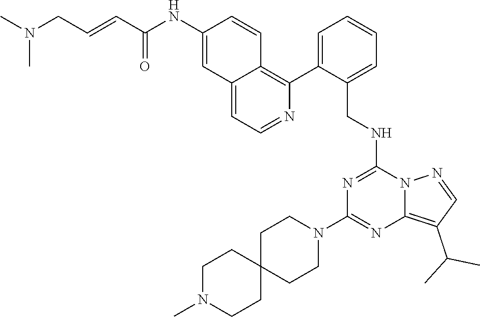 [SMILES: CC(C)c1cnn2c(NCc3ccccc3-c3nccc4cc(NC(=O)/C=C/CN(C)C)ccc34)nc(N3CCC4(CCN(C)CC4)CC3)nc12] | Yellow solid; ¹H NMR (CD₃OD, 400 MHz) δ 8.67 (1H, d, J = 1.2 Hz) 8.44 (1H, d, J = 6.4 Hz) 8.23 (1H, d, J = 6.4 Hz) 7.88 (1H, dd, J = 9.2, 1.6 Hz) 7.80-7.85 (2H, m) 7.65-7.72 (2H, m) 7.62 (1H, s) 7.58-7.61 (1H, m) 6.99- 7.09 (1H, m) 6.76 (1H, d, J = 15.56 Hz) 5.01 (2H, br d, J = 15.6 Hz) 4.73 (1H, d, J = 15.6 Hz) 4.08 (2H, d, J = 6.8 Hz) 3.77 (4H, s) 3.43 (2H, d, J = 12.0 Hz) 3.19-3.24 (2H, m) 3.08-3.16 (1H, m) 2.96 (6H, s) 2.92 (3H, s) 1.93-2.13 (2H, m) 1.84 (2H, s) 1.71-1.78 (2H, m) 1.54- 1.65 (2H, m) 1.18 (6H, dd, J = 6.8, 3.2 Hz); LCMS: 100.0%, MS (ESI): m/z 687.6 [M + H]+ |
| 111 | 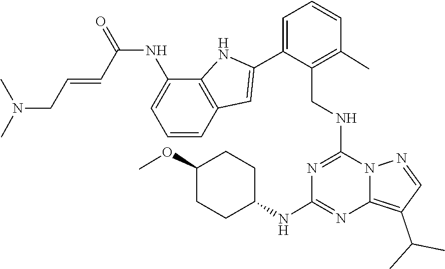 [SMILES: CO[C@H]1CC[C@H](Nc2nc(NCc3c(C)cccc3-c3cc4cccc(NC(=O)/C=C/CN(C)C)c4[nH]3)n3ncc(C(C)C)c3n2)CC1] | White powder; ¹H NMR (CD₃OD, 400 MHz): δ 7.72 (1H, s), 7.37 (3H, s), 7.25-7.34 (2H, m), 6.98 (1H, t, J = 7.80 Hz), 6.81-6.92 (1H, m), 6.70-6.78 (1H, m), 6.35 (1H, s), 5.00 (2H, s), 4.01 (2H, d, J = 7.03 Hz), 3.59-3.76 (1H, m), 3.32-3.38 (3H, m), 3.12-3.20 (1H, m) 2.94 (7H, s), 2.55 (3H, s), 1.85-2.05 (4H, m), 1.22-1.31 (10H, m); HPLC: 98.0%, MS (ESI): 650.5 m/z [M + H]+ |
| 112 | 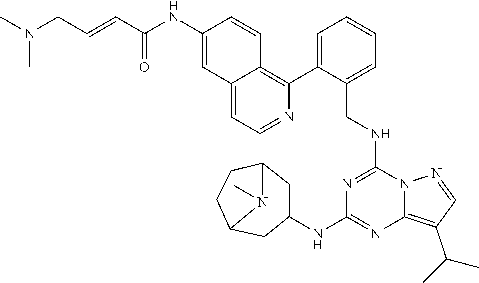 [SMILES: CC(C)c1cnn2c(NCc3ccccc3-c3nccc4cc(NC(=O)/C=C/CN(C)C)ccc34)nc(NC3CC4CCC(C3)N4C)nc12] | White powder; ¹H NMR (CD₃OD, 400 MHz): δ 8.60- 8.72 (1H, m), 8.41 (1H, d, J = 6.8 Hz), 8.20 (1H, d, J = 5.6 Hz), 7.78-7.89 (3H, m), 7.53-7.73 (4H, m), 6.96-7.10 (1H, m), 6.71 (1H, d, J = 15.2 Hz), 5.01 (2H, m), 4.72 (1H, d, J = 15.6 Hz), 4.07 (2H, d, J = 6.8 Hz), 3.90-3.99 (2H, m), 2.96 (6H, s), 2.81-2.90 (4H, m), 2.06-2.66 (8H, m), 1.23 (6H, m); LCMS: 100%, MS (ESI): m/z 659.4 [M + H]+ |
| 113 | 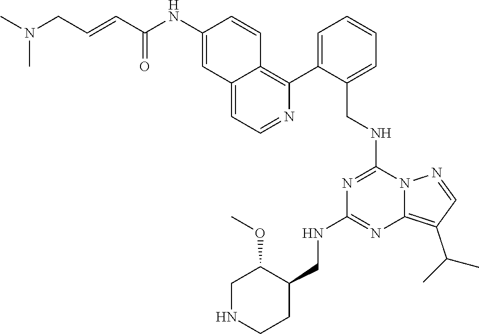 [SMILES: CO[C@H]1CNCC[C@@H]1CNc1nc(NCc2ccccc2-c2nccc3cc(NC(=O)/C=C/CN(C)C)ccc23)n2ncc(C(C)C)c2n1] | Yellow powder; ¹H NMR (400 MHz, CD₃OD) δ 8.57 (1H, s), 8.41 (1H, s), 8.06 (1H, s), 7.50-7.88 (7 H, m), 6.92-7.08 (1H, m) 6.66 (1H, d, J = 15.20 Hz), 4.96-5.19 (2H, m), 4.62-4.80 (1H, m), 4.06 (2 H, d, J = 7.20 Hz), 3.38-3.69 (7H, m), 2.81-3.03 (9H, m), 1.88-2.12 (2H, m), 1.58 (1H, s), 1.18 (6H, d, J = 6.80 Hz); HPLC: 99.8% MS (ESI): m/z 663.6 [M + H]+ |
| 114 | 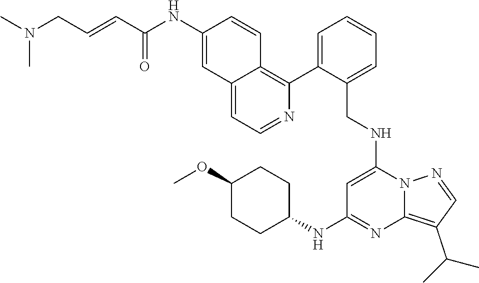 [SMILES: CO[C@H]1CC[C@H](Nc2cc(NCc3ccccc3-c3nccc4cc(NC(=O)/C=C/CN(C)C)ccc34)n3ncc(C(C)C)c3n2)CC1] | Yellow powder; ¹H NMR (400 MHz, CD₃OD) δ 8.55 (1H, s), 8.40 (1H, d, J = 6.40 Hz), 8.03 (1H, s), 7.76-7.89 (2H, m), 7.59-7.70 (2H, m), 7.43-7.57 (3H, m), 6.96- 7.03 (1H,m), 6.70 (1H, d, J = 15.20 Hz), 4.95-5.02 (2H, m) 4.65-4.75 (2H, m), 4.06 (2H, d, J = 7.20 Hz), 3.38 (3H, s) 3.21-3.28 (1H, m), 2.91-3.01 (7H, m), 2.00-2.19 (3H, m), 1.72 (1H, s), 1.29-1.48 (4 H, m) 1.18 (6 H, dd, J =14.00, 6.80 Hz); HPLC: 100% MS (ESI): m/z 647.4 [M + H]+ |
| 115 | 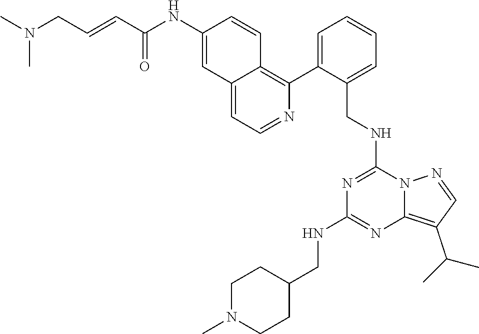 [SMILES: CC(C)c1cnn2c(NCc3ccccc3-c3nccc4cc(NC(=O)/C=C/CN(C)C)ccc34)nc(NCC3CCN(C)CC3)nc12] | Yellow powder; ¹H NMR (CD₃OD, 400 MHz): δ 8.65 (1H, s), 8.43 (1H, d, J = 6.4 Hz), 8.18 (1H, d, J = 6.8 Hz), 7.76-7.93 (3H, m), 7.54-7.74 (4H, m), 6.97-7.09 (1H, m), 6.72 (1H, d, J = 15.6 Hz), 5.05 (1H, d, J =15.6 Hz), 4.70-4.79 (1H, m), 4.07 (2H, d, J = 7.2 Hz), 3.53 (2H, d, J =10.4 Hz), 3.10-3.27 (2H, m), 3.01 (2H, m), 2.96 (6H, s), 2.86 (4H, m), 1.80-2.10 (3H, m), 1.61 (2H, m), 1.16- 1.25 (6H, m); LCMS: 100%, MS (ESI): m/z 647.4 [M + H]+ |
| 116 | 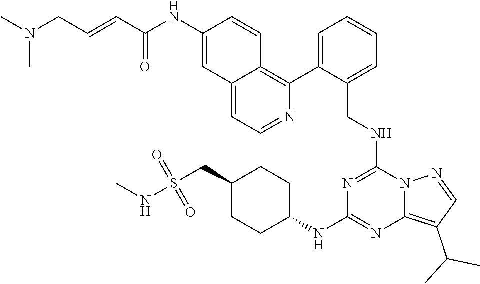 [SMILES: CNS(=O)(=O)C[C@H]1CC[C@H](Nc2nc(NCc3ccccc3-c3nccc4cc(NC(=O)/C=C/CN(C)C)ccc34)n3ncc(C(C)C)c3n2)CC1] | White powder; ¹H NMR (CD₃OD, 400 MHz) δ 8.68 (1H, s), 8.39 (1H, d, J = 6.8 Hz), 8.24 (1H, d, J = 6.8 Hz), 7.78- 7.92 (3H, m), 7.68 (1H, m), 7.52-7.65 (3H, m), 7.02 (1H, m), 6.71 (1H, d, J =14.8 Hz), 4.94-4.95 (1H, m), 4.81- 4.82 (1H, m), 4.07 (2H, d, J = 7.2 Hz), 3.05 (2H, d, J = 5.2 Hz), 2.96 (6H, s), 2.77-2.91 (2H, m), 2.74 (3H, s), 1.70- 2.16 (5H, m), 1.28-1.52 (4H, m), 1.13-1.22 (6H, m); LCMS: 100%, MS (ESI): m/z 725.4 [M + H]+ |
| 117 | 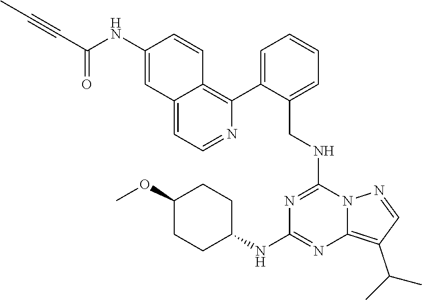 [SMILES: CC#CC(=O)Nc1ccc2c(-c3ccccc3CNc3nc(N[C@H]4CC[C@H](OC)CC4)nc4c(C(C)C)cnn34)nccc2c1] | White powder; ¹H NMR (CD₃OD, 400MHz): δ 8.47 (1H, s), 8.40 (1H, d, J = 6.4 Hz), 8.22 (1H, d, J = 6.4 Hz), 7.78- 7.88 (2H, m), 7.63-7.72 (2H, m), 7.52-7.61 (3H, m), 4.97 (1H, s), 4.75 (1H, d, J = 16 Hz), 3.45-3.60 (1H, m), 3.40 (3H, s), 3.25 (1H, s), 2.77-2.91 (1H, m), 2.16 (1H, m), 2.09 (3H, s), 2.05 (2H, m), 1.65-1.81 (1H, m), 1.27-1.52 (4H, m), 1.17-1.27 (6H, m); LCMS: 98%, MS (ESI): m/z 603.3 [M + H]+ |
| 118 | 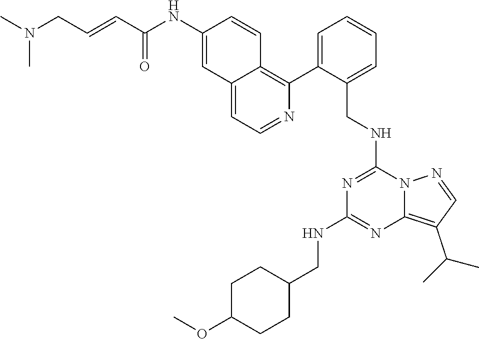 [SMILES: COC1CCC(CNc2nc(NCc3ccccc3-c3nccc4cc(NC(=O)/C=C/CN(C)C)ccc34)n3ncc(C(C)C)c3n2)CC1] | Yellow powder; ¹H NMR (400 MHz, CD₃OD) δ ppm 8.66 (1H, d, J = 1.60 Hz), 8.43 (1H, d, J = 6.40 Hz), 8.18- 8.32 (1H, m), 7.80-7.87 (3H, m) 7.65-7.75 (1H, m), 7.53- 7.63 (3H, m), 6.95-7.09 (1H, m), 6.72 (1H, d, J = 15.20 Hz), 4.92-5.01 (1H, m), 4.76-4.82 (1H, m), 4.07 (2H, d, J = 7.20 Hz), 3.36 (3H, s), 3.22-3.26 (1H, m), 3.11-3.20 (2H, m), 3.01-3.09 (2H, m), 2.96 (6H, s), 2.78-2.88 (1H, m), 1.96-2.16 (3H, m), 1.70-1.85 (2H, m), 1.32-1.51 (2H, m), 1.18 (6H, t, J = 6.40 Hz), 1.00-1.12 (2H, m); HPLC: 96.4% MS (ESI): m/z 662.4 [M + H]+ |
| 119 | 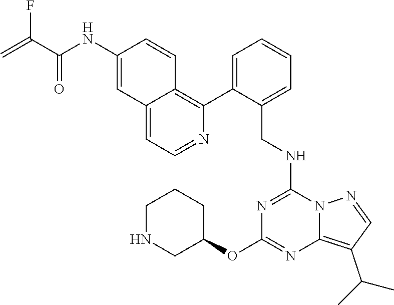 [SMILES: C=C(F)C(=O)Nc1ccc2c(-c3ccccc3CNc3nc(O[C@@H]4CCCNC4)nc4c(C(C)C)cnn34)nccc2c1] | White powder; ¹H NMR (CDCl₃, 400 MHz): δ 8.56 (1H, m), 8.40 (2H, m), 7.41-7.88 (9H, m), 5.81-6.01 (1H, m), 5.33 (1H, dd, J = 15.2, 3.6 Hz), 4.74-5.21 (2H, m), 4.19 (1H, m), 2.84-3.36 (5H, m), 1.91 (4H, m), 1.26 (6H, d, J = 6.8 Hz); LCMS: 100%, MS (ESI): m/z 581.3 [M + H]+ |
| 120 | 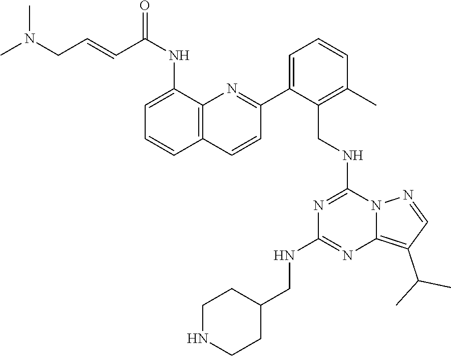 [SMILES: Cc1cccc(-c2ccc3cccc(NC(=O)/C=C/CN(C)C)c3n2)c1CNc1nc(NCC2CCNCC2)nc2c(C(C)C)cnn12] | Yellow solid; ¹H NMR (400 MHz, CD₃OD) δ = 8.74 (d, J = 7.2 Hz, 1H), 8.25 (d, J = 8.4 Hz, 1H), 7.71-7.64 (m, 2H), 7.64-7.58 (m, 2H), 7.49-7.41 (m, 2H), 7.31 (d, J = 6.8 Hz, 1H), 6.94-6.84 (m, 1H), 6.78-6.71 (m, 1H), 4.91 (s, 1H), 4.83 (br s, 1H), 4.02 (d, J=7.0 Hz, 2H), 3.37 (m, 2H), 3.11 (br d, J = 5.8 Hz, 2H), 2.97-2.84 (m, 9H), 2.62 (s, 3H), 1.84 (br d, J=13.8 Hz, 2H), 1.48-1.37 (m, 2H), 1.26 (d, J = 7.2 Hz, 6H); LCMS: 100%, MS (ESI): m/z 647.4 [M + H] + |
| 121 | 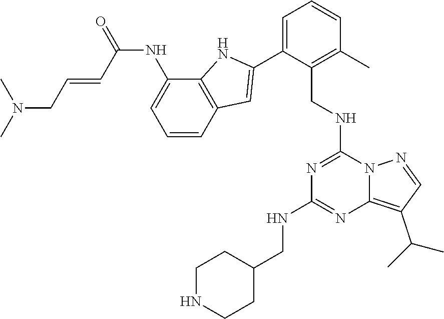 [SMILES: Cc1cccc(-c2cc3cccc(NC(=O)/C=C/CN(C)C)c3[nH]2)c1CNc1nc(NCC2CCNCC2)nc2c(C(C)C)cnn12] | White solid; ¹H NMR (400 MHz, CD₃OD) δ = 7.83 (s, 1H), 7.43 (m, 1H), 7.42-7.38 (m, 2H), 7.37-7.31 (m, 2H), 7.04 (t, J = 7.8 Hz, 1H), 6.94-6.82 (m, 1H), 6.80- 6.73 (m, 1H), 6.43 (s, 1H), 5.03 (s, 2H), 4.04 (d, J = 7.0 Hz, 2H), 3.39-3.34 (m, 2H), 3.24 (br d, J = 12.5 Hz, 2H), 3.00 (br d, J = 6.8 Hz, 1H), 2.96 (s, 6H), 2.73 (m, 2H), 2.57 (s, 3H), 1.82 (m, 3H), 1.38 (m, 2H), 1.31 (d, J = 7.0 Hz, 6H); LCMS: 100%, MS (ESI): m/z 635.4 [M + H] + |
| 122 | 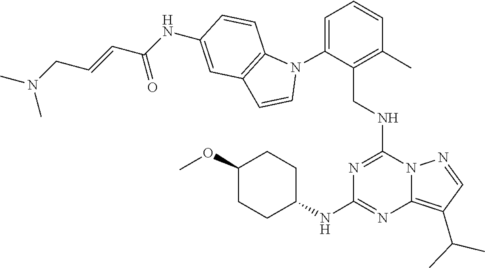 [SMILES: CO[C@H]1CC[C@H](Nc2nc(NCc3c(C)cccc3-n3ccc4cc(NC(=O)/C=C/CN(C)C)ccc43)n3ncc(C(C)C)c3n2)CC1] | White powder; ¹H NMR (400 MHz, CD₃OD) δ = 7.96 (d, J =1.6 Hz, 1H), 7.76 (s, 1H), 7.54-7.51 (m, 1H), 7.48- 7.45 (m, 1H), 7.25-7.17 (m, 2H), 6.95 (m, 1H), 6.88- 6.83 (m, 1H), 6.77 (d, J = 8.4 Hz, 1H), 6.62 (m, 1H), 6.50 (d, J = 3.2 Hz, 1H), 4.84-4.79 (m, 1H), 4.66 (m, 1H), 4.08-3.97 (m, 3H), 3.38 (s, 3H), 3.22 (br s, 1H), 2.97 (s, 6H), 2.90-2.84 (m, 1H), 2.67-2.61 (m, 3H), 2.18- 2.06 (m, 2H), 1.99 (m, 2H), 1.31-1.22 (m, 10H); LCMS: 100%, MS (ESI): m/z 650.4 [M + H]+ |
| 123 | 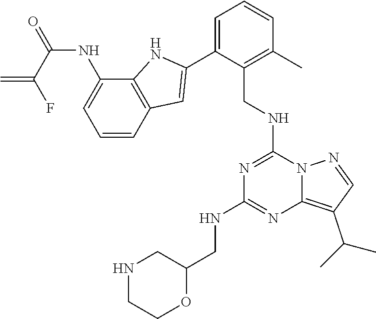 [SMILES: C=C(F)C(=O)Nc1cccc2cc(-c3cccc(C)c3CNc3nc(NCC4CNCCO4)nc4c(C(C)C)cnn34)[nH]c12] | Gray solid; ¹H NMR (400MHz, CD₃OD) δ = 7.85 (s, 1H), 7.47-7.35 (m, 4H), 7.19 (d, J = 7.9 Hz, 1H), 7.11-7.02 (m, 1H), 6.49 (br s, 1H), 5.57-5.39 (m, 1H), 5.24 (dd, J = 3.4, 15.0 Hz, 1H), 4.95 (m, 2H), 4.12-4.00 (m, 1H), 3.86 m, 1H), 3.75 (m, 1H), 3.54-3.41 (m, 2H), 3.21 (m, 2H), 3.04 (m, 2H), 2.83 (s, 1H), 2.54 (s, 3H), 1.31 (d, J = 6.9 Hz, 6H); LCMS: 100%, MS (ESI): m/z 598.3 [M + H]+ |
| 124 | 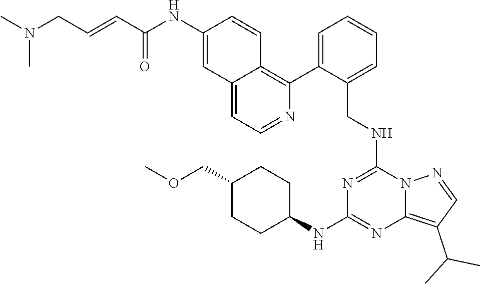 [SMILES: COC[C@H]1CC[C@H](Nc2nc(NCc3ccccc3-c3nccc4cc(NC(=O)/C=C/CN(C)C)ccc34)n3ncc(C(C)C)c3n2)CC1] | Yellow powder; ¹H NMR (400 MHz, CD3OD) δ ppm 8.68 (1H, s), 8.41 (1H, d, J = 6.40 Hz), 8.25 (1H, d, J = 6.40 Hz), 7.78-7.90 (3H, m), 7.68 (1H, t, J = 6.40 Hz), 7.51-7.61 (3H, m), 6.95-7.11 (1H, m), 6.72 (1H, d, J = 15.20 Hz), 4.95-5.02 (1H, m) 4.73-4.83 (1H, m), 4.07 (2 H, d, J = 7.20 Hz), 3.43-3.54 (1H, m), 3.37 (3H, s) 3.25-3.31 (2H, m), 2.96 (6H, s), 2.77-2.87 (1H, m) 1.55- 2.11 (5H, m), 1.2-1.44 ( H, m), 1.06-1.22 (8H, m); HPLC: 98.5% MS (ESI): m/z 662.4 [M + H]+ |
| 125 | 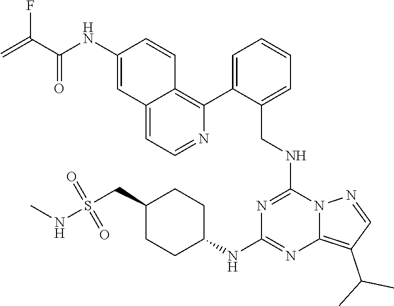 [SMILES: C=C(F)C(=O)Nc1ccc2c(-c3ccccc3CNc3nc(N[C@H]4CC[C@H](CS(=O)(=O)NC)CC4)nc4c(C(C)C)cnn34)nccc2c1] | Yellow powder; ¹H NMR (400 MHz, CD₃OD) δ 8.62 (1H, s) 8.45 (1H, d, J = 6.40 Hz), 8.28 (1H, d, J = 6.80 Hz), 7.84-7.93 (3 H, m), 7.66-7.72 (1H, m), 7.59 (2H, d, J = 8.00 Hz), 7.52 (1H, s), 5.78-5.95 (1H, m), 5.46 (1H, dd, J = 14.80, 3.60 Hz), 4.95-5.02 (1H, m), 4.71-4.82 (1H, m), 3.50-3.59 (1H, m), 3.06 (2H, d, J = 6.40 Hz), 2.78-2.87 (1H, m), 2.74 (3H, s), 1.68-1.78 (1H, m), 1.25- 1.53 (4H, m), 1.12-1.23 (6H, m), 1.87-2.18 (4H, m); HPLC: 98.6% MS (ESI): m/z 686.3 [M + H]+ |
| 126 | 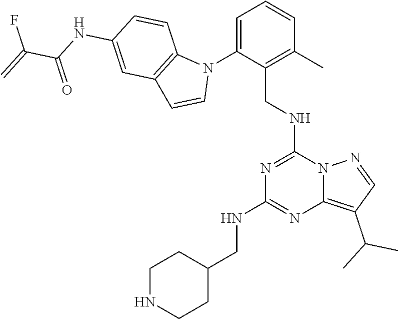 | White solid; ¹H NMR (CDCl₃, 400 MHz,) δ9.43 (1H, s) 8.53 (1H, d, J = 5.2 Hz) 8.47 (1H, d, J = 9.2 Hz) 8.30 (1H, d, J=2.8 Hz) 7.60 (1H, dd, J = 9.2, 2.8 Hz) 7.56 (1H, s) 7.46 (1H, s) 6.47 (1H, d, J=5.2 Hz) 4.07 (6H, s) 3.10- 3.13 (1H, m) 3.08 (1H, d, J = 3.2 Hz) 3.05 (2H, d, J = 8.8 Hz) 2.62-2.75 (2H, m) 1.73-1.80 (2H, m) 1.02 (3 H, t, J = 7.6 Hz); LCMS: 98.0%, MS (ESI): m/z 531.3 [M + H]+ |
| 127 | 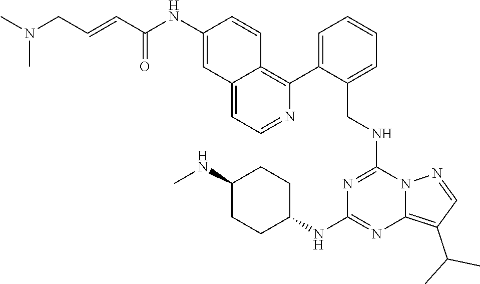 [SMILES: CN[C@H]1CC[C@H](Nc2nc(NCc3ccccc3-c3nccc4cc(NC(=O)/C=C/CN(C)C)ccc34)n3ncc(C(C)C)c3n2)CC1] | Yellow solid; ¹H NMR (400 MHz, CD₃OD) δ = 8.70 (s, 1H), 8.45 (d, J = 6.5 Hz, 1H), 8.26 (d, J = 6.8 Hz, 1H), 7.91 (br s, 2H), 7.81 (dd, J = 2.0, 9.0 Hz, 1H), 7.74-7.68 (m, 1H), 7.64-7.58 (m, 2H), 7.56 (s, 1H), 7.04 (td, J = 7.2, 15.0 Hz, 1H), 6.73 (d, J = 15.3 Hz, 1H), 5.08 (m, 1H), 4.85- 4.78 (m, 1H), 4.09 (d, J = 7.3 Hz, 2H), 3.69 (m, 1H), 3.17-3.06 (m, 1H), 2.98 (s, 6H), 2.91-2.81 (m, 1H), 2.78 (s, 3H), 2.28 m, 1H), 2.19 (m, 2H), 1.85 (m, 1H), 1.77-1.66 (m, 1H), 1.58 (m, 2H), 1.52-1.38 (m, 1H), 1.23-1.16 (m, 6H); LCMS: 100%, MS (ESI): m/z 647.4 [M + H]+ |
| 128 | 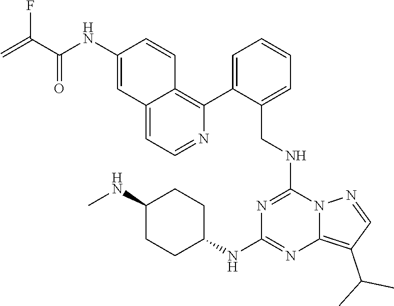 [SMILES: C=C(F)C(=O)Nc1ccc2c(-c3ccccc3CNc3nc(N[C@H]4CC[C@H](NC)CC4)nc4c(C(C)C)cnn34)nccc2c1] | Yellow powder; ¹H NMR (400 MHz, CD₃OD) δ 8.59 (1H, s), 8.45 (1H, d, J = 6.00 Hz), 8.20 (1H, d, J = 5.20 Hz), 7.77-7.96 (3H, m), 7.64-7.71 (1H, m), 7.48- 7.61 (3H, m), 5.73-5.94 (1H, m), 5.45 (1H, dd, J = 14.80, 3.60 Hz), 5.05-5.15 (1H, d, J = 15.56 Hz), 4.70-4.78 (1H, m), 3.60-3.75 (1H, m), 3.04-3.16 (1H, m), 2.82-2.88 (1H, m), 2.76 (3H, s), 2.07-2.35 (3H, m), 1.37-1.92 (5H, m), 1.18 (6H, d, J = 6.80 Hz); HPLC: 100% MS (ESI): m/z 608.3 [M + H]+ |
| 129 | 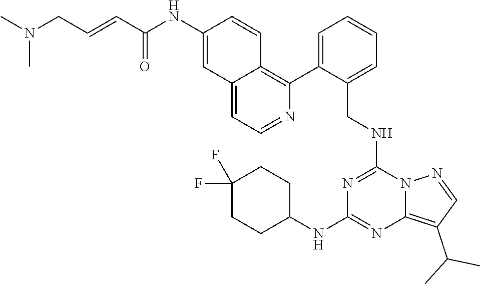 [SMILES: CC(C)c1cnn2c(NCc3ccccc3-c3nccc4cc(NC(=O)/C=C/CN(C)C)ccc34)nc(NC3CCC(F)(F)CC3)nc12] | Light yellow solid; ¹H NMR (400MHz, CD₃OD) δ = 8.67 (d, J = 2.0 Hz, 1H), 8.42 (d, J = 6.8 Hz, 1H), 8.24 (br d, J = 6.6 Hz, 1H), 7.90-7.78 (m, 3H), 7.69 (t, J = 7.4 Hz, 1H), 7.64-7.55 (m, 3H), 7.07-6.98 (m, 1H), 6.71 (d, J = 15.4 Hz, 1H), 5.06-4.95 (m, 1H), 4.79 (br s, 1H), 4.07 (d, J = 7.3 Hz, 2H), 2.96 (s, 6H), 2.84 (br d, J = 6.3 Hz, 1H), 2.18-1.90 (m, 5H), 1.82-1.55 (m, 3H), 1.22-1.15 (m, 6H); LCMS: 94.6%, MS (ESI): 654.6 m/z [M + H]+ |
| 130 | 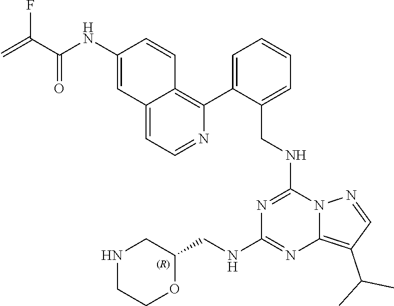 | R form; white powder; ¹H-NMR (CD₃OD, 400 MHz): δ 8.62-8.65 (1H, m), 8.44-8.49 (1H, m), 8.20-8.24 (1H, m), 7.79-7.90 (3H, m), 7.65-7.71 (2H, m), 7.54-7.61 (2H, m), 5.85 (1H, dd, J = 46.4, 3.6 Hz), 5.44 (1H, dd, J = 14.8, 3.6 Hz), 5.03-5.13 (1H, m), 4.65-4.76 (1H, m), 3.81-4.12 (3H, m), 3.32-3.61 (3H, m), 2.78-3.21 (4H, m), 1.16-1.21 (6H, m); HPLC: 97.5%, MS (ESI): 596.5 m/z [M + H]+ |
| 131 | 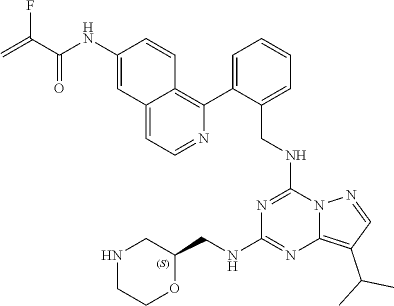 | S form; white powder; ¹H-NMR (CD₃OD, 400 MHz): δ 8.62-8.65 (1H, m), 8.44-8.49 (1H, m), 8.20-8.24 (1H, m), 7.79-7.90 (3H, m), 7.65-7.71 (2H, m), 7.54-7.61 (2H, m), 5.85 (1H, dd, J = 46.4, 3.6 Hz), 5.44 (1H, dd, J = 14.8, 3.6 Hz), 5.03-5.13 (1H, m), 4.65-4.76 (1H, m), 3.81-4.12 (3H, m), 3.32-3.61 (3H, m), 2.78-3.21 (4H, m), 1.16-1.21 (6H, m); HPLC: 95.4%, MS (ESI): 596.5 m/z [M + H]+ |
| 132 | 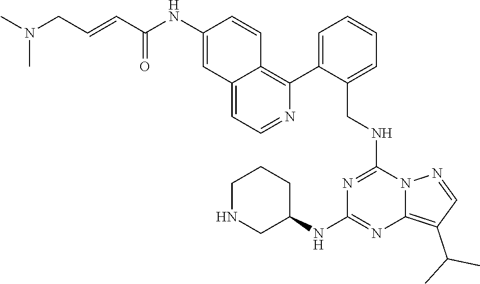 [SMILES: CC(C)c1cnn2c(NCc3ccccc3-c3nccc4cc(NC(=O)/C=C/CN(C)C)ccc34)nc(N[C@@H]3CCCNC3)nc12] | White solid; ¹H NMR (400 MHz, CD₃OD) δ ppm 1.19- 1.28 (m, 6H) 1.69-2.05 (m, 2H) 2.07-2.32 (m, 2H) 2.84-2.94 (m, 1H) 2.98 (s, 6H) 3.04-3.12 (m, 1H) 3.36-3.60 (m, 2H) 4.09 (d, J = 7.03 Hz, 2H) 4.32 (br s, 1H) 4.70 (br d, J = 16.06 Hz, 0.5 H) 4.94-5.10 (m, 2H) 5.35 (br d, J = 14.81 Hz, 0.5H) 6.68-6.77 (m, 1H) 7.00-7.10 (m, 1H) 7.58-7.67 (m, 2H) 7.67-7.77 (m, 2H) 7.79-7.97 (m, 3 ) 8.20-8.33 (m, 1H) 8.47 (dd, J = 16.31, 6.78 Hz, 1H) 8.71 (s, 1H); LCMS: 100.0%, MS (ESI): m/z 619.3 [M + H]+ |
| 133 | 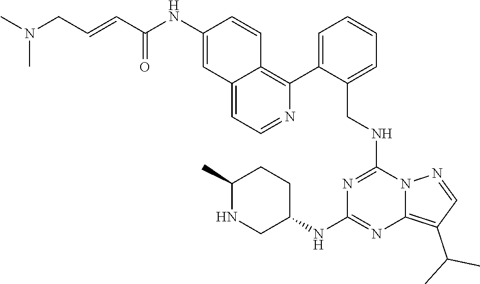 [SMILES: CC(C)c1cnn2c(NCc3ccccc3-c3nccc4cc(NC(=O)/C=C/CN(C)C)ccc34)nc(N[C@H]3CC[C@H](C)NC3)nc12] | White powder; ¹H NMR (400 MHz, CD₃OD) δ 8.67 (s, 1H), 8.40-8.50 (m, 1H), 8.13-8.29 (m, 1H) 7.82-7.88 (m, 2H), 7.70-7.76 (m, 1H), 7.60-7.67 (m, 1H), 7.51-7.58 (m, 1H), 7.41-7.48 (m,1H), 7.30-7.39 (m, 1H), 6.95-7.11 (m, 1H), 6.64-6.80 (m, 1H), 5.30-5.42 (m, 1H), 4.99- 5.09 (m, 1H), 4.96-5.09 (m, 1H), 4.20-4.42 (m, 1H), 4.07 (d, J = 7.20 Hz, 2H), 2.80-3.05 (m, 9H), 2.15-2.25 (m, 1H), 1.99-2.08 (m, 1H), 1.41-1.47 (m, 3H), 1.31-1.36 (m, 2H), 1.21-1.26 (m, 6 H); HPLC: 97.5% MS (ESI): m/z 633.4 [M + H]+ |
| 134 | 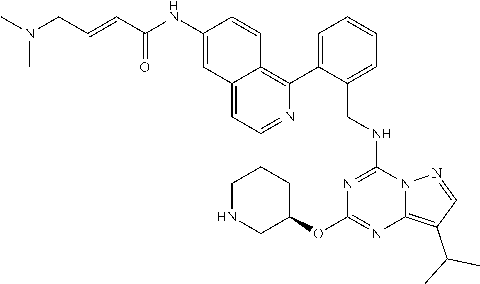 [SMILES: CC(C)c1cnn2c(NCc3ccccc3-c3nccc4cc(NC(=O)/C=C/CN(C)C)ccc34)nc(O[C@@H]3CCCNC3)nc12] | Yellow powder; ¹H NMR (CD₃OD, 400 MHz): δ 8.61 (1H, dd, J = 14.8, 2 Hz), 8.39 (1H, m), 8.16 (1H, t, J = 6.4 Hz), 7.77-7.85 (2H, m), 7.62-7.77 (3H, m), 7.50-7.61 (2H, m), 6.97-7.08 (1H, m), 6.72 (1H, d, J = 15.6 Hz), 5.23 (1H, m), 5.00 (1H, t, J = 15.6 Hz), 4.62 (1H, dd, J = 15.6, 2.8 Hz), 4.07 (2H, d, J = 7.2 Hz), 3.37-3.56 (2H, m), 3.11-3.28 (2H, m), 2.96 (6H, s), 2.82-2.91 (1H, m), 1.78-2.21 (4H, m), 1.12-1.27 (6H, m); LCMS: 99%, MS (ESI): m/z 620.3 [M + H]+ |
| 135 | 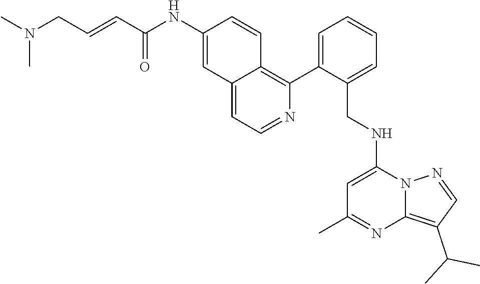 [SMILES: Cc1cc(NCc2ccccc2-c2nccc3cc(NC(=O)/C=C/CN(C)C)ccc23)n2ncc(C(C)C)c2n1] | Brown powder; ¹H NMR (400 MHz, CD₃OD) δ 8.40- 8.49 (m, 2H), 7.86 (s, 1H), 7.80 (d, J = 6.00 Hz, 1H), 7.71-7.76 (m, 1H), 7.67 (t, J = 7.60 Hz, 1H), 7.60 (t, J = 7.20 Hz, 1H), 7.50 (s, 2H), 7.43 (d, J = 6.80 Hz, 1H), 6.90-7.05 (m, 1H), 6.66 (d, J = 15.20 Hz, 1H), 5.80 (s, 1H), 4.90-5.05 (m, 1H) 4.55-4.65 (m, 1H), 4.05 (d, J = 7.20 Hz, 2H), 3.03-3.14 (m, 1H), 2.96 (s, 6H), 2.46 (s, 3H), 1.26 (d, J = 6.80 Hz, 6H); HPLC: 100% MS (ESI): m/z 534.3 [M + H]+ |
| 136 | 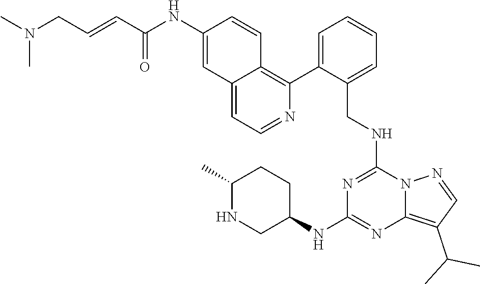 [SMILES: CC(C)c1cnn2c(NCc3ccccc3-c3nccc4cc(NC(=O)/C=C/CN(C)C)ccc34)nc(N[C@@H]3CC[C@@H](C)NC3)nc12] | Yellow solid; ¹H NMR (400 MHz, CD₃OD) δ ppm 1.20- 1.25 (m, 6 H) 1.43 (br s, 3H) 1.63-1.90 (m, 2H) 2.06 (s, 3 H) 2.11-2.26 (m, 1H) 2.79-2.93 (m, 2H) 2.98 (s, 6 H) 3.20-3.28 (m, 1H) 4.09 (d, J = 7.28 Hz, 2H) 4.93- 5.39 (m, 2 H) 6.71 (dd, J = 15.31, 6.53 Hz, 1H) 6.94- 7.12 (m, 1H) 7.57 (br s, 1H) 7.68 (br s, 3H) 7.82 (br s, 2H) 8.12 (br s, 1H) 8.38 (br s, 1H) 8.64 (s, 1H); LCMS: 100.0%, MS (ESI): m/z 633.4 [M + H]+ |
| 137 | 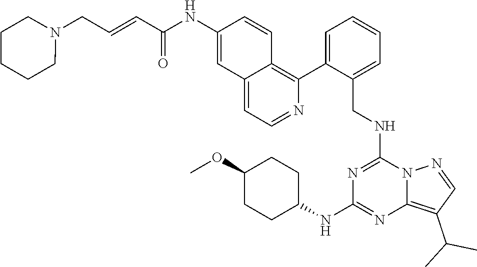 | Yellow powder; ¹H NMR (400 MHz, CD₃OD) δ = 8.49 (s, 1H), 8.32 (br d, J = 6.3 Hz, 1H), 7.86 (br d, J = 6.0 Hz, 1H), 7.76-7.68 (m, 2H), 7.62-7.54 (m, 2H), 7.51 (br s, 2H), 7.44 (br d, J = 7.8 Hz, 1H), 7.05-6.94 (m, 1H), 6.64 (d, J = 15.1 Hz, 1H), 5.00 (br d, J = 15.1 Hz, 1H), 4.57 (br s, 1H), 4.02 (d, J = 7.3 Hz, 2H), 3.55 (br s, 3H), 3.38 (s, 3H), 3.28-3.21 (m, 1H), 3.23 (br s, 1H), 3.12-2.96 (m, 1H), 3.03 (br s, 1H), 2.84 (br s, 1H), 2.20-1.93 (m, 5H), 1.85 (br s, 3H), 1.56 (br s, 1H), 1.45-1.25 (m, 5H), 1.19 (d, J = 7.0 Hz, 6H); LCMS: 100%, MS (ESI): 688.4 m/z [M + H]+ |
| 138 | 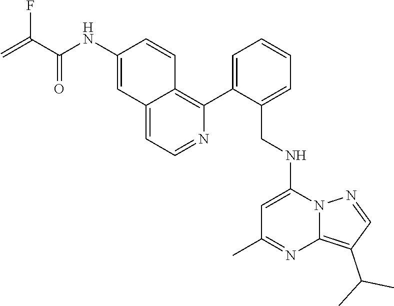 [SMILES: C=C(F)C(=O)Nc1ccc2c(-c3ccccc3CNc3cc(C)nc4c(C(C)C)cnn34)nccc2c1] | White solid; ¹H NMR (400 MHz, CD₃OD) δ = 8.57 (s, 1H), 8.47 (d, J = 6.5 Hz, 1H), 8.23 (br s, 1H), 7.93-7.79 (m, 3H), 7.78-7.65 (m, 2H), 7.59 (br d, J = 9.0 Hz, 2H), 5.95-5.86 (m, 2H), 5.81 (d, J = 3.6 Hz, 1H), 5.46 (dd, J = 3.6, 14.9 Hz, 1H), 4.95-4.94 (m, 1H), 4.96-4.93 (m, 1H), 4.97-4.93 (m, 1H), 4.93 (br s, 1H), 4.95-4.92 (m, 1H), 4.95-4.91 (m, 1H), 4.87-4.86 (m, 1H), 4.86-4.86 (m, 1H), 4.83 (br s, 1H), 4.84-4.82 (m, 1H), 4.85-4.81 (m, 1H), 4.84-4.81 (m, 1H), 3.04 (td, J = 7.0, 13.7 Hz, 1H), 2.49 (s, 3H), 1.25 (dd, J = 4.3, 6.8 Hz, 6H); LCMS: 100%, MS (ESI): 495.1 m/z [M + H]+ |
| 139 | 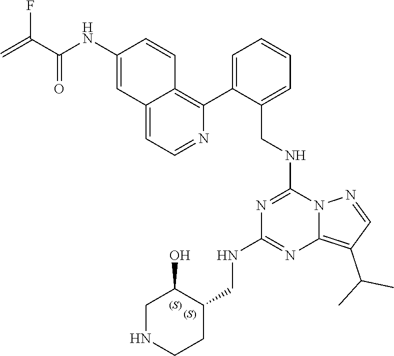 | 3S, 4S form, white solid; ¹H NMR (400 MHz, CD₃OD) δ ppm 1.11-1.25 (m, 6 H) 1.28-1.64 (m, 1H) 1.53 (br s, 1H) 1.87 (br d, J = 11.04 Hz, 1H) 1.96-2.14 (m, 1H) 2.88 (br d, J = 6.02 Hz, 3 H) 3.35-3.78 (m, 5 H) 4.57-4.91 (m, 2 H) 5.11 (br t, J = 15.56 Hz, 2 H) 5.46 (dd, J = 14.93, 3.39 Hz, 1H) 5.81 (br s, 1H) 5.92 (br s, 1H) 7.52-7.93 (m, 7 H) 8.19 (br s, 1H) 8.47 (br s, 1H) 8.62 (br s, 1H); LCMS: 100%, MS (ESI): 610.1 m/z [M + H]+ |
| 140 | 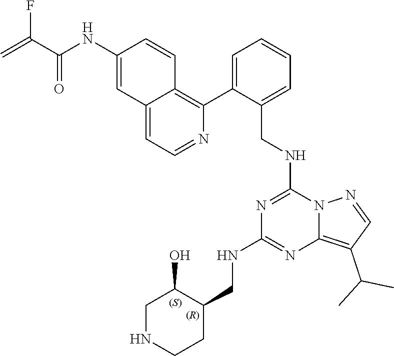 | 3S, 4R form, yellow powder; ¹H NMR (400 MHz, CD₃OD) δ ppm 8.57 (s, 1H), 8.44 (s, 1H), 8.11 ( d, J = 6.40 Hz, 1H), 7.74-7.88 (m, 3H), 7.58-7.70 (m, 2H), 7.51-7.56 (m, 2H), 5.75-5.95 (m, 1H), 5.44 (dd, J = 14.80, 3.60 Hz, 1H), 4.99-5.17 (m, 1H), 4.65 ( d, J = 14.40 Hz, 1H), 4.05 ( d, J = 12.80 Hz, 1H), 3.34-3.48 (m, 2H), 3.20-3.30 (m, 2H), 2.82-3.15 (m, 3H), 1.78-2.01 (m, 2H), 1.63-1.73 (m, 1H), 1.16-1.22 (m, 6H); HPLC: 96.2% MS (ESI): m/z 610.3 [M + H]+ |
| 141 | 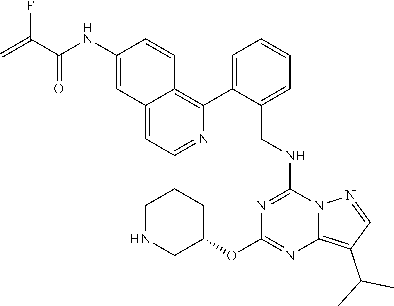 [SMILES: C=C(F)C(=O)Nc1ccc2c(-c3ccccc3CNc3nc(O[C@H]4CCCNC4)nc4c(C(C)C)cnn34)nccc2c1] | Yellow powder; ¹H NMR (400 MHz, CD₃OD) δ ppm 8.58 (dd, J = 19.60, 2.00 Hz, 1H), 8.36-8.43 (m, 1H), 8.13 (d, J = 6.40 Hz, 1H), 7.72-7.82 (m, 3H), 7.59-7.69 (m, 2H), 7.51-7.57 (m, 2H), 5.74-5.95 (m, 1H), 5.44 (dd, J = 14.80, 3.60 Hz, 1H), 5.20 ( s, 1H), 5.03 (d, J = 15.20 Hz, 1H), 4.56 (t, J = 16.00 Hz, 1H), 3.37-3.54 (m, 2H), 3.10-3.27 (m, 2H), 2.80-2.92 (m, 1H), 1.84-2.15 (m, 4H) 1.16-1.26 (m, 6H); HPLC: 99.3% MS (ESI): m/z 581.1 [M + H]+ |
| 142 | 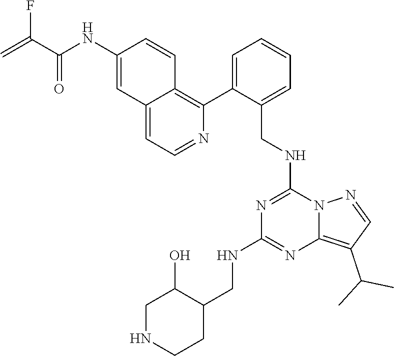 [SMILES: C=C(F)C(=O)Nc1ccc2c(-c3ccccc3CNc3nc(NCC4CCNCC4O)nc4c(C(C)C)cnn34)nccc2c1] | 3R,4R and 3S, 4S form mixture; yellow solid; ¹H NMR (400 MHz, CD₃OD) δ ppm 8.59 (s, 1H), 8.46 ( s, 1H), 8.05-8.25 (m, 1H), 7.75-7.87 (m, 3H), 7.62-7.70 (m, 2H), 7.49-7.58 (m, 2H), 5.71-5.98 (m, 1H), 5.44 (dd, J = 14.80, 3.60 Hz, 1H), 5.12 (d, J = 14.80 Hz, 1H), 4.50- 4.65 (m, 1H), 3.51-3.79 (m, 2H). 3.35-3.46 (m, 3H), 2.95-3.05 (m, 1H), 2.75-2.84 (m, 2H), 2.02 (d, J = 11.20 Hz, 1H), 1.75-1.84 (m, 1H), 1.50-1.62 (m, 1H), 1.15-1.30 (m, 6H); HPLC: 99.2% MS (ESI): m/z 610.3 [M + H]+ |
| 143 | 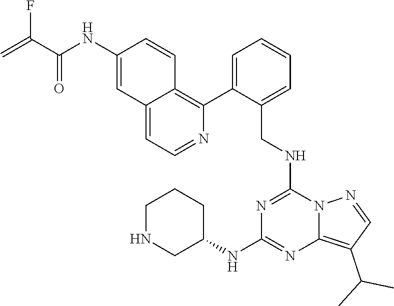 [SMILES: C=C(F)C(=O)Nc1ccc2c(-c3ccccc3CNc3nc(N[C@H]4CCCNC4)nc4c(C(C)C)cnn34)nccc2c1] | Yellow solid; ¹H NMR (400 MHz, DMSO-d6) δ ppm 10.54 (s, 1H) 8.85 (br s, 2 H) 8.52 (d, J = 5.77 Hz, 1H) 8.44-8.49 (m, 1H) 8.47 (d, J = 1.76 Hz, 1H) 8.13 (br s, 1H) 7.86 (dd, J = 9.03, 2.01 Hz, 1H) 7.81 (d, J = 6.02 Hz, 1H) 7.58-7.64 (m, 3 H) 7.51-7.57 (m, 1H) 7.45-7.51 (m, 1H) 7.39 (d, J = 7.53 Hz, 1H) 6.58 (br s, 1H) 5.73- 5.91 (m, 1H) 5.47 (dd, J = 15.69, 3.89 Hz, 1H) 4.49 (br s, 2 H) 4.04 (br s, 1H) 3.22-3.28 (m, 2 H) 2.75-2.99 (m, 4 H) 1.93 (m, 2 H) 1.76 (m, 1H) 1.60 (m, 1H) 1.24 (d, J = 7.03 Hz, 6 H); HPLC: 95.8%, MS (ESI): 580.1 m/z [M + H]+ |
| 144 | 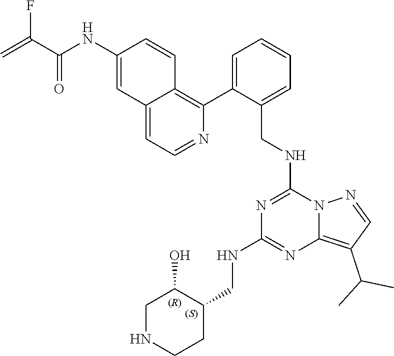 | 3R, 4S form white solid; ¹H NMR (400 MHz, CD₃OD) δ ppm 8.67 (s, 1H) 8.52 (d, J = 6.78 Hz, 1H) 8.31 (d, J = 6.53 Hz, 1H) 7.80-8.00 (m, 3 H) 7.54-7.76 (m, 4H) 5.80-5.98 (m, 1H) 5.48 (dd, J = 14.81, 3.76 Hz, 1H) 5.12 (br dd, J = 15.56, 7.28 Hz, 1H) 4.74 (br dd, J = 15.69, 2.89 Hz, 1H) 4.13 (br s, 1H) 3.35-3.54 (m, 3H) 2.96- 3.23 (m, 2H) 2.87 (dt, J = 13.61, 6.87 Hz, 1H) 1.59-2.12 (m, 3H) 1.14-1.28 (m, 6H) 1.14-1.28 (m, 1H); HPLC: 97.8% (254 nm), MS (ESI): m/z 610.2 [M + H]+ |
| 145 | 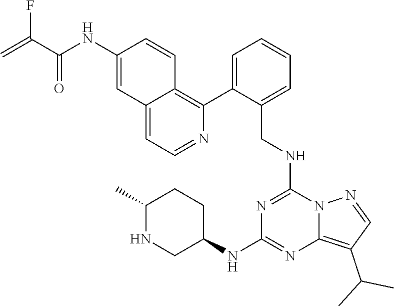 [SMILES: C=C(F)C(=O)Nc1ccc2c(-c3ccccc3CNc3nc(N[C@@H]4CC[C@@H](C)NC4)nc4c(C(C)C)cnn34)nccc2c1] | Yellow solid; ¹H NMR (400 MHz, CD₃OD) δ = 8.67 (br s, 1H), 8.52-8.45 (m, 1H), 8.33-8.20 (m, 1H), 7.95 (br s, 1H), 7.90-7.66 (m, 4H), 7.65-7.57 (m, 2H), 5.96- 5.79 (m, 1H), 5.46 (dd, J = 4.0, 13.6 Hz, 1H), 5.02 (br s, 1H), 5.15-4.93 (m, 1H), 4.62 (br d, J = 15.3 Hz, 1H), 4.34 (br s, 1H), 3.62-3.35 (m, 1H), 3.29-3.21 (m, 1H), 3.09- 2.79 (m, 2H), 2.35-2.10 (m, 1H), 2.08-1.65 (m, 3H), 1.44 (br d, J = 6.3 Hz, 3H), 1.25-1.17 (m, 6H); LCMS: 100%, MS (ESI): m/z 594.3 [M + H]+ |
| 146 | 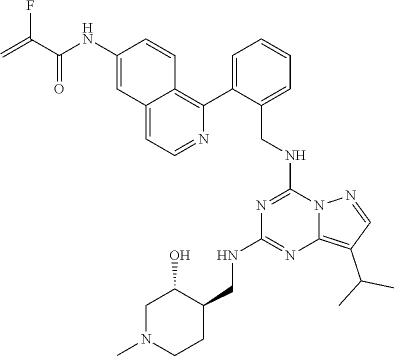 [SMILES: C=C(F)C(=O)Nc1ccc2c(-c3ccccc3CNc3nc(NC[C@H]4CCN(C)C[C@@H]4O)nc4c(C(C)C)cnn34)nccc2c1] | White powder; ¹H NMR (DMSO-d6,400 MHz) δ10.43 (1H, s) 8.51 (1H, d, J = 6.0) 8.41 (1H, d, J = 2.0 Hz) 7.91 (1H, s) 7.73-7.81 (2H, m) 7.56-7.62 (3H, m) 7.41- 7.52 (2 H, m) 7.31-7.37 (1H, m) 6.22 (1H, s) 5.68- 5.86 (1H, m) 5.44 (1H, dd, J = 15.6, 3.6 Hz) 4.89 (1H, S) 4.45 (2H, s) 3.31-3.35 (2H, m) 2.88 (2H, dq, J = 14.0, 7.2 Hz) 2.70-2.80 (1H, m) 2.26 (3H, s) 1.80- 2.05 (2H, m) 1.62 (1H, d, J = 13.2 Hz) 1.23-1.43 (2H, m) 1.21 (6H, d, J = 6.8 Hz); LCMS: 100%, MS (ESI): m/z 624.3 [M + H]+ |
| 147 | 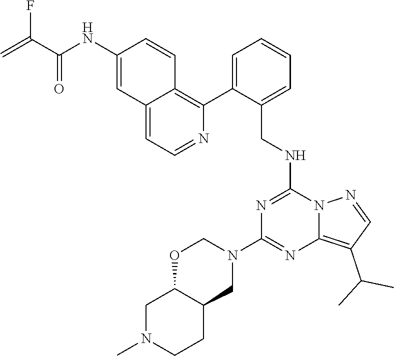 [SMILES: C=C(F)C(=O)Nc1ccc2c(-c3ccccc3CNc3nc(N4CO[C@H]5CN(C)CC[C@@H]5C4)nc4c(C(C)C)cnn34)nccc2c1] | White powder; ¹H NMR (DMSO-d6, 400 MHz) δ10.72 (1H, s) 8.79 (1H, d, J = 6.0 Hz) 8.71 (1H, d, J = 2.4 Hz) 8.40 (1H, s) 8.06 (1H, dd, J = 9.2, 2.0 Hz) 8.01 (1H, d, J = 6.0 Hz) 7.93 (1H, s) 7.82-7.90 (2H, m) 7.70-7.82 (2H, m) 7.64 (1H, dd, J = 7.2, 1.6 Hz) 6.18 (1H, d, J = 9.6 Hz) 5.99-6.15 (1H, m) 5.76 (1H, , J = 15.6, 3.6 Hz) 4.69- 4.84 (3H, m) 4.58-4.66 (1H, m) 3.54-3.68 (2H, m) 3.12-3.26 (2H, m) 3.04 (1H, d, J=11.2 Hz) 2.91-2.99 (1H, m) 2.50 (3H, s) 2.13-2.24 (1H, m) 2.05 (1H, t, J = 10.0 Hz) 1.70 (1H, d, J = 10.4 Hz) 1.56 (1H, s) 1.54- 1.59 (1H, m) 1.53 (6H, d, J = 7.2 Hz); LCMS: 100%, MS (ESI): m/z 636.3 [M + H]+ |
| 148 | 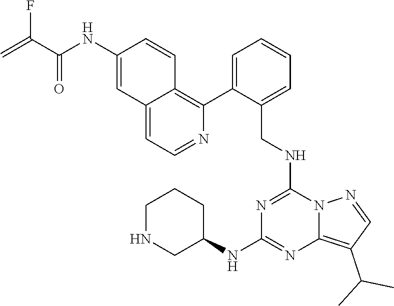 [SMILES: C=C(F)C(=O)Nc1ccc2c(-c3ccccc3CNc3nc(N[C@@H]4CCCNC4)nc4c(C(C)C)cnn34)nccc2c1] | White powder; ¹H NMR (400 MHz, DMSO-d6) δ ppm 1.18 (6H, d, J = 6.78 Hz) 1.61 (1H, br d, J = 9.03 Hz) 1.81- 1.99 (3H, m) 2.86 (4H, dt, J = 13.68, 6.96 Hz) 4.10 (1H, br s) 4.67 (2H, br s) 5.51 (1H, dd, J = 15.56, 3.76 Hz) 5.79-5.98 (1H, m) 7.47 (1H, d, J = 7.53 Hz) 7.53-7.59 (3H, m) 7.68 (1H, t, J = 7.40 Hz) 7.73-7.79 (1H, m) 7.93 (1H, dd, J = 9.29, 2.01 Hz) 8.12 (1H, d, J = 6.27 Hz) 8.47 (1H, d, J= 6 .27 Hz) 8.56 (1H, d, J = 1.76 Hz) 9.11 (2H, br s) 10.80 (1H, br s); LCMS : 99.72%, MS (ESI): m/z 580.3 [M + H]+ |
| 149 | 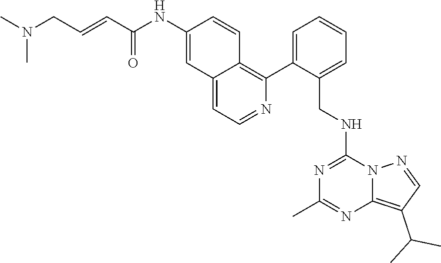 [SMILES: Cc1nc(NCc2ccccc2-c2nccc3cc(NC(=O)/C=C/CN(C)C)ccc23)n2ncc(C(C)C)c2n1] | White solid; ¹H NMR (400 MHz, CD₃OD) δ ppm 8.27 (d, J = 6.02 Hz, 1H) 8.24 (d, J = 1.76 Hz, 1H) 7.59-7.64 (m, 1H) 7.57 (t, J = 2.89 Hz, 2H) 7.54 (td, J = 7.59, 1.38 Hz, 1H) 7.46 (td, J = 7.53, 1.25 Hz, 1H) 7.40-7.44 (m, 1H) 7.34-7.39 (m, 1H) 7.29 (dd, J = 7.53, 1.00 Hz, 1H) 6.81-6.93 (m, 1H) 6.53 (d, J = 15.06 Hz, 1H) 5.03 (br d, J = 14.81 Hz, 1H) 4.32 (d, J = 14.81 Hz, 1H) 3.96 (dd, J = 7.28, 1.00 Hz, 2H) 2.96 (quin, J = 6.90 Hz, 1H) 2.88 (s, 6H) 2.12 (s, 3H) 1.15 (t, J = 7.03 Hz, 6H); HPLC: 98.1%, MS (ESI): 535.1 m/z [M + H]+ |
| 150 | 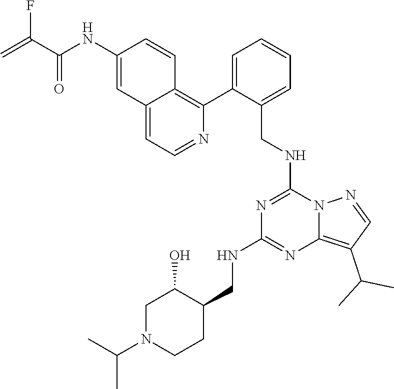 [SMILES: C=C(F)C(=O)Nc1ccc2c(-c3ccccc3CNc3nc(NC[C@H]4CCN(C(C)C)C[C@@H]4O)nc4c(C(C)C)cnn34)nccc2c1] | White powder; ¹H NMR (400 MHz, DMSO-d6) δ 11.11 (1H, s), 10.67-11.00 (1H, d, J = 44.0 Hz), 9.63 (1H, s), 8.32-8.78 (3H, m), 7.32-8.00 (7H, m), 5.95 (1H, dd, J = 15.60, 4.00 Hz), 5.60 (1H, dd, J = 15.60, 4.00 Hz), 4.79-5.01 (1H, m), 4.44-4.65 (1H, m), 3.80-3.95 (1H, m), 3.04-3.41 (5 H, m), 2.75-2.90 (2 H, m), 2.60- 2.70 (1H, m), 1.57-1.93 (3H, m), 1.28 (6 H, br d, J = 6.40 Hz) 1.13 (6 H, t, J = 6.40 Hz); HPLC: 97.9%, MS (ESI): m/z 652.4 [M + H]+ |
| 151 | 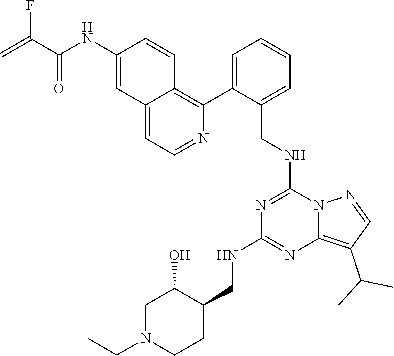 [SMILES: C=C(F)C(=O)Nc1ccc2c(-c3ccccc3CNc3nc(NC[C@H]4CCN(CC)C[C@@H]4O)nc4c(C(C)C)cnn34)nccc2c1] | White powder; ¹H NMR (400 MHz, DMSO-d6) δ 10.90- 11.31 (2H, m), 10.11 (1H, br s), 9.40 (1H, br s), 8.59- 8.69 (1H, m), 8.44-8.55 (1H, m), 8.17-8.29 (1H, m), 7.95-8.09 (1H, m), 7.83 (1H, d, J = 7.6 Hz), 7.73 (1H, t, J = 7.6 Hz), 7.46-7.66 (4H, m), 5.83-6.12 (1H, m), 5.53 (1H, dd, J = 15.6, 4.0 Hz), 3.75-3.94 (1H, m), 3.64 (1H, d, J = 12.0 Hz), 3.32-3.45 (2H, m), 3.02-3.24 (3H, m), 2.88-2.96 (1H, m), 2.58-2.84 (2H, m), 1.92 (1H, d, J = 10.0 Hz), 1.60-1.75 (2H, m), 1.33 (3H, t, J = 4.0 Hz), 1.19 (6 H, d, J = 7.2 Hz); HPLC: 100%, MS (ESI): m/z 638.4 [M + H]+ |
| 152 | 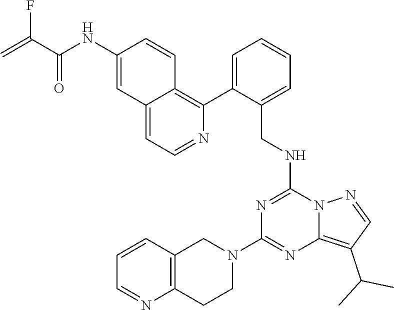 [SMILES: C=C(F)C(=O)Nc1ccc2c(-c3ccccc3CNc3nc(N4CCc5ncccc5C4)nc4c(C(C)C)cnn34)nccc2c1] | White powder; ¹H NMR (400 MHz, DMSO-d6) δ ppm 1.25 (6 H, d, J = 6.78 Hz) 2.86 (2H, t, J = 6.02 Hz) 2.93 (1H, dt, J = 13.80, 6.90 Hz) 3.89 (2H, t, J = 6.02 Hz) 4.52 (2H, br s) 4.70 (2H, s) 5.44 (1H, dd, J = 15.56, 3.76 Hz) 5.68-5.88 (1H, m) 7.18 (1H, dd, J = 7.78, 4.77 Hz) 7.35 (1H, dd, J = 7.40, 1.38 Hz) 7.41-7.53 (3H, m) 7.56 (1H, d, J = 9.29 Hz) 7.59-7.63 (2H, m) 7.68 (1H, d, J = 5.77 Hz) 7.77 (1H, dd, J = 9.16, 2.13 Hz) 8.08 (1H, br s) 8.33- 8.37 (1H, m) 8.42 (1H, d, J = 2.01 Hz) 8.47 (1H, d, J = 5.77 Hz) 10.41 (1H, br s); LCMS: 100% MS (ESI): m/z 614.3 [M + H]+ |
| 153 | 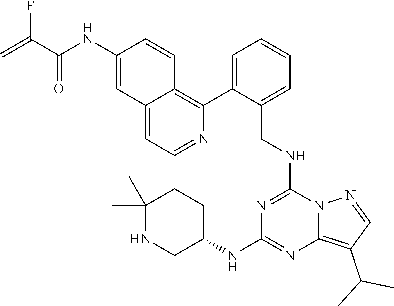 [SMILES: C=C(F)C(=O)Nc1ccc2c(-c3ccccc3CNc3nc(N[C@H]4CCC(C)(C)NC4)nc4c(C(C)C)cnn34)nccc2c1] | Yellow powder; ¹H NMR (400 MHz, DMSO-d6) δ 10.75 (1H, s), 9.05-9.30 (2H, m), 8.56 (1H, d, J = 1.6 Hz), 8.48 (1H, d, J = 6.4 Hz), 8.07 (1H, d, J = 6.4 Hz) 7.94 (1H, dd, J = 9.2, 2.0 Hz), 7.73 (1H, d, J = 7.6 Hz), 7.51-7.68 (4H, m), 7.50-7.51 (1H, m), 7.44-7.47 (1H, m), 5.85 (1H, dd, J = 48.0, 4.0 Hz), 5.50 (1H, dd, J = 16.0, 4.0 Hz), 4.50-4.75 (2H, m), 4.01-4.23 (1H, m), 2.80-3.00 (3H, m), 1.71-1.89 (4H, m), 1.38 (6H, d, J = 16.0 Hz), 1.19 (6H, d, J = 8.0 Hz); HPLC: 98.5%, MS (ESI): m/z 608.3 [M + H]+ |
| 154 | 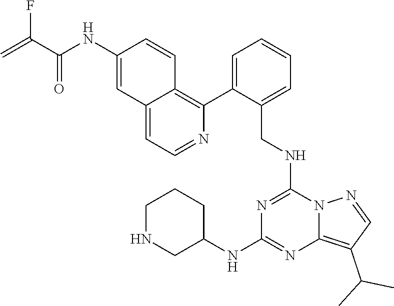 [SMILES: C=C(F)C(=O)Nc1ccc2c(-c3ccccc3CNc3nc(NC4CCCNC4)nc4c(C(C)C)cnn34)nccc2c1] | Yellow powder; ¹H NMR (400 MHz, DMSO-d6) δ ppm 10.71 (1H, s) 9.04 (2H, br s) 8.55 (1H, d, J = 1.76 Hz) 8.49 (1H, d, J = 6.27 Hz) 8.02 (1H, d, J = 6.27 Hz) 7.92 (1H, dd, J = 9.16, 1.88 Hz) 7.68-7.74 (1H, m) 7.50-7.67 (4 H, m) 7.42-7.47 (1H, m) 5.75-5.95 (1H, m) 5.50 (1H, dd, J = 15.56, 4.02 Hz) 4.60 (2H, br s) 4.07 (1H, br s) 2.88 (4H, dt, J = 13.80, 6.90 Hz) 1.93 (2H, br d, J = 10.29 Hz) 1.80 (1H, br d, J = 13.30 Hz) 1.61 (1H, br d, J = 9.54 Hz) 1.21 (7H, d, J = 7.03 Hz); LCMS: 100%, MS (ESI): m/z 580.1 [M + H]+ |
| 155 | 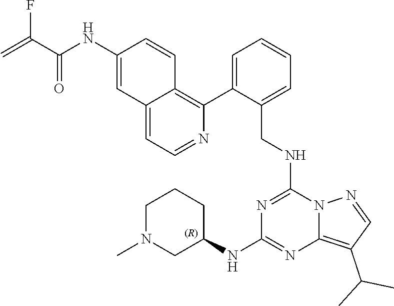 | Pink powder; ¹H NMR (400 MHz, DMSO-d6) δ ppm 1.20 (6H, br d, J = 6.78 Hz) 1.38-1.82 (4 H, m) 1.83- 2.44 (6H, m) 2.76-3.00 (2H, m) 3.49-3.93 (1H, m) 4.42 (2H, br s) 5.52 (1H, dd, J = 15.81, 3.76 Hz) 5.73- 5.89 (1H, m) 5.94-6.77 (1H, m) 7.34 (1H, br d, J = 7.28 Hz) 7.41-7.55 (3H, m) 7.63 (2H, s) 7.76-7.86 (2H, m) 8.26-8.61 (3H, m) 10.70 (1H, br s); LCMS: 100%, MS (ESI): m/z 594.3 [M + H]+ |
| 156 | 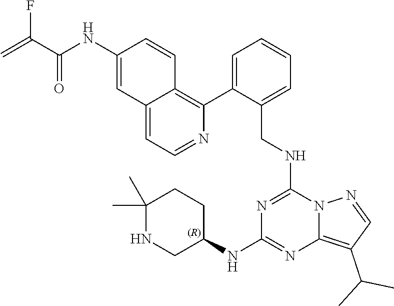 | Yellow solid; ¹H NMR (DMSO-d6,400 MH) δ 8.52 (1H, d, J = 5.6 Hz) 8.44 (1H, d, J = 2.0 Hz) 7.92 (1H, s) 7.83 (1H, dd, J = 9.2, 2.4 Hz) 7.74 (1H, d, J = 5.6 Hz) 7.55- 7.66 (3H, m) 7.40-7.53 (2H, m) 7.35 (1H, dd, J = 7.6, 1.2 Hz) 6.04 (1H, s) 5.68-5.87 (1H, m) 5.45 (1H, dd, J = 15.6, 3.6 Hz) 4.46 (2H, s) 3.59 (1H, s) 2.89 (2H, dt, J = 14.0, 6.8 Hz) 2.55-2.61 (1H, m) 1.69 (1H, s) 1.57 (1H, q, J = 9.2 Hz) 1.42-1.51 (1H, m) 1.25-1.36 (1H, m) 1.22 (6H, d, J = 6.8 Hz) 1.06 (6H, d, J = 4.8 Hz); LCMS: 100%, MS (ESI): m/z 608.3 [M + H]+ |
| 157 | 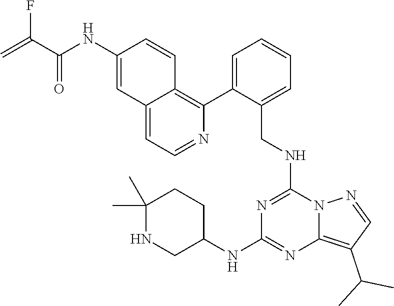 [SMILES: C=C(F)C(=O)Nc1ccc2c(-c3ccccc3CNc3nc(NC4CCC(C)(C)NC4)nc4c(C(C)C)cnn34)nccc2c1] | White powder; ¹H NMR (DMSO-d6,400 MHz) δ8.52 (1H, d, J = 6.0 Hz) 8.44 (1H, d, J =1.2 Hz) 7.93 (1H, s) 7.83 (1H, dd, J = 8.8, 2.0 Hz) 7.74 (1H, d, J = 5.6 Hz) 7.56- 7.65 (3H, m) 7.41-7.52 (2H, m) 7.35 (1H, d, J = 6.4 Hz) 6.05 (1H, s) 5.69-5.87 (1H, m) 5.45 (1H, dd, J = 15.6, 3.6 Hz) 4.46 (2H, s) 3.59 (1H, s) 2.86-2.93 (2H, m) 2.57 (1H, dd, J = 12.4, 7.2 Hz) 1.69 (1H, s) 1.51-1.63 (1H, m) 1.43-1.51 (1H, m) 1.25-1.33 (1H, m) 1.22 (6H, d, J = 7.2 Hz) 1.06 (6H, d, J = 4.0 Hz); LCMS: 98.4%, MS (ESI): m/z 608.3 [M + H]+ |
| 158 | 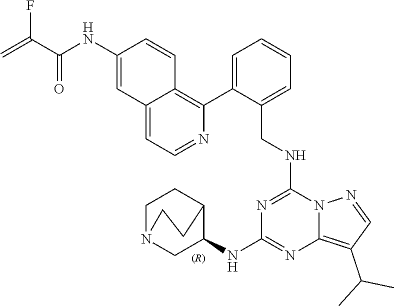 | Yellow powder; ¹H NMR (400 MHz, DMSO-d6) δ ppm 1.19-1.28 (8H, m) 1.73 (1H, br s) 1.90 (2H, br s) 1.99- 2.23 (3H, m) 2.87 (1H, dt, J = 14.05, 7.03 Hz) 3.11- 3.14 (3H, m) 3.54-3.69 (1H, m) 4.11 (1H, br s) 4.56 (2H, br s) 5.50 (1H, dd, J = 15.56, 4.02 Hz) 5.72-5.95 (1H, m) 7.28-7.34 (1H, m) 7.40-7.44 (1H, m) 7.51 (1H, t, J = 7.28 Hz) 7.57-7.64 (4H, m) 7.85-7.94 (2H, m) 8.33 (1H, s) 8.44-8.55 (2H, m) 9.99-10.30 (1H, m) 10.66 (1H, br s); LCMS: 99.0%, MS (ESI): m/z 606.3 [M + H]+ |
| 159 | 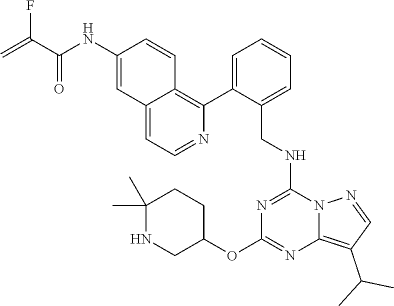 [SMILES: C=C(F)C(=O)Nc1ccc2c(-c3ccccc3CNc3nc(OC4CCC(C)(C)NC4)nc4c(C(C)C)cnn34)nccc2c1] | Yellow powder; ¹H NMR (400 MHz, DMSO-d6) δ ppm 1.22 (6H, d, J = 6.78 Hz) 1.26 (1H, s) 1.38-1.44 (6H, m) 1.70-1.89 (3H, m) 1.99-2.07 (1H, m) 2.90 (1H, dt, J = 13.80, 6.90 Hz) 3.14 (1H, br s) 3.37 (1H, br d, J = 11.54 Hz) 5.06-5.20 (1H, m) 5.53 (1H, dd, J = 15.69, 3.89 Hz) 5.84-5.99 (1H, m) 7.46-7.51 (1H, m) 7.53- 7.64 (2H, m) 7.64-7.75 (3H, m) 7.97 (1H, br d, J = 9.29 Hz) 8.14 (1H, d, J = 6.53 Hz) 8.50 (1H, d, J = 6.27 Hz) 8.60 (1H, d, J = 2.01 Hz) 8.63 (1H, br s) 9.04 (1H, br s) 9.52 (1H, br s) 10.87 (1H, s); LCMS : 98.77%, MS (ESI): m/z 609.2 [M + H]+ |
| 160 | 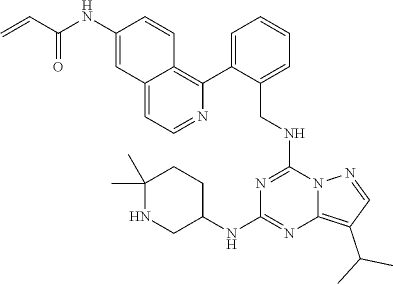 [SMILES: C=CC(=O)Nc1ccc2c(-c3ccccc3CNc3nc(NC4CCC(C)(C)NC4)nc4c(C(C)C)cnn34)nccc2c1] | Yellow powder; ¹H NMR (DMSO-d6, 400 MHz) δ 10.39 (1H, br s), 8.49 (1H, d, J = 4.0 Hz), 8.45 (1H, d, J = 4.0 Hz), 8.04 (1H, br s), 7.73 (1H, d, J = 4.0 Hz), 7.67-7.71 (1H, m), 7.55-7.63 (3H, m), 7.43-7.53 (2H, m), 7.35 (1H, dd, J = 8.0, 4.0 Hz), 6.47-6.66 (1H, m) 6.23-6.38 (2H, m), 5.81 (1H, dd, J = 8.0, 2.0 Hz), 4.47 (2H, br s), 3.81 (1H, br s), 2.78-2.90 (3H, m), 1.63-1.79 (2H, m), 1.43-1.53 (2H, m), 1.13-1.29 (12H, m); HPLC: 97.1%, MS (ESI): m/z 590.3 [M + H]+ |
| 161 | 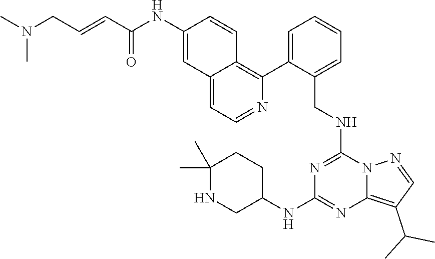 [SMILES: CC(C)c1cnn2c(NCc3ccccc3-c3nccc4cc(NC(=O)/C=C/CN(C)C)ccc34)nc(NC3CCC(C)(C)NC3)nc12] | White powder; ¹H NMR (DMSO-d6, 400 MHz) δ ppm 10.30 (1H, s) 8.48 (1H, d, J = 5.6 Hz) 8.43 (1H, s) 8.01 (1H, s) 7.65-7.74 (2H, m) 7.54-7.62 (3H, m) 7.42- 7.52 (2H, m) 7.35 (1H, d, J = 6.8 Hz) 6.78-6.90 (1H, m) 6.35 (1H, d, J = 15.6 Hz) 6.18 (1H, s) 4.46 (2H, s) 3.69 (1H, s) 2.85-2.95 (1H, m) 2.67 (2H, s) 2.50 (17 H, s) 2.22 (6H, s) 1.90 (1H, s) 1.71 (1H, s) 1.49-1.66 (3H, m) 1.40 (1H, d, J = 9.6 Hz) 1.22 (6H, d, J = 6.8 Hz) 1.13 (6 H, s); LCMS: 100%, MS (ESI): m/z 647.3 [M + H]+ |
| 162 | 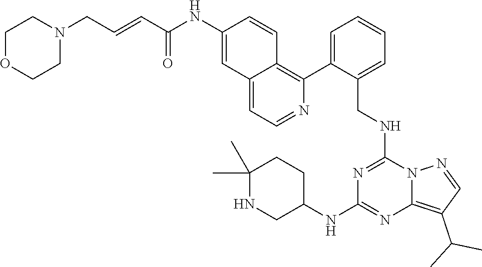 [SMILES: CC(C)c1cnn2c(NCc3ccccc3-c3nccc4cc(NC(=O)/C=C/CN5CCOCC5)ccc34)nc(NC3CCC(C)(C)NC3)nc12] | White powder; ¹H NMR (DMSO-d6, 400 MHz) δ ppm 10.33 (1H, s) 8.48 (1H, d, J = 5.6 Hz) 8.43 (1H, d, J = 2.0 Hz) 8.06 (1H, s) 7.66-7.74 (2H, m) 7.54-7.62 (3H, m) 7.42-7.52 (2H, m) 7.35 (1H, dd, J = 7.6, 1.2Hz) 6.84 (1H, dt, J = 15.6, 6.0 Hz) 6.30-6.42 (2H, m) 4.46 (2H, s) 3.85 (1H, s) 3.59-3.66 (4 H, m) 2.90 (1H, dt, J = 13.6, 6.8 Hz) 2.43-2.46 (4 H, m) 1.91 (1H, s) 1.78 (1H, s) 1.65 (2H, d, J = 13.2 Hz) 1.54 (1H, d, J = 10.4 Hz) 1.22- 1.27 (12H, m); LCMS: 100%, MS (ESI): m/z 689.4 [M + H]+ |
| 163 | 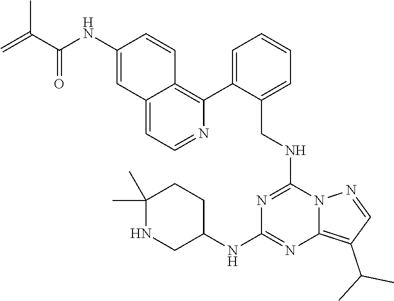 [SMILES: C=C(C)C(=O)Nc1ccc2c(-c3ccccc3CNc3nc(NC4CCC(C)(C)NC4)nc4c(C(C)C)cnn34)nccc2c1] | White powder; ¹H NMR (400 MHz, DMSO-d6) δ 9.99 (1H, s), 8.49 (1H, d, J = 4.0 Hz), 8.43 (1H, s), 7.99 (1H, s), 7.80 (1H, d, J = 8.0 Hz), 7.72 (1H, d, J = 4.0 Hz), 7.55- 7.63 (3H, m), 7.41-7.52 (2H, m), 7.35 (1H, d, J = 8.0 Hz,), 6.13 (1H, s), 5.89 (1H, s), 5.57 (s, 1H), 4.46 (2H, s), 3.63 (1H, s), 2.83-3.00 (2H, m), 2.60-2.70 (1H, m), 2.01 (3H, s), 1.62-1.69 (1H, m), 1.56-1.59 (1H, m), 1.46-1.55 (1H, m), 1.30-1.35 (1H, m), 1.23 (6 H, d, J = 4.0 Hz), 1.09 (6 H, s); LCMS: 100%, MS (ESI): m/z 604.3 [M + H]+ |
| 164 | 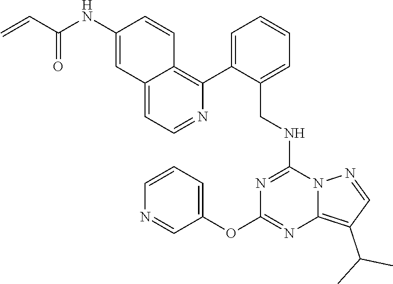 [SMILES: C=CC(=O)Nc1ccc2c(-c3ccccc3CNc3nc(Oc4cccnc4)nc4c(C(C)C)cnn34)nccc2c1] | Yellow powder; ¹H NMR (400 MHz, DMSO-d6) δ ppm 10.29 (s, 1H) 8.72 (br s, 1H) 8.28-8.52 (m, 4 H) 7.76- 7.90 (m, 1H) 7.68 (d, J = 5.60 Hz, 1H) 7.55-7.65 (m, 2H) 7.49-7.53 (m, 3H) 7.43-7.48 (m, 1H) 7.40 (dd, J = 8.40, 4.64 Hz, 1H) 7.33 (d, J = 6.40 Hz, 1H) 6.40- 6.59 (m, 1H) 6.26-6.39 (m, 1H) 5.69-5.88 (m, 1H) 4.49 (br s, 2H) 1.17 (d, J = 7.20 Hz, 6 H); LCMS: 100%, MS (ESI): m/z 557.5 [M + H]+ |
| 165 | 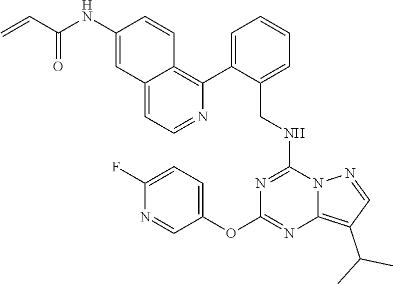 | Yellow powder; ¹H NMR (400 MHz, DMSO-d6) δ ppm 10.40 (br s, 1H) 8.37-8.45 (m, 2H) 8.19-8.26 (m, 1H) 8.10 (d, J = 1.20 Hz, 1H) 7.77-7.87 (m, 2H) 7.67 (d, J = 5.60 Hz, 1H) 7.57-7.62 (m, 1H) 7.41-7.54 (m, 4 H) 7.37 (m, 1H) 7.26-7.37 (m, 1H) 7.13 (dd, J = 8.80, 3.20 Hz, 1H) 6.42-6.60 (m, 1H) 6.25-6.40 (m, 1H) 5.72- 5.84 (m, 1H) 4.47 (br s, 2H) 2.77-2.93 (m, 1H) 1.16 (d, J = 7.20 Hz, 6 H); LCMS: 100%, MS (ESI): m/z 1011.5 [M + H]+ |
| 166 | 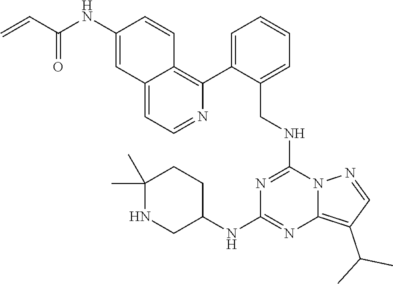 [SMILES: C=CC(=O)Nc1ccc2c(-c3ccccc3CNc3nc(NC4CCC(C)(C)NC4)nc4c(C(C)C)cnn34)nccc2c1] | White powder; ¹H NMR (400 MHz, DMSO-d6) δ10.39 (1H, s), 8.53 (1H, d, J = 4.0 Hz), 8.47 (1H, s), 7.76 (1H, d, J = 4.0 Hz), 7.68-7.73 (1H, m), 7.60-7.64 (1H, m), 7.43-7.57 (4 H, m), 7.37 (1H, d, J = 8.0 Hz), 7.12-7.24 (1H, m), 6.47-6.59 (1H, m), 6.27-6.39 (1H, m), 6.17 (1H, br d, J = 8.0 Hz), 5.82 (1H, d, J = 12.0 Hz), 5.03 (1H, s), 4.25 (2H, br d, J = 4.0 Hz), 3.86 (1H, br s), 2.90- 2.97 (2H, m), 2.73-2.88 (1H, m), 1.45-1.90 (4 H, m), 1.25 (6 H, d, J = 8.0 Hz), 1.21 (6 H, s); HPLC: 98.8% (254 nm), MS (ESI): m/z 589.3 [M + H]+ |
| 167 | 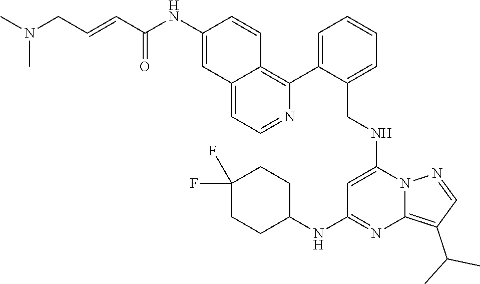 [SMILES: CC(C)c1cnn2c(NCc3ccccc3-c3nccc4cc(NC(=O)/C=C/CN(C)C)ccc34)cc(NC3CCC(F)(F)CC3)nc12] | White powder; ¹H NMR (400 MHz, DMSO-d6) δ ppm 1.25 (6 H, d, J = 6.78 Hz) 1.52-1.64 (3H, m) 1.92 (3H, br s) 1.99-2.09 (3H, m) 2.22 (5 H, s) 2.96 (1H, dt, J = 13.55, 6.78 Hz) 3.87 (2H, br s) 4.24 (2H, br d, J = 6.27 Hz) 5.05 (1H, s) 6.21 (1H, br d, J = 6.78 Hz) 6.32- 6.38 (1H, m) 6.84 (1H, dt, J = 15.43, 5.96 Hz) 7.11 (1H, br s) 7.35-7.38 (1H, m) 7.43-7.48 (1H, m) 7.48- 7.51 (1H, m) 7.52 (1H, s) 7.53-7.57 (1H, m) 7.58-7.61 (1H, m) 7.65-7.69 (1H, m) 7.74 (1H, d, J = 5.52 Hz) 8.45 (1H, d, J = 2.01 Hz) 8.51 (1H, d, J = 5.77 Hz) 10.29 (1H, s); LCMS: 97.46%, MS (ESI): m/z 653.3 [M + H]+ |
| 168 | 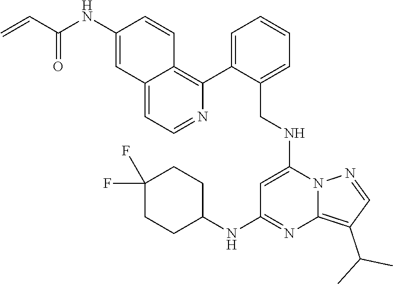 [SMILES: C=CC(=O)Nc1ccc2c(-c3ccccc3CNc3cc(NC4CCC(F)(F)CC4)nc4c(C(C)C)cnn34)nccc2c1] | Pink powder; ¹H NMR (400 MHz, DMSO-d6) δ 10.37 (1H, s), 8.53 (1H, d, J = 4.0 Hz), 8.47 (1H, d, J = 2.0 Hz), 7.76 (1H, d, J = 8.0 Hz), 7.67-7.72 (1H, m), 7.59-7.65 (1H, m), 7.43-7.58 (4 H, m), 7.37 (1H, d, J = 8.0 Hz) 7.08-7.15 (1H, m), 6.47-6.60 (1H, m), 6.30-6.42 (1H, m), 6.22 (1H, d, J = 8.0 Hz), 5.82 (1H, dd, J = 10.00, 2.0 Hz), 5.06 (1H, s), 4.25 (2H, d, J = 8.0 Hz), 3.82- 3.94 (1H, m), 2.91-3.00 (1H, m), 1.85-2.10 (6 H, m), 1.51-1.65 (2H, m), 1.26 (6 H, d, J = 4.0 Hz); HPLC: 98.0% (254 nm), MS (ESI): m/z 596.3 [M + H]+ |
| 169 | 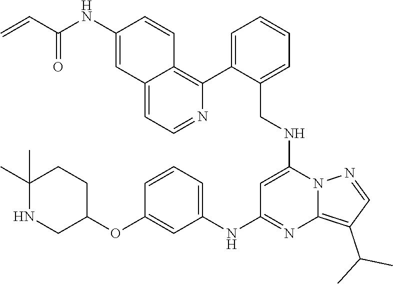 [SMILES: C=CC(=O)Nc1ccc2c(-c3ccccc3CNc3cc(Nc4cccc(OC5CCC(C)(C)NC5)c4)nc4c(C(C)C)cnn34)nccc2c1] | White powder; ¹H NMR (DMSO-d6, 400 MHz) δ10.36 (1H, s) 8.72 (1H, s) 8.52 (1H, d, J = 5.6 Hz) 8.46 (1H, d, J = 2.0 Hz) 7.72-7.77 (2H, m) 7.62-7.70 (3H, m) 7.45-7.57 (3H, m) 7.38 (1H, d, J = 7.2Hz) 7.32 (1H, s) 7.08-7.12 (2H, m) 6.46-6.56 (2H, m) 6.30-6.40 (1H, m) 5.78-5.85 (1H, m) 5.35 (1H, s) 4.32 (2H, d, J = 6.0 Hz) 4.18-4.25 (1H, m) 2.81 (1H, dd, J = 13.2, 7.2 Hz) 1.90-1.96 (1H, m) 1.67-1.79 (1H, m) 1.56-1.65 (1H, m) 1.35-1.42 (1H, m) 1.32 (6 H, dd, J = 6.8, 0.8 Hz) 1.26 (1H, d, J = 3.2Hz) 1.11 (6 H, d, J = 12.0 Hz); LCMS: 99.6%, MS (ESI): m/z 681.4 [M + H]+ |
| 170 | 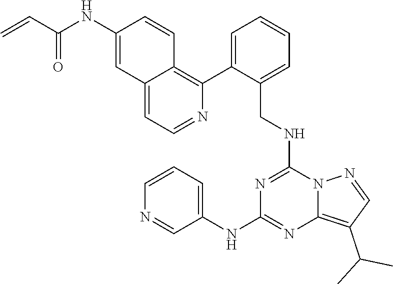 [SMILES: C=CC(=O)Nc1ccc2c(-c3ccccc3CNc3nc(Nc4cccnc4)nc4c(C(C)C)cnn34)nccc2c1] | White powder; ¹H NMR (400 MHz, DMSO-d6) δ ppm 10.39 (s, 1H) 9.10 (br s, 1H) 8.99 (d, J = 2.51 Hz, 1H) 8.54 (d, J = 5.77 Hz, 1H) 8.48 (s, 1H) 8.35 (br s, 1H) 8.22-8.26 (m, 1H) 8.20-8.22 (m, 1H) 7.80 (s, 1H) 7.75 (d, J = 5.77 Hz, 1H) 7.68-7.73 (m, 2H) 7.67 (s, 1H) 7.59 (td, J = 7.47, 1.38 Hz, 1H) 7.52-7.57 (m, 1H) 7.44 (dd, J = 7.40, 1.38 Hz, 1H) 7.30 (dd, J = 8.41, 4.64 Hz, 1H) 6.52-6.64 (m, 1H) 6.36-6.47 (m, 1H) 5.85-5.93 (m, 1H) 4.65 (br d, J = 5.02Hz, 2H) 3.02-3.10 (m, 1H) 1.37 (d, J = 6.78 Hz, 6 H); HPLC: 98.7%, MS (ESI): 556.1 m/z [M + H]+ |
| 171 | 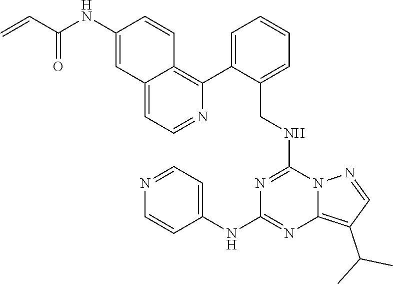 [SMILES: C=CC(=O)Nc1ccc2c(-c3ccccc3CNc3nc(Nc4ccncc4)nc4c(C(C)C)cnn34)nccc2c1] | White powder; ¹H NMR (DMSO-d6, 400 MHz) δ8.85 (2H, d, J = 7.6 Hz) 8.23-8.30 (2H, m) 7.98 (1H, s) 7.67 (1H, d, J = 7.2Hz) 7.39-7.56 (5 H, m) 7.32 (1H, d, J = 7.2 Hz) 7.01 (2H, d, J = 7.2Hz) 6.49-6.59 (1H, m) 6.33 (1H, dd, J = 16.8, 2.0 Hz) 5.80 (1H, dd, J = 10.0, 2.0 Hz) 4.71 (2H, s) 1.31 (6 H, d, J = 6.8 Hz); LCMS: 97.2%, MS (ESI): m/z 556.3 [M + H]+ |
| 172 | 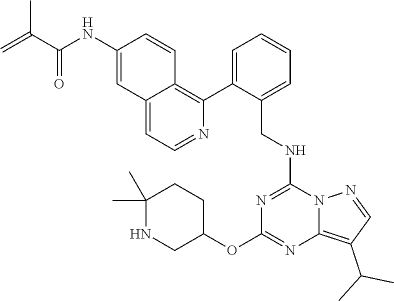 [SMILES: C=C(C)C(=O)Nc1ccc2c(-c3ccccc3CNc3nc(OC4CCC(C)(C)NC4)nc4c(C(C)C)cnn34)nccc2c1] | White powder; ¹H NMR (400 MHz, DMSO-d6) δ ppm 1.09 (6 H, d, J = 8.78 Hz) 1.25 (6 H, d, J = 7.03 Hz) 1.32- 1.40 (1H, m) 1.51-1.58 (1H, m) 1.64-1.74 (1H, m) 1.84- 1.95 (1H, m) 2.01 (3H, s) 2.76 (1H, dd, J = 12.92, 7.40 Hz) 2.94-3.00 (2H, m) 4.51 (2H, br s) 4.69 (1H, tt, J = 7.78, 4.02 Hz) 5.57 (1H, s) 5.89 (1H, s) 7.34 (1H, d, J = 7.03 Hz) 7.43-7.52 (2H, m) 7.53-7.60 (2H, m) 7.68 (1H, d, J = 5.77 Hz) 7.73-7.79 (2H, m) 8.40 (1H, s) 8.45 (1H, d, J = 5.52 Hz) 9.97 (1H, br s); LCMS: 100%, MS (ESI): m/z 605.4 [M + H]+ |
| 173 | 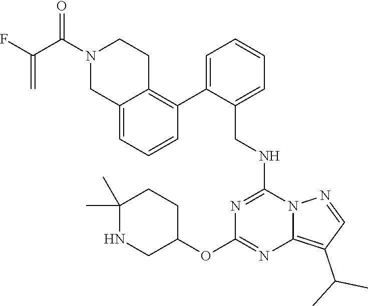 [SMILES: C=C(F)C(=O)N1CCc2c(cccc2-c2ccccc2CNc2nc(OC3CCC(C)(C)NC3)nc3c(C(C)C)cnn23)C1] | White powder; ¹H NMR (400 MHz, DMSO-d6) δ ppm 8.65 (br s, 1H) 7.87 (s, 1H) 7.41-7.48 (m, 1H) 7.31- 7.41 (m, 2H) 7.19-7.25 (m, 1H) 7.15-7.19 (m, 1H) 7.06-7.15 (m, 2H) 5.13-5.32 (m, 2H) 5.08 (br s, 1H) 4.76 (br d, J = 17.07 Hz, 1H) 4.53 (m 2H) 4.32-4.45 (m, 1H) 3.74 (m, 1H) 3.50 (m, 1H) 3.36 (m, 1H) 3.15-3.18 (m, 1H) 2.99-3.03 (m, 1H) 2.54 (m, 2H) 2.04 (m, 1H) 1.72-1.90 (m, 2H) 1.56-1.67 (m, 1H) 1.34 (m, 6 H) 1.29 (d, J = 7.03Hz, 6 H); HPLC: 100%, MS (ESI): 598.4 m/z [M + H]+ |
| 174 | 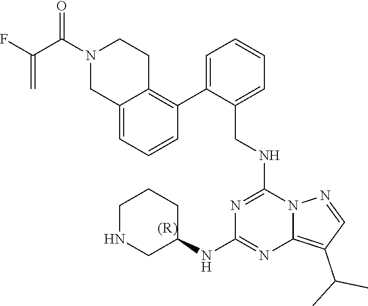 | White powder; ¹H NMR (400 MHz, DMSO-d6) δ ppm 9.23 (br s, 1H) 9.09 (br s, 1H) 8.15-8.83 (m, 1H) 7.75 (s, 1H) 7.47 (m, 1H) 7.31-7.42 (m, 2H) 7.19-7.26 (m, 1H) 7.06-7.19 (m, 3H) 5.10-5.32 (m, 2H) 4.74 (m, 1H) 4.32-4.70 (m, 3H) 4.16 (br s, 1H) 3.65-3.75 (m, 2H) 3.34 (m, 1H) 3.15 (m, 1H) 2.98 (dt, J = 13.80, 6.90 Hz, 1H) 2.87 (m, 2H) 2.53-2.60 (m, 1H) 2.42-2.48 (m, 1H) 1.85-2.06 (m, 2H) 1.71-1.85 (m, 1H) 1.55- 1.69 (m, 1H) 1.27 (d, J = 6.78 Hz, 6 H); HPLC: 100%, MS (ESI): 569.1 m/z [M + H]+ |
| 175 | 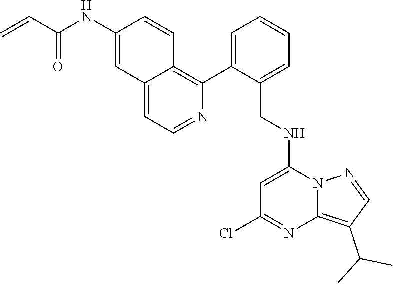 [SMILES: C=CC(=O)Nc1ccc2c(-c3ccccc3CNc3cc(Cl)nc4c(C(C)C)cnn34)nccc2c1] | White solid; ¹H NMR (400 MHz, CDCl₃) δ ppm 8.59 (1H, d, J = 5.52Hz) 8.36 (1H, d, J = 1.76 Hz) 7.72 (1H, s) 7.39-7.67 (7 H, m) 7.31 (1H, dd, J = 9.03, 2.01 Hz) 7.03 (1H, br t, J = 6.15 Hz) 6.52 (1H, dd, J = 16.81, 1.00 Hz) 6.24-6.34 (1H, m) 5.87 (1H, dd, J = 10.29, 1.00 Hz) 5.61 (1H, s) 4.28-4.59 (2H, m) 3.13-3.23 (1H, m) 1.28 (7 H, d, J = 6.78 Hz); LCMS: 100.0%, MS (ESI): m/z 497.2 [M + H] + |
| 176 | 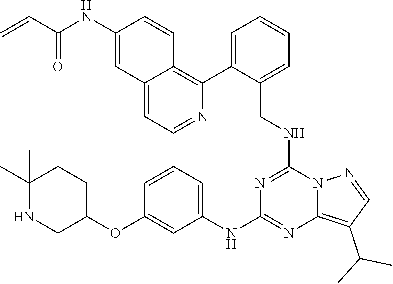 [SMILES: C=CC(=O)Nc1ccc2c(-c3ccccc3CNc3nc(Nc4cccc(OC5CCC(C)(C)NC5)c4)nc4c(C(C)C)cnn34)nccc2c1] | White powder; ¹H NMR (400 MHz, DMSO-d6) δ ppm 10.35 (br s, 1H) 8.77 (s, 1H) 8.48 (d, J = 5.77 Hz, 1H) 8.42 (s, 1H) 8.22 (br s, 1H) 7.67-7.72 (m, 2H) 7.59- 7.66 (m, 2H) 7.49-7.55 (m, 1H) 7.44-7.49 (m, 1H) 7.37 (br d, J = 7.28 Hz, 1H) 7.23 (br d, J = 9.29 Hz, 1H) 7.10 (t, J = 8.16 Hz, 1H) 5.82 (br dd, J = 10.16, 1.88 Hz, 1H) 4.57 (br s, 2H) 4.21 (br d, J = 3.76 Hz, 1H) 3.00- 3.06 (m, 2H) 2.79 (br dd, J = 12.80, 7.03Hz, 1H) 1.89- 2.07 (m, 2H) 1.67-1.77 (m, 1H) 1.54-1.64 (m, 1H) 1.18-1.35 (m, 8 H) 1.10 (d, J = 13.80 Hz, 4 H); HPLC: 96.8%, MS (ESI): 682.3 m/z [M + H]+ |
| 177 | 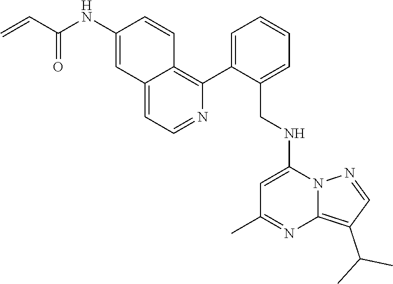 [SMILES: C=CC(=O)Nc1ccc2c(-c3ccccc3CNc3cc(C)nc4c(C(C)C)cnn34)nccc2c1] | Yellow solid; ¹H NMR (DMSO-d6, 400 MHz) δ10.37 (1H, s) 8.54 (1H, d, J = 5.77 Hz) 8.48 (1H, d, J = 1.76 Hz) 7.70-7.79 (3H, m) 7.58-7.69 (3H, m) 7.48 (2H, dtd, J = 18.54, 7.42, 7.42, 1.51 Hz) 7.38 (1H, dd, J = 7.53, 1.51 Hz) 6.47-6.56 (1H, m) 6.31-6.40 (1H, m) 5.82 (1H, dd, J = 10.16, 1.88 Hz) 5.52 (1H, s) 4.33 (2H, d, J = 6.53 Hz) 2.21 (3H, s) 1.27 (7 H, d, J = 6.78 Hz); LCMS: 100%, MS (ESI): m/z 477.1 [M + H]+ |
| 178 | 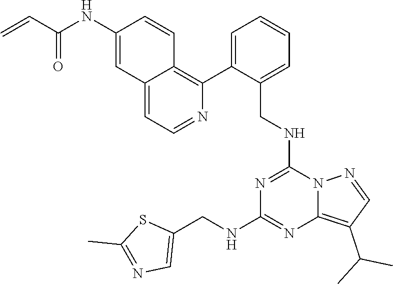 [SMILES: C=CC(=O)Nc1ccc2c(-c3ccccc3CNc3nc(NCc4cnc(C)s4)nc4c(C(C)C)cnn34)nccc2c1] | White solid; ¹H NMR (400 MHz, DMSO-d6) δ ppm 10.32 (1H, br s) 8.36-8.56 (3H, m) 8.14 (2H, br s) 7.68 (1H, d, J = 5.77 Hz) 7.61-7.65 (2H, m) 7.52-7.60 (2H, m) 7.40-7.51 (2H, m) 7.30-7.39 (2H, m) 6.94 (2H, br s) 6.26-6.59 (4 H, m) 5.81 (1H, dd, J = 10.16, 1.88 Hz) 4.38-4.53 (4 H, m) 2.63-2.73 (1H, m) 2.52 (3H, br s) 1.18-1.30 (6 H, m); LCMS: 100.0%, MS (ESI): m/z 590.1 [M + H] + |
| 179 | 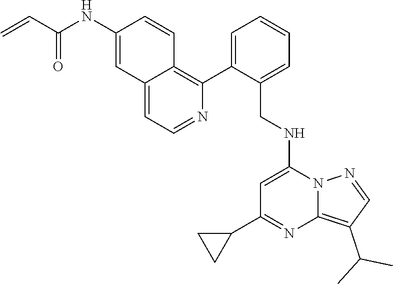 [SMILES: C=CC(=O)Nc1ccc2c(-c3ccccc3CNc3cc(C4CC4)nc4c(C(C)C)cnn34)nccc2c1] | White powder; ¹H NMR (400 MHz, DMSO-d6) δ ppm 10.37 (1H, s) 8.54 (1H, d, J = 5.77 Hz) 8.46 (1H, d, J = 1.51 Hz) 7.77 (1H, d, J = 5.77 Hz) 7.54-7.75 (5H, m) 7.43-7.53 (2H, m) 7.33-7.40 (1H, m) 6.45-6.56 (1H, m) 6.29-6.39 (1H, m) 5.81 (1H, dd, J = 10.04, 1.76 Hz) 5.55 (1H, s) 4.33 (2H, br d, J = 6.02 Hz) 2.95-3.06 (1H, m) 1.73 (1H, quin, J = 6.40 Hz) 1.25 (6H, d, J = 7.03 Hz) 0.86 (4 H, d, J = 6.53 Hz); HPLC: 98.32%, MS (ESI): m/z 503.1 [M + H] + |
| 180 | 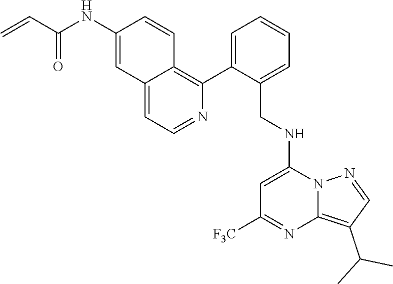 [SMILES: C=CC(=O)Nc1ccc2c(-c3ccccc3CNc3cc(C(F)(F)F)nc4c(C(C)C)cnn34)nccc2c1] | White powder; ¹H NMR (400 MHz, DMSO-d6) δ ppm 1.30 (6 H, d, J = 6.78 Hz) 3.15 (1H, dt, J = 13.74, 7.06 Hz) 4.50 (2H, br s) 5.79-5.83 (1H, m) 6.12 (1H, s) 6.31- 6.37 (1H, m) 6.46-6.55 (1H, m) 7.37 (1H, dd, J = 7.40, 1.38 Hz) 7.45-7.54 (3H, m) 7.57-7.62 (1H, m) 7.64 (1H, d, J = 7.28 Hz) 7.71 (1H, d, J = 5.77 Hz) 7.99 (1H, s) 8.42 (1H, d, J = 1.76 Hz) 8.49 (1H, d, J = 5.77 Hz) 10.32 (1H, s); HPLC: 99.78% MS (ESI): m/z 531.2 [M + H]+ |
| 181 | 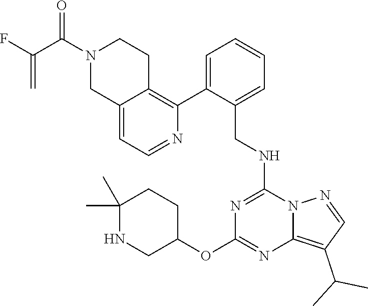 [SMILES: C=C(F)C(=O)N1CCc2c(ccnc2-c2ccccc2CNc2nc(OC3CCC(C)(C)NC3)nc3c(C(C)C)cnn23)C1] | White powder; ¹H NMR (400 MHz, DMSO-d6) δ ppm 8.37 (2H, d, J = 5.2 Hz), 7.80 (1H, s), 7.43-7.54 (1H, m), 7.32-7.43 (2H, m), 7.22 (1H, dd, J = 8.0, 2.0 Hz), 7.17 (1H, d, J = 4.0 Hz), 5.11-5.34 (2H, m), 4.58-4.81 (3H, m), 4.49 (2H, br s), 3.61 (2H, br s), 2.91-3.06 (2H, m), 2.70-2.81 (1H, m), 2.57-2.63 (2H, m), 1.91- 2.09 (2H, m), 1.63-1.76 (1H, m), 1.50-1.57 (1H, m), 1.32-1.41 (1H, m), 1.26 (6 H, d, J = 8.0 Hz), 1.07 (6H, d, J = 12.0 Hz); HPLC (254 nm): 98.4%, MS (ESI): m/z 599.3 [M + H]+ |
| 182 | 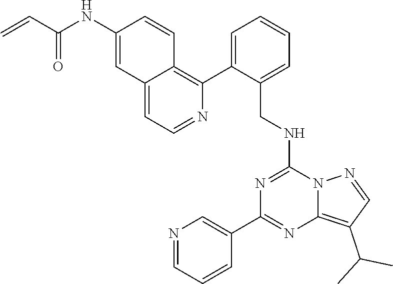 [SMILES: C=CC(=O)Nc1ccc2c(-c3ccccc3CNc3nc(-c4cccnc4)nc4c(C(C)C)cnn34)nccc2c1] | White powder; ¹H NMR (400 MHz, DMSO-d6) δ 9.27 (1H, br s), 8.62 (1H, br d, J = 8.0 Hz), 8.38 (1H, br d, J = 8.0 Hz), 8.25-8.33 (2H, m), 7.88 (1H, br s), 7.67 (1H, br d, J = 8.0 Hz), 7.44-7.55 (5 H, m), 7.32 (1H, br d, J = 8.0 Hz), 6.45-6.52 (2H, m), 6.27-6.35 (1H, m), 5.75-5.85 (1H, m), 2.85-2.95 (1H, m), 4.65-4.76 (2H, m) 1.33 (6 H, d, J = 8.0 Hz); HPLC: 98.8%(254 nm), MS (ESI): m/z 541.3 [M + H]+ |
| 183 | 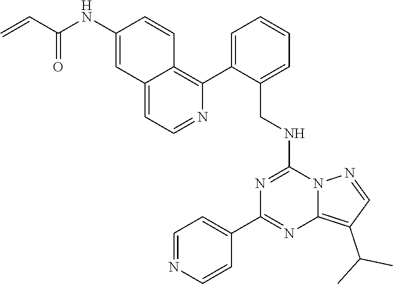 [SMILES: C=CC(=O)Nc1ccc2c(-c3ccccc3CNc3nc(-c4ccncc4)nc4c(C(C)C)cnn34)nccc2c1] | Yellow powder; ¹H NMR (400 MHz, DMSO-d6) δ 8.60- 8.72 (2H, m), 8.39 (1H, d, J = 4.0 Hz), 8.32 (1H, d, J = 1.6 Hz), 7.88-7.98 (2H, m), 7.67 (1H, d, J = 8.0 Hz), 7.58-7.64 (1H, m), 7.29-7.57 (6 H, m), 6.42-6.58 1H, m), 6.28-6.39 (1H, m), 5.81 (1H, dd, J = 12.0, 2.0 Hz), 5.14 (1H, s), 5.11-5.19 (1H, m), 4.65-4.76 (2H, m), 3.25-3.33 (1H, m), 2.91-2.99 (1H, m), 1.34 (6 H, d, J = 8.0 Hz); HPLC: 97.6%(254 nm), MS (ESI): m/z 541.3 [M + H]+ |
| 184 | 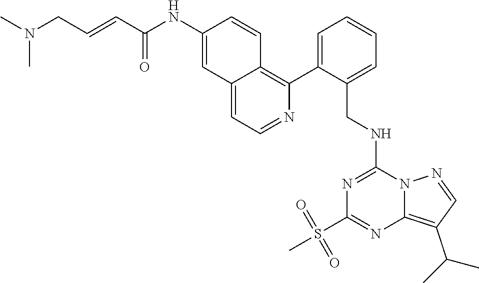 [SMILES: CC(C)c1cnn2c(NCc3ccccc3-c3nccc4cc(NC(=O)/C=C/CN(C)C)ccc34)nc(S(C)(=O)=O)nc12] | White solid; ¹H NMR (400 MHz, DMSO-d6) δ ppm 10.35 (1H, br s) 9.22 (1H, br s) 8.45 (1H, d, J = 5.77 Hz) 8.36 (1H, s) 8.05 (1H, s) 7.66 (1H, d, J = 6.02Hz) 7.55- 7.62 (2H, m) 7.44-7.54 (2H, m) 7.30-7.40 (2H, m) 6.84 (1H, dt, J = 15.43, 6.46 Hz) 6.45 (1H, s) 6.41 (1H, s) 6.38-6.47 (1H, m) 4.65 (2H, br d, J = 14.56 Hz) 3.41- 3.56 (2H, m) 3.13-3.14 (1H, m) 3.14 (2H, s) 3.01- 3.03 (2H, m) 2.95-3.03 (7 H, m) 1.29 (6 H, d, J = 6.78 Hz); LCMS: 97%, MS (ESI): m/z 599.1 [M + H]+ |
| 185 | 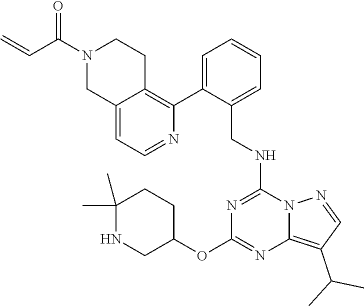 [SMILES: C=CC(=O)N1CCc2c(ccnc2-c2ccccc2CNc2nc(OC3CCC(C)(C)NC3)nc3c(C(C)C)cnn23)C1] | White powder; ¹H NMR (400 MHz, DMSO-d6) δ8.51 (1H, br s), 8.37 (1H, d, J = 4.0 Hz), 7.82 (1H, s), 7.43- 7.49 (1H, m), 7.33-7.48 (1H, m), 7.22 (1H, br d, J = 12.0 Hz), 7.16 (1H, d, J = 4.0 Hz), 6.70-6.85 (1H, m), 6.12 (1H, d, J = 16.0 Hz), 5.69 (1H, br d, J = 8.0 Hz), 4.90 (1H, br s), 4.67 (2H, br s), 4.45-4.55 (2H, m), 3.61 (2H, br s), 2.99 (3H, br d, J = 7.28 Hz), 1.95-2.05 (2H, m), 1.77-1.82 (1H, m), 1.58-1.68 (1H, m), 1.42-1.54 (2H, m), 1.12-1.30 (12H, m); HPLC (254 nm): 98.8%, MS (ESI): m/z 581.4 [M + H]+ |
| 186 | 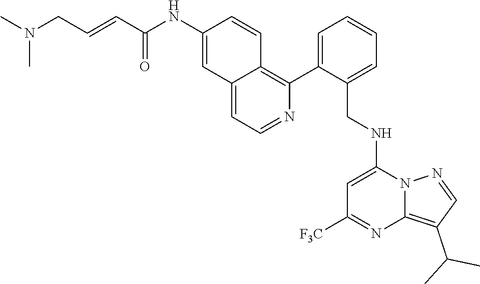 [SMILES: CC(C)c1cnn2c(NCc3ccccc3-c3nccc4cc(NC(=O)/C=C/CN(C)C)ccc34)cc(C(F)(F)F)nc12] | White powder; ¹H NMR (400 MHz, DMSO-d6) δ ppm 1.29 (5 H, d, J = 6.78 Hz) 2.22 (6 H, s) 2.93 (1H, br s) 3.30 (2H, br s) 4.49 (2H, br s) 6.09 (1H, s) 6.33 (1H, br d, J = 15.31 Hz) 6.84 (1H, dt, J = 15.37, 5.87 Hz) 7.35 (1H, d, J = 7.28 Hz) 7.44-7.54 (3H, m) 7.54-7.59 (1H, m) 7.64 (1H, d, J = 7.53 Hz) 7.69 (1H, d, J = 5.52 Hz) 7.98 (1H, s) 8.39 (1H, s) 8.47 (1H, d, J = 5.52 Hz) 10.24 (1H, br s); LCMS : 95.92%, MS (ESI): m/z 588.3 [M + H]+ |
| 187 | 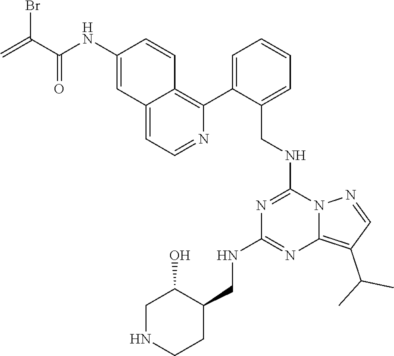 [SMILES: C=C(Br)C(=O)Nc1ccc2c(-c3ccccc3CNc3nc(NC[C@H]4CCNC[C@@H]4O)nc4c(C(C)C)cnn34)nccc2c1] | Yellow solid; ¹H NMR (DMSO-d6, 400 MHz) δ10.50 (1H, s) 8.48-8.68 (2H, m) 8.40 (2H, s) 8.21 (1H, s) 7.82 (1H, d, J = 6.0 Hz) 7.76 (1H, dd, J = 8.8, 2.4 Hz) 7.61 (3H, t, J = 5.2Hz) 7.54 (1H, t, J = 7.2Hz) 7.48 (1H, t, J = 7.2Hz) 7.38 (1H, d, J = 7.6 Hz) 6.82 (1H, d, J = 2.8 Hz) 6.36 (1H, d, J = 3.2 Hz) 5.34 (1H, t, J = 4.4 Hz) 4.49 (2H, s) 3.54 (1H, td, J = 9.2, 4.0 Hz) 3.43 (2H, d, J = 14.4 Hz) 2.87-2.91 (1H, m) 2.81 (1H, s) 2.57 (1H, s) 1.95- 2.07 (2H, m) 1.84 (1H, d, J = 12.8 Hz) 1.71 (1H, s) 1.40- 1.52 (2H, m) 1.26 (6 H, s); LCMS: 96.6%, MS (ESI): m/z 672.2 [M + H]+ |
| 188 | 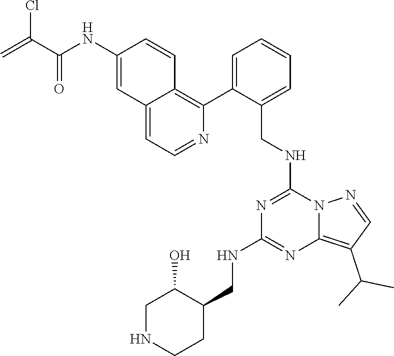 [SMILES: C=C(Cl)C(=O)Nc1ccc2c(-c3ccccc3CNc3nc(NC[C@H]4CCNC[C@@H]4O)nc4c(C(C)C)cnn34)nccc2c1] | Yellow solid; ¹H NMR (DMSO-d6, 400 MHz) δ10.46 (1H, s) 8.61 (1H, s) 8.51 (1H, d, J = 6.0 Hz) 8.43 (2H, d, J = 1.6 Hz) 7.75-7.87 (2H, m) 7.62 (2H, t, J = 4.2 Hz) 7.43-7.59 (2H, m) 7.39 (1H, d, J = 6.4 Hz) 6.51 (1H, d, J = 2.4 Hz) 6.14 (1H, d, J = 2.8 Hz) 4.50 (2H, s) 3.51- 3.57 (1H, m) 3.44 (1H, d, J = 10.8 Hz) 3.26-3.35 (1H, m) 3.24 (1H, d, J = 10.8 Hz) 3.17 (1H, d, J = 12.8 Hz) 2.85- 2.92 (1H, m) 2.81 (1H, s) 1.94-2.12 (1H, m) 1.84 (1H, d, J = 13.2Hz) 1.69 (1H, d, J = 10.8 Hz) 1.33-1.51 (1H, m) 1.26 (1H, s) 1.21 (6 H, d, J = 7.2Hz); LCMS: 99.7%, MS (ESI): m/z 626.3 [M + H]+ |
| 189 | 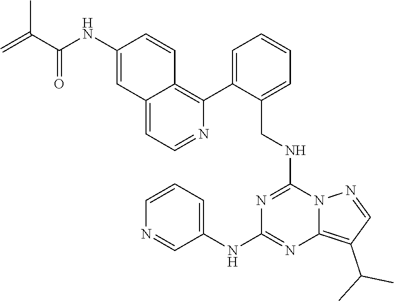 [SMILES: C=C(C)C(=O)Nc1ccc2c(-c3ccccc3CNc3nc(Nc4cccnc4)nc4c(C(C)C)cnn34)nccc2c1] | White solid; ¹H NMR (400 MHz, DMSO-d6) δ ppm 9.95 (1H, br s) 9.02 (1H, br d, J = 5.52Hz) 8.90 (1H, d, J = 2.26 Hz) 8.46 (1H, d, J = 5.77 Hz) 8.39 (1H, d, J = 1.76 Hz) 8.27 (1H, br s) 8.11-8.19 (2H, m) 7.71-7.79 (2H, m) 7.66 (1H, d, J = 5.77 Hz) 7.60 (2H, d, J = 8.78 Hz) 7.43-7.54 (2H, m) 7.36 (1H, dd, J = 7.40, 1.38 Hz) 7.22 (1H, dd, J = 8.41, 4.64 Hz) 5.88 (1H, s) 5.57 (1H, s) 4.57 (2H, br d, J = 5.02 Hz) 2.99 (1H, dt, J = 13.87, 7.00 Hz) 2.01 (3H, s) 1.29 (6H, d, J = 6.78 Hz); LCMS: 99%, MS (ESI): m/z 570.3 [M + H]+ |
| 190 | 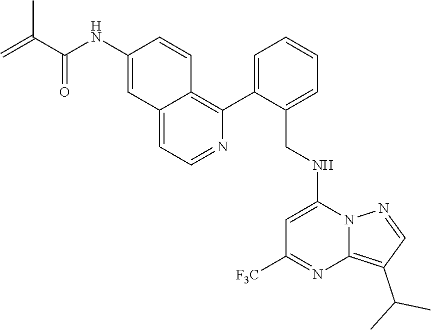 [SMILES: C=C(C)C(=O)Nc1ccc2c(-c3ccccc3CNc3cc(C(F)(F)F)nc4c(C(C)C)cnn34)nccc2c1] | Yellow powder; ¹H NMR (DMSO-d6, 400 MHz) δ9.97 (1H, s) 8.48 (2H, d, J = 5.6 Hz) 8.41 (1H, s) 8.00 (1H, s) 7.69-7.76 (2H, m) 7.64 (1H, d, J = 7.2Hz) 7.44-7.54 (3H, m) 7.34-7.38 (1H, m) 6.14 (1H, s) 5.89 (1H, s) 5.57 (1H, s) 4.49 (2H, s) 3.04-3.08 (1H, m) 2.01 (3 H, s) 1.30 (6 H, d, J = 7.2Hz); LCMS: 97.9%, MS (ESI): m/z 545.2 [M + H]+ |
| 191 | 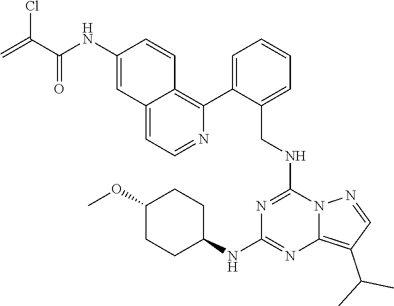 [SMILES: C=C(Cl)C(=O)Nc1ccc2c(-c3ccccc3CNc3nc(N[C@H]4CC[C@H](OC)CC4)nc4c(C(C)C)cnn34)nccc2c1] | White solid; ¹H NMR (400 MHz, DMSO-d6) δ ppm 10.38 (1H, br s) 8.50 (1H, d, J = 5.52Hz) 8.40 (1H, s) 7.93 (1H, br s) 7.72-7.80 (2H, m) 7.54-7.65 (3H, m) 7.41-7.52 (2H, m) 7.34 (1H, d, J = 7.28 Hz) 6.49 (1H, d, J = 2.51 Hz) 6.11 (1H, d, J = 2.51 Hz) 5.91 (1H, br s) 4.45 (2H, br s) 3.55 (1H, br s) 3.24 (3H, s) 3.08 (1H, br s) 2.88 (1H, quin, J = 6.96 Hz) 1.83-2.01 (4 H, m) 1.25 (2H, br d, J = 7.03Hz) 1.21 (6 H, d, J = 6.78 Hz) 1.13- 1.18 (2H, m); LCMS: 100%, MS (ESI): m/z 625.1 [M + H]+ |
| 192 | 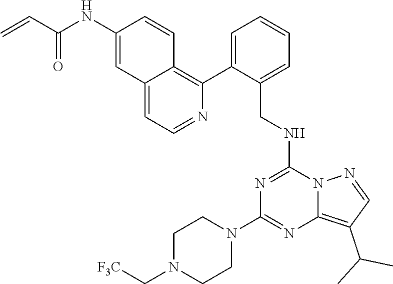 [SMILES: C=CC(=O)Nc1ccc2c(-c3ccccc3CNc3nc(N4CCN(CC(F)(F)F)CC4)nc4c(C(C)C)cnn34)nccc2c1] | White powder; ¹H NMR (400 MHz, DMSO-d6) δ 10.33 (1H, br s), 8.47 (1H, d, J = 5.6 Hz), 8.42 (1H, s), 8.15 (1H, br s), 7.71 (1H, d, J = 5.6 Hz), 7.52-7.67 (3H, m), 7.40-7.51 (2H, m), 7.32-7.37 (1H, m), 6.45-6.59 (1H, m), 6.26-6.42 (1H, m), 5.76-5.88 (1H, m), 4.46 (2H, br s), 3.44-3.61 (4 H, m), 3.13-3.20 (2H, m), 2.85- 2.95 (1H, m), 2.53-2.59 (4 H, m) 1.16-1.30 (6 H, m); HPLC (254 nm): 97.9%, MS (ESI): m/z 630.1 [M + H]+ |
| 193 | 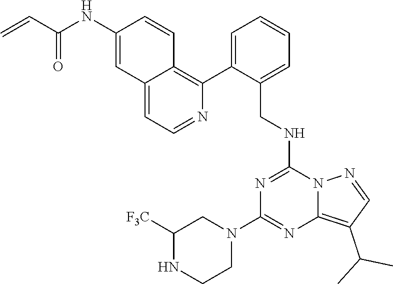 [SMILES: C=CC(=O)Nc1ccc2c(-c3ccccc3CNc3nc(N4CCNC(C(F)(F)F)C4)nc4c(C(C)C)cnn34)nccc2c1] | Yellow powder; ¹H NMR (400 MHz, DMSO-d6) δ 10.34 (1H, br s), 8.40-8.55 (2H, m), 8.11 (1H, br s), 7.70 (1H, d, J = 4.8 Hz), 7.41-7.64 (5 H, m), 7.31-7.37 (1H, m), 6.46-6.61 (1H, m), 6.26-6.39 (1H, m), 5.82 (1H, dd, J = 10.0, 2.0 Hz), 4.48 (2H, br s), 4.22-4.35 (1H, m), 3.96 (1H, d, J = 12.0 Hz), 3.22-3.35 (1H, m), 2.83- 2.96 (2H, m), 2.53-2.73 (3H, m), 1.23 (6 H, d, J = 8.0 Hz); HPLC (254 nm): 98.2%, MS (ESI): m/z 616.3 [M + H]+ |
| 194 | 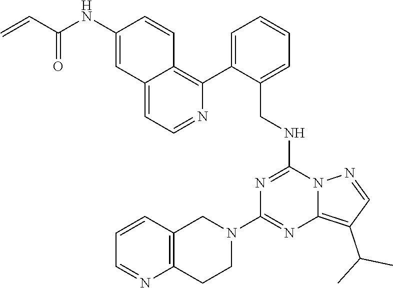 [SMILES: C=CC(=O)Nc1ccc2c(-c3ccccc3CNc3nc(N4CCc5ncccc5C4)nc4c(C(C)C)cnn34)nccc2c1] | Yellow solid; ¹H NMR (DMSO-d6, 400 MHz) δ 10.34 (1H, s) 8.40-8.49 (2H, m) 8.36 (1H, d, J = 4.52Hz) 8.14 (1H, br s) 7.67 (1H, d, J = 5.77 Hz) 7.59-7.63 (1H, m) 7.53-7.57 (1H, m) 7.42-7.53 (3H, m) 7.35 (1H, d, J = 6.27 Hz) 7.19 (1H, dd, J = 7.53, 4.77 Hz) 6.46-6.55 (1H, m) 6.30-6.37 (1H, m) 5.81 (1H, dd, J = 10.29, 1.51 Hz) 4.71 (2H, s) 4.52 (2H, s) 3.89 (2H, t, J = 5.90 Hz) 2.94 (1H, dt, J = 13.68, 6.71 Hz) 2.86 (2H, t, J = 5.77 Hz) 1.25 (6 H, d, J = 6.78 Hz); LCMS: 97.7%, MS (ESI): m/z 596.4 [M + H]+ |
| 195 | 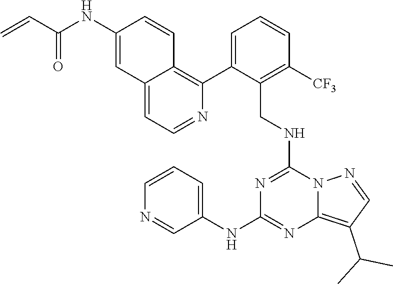 [SMILES: C=CC(=O)Nc1ccc2c(-c3cccc(C(F)(F)F)c3CNc3nc(Nc4cccnc4)nc4c(C(C)C)cnn34)nccc2c1] | White powder; ¹H NMR (400 MHz, DMSO-d6) δ 10.23 (1H, s) 8.95 (1H, s) 8.88 (1H, d, J = 2.0 Hz) 8.29 (1H, d, J = 5.6 Hz) 8.25 (1H, s) 8.06-8.18 (2H, m) 7.97 (1H, d, J = 7.6 Hz) 7.83 (1H, s) 7.70-7.76 (1H, m) 7.63-7.68 (1H, m) 7.58 (1H, s) 7.45-7.55 (3H, m) 7.24 (1H, dd, J = 8.0, 4.4 Hz) 7.21-7.28 (1H, m) 6.42-6.53 (1H, m) 6.25-6.36 (1H, m) 5.79 (1H, dd, J = 10.4, 1.6 Hz) 5.24 (1H, d, J = 14.0 Hz) 4.26 (1H, d, J = 14.4 Hz) 2.90 (1H, m) 1.24 (6 H, d, J = 6.8 Hz); HPLC (254 nm): 98.2%, MS (ESI): m/z 624.2 [M + H]⁺ |
| 196 | 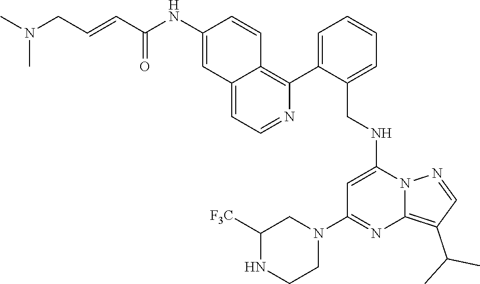 [SMILES: CC(C)c1cnn2c(NCc3ccccc3-c3nccc4cc(NC(=O)/C=C/CN(C)C)ccc34)cc(N3CCNC(C(F)(F)F)C3)nc12] | Yellow powder; ¹H NMR (400 MHz, DMSO-d6) δ ppm 10.46 (1H, br s) 8.39-8.51 (2H, m) 7.66-7.78 (2H, m) 7.58-7.64 (2H, m) 7.40-7.56 (4 H, m) 7.32-7.37 (1H, m) 6.84 (1H, dt, J = 15.43, 5.96 Hz) 6.40 (1H, br d, J = 15.56 Hz) 5.34 (1H, s) 4.26-4.34 (3H, m) 3.76 (1H, br d, J = 12.30 Hz) 3.27-3.39 (4 H, m) 2.92-3.05 (2H, m) 2.66-2.73 (2H, m) 2.23 (6 H, s) 1.91-2.09 (1H, m) 1.25 (6 H, d, J = 6.78 Hz); LCMS: 98.05 %, MS (ESI): m/z 672.3 [M + H]⁺ |
| 197 | 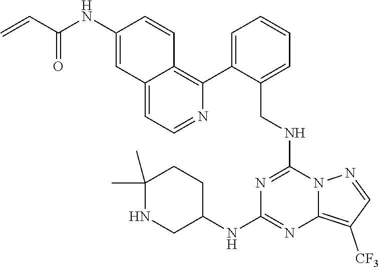 [SMILES: C=CC(=O)Nc1ccc2c(-c3ccccc3CNc3nc(NC4CCC(C)(C)NC4)nc4c(C(F)(F)F)cnn34)nccc2c1] | Off-white powder; ¹H NMR (400 MHz, DMSO-d6) δ ppm 10.32 (1H, br s) 8.47 (1H, br d, J = 5.77 Hz) 8.41 (1H, s) 7.92-8.05 (1H, m) 7.62-7.77 (2H, m) 7.58 (1H, br d, J = 7.78 Hz) 7.41-7.54 (2H, m) 7.35 (1H, br d, J = 7.03Hz) 6.50 (1H, br dd, J = 16.94, 10.16 Hz) 6.33 (1H, br dd, J = 17.07, 1.76 Hz) 5.75-5.86 (1H, m) 4.50 (2 H, br s) 3.19 (3H, br s) 3.04 (3H, br s) 2.45 (2H, br s) 1.94-2.10 (2H, m) 1.49 (2H, br s) 1.29-1.36 (1H, m) 1.26 (3H, s) 1.08 (3H, s); LCMS: 100%, MS (ESI): m/z 616.4 [M + H]⁺ |
| 198 | 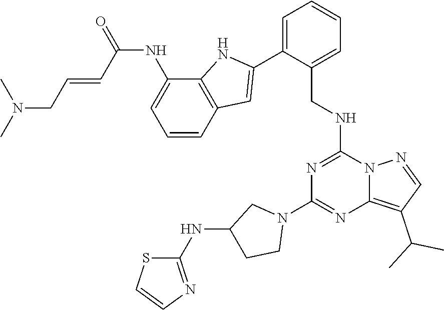 [SMILES: CC(C)c1cnn2c(NCc3ccccc3-c3cc4cccc(NC(=O)/C=C/CN(C)C)c4[nH]3)nc(N3CCC(Nc4nccs4)C3)nc12] | Off-white powder; ¹H NMR (400 MHz, DMSO-d6) δ ppm 11.50 (br s, 1H) 10.43 (br s, 1H) 8.53 (br s, 1H) 7.75 (br d, J = 8.28 Hz, 1H) 7.71 (s, 1H) 7.55-7.64 (m, 2H) 7.37 (m, 3H) 7.13 (br d, J = 3.51 Hz, 1H) 7.01 (br t, J = 7.78 Hz, 1H) 6.79-6.87 (m, 2H) 6.71 (br d, J = 3.26 Hz, 1H) 6.64 (d, J = 2.01 Hz, 1H) 4.96 (br d, J = 6.53Hz, 2H) 4.25 (br s, 1H) 3.95 (br d, J = 5.77 Hz, 2H) 3.66 (br s, 1H) 3.45-3.51 (m, 1H) 3.40 (m, 2H) 2.93-3.00 (m, 1H) 2.82 (s, 6 H) 2.13 (m, 1H) 1.89 (m, 1H) 1.25 (d, J = 7.03Hz, 6 H); HPLC: 96.6%, MS (ESI): 698.4 m/z [M + Na]⁺ |

This patent application presents the utility and outstanding activities achieved by combinations of CDK7 inhibitors with other anti-cancer therapies, in particular other anti-cancer agents. Considering the effect of CDK7 inhibitors on arresting cell cycle and inducing replicative stress and genomic instability, the combination of CDK7 inhibitors with other anti-cancer therapies improve anti-tumor efficacy as a new approach to cancer therapy.

Furthermore, reference is made to the figures, wherein:

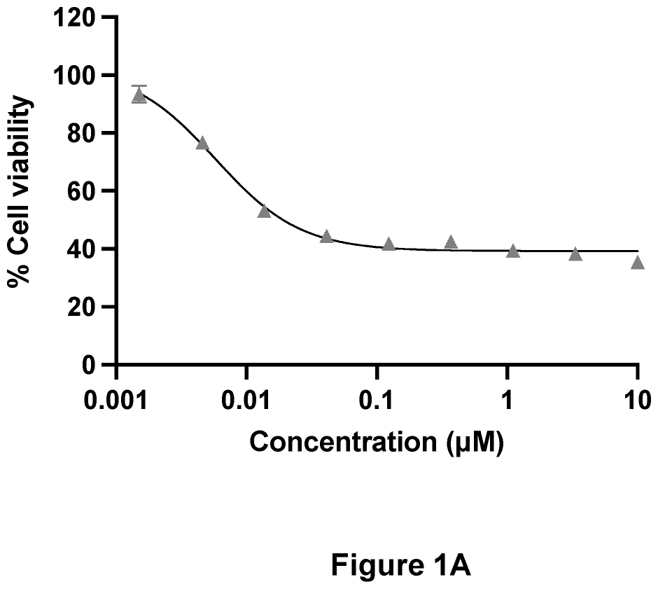

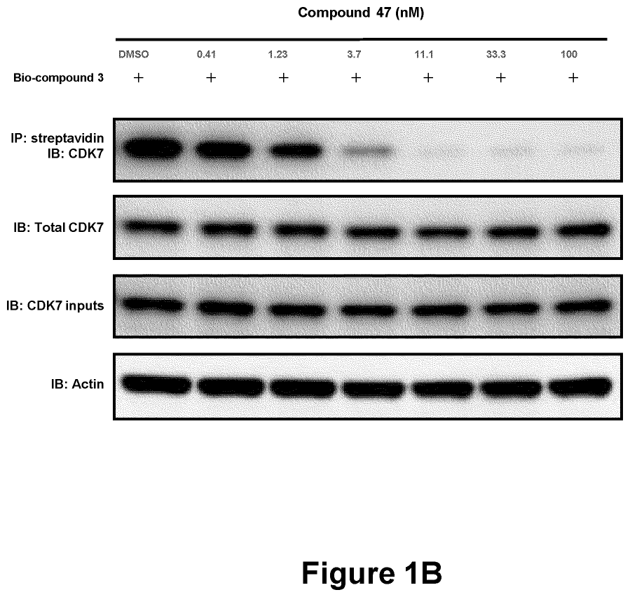

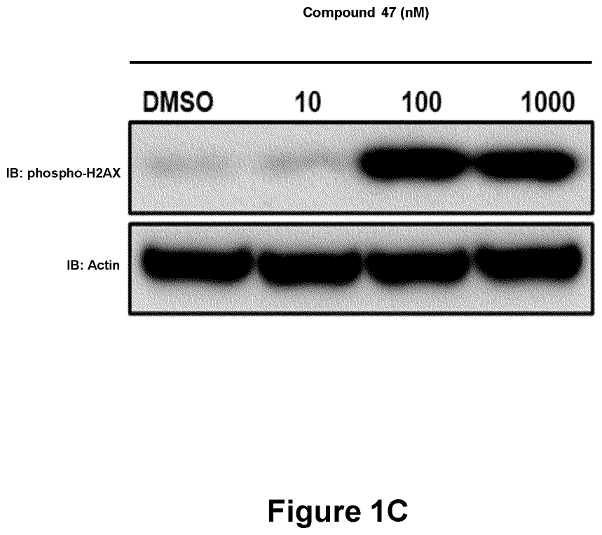

FIGS. 1A, 1B, and 1C show the effect of CDK7 inhibitor on RENCA cells.

FIG. 1A shows the results of examining RENCA cell viability in the presence of CDK7 inhibitor.

FIG. 1B shows the results of CDK7 engagement analysis by CDK7 inhibitor.

FIG. 1C shows the western blot results of phosphorylated form of H2AX by CDK7 inhibitor treatment in RENCA cells.

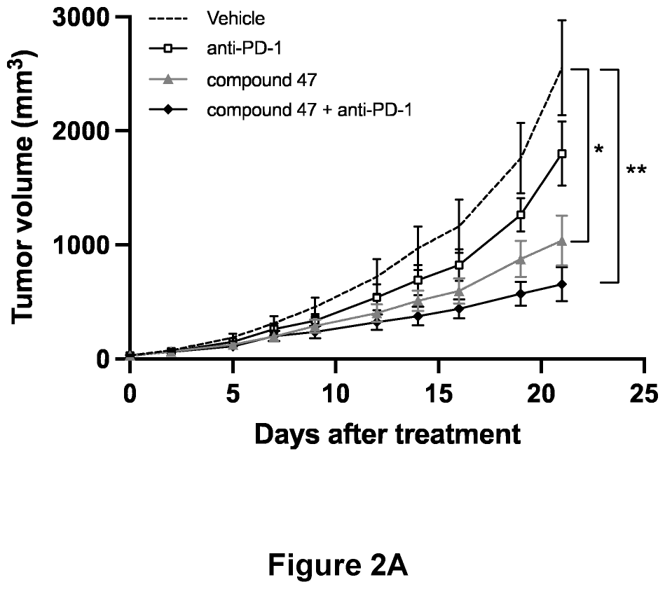

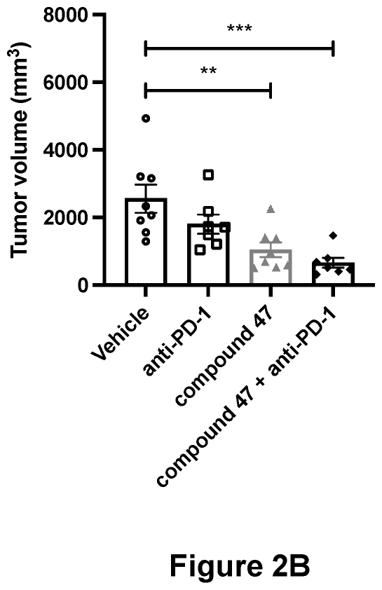

FIGS. 2A and 2B show the effect of the combination treatment of CDK7 inhibitor with anti-PD-1 antibody in a RENCA syngeneic mouse tumor model.

FIG. 2A shows RENCA cell tumor growth in each treatment group as MEAN f SEM.

FIG. 2B shows tumor volume data on day 21.

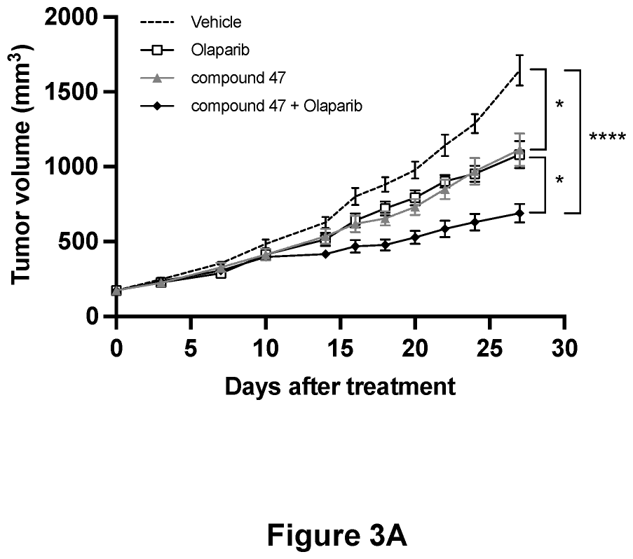

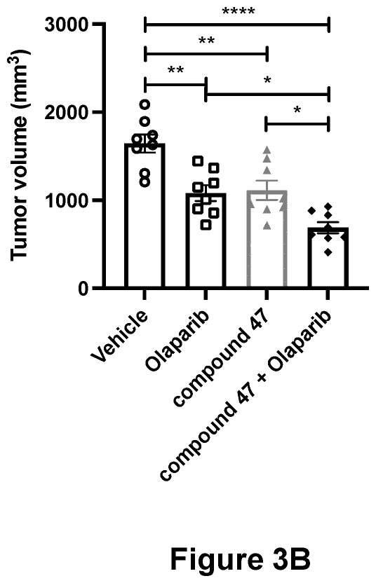

FIGS. 3A and 3B show the effect of the combination treatment of CDK7 inhibitor with olaparib in a OVCAR3 high-grade serous ovarian cancer cell line-derived xenograft model.

FIG. 3A shows OVCAR3 cell tumor growth in each treatment group as MEAN f SEM.

FIG. 3B shows tumor size data on day 27.

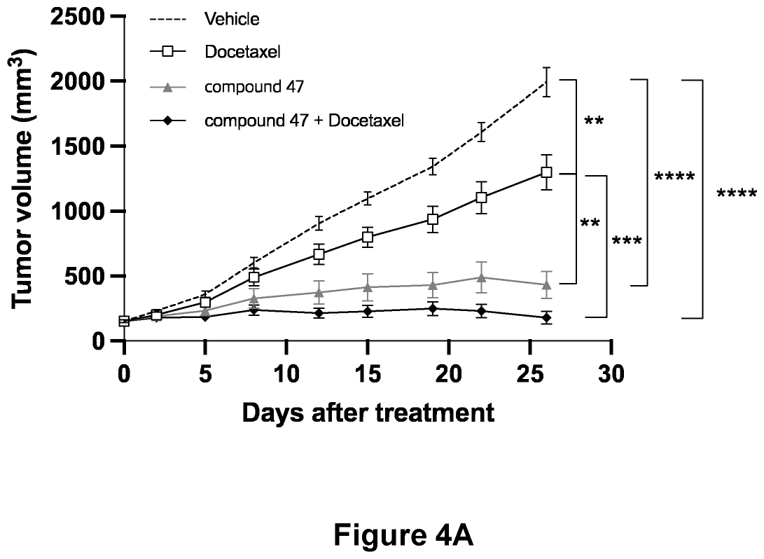

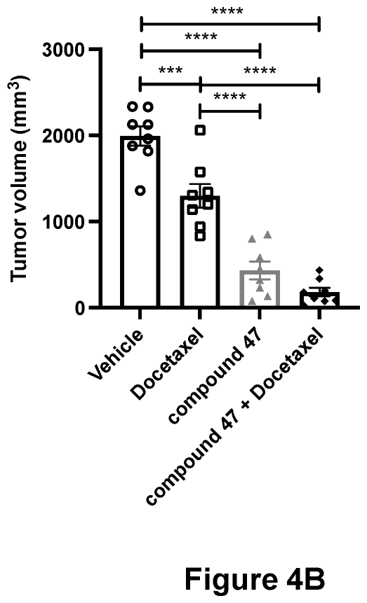

FIGS. 4A and 4B show the effect of the combination treatment of CDK7 inhibitor with docetaxel in a DU145 castration-resistant prostate cancer cell line-derived xenograft model.

FIG. 4A shows DU145 cell tumor growth in each treatment group as MEAN f SEM.

FIG. 4B shows tumor size data on day 26.

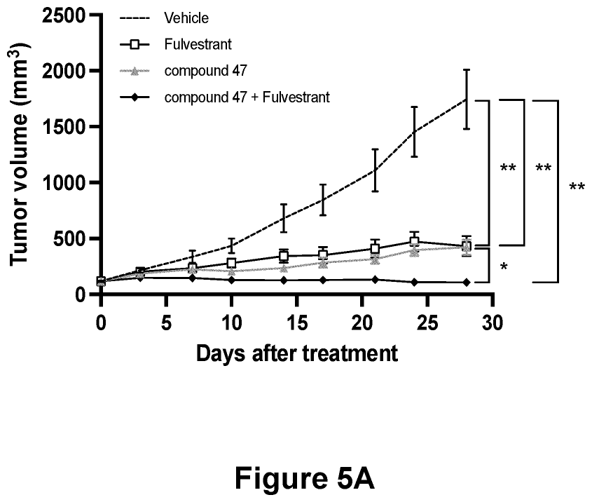

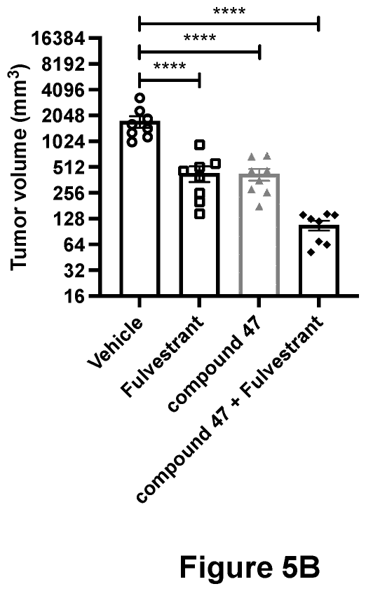

FIGS. 5A and 5B show the effect of the combination treatment of CDK7 inhibitor with fulvestrant in a MCF7 breast cancer cell line-derived xenograft model.

FIG. 5A shows MCF7 cell tumor growth in each treatment group as MEAN f SEM.

FIG. 5B shows tumor volume data on day 28.

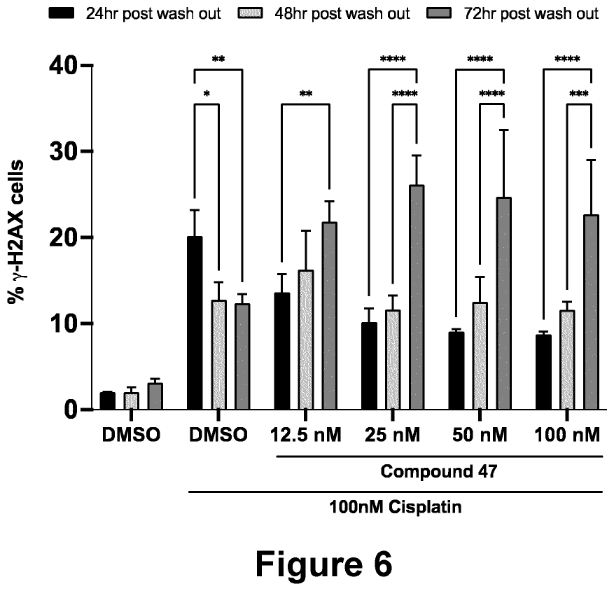

FIG. 6 shows the average percentage of g-H2AX-positive OVCAR3 cells treated with a combination of compound 47 and cisplatin.

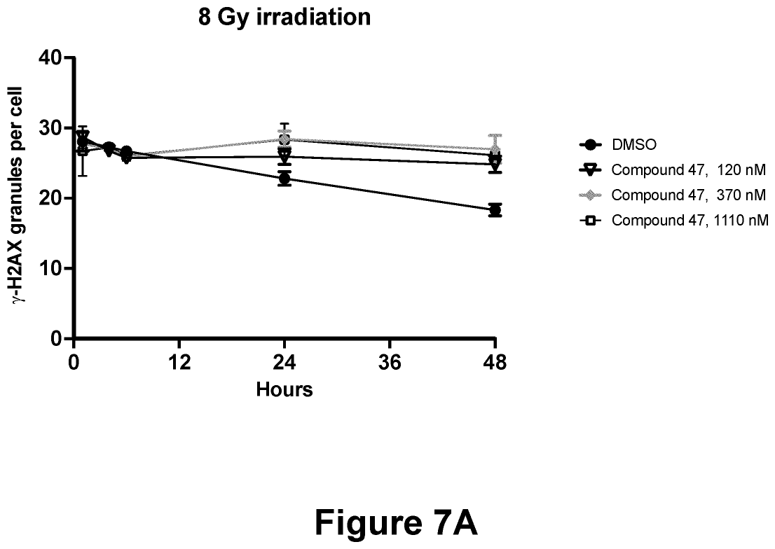

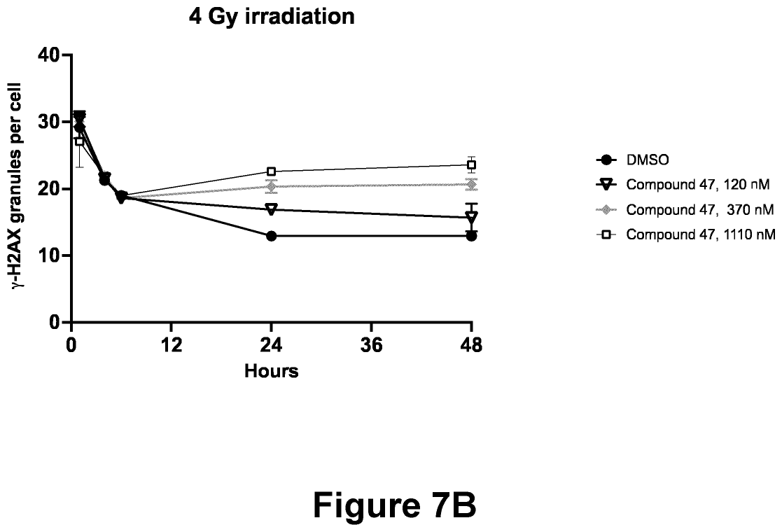

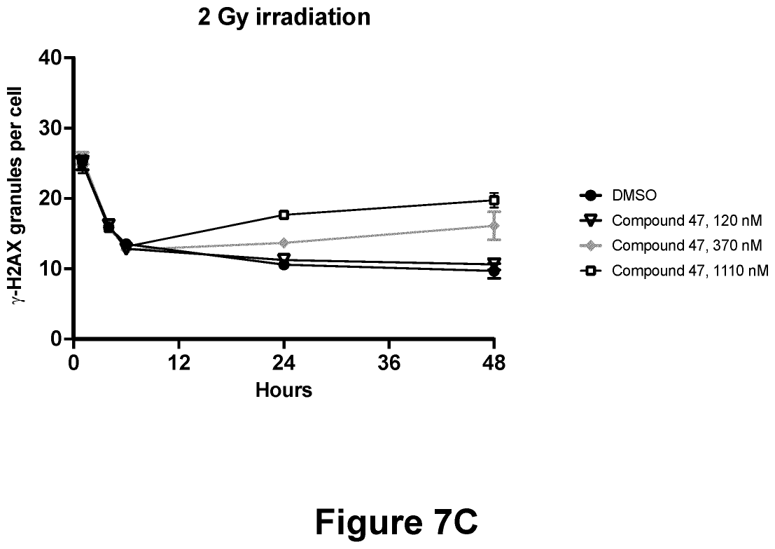

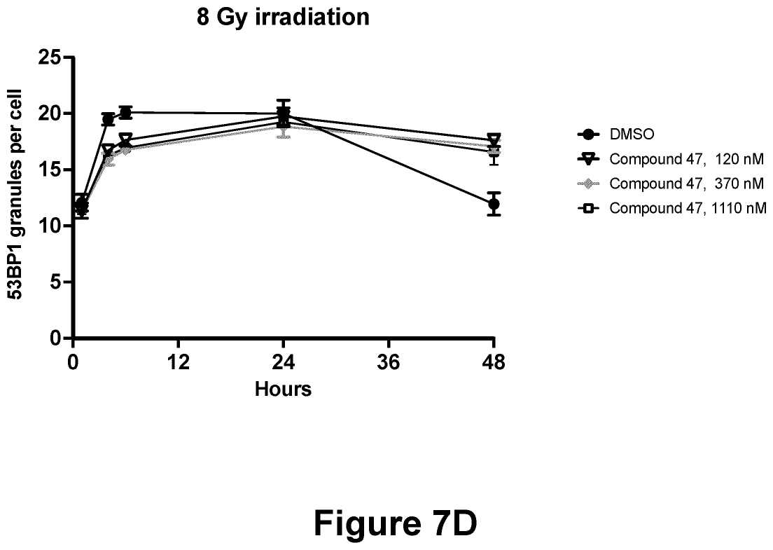

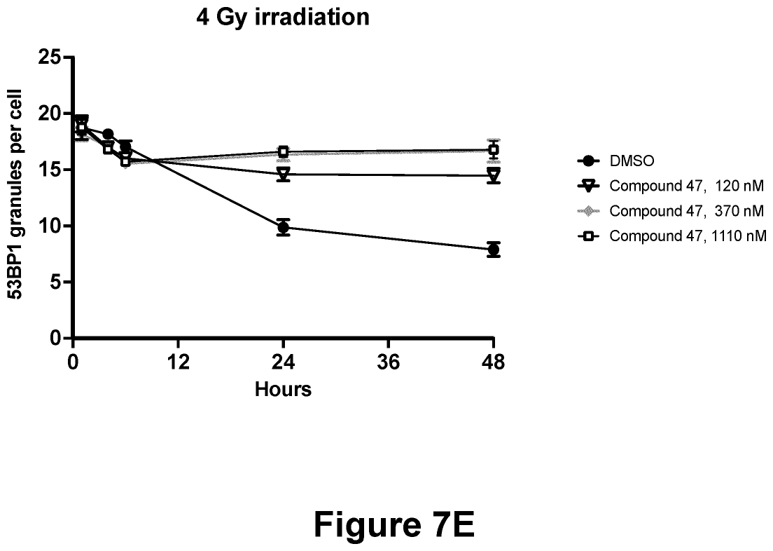

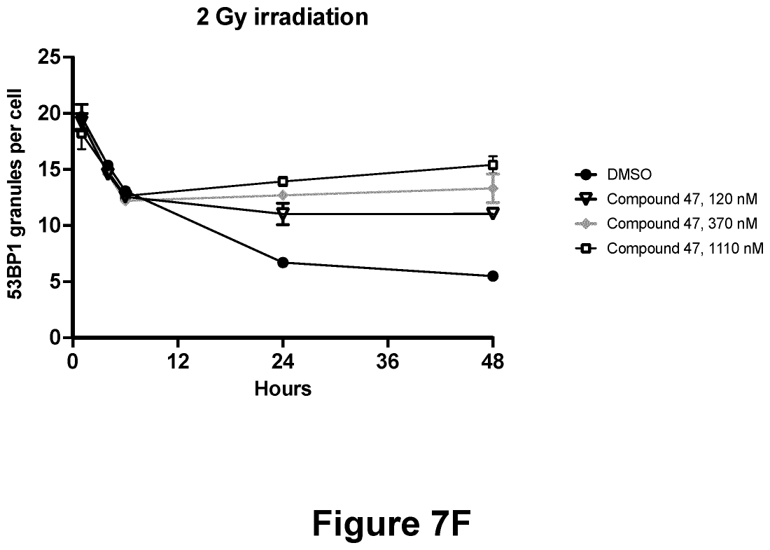

FIG. 7 shows the average granule number of g-H2AX (FIG. 7A to 7C) or of 53BP1 (FIG. 7D to 7F) of PC3 cells treated with a combination of compound 47 and X-ray radiation.

### EXAMPLE 1. THE EFFECT OF CDK7 INHIBITOR ON RENCA CELLS

### Cell Viability Assay

RENCA renal adenocarcinoma were treated with various concentrations of compound 47 for 72 hours. Cell viability was measured using the CellTiter-Glo assay system (Promega). Luminescence units were normalized to those of untreated cells and are presented as the percentage of cell viability. IC₅₀ was calculated using the dose-response curve generated by GraphPad Prism. FIG. 1A shows the effect of compound 47 on RENCA cell viability. Growth of RENCA cells was inhibited by compound 47 in a dose dependent manner with an IC₅₀ of 20 nM.

### Target Occupancy Assay

RENCA cells were treated with various concentrations of compound 47 for 4 hours. Cells were washed with ice-cold PBS twice and then lysed with ice-cold lysis buffer (0.025M Tris, 0.15M NaCl, 0.001M EDTA, 1% NP-40, 5% glycerol, pH 7.4) with protease and phosphatase cocktails (Sigma-Aldrich). Lysed cells were centrifuged at 12,000 rpm at 4′C for 10 minutes and then the supernatant was collected. Protein concentrations were determined using a BCA protein quantification kit (Thermo Fisher Scientific, #23227). Equal amounts of protein were incubated with 1 μM of biotinylated compound 3 (Bio-compound 3; biotinylated analog of compound 47) at 4′C overnight and then immunoprecipitated with streptavidin-agarose bead. Pull-downed proteins were eluted, loaded to SDS-PAGE, transferred to a PVDF membrane (MilliporeSigma), and then treated with anti-CDK7 antibody. CDK7 protein was detected by HRP-conjugated secondary antibody. The image was obtained by ImageQuant™ LAS4000. FIG. 1B shows CDK7 occupancy by compound 47 in a dose dependent manner in RENCA cells.

### Western Blot Analysis

RENCA cells were treated with various concentrations of compound 47 in 5% CO₂ at 37′C for 48 hours. Cells were washed with ice-cold PBS twice and lysis buffer was added. Cells were collected and kept on the ice for 30 min. Lysed cells were centrifuged at 12,000 rpm at 4′C for 10 minutes, and then the supernatant was collected. Protein concentrations were quantified using a BCA protein Quantification kit. Equal amounts of protein were fractionated by SDS-PAGE, transferred to a PVDF membrane, and then treated with anti-phospho-H2AX (Ser139) antibody. The phosphorylated form of H2AX protein was detected by HRP-conjugated secondary antibody and the signal was obtained with Super Signal Western blot enhancer. The image was acquired by ImageQuant™ LAS 4000. FIG. 1C shows the effect of compound 47 on phosphorylated form of the histone variant H2AX, a marker for the early cellular response to the DNA double-strand breaks. Compound 47 induced phosphorylation of H2AX at the Serine-139 residue in a dose dependent manner, suggesting that compound 47 induces DAN double-strand breaks in RENCA cells by preventing homologous recombination repair and DNA mismatch repair pathways.

### EXAMPLE 2. THE EFFECT OF COMBINATION OF CDK7 INHIBITOR AND ANTI-PD1 IN A RENCA SYNGENEIC RENAL TUMOR MOUSE MODEL

RENCA cells (1×10⁵) were subcutaneously implanted in the right flank of BALB/c mice. Tumor-bearing mice were randomized and then treated with compound 47 (10 mg/kg, intraperitoneally every day), anti-PD-1 antibody (10 mg/kg, intraperitoneally twice a week, clone: RMP1-14, Bio X Cell), or both compound 47 and anti-PD-1 antibody when the average tumor volume reached 31 mm³ (N=7 or 8 per group). Control mice were treated with vehicle and rat IgG2a isotype control antibody (clone: 2A3, Bio X Cell). Tumor volume and body weight were measured twice per week. FIG. 2A shows RENCA tumor cell growth in each treatment group. Compound 47 treatment induced 50.2% tumor growth inhibition (TGI) and anti-PD-1 treatment group showed 13.2% TGI. However, combination of compound 47 with anti-PD-1 antibody improved TGI (66%). FIG. 2B shows tumor volume on day 21. On day 21, there was a significant decrease in tumor volume in the group with the combination of compound 47 and anti-PD-1 antibody.

### EXAMPLE 3. THE EFFECT OF COMBINATION OF CDK7 INHIBITOR WITH OLAPARIB IN A OVCAR3 HIGH-GRADE SEROUS OVARIAN CANCER CELL LINE-DERIVED XENOGRAFT MODEL

OVCAR3 cells (1×10⁷) mixed with matrigel (50:50) were subcutaneously implanted in the right flank of female BALB/c nude mice. Tumor-bearing mice were randomized and treated with compound 47 (3 mg/kg, intraperitoneally every day), olaparib (100 mg/kg, orally every day), or both compound 47 and olaparib when the average tumor volume reached 173 mm³ (N=8 per group). Tumor volume and body weight were measured twice per week. FIG. 3A shows OVCAR3 tumor cell growth in each treatment group. Compound 47 treatment induced 36% TGI and olaparib treatment group showed 38% TGI. However, combination of compound 47 with olaparib improved TGI (65%). FIG. 3B shows tumor volume on day 27. On day 27, combination treatment with compound 47 and olaparib significantly reduced tumor volume compared to either olaparib alone group or compound 47 alone group.

### EXAMPLE 4. THE EFFECT OF COMBINATION OF CDK7 INHIBITOR AND DOCETAXEL IN A DU145 CASTRATION-RESISTANT PROSTATE CANCER CELL LINE-DERIVED XENOGRAFT MODEL

DU145 cells (1×10⁷) mixed with matrigel (50:50) were subcutaneously implanted in the right flank of male BALB/c nude mice. Tumor-bearing mice were randomized and then treated with compound 47 (3 mg/kg, intraperitoneally every day), docetaxel (15 mg/kg, intraperitoneally once a week), or both compound 47 and docetaxel when the average tumor volume reached 154 mm³ (N=8 per group). Tumor volume and body weight were measured twice per week. FIG. 4A shows DU145 tumor cell growth in each treatment group. Compound 47 treatment induced 61% TGI and docetaxel treatment group showed 25% TGI. However, combination of compound 47 with docetaxel improved TGI (81%). FIG. 4B shows tumor volume measurement on day 26. On day 26, tumor size was significantly reduced in the compound 47 and docetaxel combination group compared to the docetaxel alone group.

### EXAMPLE 5. THE EFFECT OF COMBINATION OF CDK7 INHIBITOR AND FULVESTRANT IN A MCF7 HUMAN BREAST ADENOCARCINOMA XENOGRAFT MODEL

MCF7 cells (1×10⁷) mixed with matrigel (50:50) were subcutaneously implanted in the right flank of female BALB/c nude mice. Tumor-bearing mice were randomized and treated with compound 47 (3 mg/kg, intraperitoneally every day), fulvestrant (2.5 mg/dose, subcutaneously every day), or both compound 47 and fulvestrant when the average tumor volume reached 117 mm³ (N=8 per group). Tumor volume and body weight were measured twice per week. FIG. 5A shows MCF7 tumor cell growth in each treatment group. Both compound 47 treatment group and fulvestrant treatment group showed 81% TGI, respectively. However, combination of compound 47 with fulvestrant improved TGI (101%). FIG. 5B shows tumor size on day 28. On day 28, tumor volume of the compound 47 with fulvestrant combination group was reduced compared to the compound 47 alone group or the fulvestrant treatment group.

### EXAMPLE 6. COMBINATION EFFECT OF CDK7 INHIBITOR AND CISPLATIN IN OVCAR3 HUMAN HIGH SEROUS OVARIAN CANCER CELL LINE

OVCAR3 cells were treated with DMSO or 100 nM of cisplatin for 24 hours. Cells were then washed with culture media and treated with Compound 47 at various concentrations. Cells were collected 24, 48 and 72 hours after cisplatin wash and then stained with 4′,6-diamidino-2-phenylindole (DAPI) and anti-phospho-histone H2AX (Ser129). g-H2AX foci inside the nucleus were identified by Alexa-568 staining. Fluorescence images of g-H2AX foci were captured using a confocal imaging system, CQ1 (×40 objective). FIG. 6 shows the average percentage f SD of g-H2AX-positive cells, and it becomes evident that Compound 47 increased the percentage of g-H2AX foci in a time- and dose-dependent manner, indicating that Compound 47 sustains DNA damage induced by cisplatin treatment.

### EXAMPLE 7. COMBINATION EFFECT OF CDK7 INHIBITOR AND X-RAY IRRADIATION IN PC3 HUMAN CASTRATION-RESISTANT PROSTATE CANCER CELL LINE

PC3 cells were treated with DMSO or various concentrations of Compound 47 for 1 hour. Cells were then irradiated with X-ray at set of 225 KV, 17.7 mA for 5 min using a Faxitron Specimen Radiography System (8 Gy, 4 Gy and 2 Gy irradiation, FIGS. 7A-7C, and 7D-7F, respectively). Cells were collected at 1-, 4-, 6-, 24- and 48-hour post X-ray irradiation and stained with HOECHST and anti-phospho-histone H2AX (Ser129) or 53BP1. g-H2AX or 53BP1 foci inside the nucleus were identified with Alexa Flour488. Fluorescence images of the foci were obtained using Opera Phenix 3 High-content screening system (×40 objective) and the average granule number f SD of g-H2AX (FIG. 7A to 7C) or of 53BP1 (FIG. 7D to 7F) per cell was determined using MetaXpress software. As shown in FIG. 7A to 7F, Compound 47 showed significant DNA damage sustaining effects 24 hours after X-ray irradiation in a manner dependent on the irradiation dose or the compound concentration.

1. A combination of an inhibitor of cyclin-dependent kinase 7 and an anti-cancer agent which is different from said inhibitor of cyclin-dependent kinase 7,
wherein said inhibitor of cyclin-dependent kinase 7 is a compound having the general formula I 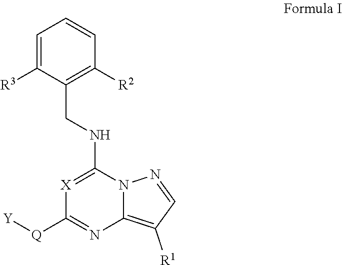
wherein
X is, independently at each occurrence, selected from CH and N;
Q is either absent or independently, at each occurrence, selected from the group consisting of —NH—, —NH(CH₂)—, —NH(CH₂)₂—, —NH(C═O)—, —NHSO₂—, —O—, —O(CH₂)—, —(C═O)—, —(C═O)NH— and —(C═O)(CH₂)—;
Y is, independently at each occurrence, selected from the group consisting of halogen, C1-C3 haloalkyl, C3-C8 cycloalkyl, aryl, heteroaryl, heterocyclyl, —S(═O)₂R⁴, C1-C6 alkyl and C1-C6 alkyl substituted with one or two of —OR⁶, —N(R⁶)R⁶, aryl, heteroaryl and heterocyclyl;
wherein C3-C8 cycloalkyl is optionally substituted with one or two of R⁴, R⁵ and —(C═O)R⁶, wherein heterocyclyl is optionally substituted with one or two of R⁴, R⁵ and —(C═O)R⁶, and wherein aryl or heteroaryl is optionally substituted with one or two of R⁴, C1-C6 alkyl, —OR⁶, —N(R⁶)R⁶, —(C═O)R⁶, halogen, heteroaryl and heterocyclyl;
R¹ is, at each occurrence, independently selected from the group consisting of halogen, C1-C6 alkyl, C3-C10 cycloalkyl, —CN, —(C═O)CH₃ and C1-C3 haloalkyl, any of which is optionally substituted;
R² is, at each occurrence, independently selected from any structure of the following group A 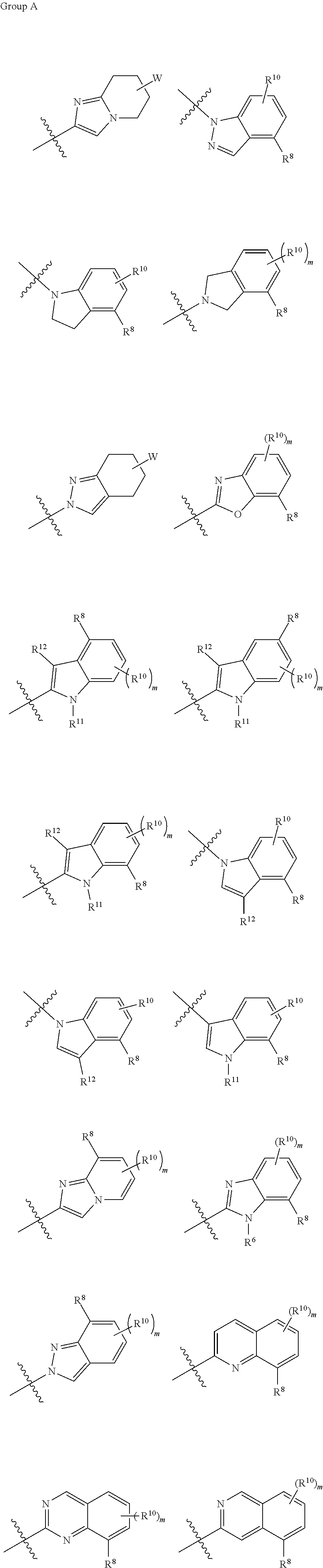 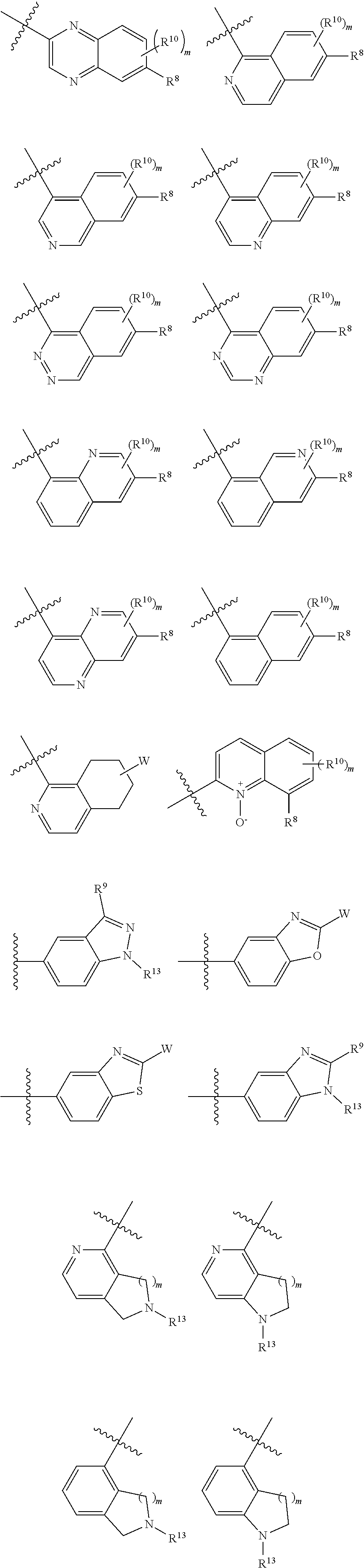
wherein m is, independently at each occurrence, selected from 1, 2 and 3;
W is any structure of the following group B; 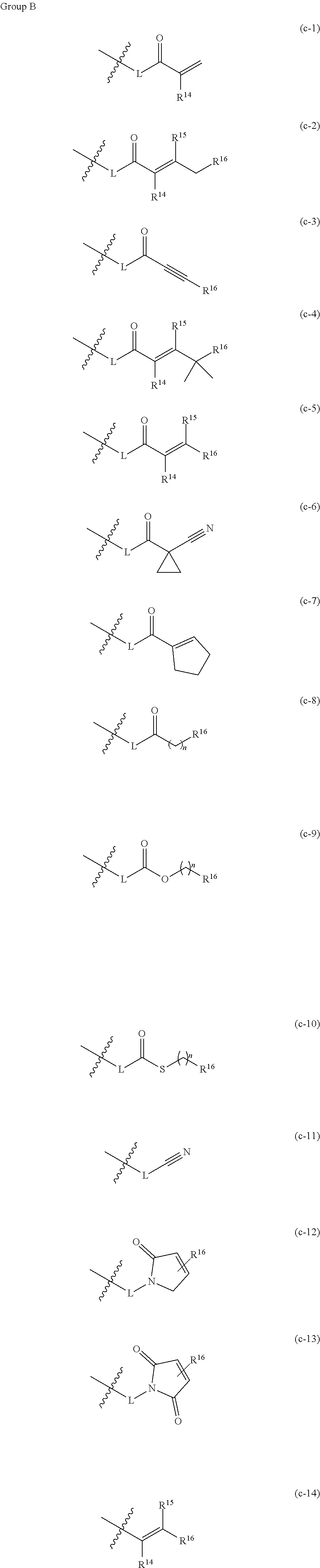
L is absent or, at each occurrence, independently selected from the group consisting of —O— and —NH—;
wherein n is, independently at each occurrence, selected from 1, 2 and 3;
R³ is, at each occurrence, independently selected from the group consisting of hydrogen, halogen, C1-C6 alkyl, C1-C3 haloalkyl, —OR⁶, —CN and C1-C6 alkyl substituted with —OH, —OR⁶ or —NHR⁶;
R⁴ is either absent or independently, at each occurrence, selected from the group consisting of hydrogen, —OR⁶, halogen, C1-C3 haloalkyl, —CN, —N(R⁶)R⁶, (═O), —NH(C═O)R⁶, —(C═O)NH₂, —S(═O)₂N(R⁶)R⁶, aryl, heteroaryl, heterocyclyl, C1-C6 alkyl and C1-C6 alkyl substituted with —OR⁶, —NH₂ or —S(═O)₂N(R⁶)R⁶;
R⁵ is, independently, at each occurrence, selected from the group consisting of hydrogen, halogen, C1-C3 haloalkyl, —CN, —OR⁶, —N(R⁶)R⁶, (═O), S(═O)₂N(R⁶)R⁶, aryl, heteroaryl, heterocyclyl, C1-C6 alkyl and C1-C6 alkyl substituted with —OH, —NH₂ or —S(═O)₂N(R⁶)R⁶;
wherein both R⁴ and R⁵ are (═O) if attached to a single sulfur atom that forms part of Y being a heterocycle;
or wherein R⁴ and R⁵, together with the structure to which they are attached, form an aromatic ring, a heteroaromatic ring, a saturated or unsaturated heterocyclic ring, or a fused or bridged ring structure of any of an aromatic ring, a heteroaromatic ring, and a saturated or unsaturated heterocyclic ring;
R⁶ is, at each occurrence, independently selected from the group consisting of hydrogen, C1-C6 alkyl, C3-C6 cycloalkyl, C1-C3 haloalkyl, heteroaryl, heterocyclyl, heteroaryl substituted with one or two of halogen, —OR⁷, —N(R⁷)R⁷, C1-C6 alkyl and C1-C6 alkyl substituted with —OH, —NH₂; heterocyclyl substituted with one or two of halogen, —OR⁷, —N(R⁷)R⁷, C1-C6 alkyl and C1-C6 alkyl substituted with —OH or —NH₂;
R⁷ is, at each occurrence, independently selected from the group consisting of hydrogen, C1-C6 alkyl, C3-C10 cycloalkyl and W, as defined above;
R⁸ is, at each occurrence, independently selected from hydrogen and W, as defined above;
wherein if R⁷ is W, R⁸ is hydrogen;
R⁹ is, at each occurrence, independently selected from hydrogen and W, as defined above;
R¹⁰ is, at each occurrence, independently selected from the group consisting of hydrogen, halogen, C1-C6 alkyl, C1-C3 haloalkyl, —NH₂, —OR⁶, —CN and W, as defined above;
wherein if R¹⁰ is W, R⁹ is hydrogen;
R¹¹ is, at each occurrence, independently selected from the group consisting of hydrogen, C1-C6 alkyl and C1-C3 haloalkyl;
R¹² is, at each occurrence, independently selected from the group consisting of hydrogen, halogen, C1-C6 alkyl, C1-C3 haloalkyl, —NH₂, —OR⁶ and —CN;
R¹³ is, at each occurrence, independently selected from the group consisting of hydrogen, C1-C6 alkyl, C3-C10 cycloalkyl and W, as defined above;
wherein if R¹³ is W, R⁹ is hydrogen;
R¹⁴ and R¹⁵ are, at each occurrence, independently selected from the group consisting of hydrogen, halogen, C1-C6 alkyl, C1-C3 haloalkyl, —OR⁶, heterocyclyl and —CN; and
R¹⁶ is, at each occurrence, independently selected from the group consisting of hydrogen, halogen, C1-C6 alkyl, C3-C10 cycloalkyl, —N(R⁶)₂, —NR¹³R¹⁴, —NR¹³CH₂(CO)NH₂, heterocyclyl, —OR⁶ and —CN,
or an enantiomer, stereoisomeric form, mixture of enantiomers, diastereomer, mixture of diastereomer, racemate of the above mentioned compounds or a pharmaceutically acceptable salt thereof.

2. The combination according to claim 1, wherein said anti-cancer agent is selected from
a) target-specific compounds selected from the group consisting of immune checkpoint inhibitors; poly-ADP-ribose-polymerase (PARP) inhibitors; monoclonal antibodies and antibody fragments not directed at immune checkpoints; tyrosine kinase inhibitors; immunotoxins; MEK inhibitors; KRAS inhibitors; c-MET inhibitors; FGFR inhibitors; proteasome inhibitors; cyclin-dependent kinase inhibitors; mT OR inhibitors; retinoids; immunomodulatory agents; histone deacetylase inhibitors; proteolysis targeting chimera compounds (PROTACs); siRNA; antibody-drug-conjugates (ADCs); antibody-siRNA-conjugates (ARCs); DNA damage response inhibitors, and target-specific fusion proteins; and
b) cytotoxic non-specific compounds selected from taxanes, alkylating agents, nucleoside analogues, antifolates, topoisomerase inhibitors, anthracyclines, podophyllotoxins, vinca alkaloids, and platinum compounds;
c) hormonal anti-cancer agents selected from hormones; hormone antagonists; hormone receptor antagonists; hormone receptor degraders and aromatase inhibitors; and
d) radiopharmaceuticals.

3. The combination according to claim 1, wherein said combination is a composition in which said inhibitor of cyclin-dependent kinase 7 and said anti-cancer agent are present together, being either physically mixed with each other or being kept separate from each other by at least one physical separation barrier between said inhibitor of cyclin-dependent kinase 7 and said anti-cancer agent wherein said at least one physical separation barrier forms part of said combination.

4. The combination according to claim 1, wherein said anti-cancer agent is a target-specific compound selected from monoclonal antibodies directed at immune checkpoints; poly-ADP-ribose-polymerase (PARP) inhibitors; other monoclonal antibodies not directed at immune checkpoints; tyrosine kinase inhibitors; DNA damage response inhibitors; and antibody-cytokine fusion proteins.

5. The combination of claim 4, wherein said target-specific compound is selected from anti-PD1 antibodies, anti-PD-L1 antibodies, anti-CTLA-4 antibodies, anti-LAG-3 antibodies, anti-VEGF antibodies, anti-VEGFR antibodies, anti-EGFR antibodies, anti-HER2 antibodies, anti-CD52 antibodies, anti-CD33 antibodies, anti-CD30 antibodies, anti-CD20 antibodies, anti-TIM3 antibodies, anti-TIGIT antibodies, anti-41BB antibodies, anti-OX40 antibodies, anti-CD40 antibodies, anti-CD27 antibodies, anti-GITR antibodies, anti-ICOS antibodies, anti-Siglec antibodies, and anti-PVRIG antibodies.

6. The combination of claim 5, wherein said target-specific compound is selected from anti-human-PD1 antibodies, in particular pembrolizumab, nivolumab, cemiplimab, spartalizumab, atezolizumab, avelumab, durvalumab, ipilimumab, tremelimumab, relatlimab, bevacizumab, ramucirumab, cetuximab, panitumumab, pertuzumab, trastuzumab, trastuzumab-emtansine, alemtuzumab, gemtuzumab, gemtuzumab-ozoamicin, brentuximab, brentuximab-vedotin, ibritumomab, ibritumomab-tiuxetan, rituximab, obinutuzmab, tositumomab, ofatumumab, pidilizumab, toripalimab, sintilimab, camrelizumab, tislelizumab, zimberelimab, prolgolimab, dostarlimab; wherein, preferably, said target-specific compound is pembrolizumab.

7. The combination of claim 4, wherein said target-specific compound is selected from poly-ADP-ribose-polymerase (PARP) inhibitors; tyrosine kinase inhibitors; MEK inhibitors KRAS inhibitors; c-MET inhibitors; FGFR inhibitors; and DNA damage response inhibitors selected from WEE1 inhibitors and ATR inhibitors.

8. The combination of claim 1, wherein said anti-cancer agent is a cytotoxic non-specific compound selected from
a) taxanes;
b) alkylating agents;
c) nucleoside analogues;
d) antifolates;
e) topoisomerase inhibitors;
f) anthracyclines;
g) podophyllotoxins;
h) vinca alkaloids; and
i) platinum compounds.

9. The combination of claim 1, wherein the compound is a compound having the general formula Ia 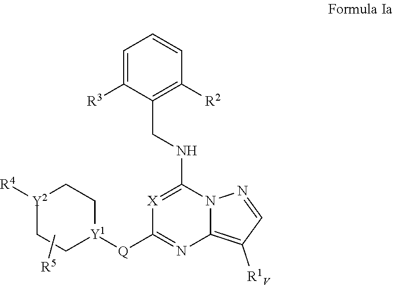
wherein
X is, independently at each occurrence, selected from CH and N;
Y¹ is, independently at each occurrence, selected from CH, C(OH) and N;
Y² is, independently at each occurrence, selected from CH, C(OH) and N;
Q is absent or, at each occurrence, independently selected from the group consisting of —NH—, —NH(CH₂)—, —NH(C═O)—, —NHSO₂—, —O—, —O(CH₂)—, —(C═O)— and —(C═O)(CH₂)—;
R¹ is, at each occurrence, independently selected from the group consisting of halogen, C1-C6 alkyl, C3-C10 cycloalkyl, —CN, —(C═O)CH₃ and C1-C3 haloalkyl, any of which is optionally substituted;
R² is, at each occurrence, independently selected from any structure of the following group A, 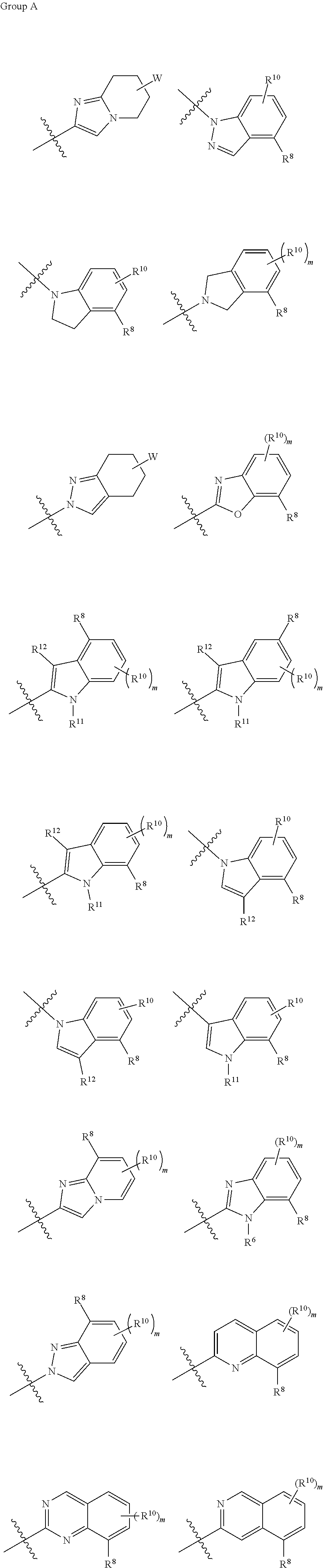 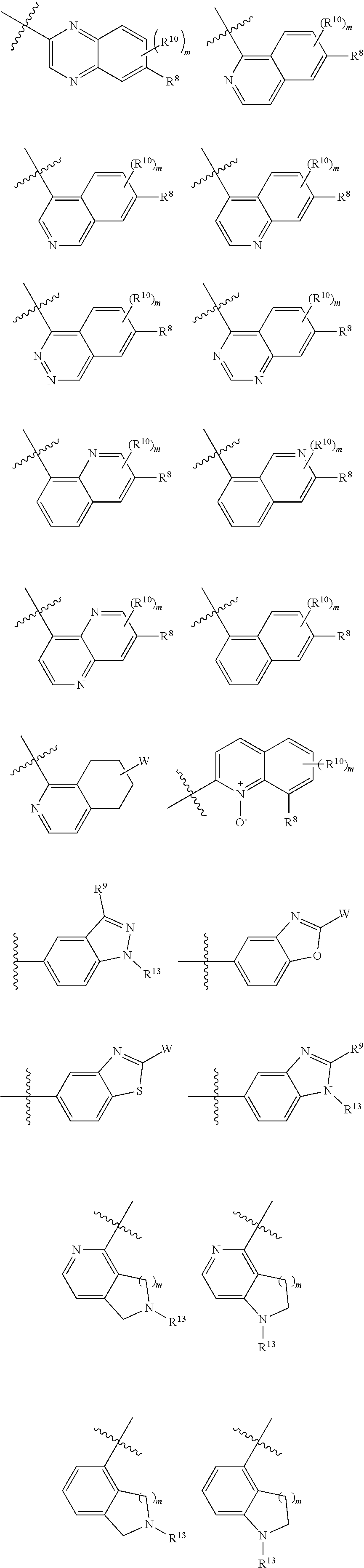
wherein m=1, 2 or 3;
W is any structure of the following group B′; 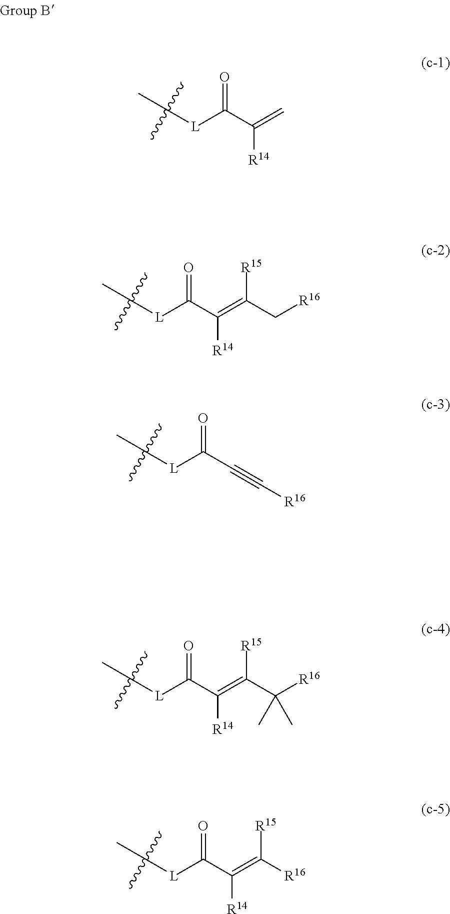
L is absent or, at each occurrence, independently selected from the group consisting of —O— and —NH—;
R³, is, at each occurrence, independently selected from the group consisting of hydrogen, halogen, C1-C6 alkyl, C1-C3 haloalkyl, —OR⁶, —CN and C1-C6 alkyl substituted with —OH, —OR⁶ or —NHR⁶;
R⁴ is either absent or independently, at each occurrence, selected from the group consisting of hydrogen, —OR⁶, halogen, C1-C3 haloalkyl, —CN, —N(R⁶)R⁶, (═O), —NH(C═O)R⁶, —(C═O)NH₂, —S(═O)₂N(R⁶)R⁶, aryl, heteroaryl, heterocyclyl, C1-C6 alkyl and C1-C6 alkyl substituted with —OR⁶, —NH₂ or —S(═O)₂N(R⁶)R⁶;
R⁵ is, independently, at each occurrence, selected from the group consisting of hydrogen, halogen, C1-C3 haloalkyl, —CN, —OR⁶, —N(R⁶)R⁶, (═O), S(═O)₂N(R⁶)R⁶, aryl, heteroaryl, heterocyclyl, C1-C6 alkyl and C1-C6 alkyl substituted with —OH, —NH₂ or —S(═O)₂N(R⁶)R⁶;
wherein both R⁴ and R⁵ are (═O) if attached to a single sulfur atom that forms part of Y being a heterocycle;
or wherein R⁴ and R⁵, together with the structure to which they are attached, form an aromatic ring, a heteroaromatic ring, a saturated or unsaturated heterocyclic ring, or a fused or bridged ring structure of any of an aromatic ring, a heteroaromatic ring, and a saturated or unsaturated heterocyclic ring;
R⁶ is, at each occurrence, independently selected from the group consisting of hydrogen, C1-C6 alkyl, C3-C6 cycloalkyl, C1-C3 haloalkyl, heteroaryl, heterocyclyl, heteroaryl substituted with one or two of halogen, —OR⁷, —N(R⁷)R⁷, C1-C6 alkyl and C1-C6 alkyl substituted with —OH, —NH₂; heterocyclyl substituted with one or two of halogen, —OR⁷, —N(R⁷)R⁷, C1-C6 alkyl and C1-C6 alkyl substituted with —OH or —NH₂;
R⁷ is, at each occurrence, independently selected from the group consisting of hydrogen, C1-C6 alkyl, C3-C10 cycloalkyl and W, as defined above;
R⁸ is, at each occurrence, independently selected from hydrogen and W, as defined above;
wherein if R⁷ is W, R⁸ is hydrogen;
R⁹ is, at each occurrence, independently selected from hydrogen and W, as defined above;
R¹⁰ is, at each occurrence, independently selected from the group consisting of hydrogen, halogen, C1-C6 alkyl, C1-C3 haloalkyl, —NH₂, —OR⁶, —CN and W, as defined above;
wherein if R¹⁰ is W, R⁸ is hydrogen;
R¹¹ is, at each occurrence, independently selected from the group consisting of hydrogen, C1-C6 alkyl and C1-C3 haloalkyl;
R¹² is, at each occurrence, independently selected from the group consisting of hydrogen, halogen, C1-C6 alkyl, C1-C3 haloalkyl, —NH₂, —OR⁶ and —CN;
R¹³ is, at each occurrence, independently selected from the group consisting of hydrogen, C1-C6 alkyl, C3-C10 cycloalkyl and W, as defined above;
wherein if R¹³ is W, R⁹ is hydrogen;
R¹⁴ and R¹⁵ are, at each occurrence, independently selected from the group consisting of hydrogen, halogen, C1-C6 alkyl, C1-C3 haloalkyl, —OR⁶, heterocyclyl and —CN; and
R¹⁶ is, at each occurrence, independently selected from the group consisting of hydrogen, halogen, C1-C6 alkyl, C3-C10 cycloalkyl, —N(R⁶)₂, —NR¹³R¹⁴, heterocyclyl, —OR⁶ and —CN;
or an enantiomer, stereoisomeric form, mixture of enantiomers, diastereomer, mixture of diastereomer, racemate of the above mentioned compounds or a pharmaceutically acceptable salt thereof.

10. The combination of claim 1, wherein at least one of R², R⁷, R⁸, R⁹, R¹⁰ and R¹³ is W, as defined in claim 1, or is a structure containing W, as defined in claim 1.

11. The combination of claim 1, wherein R¹ is C1-C6 alkyl or C1-C3 haloalkyl.

12. The combination of claim 1, wherein R² is

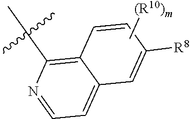

13. The combination of claim 12, wherein R¹⁰ is hydrogen; m is 1; R⁸ is W; W is (c-1) or (c-2) or (c-3); L is —NH—; R¹⁴ and R¹⁵ are, independently, at each occurrence, hydrogen, halogen, or C1-C6 alkyl; and wherein R¹⁶ is hydrogen, halogen, C1-C6 alkyl, —N(R⁶)₂, or —NR¹³R¹⁴.

14. The combination of claim 1, wherein said compound is a compound having a structure selected from structures 1-198, as defined in the column entitled “Structure” of table 1 of the description.

15. The combination of claim 14, wherein said compound is a compound having a structure selected from compounds 3, 14, 47, and 156.

16-21. (canceled)

22. A method of prevention and/or treatment of cancer in a patient, said method comprising administering a combination of an inhibitor of cyclin-dependent kinase 7 with an anti-cancer agent, said combination being as defined in claim 1, to a patient having, or suspected of having, cancer.

23. (canceled)

24. A pharmaceutical composition comprising a combination, as defined in claim 1, for preventing and/or treating cancer in a patient having, or suspected of having, cancer.

25. The method according to claim 22, wherein, said inhibitor of cyclin-dependent kinase 7 is administered before or after administration of said anti-cancer agent to said patient, or wherein both said inhibitor of cyclin-dependent kinase 7 and said anti-cancer agent are administered concomitantly or synchronously or in a temporally overlapping manner to said patient, or wherein said inhibitor of cyclin-dependent kinase 7 is administered adjunctively to said anti-cancer agent to said patient, or wherein said anti-cancer agent is administered adjunctively to said inhibitor of cyclin-dependent kinase 7, to said patient.

26. The method for use according to claim 22, wherein said method of prevention and/or treatment comprises administering said combination in conjunction with radiation therapy.

27. The method according to claim 22, wherein said cancer is a cancer selected from the group comprising or consisting of: renal cell carcinoma (RCC), kidney cancer, hereditary papillary renal cancer, sporadic papillary renal cancer, non-squamous non-small-cell lung carcinoma (non-squamous NSCLC), squamous non-small-cell lung carcinoma (squamous NSCLC), small-cell lung carcinoma (SCLC), triple-negative breast cancer, colorectal cancer, melanoma, pancreatic ductal adenocarcinoma, esophageal cancer, head and neck squamous cell carcinoma (HNSCC), urothelial cancer, adenocarcinoma, choroidal melanoma, acute leukemia, acoustic neurinoma, ampullary carcinoma, anal carcinoma, astrocytoma, basal cell carcinoma, pancreatic cancer, Desmoid tumor, bladder cancer, bronchial carcinoma, estrogen dependent and independent breast cancer, Burkitt's lymphoma, corpus cancer, Carcinoma unknown primary tumor (CUP-syndrome), small intestine cancer, small intestinal tumors, ovarian cancer, endometrial carcinoma, ependymoma, epithelial cancer types, Ewing's tumors, gastrointestinal tumors, gastric cancer, gallbladder cancer, gall bladder carcinomas, uterine cancer, cervical cancer, cervix, glioblastomas, gynecologic tumors, ear, nose and throat tumors, hematologic tumor, hairy cell leukemia, urethral cancer, skin cancer, skin testis cancer, brain tumors (gliomas), brain metastases, testicle cancer, hypophysis tumor, carcinoids, Kaposi's sarcoma, laryngeal cancer, germ cell tumor, bone cancer, head and neck tumors (tumors of the ear, nose and throat area), colon carcinoma, craniopharyngiomas, oral cancer (cancer in the mouth area and on lips), cancer of the central nervous system, liver cancer, liver metastases, leukemia, eyelid tumor, lung cancer, lymphomas, stomach cancer, malignant melanoma, malignant neoplasia, malignant tumors gastrointestinal tract, breast carcinoma, rectal cancer, medulloblastomas, meningiomas, Hodgkin's/Non-Hodgkin's lymphoma, mycosis fungoides, nasal cancer, neurinoma, neuroblastoma, oligodendroglioma, osteolytic carcinomas and osteoplastic carcinomas, osteosarcomas, ovarian carcinoma, pancreatic carcinoma, penile cancer, plasmacytoma, prostate cancer, pharyngeal cancer, rectal carcinoma, retinoblastoma, vaginal cancer, thyroid carcinoma, T-cell lymphoma, thymoma, tube carcinoma, eye tumors, urethral cancer, urologic tumors, urothelial carcinoma, vulva cancer, wart appearance, soft tissue tumors, soft tissue sarcoma, Nephroblastoma, cervical carcinoma, tongue cancer, invasive ductal carcinoma, invasive lobular carcinoma, ductal carcinoma in situ, lobular carcinoma in situ, small-cell lung carcinoma, non-small-cell lung carcinoma, bronchial adenoma, pleuropulmonary blastoma, mesothelioma, brain stem glioma, hypothalamic glioma, cerebellar astrocytoma, cerebral astrocytoma, neuroectodermal tumor, pineal tumors, sarcoma of the uterus, salivary gland cancers, anal gland adenocarcinomas, mast cell tumors, pelvis tumor, ureter tumor, intraocular melanoma, hepatocellular carcinoma, cholangiocarcinoma, mixed hepatocellular cholangiocarcinoma, squamous cell carcinoma, Merkel cell skin cancer, non-melanoma skin cancer, hypopharyngeal cancer, nasopharyngeal cancer, oropharyngeal cancer, oral cavity cancer, squamous cell cancer, oral melanoma, AIDS-related lymphoma, cutaneous T-cell lymphoma, lymphoma of the central nervous system, malignant fibrous histiocytoma, lymph sarcoma, rhabdomyosarcoma, malignant histiocytosis, fibroblastic sarcoma, hemangiosarcoma, hemangiopericytoma, leiomyosarcoma (LMS), canine mammary carcinoma, and feline mammary carcinoma.
